(19) United States
(12) Patent Application Publication
(10) Pub. No.: US 2018/0119123 A1
(43) Pub. Date: May 3, 2018

(54) CRISPR/CAS-RELATED METHODS AND COMPOSITIONS FOR TREATING HIV INFECTION AND AIDS

(71) Applicant: EDITAS MEDICINE, INC., Cambridge, MA (US)

(72) Inventors: Jennifer Leah Gori, Jamaica Plain, MA (US); G. Grant Welstead, Cambridge, MA (US); Penrose Odonnell, Yarmouth, ME (US)

(73) Assignee: EDITAS MEDICINE, INC., Cambridge, MA (US)

(21) Appl. No.: 15/809,549

(22) Filed: Nov. 10, 2017

Related U.S. Application Data
(63) Continuation of application No. PCT/US2016/031922, filed on May 11, 2016.
(60) Provisional application No. 62/159,778, filed on May 11, 2015.

(51) Int. Cl. C12N 9/22; A61K 38/46; C12N 15/113

(52) U.S. Cl. CPC C12N 9/22; A61K 48/00; C12N 15/1138; A61K 38/465

(57) ABSTRACT

CRISPR/CAS-related systems, compositions and methods for editing CCR5 and/or CXCR4 genes in human cells are described, as are cells and compositions including cells edited according to the same.

### CROSS-REFERENCES TO RELATED APPLICATIONS

This application is a Continuation of International Patent Application No. PCT/US16/031922, filed May 11, 2016, which claims priority to United States Provisional Application No. 62/159,778, filed May 11, 2015, the contents of each of which are hereby incorporated by reference in their entirety herein, and to each of which priority is claimed.

### SEQUENCE LISTING

The specification further incorporates by reference the Sequence Listing submitted herewith via EFS on Nov. 10, 2017. Pursuant to 37 C.F.R. § 1.52(e)(5), the Sequence Listing text file, identified as 084177.0122USSEQ.txt, is 1,723,075 bytes and was created on Nov. 10, 2017. The entire contents of the Sequence Listing are hereby incorporated by reference. The Sequence Listing does not extend beyond the scope of the specification and thus does not contain new matter.

### FIELD OF THE INVENTION

The disclosure relates to CRISPR/CAS-related methods, compositions and genome editing systems for editing of a target nucleic acid sequence, e.g., editing a CCR5 gene and/or a CXCR4 gene, and applications thereof in connection with Human Immunodeficiency Virus (HIV) infection and Acquired Immunodeficiency Syndrome (AIDS).

### BACKGROUND

Human Immunodeficiency Virus (HIV) is a virus that causes severe immunodeficiency. In the United States, more than 1 million people are infected with the virus. Worldwide, approximately 30-40 million people are infected.

HIV preferentially infects macrophages and CD4 T lymphocytes. It causes declining CD4 T cell counts, severe opportunistic infections and certain cancers, including Kaposi's sarcoma and Burkitt's lymphoma. Untreated HIV infection is a chronic, progressive disease that leads to acquired immunodeficiency syndrome (AIDS) and death in nearly all subjects.

HIV was untreatable and invariably led to death in all subjects until the late 1980's. Since then, antiretroviral therapy (ART) has dramatically slowed the course of HIV infection. Highly active antiretroviral therapy (HAART) is the use of three or more agents in combination to slow HIV. Treatment with HAART has significantly altered the life expectancy of those infected with HIV. A subject in the developed world who maintains their HAART regimen can expect to live into his or her 60's and possibly 70's. However, HAART regimens are associated with significant, long-term side effects. The dosing regimens are complex and associated with strict dietary requirements. Compliance rates with dosing can be lower than 50% in some populations in the United States. In addition, there are significant toxicities associated with HAART treatment, including diabetes, nausea, malaise and sleep disturbances. A subject who does not adhere to dosing requirements of HAART therapy may have a return of viral load in their blood and is at risk for progression of the disease and its associated complications.

HIV is a single-stranded RNA virus that preferentially infects CD4 T lymphocytes. The virus must bind to receptors and coreceptors on the surface of CD4 cells to enter and infect these cells. This binding and infection step is vital to the pathogenesis of HIV. The virus attaches to the CD4 receptor on the cell surface via its own surface glycoproteins, gp120 and gp41. Gp120 binds to a CD4 receptor and must also bind to another coreceptor in order for the virus to enter the host cell. In macrophage-(M-tropic) viruses, the coreceptor is CCR5, also referred to as the CCR5 receptor. CCR5 receptors are expressed by CD4 cells, T cells, gut-associated lymphoid tissue (GALT), macrophages, dendritic cells and microglia. HIV establishes initial infection most commonly via CCR5 co-receptors (M-tropic HIV). In thymic-(T-tropic) viruses, the virus uses CXCR4 as the primary co-receptor to infect T cells. CXCR4 is a chemokine receptor present on CD4 T cells, CD8 T cells, B cells, neutrophils and eosinophils, and hematopoietic stem cells (HSCs) that allows blood cells to migrate toward and bind to the chemokine SDF-1. In the later stages of infection, 50-60% of subjects have T-tropic viruses that infect T cells through CXCR4 receptors. Subjects may be infected with M-tropic viruses, T-tropic viruses, and/or dual tropic viruses (i.e., viruses that can utilize either CCR5 or CXCR4 co-receptor to gain entry into cells).

Most initial HIV infections and early stage HIV is due to entry and propogation of M-tropic virus. CCR5-Δ32 mutation (also refered to as CCR5 delta 32 mutation) results in the expression of a truncated CCR5 receptor that lacks an extracellular domain of the receptor, thus preventing M-tropic HIV-1 viral variants from entering the cell. Individuals carrying two copies of the CCR5-Δ32 allele are resistant to HIV infection and CCR5-Δ32 heterozyous carriers have slow progression of the disease.

CCR5 antagonists (e.g., maraviroc) exist and are used in the treatment of HIV. However, current CCR5 antagonists decrease HIV progression but cannot cure the disease. In addition, there are considerable risks of side effects of these CCR5 antagonists, including severe liver toxicity.

As HIV progresses to later stage, the virus often becomes predominantly T-tropic. In later stage HIV infections, many subjects have T-tropic viruses, which infect T cells via CXCR4 coreceptors. CXCR4 receptor tropism is associated with lower CD4 counts, and, often, later stage, more advanced disease progression. There is no known protective mutation in the CXCR4 gene that is equivalent to the CCR5-Δ32 mutation.

In spite of considerable advances in the treatment of HIV, there remain considerable needs for agents that could prevent, treat, and eliminate HIV infection or AIDS. Therapies that are free from significant toxicities and involve a single or multi-dose regimen (versus current daily dose regimen for the lifetime of a patient) would be superior to current HIV treatment. A reduction or elimination of CCR5, CXCR4, or both CCR5 and CXCR4 gene expression in myeloid and lymphoid cells can prevent HIV infection and progression, and can cure the disease.

### SUMMARY OF THE DISCLOSURE

The methods, genome editing systems, and compositions discussed herein, allow for the prevention and treatment of HIV infection and AIDS, by gene editing, e.g., using CRISPR-Cas9 mediated methods to alter a CCR5 gene. The CCR5 gene is also known as CKR5, CCR-5, CD195, CKR-5, CCCKR5, CMKBR5, IDDM22, or CC-CKR-5. In cetain embodiments, altering the C-C chemokine receptor type 5 (CCR5) gene comprises reducing or eliminating (1) CCR5 gene expression, (2) CCR5 protein function, and/or (3) the level of CCR5 protein. Altering the CCR5 gene can be achieved by one or more approaches described in Section 4. In certain embodiments, altering the CCR5 gene can be achieved by (1) introducing one or more mutations in the CCR5 gene, e.g., by introducing one or more protective mutations (such as a CCR5 delta 32 mutation), (2) knocking out the CCR5 gene and/or (3) knocking down the CCR5 gene.

The methods, genome editing systems, and compositions discussed herein, allow for the prevention and treatment of HIV infection and AIDS, by gene editing, e.g., using CRISPR-Cas9 mediated methods to alter a CXCR4 gene. The CXCR4 gene is also known as CD184, D2S201E, FB22, HM89, HSY3RR, LAP-3, LAP3, LCR1, LESTR, NPY3R, NPYR, NPYRL, NPYY3R, WHIM or WHIMS. In cetain embodiments, altering the CXCR4 gene comprises reducing or eliminating (1) CXCR4 gene expression, (2) CXCR4 protein function, (3) altering the amino acid sequence to prevent HIV interaction with the protein, and/or (4) the level of CXCR4 protein. Altering the CXCR4 gene can be achieved by one or more approaches described in Section 5. In certain embodiments, altering the CXCR4 gene can be achieved by (1) knocking out the CXCR4 gene, (2) knocking down the CXCR4 gene, and/or (3) introducing one or more mutations in the CXCR4 gene (e.g., introducing one or more single base or two base substitutions).

The methods, genome editing systems, and compositions discussed herein, allow for the prevention and treatment of HIV infection and AIDS, by gene editing, e.g., using CRISPR-Cas9 mediated methods to alter each of two genes: the gene for C-C chemokine receptor type 5 (CCR5) and the gene for chemokine (C-X-C motif) receptor 4 (CXCR4). Alteration of two or more genes (e.g., CCR5 and CRCX4) (e.g., in the same cell or cells or in different cells) is referred to herein as “multiplexing”. In certain embodiments, multiplexing comprises modification of at least two genes (e.g., CCR5 and CRCX4) in the same cell or cells.

The methods, genome editing systems, and compositions discussed herein, provide for prevention or reduction of HIV infection and/or prevention or reduction of the ability for HIV to enter host cells, e.g., in subjects who are already infected. Exemplary host cells for HIV include, but are not limited to, CD4 cells, CD8 cells, T cells, B cells, gut associated lymphatic tissue (GALT), macrophages, dendritic cells, myeloid progenitor cells, lymphoid progenitor cells, neutrophils, eosinophils, and microglia. Viral entry into the host cells requires interaction of the viral glycoproteins gp41 and gp120 with both the CD4 receptor and a co-receptor, e.g., CCR5, e.g., CXCR4. If a co-receptor, e.g., CCR5, e.g., CXCR4, is not present on the surface of the host cells, the virus cannot bind and enter the host cells. The progress of the disease is thus impeded. In certain embodiments, by altering the CCR5 gene, e.g., introducing one or more mutations in the CCR5 gene, e.g., by introducing one or more protective mutations (such as a CCR5 delta 32 mutation), knocking out the CCR5 gene, and/or knocking down the CCR5 gene, entry of the HIV virus into the host cells is reduced or prevented. In certain embodiments, by altering the CXCR4 gene, e.g., knocking out the CXCR4 gene, knocking down the CXCR4 gene, and/or introducing one or more mutations in the CXCR4 gene, entry of the HIV virus into the host cells is reduced or prevented. In certain embodiments, by multiplexing the alteration of both CCR5 and CXCR4, entry of the HIV virus into the host cells is reduced or prevented. Examplary multiplexing alterations of CCR5 and CXCR4 genes are described in Section 6. Examplary multiplexing alterations of CCR5 and CXCR4 genes include, but are not limited to: (1) introducing one or more mutations in the CCR5 gene, e.g., by introducing one or more protective mutations (such as a CCR5 delta 32 mutation), and knocking out the CXCR4 gene; (2) introducing one or more mutations in the CCR5 gene, e.g., by introducing one or more protective mutations (such as a CCR5 delta 32 mutation), and knocking down the CXCR4 gene; (3) knocking out both CCR5 and CXCR4 genes; (4) knocking down both CCR5 and CXCR4 genes; (5) knocking out the CCR5 gene and knocking down the CXCR4 gene; (6) knocking down the CCR5 gene and knocking out the CXCR4 gene; (7) introducing one or more mutations in the CCR5 gene, e.g., by introducing one or more protective mutations (such as a CCR5 delta 32 mutation), and introducing one or more mutations in the CXCR4 gene (e.g., introducing one or more single or two base substitutions); (8) knocking out the CCR5 gene and introducing one or more mutations in the CXCR4 gene (e.g., introducing one or more single or two base substitutions); and/or (9) knocking down the CCR5 gene and introducing one or more mutations in the CXCR4 gene (e.g., introducing one or more single or two base substitutions).

In certain embodiments, altering, e.g., introducing one or more mutations in the CCR5 gene, e.g., by introducing one or more protective mutations (such as a CCR5 delta 32 mutation), knocking out or knocking down the CCR5 gene in a subject's CD4 cells, T cells, gut associated lymphatic tissue (GALT), macrophages, dendritic cells, myeloid progenitor cells, lymphoid progenitor cells, microglia, or HSCs (i.e., the parent cells that give rise to the above indicated myeloid, lymphoid and microglial cells) can reduce or prevent M-tropic HIV virus particles from infection and propogation within host cells. In certain embodiments, altering, e.g., introducing one or more mutations in the CXCR4 gene (e.g., introducing one or more single or two base substitutions), knocking out or knocking down the CXCR4 gene in a subject's CD4 cells, CD8 T cells, B cells, neutrophils and eosinophils, or HSCs (i.e., the parent cells that give rise to the above indicated myeloid, lymphoid cells and microglia) can reduce or prevent T-tropic HIV virus particles from infection and propogation within host cells. In the later stages of HIV infection, subjects are often infected with both M-tropic and T-tropic viruses. In certain embodiments, the knockout or knockdown of CXCR4 in a subject's lymphoid and myeloid cells can reduce or prevent the drop in T-cells associated with later stage, often more severe HIV. In certain embodiments, altering both CCR5 and CXCR4 genes in a subject's CD4 cells and lymphoid and myeloid progenitor cells, and/or HSCs can reduce or prevent HIV infection and propagation within the host. In certain embodiments, knock-out or knock down of one or both of these receptors in the host can effectively render the host immune to HIV.

In certain embodiments, altering both CCR5 and CXCR4 genes in myeloid and lymphoid cells, and HSCs reduces or prevents HIV infection and/or treats HIV disease. In certain embodiments, both T-tropic and M-tropic viral entry into myeloid and lymphoid cells are prevented or reduced by altering both CCR5 and CXCR4 genes. In certain embodiments, a subject who has HIV and is treated with alteration of CCR5 and CXCR4 genes would be expected to clear HIV and effectively be cured. In certain embodiments, a subject who does not yet have HIV and is treated with altering both CCR5 and CXCR4 genes would be expected to be immune to HIV.

The methods, genome editing systems, and compositions discussed herein, provide for treating or delaying the onset or progression of HIV infection or AIDS by gene editing, e.g., using CRISPR-Cas9 mediated methods to alter a CCR5 gene. In certain embodiments, altering the CCR5 gene comprises reducing or eliminating (1) CCR5 gene expression, (2) CCR5 protein function, and/or (3) the level of CCR5 protein.

The methods, genome editing systems, and compositions discussed herein, provide for treating or delaying the onset or progression of HIV infection or AIDS by gene editing, e.g., using CRISPR-Cas9 mediated methods to alter a CXCR4 gene. In certain embodiments, altering the CXCR4 gene comprises reducing or eliminating (1) CXCR4 gene expression, (2) CXCR4 protein function, and/or (3) the level of CXCR4 protein.

The methods, genome editing systems, and compositions discussed herein, provide for treating or delaying the onset or progression of HIV infection or AIDS by gene editing, e.g., using CRISPR-Cas9 mediated methods to alter two genes in a single cell or cells, e.g., a CCR5 gene and a CXCR4 gene. In certain embodiments, altering the CCR5 gene and the CXCR4 gene comprises reducing or eliminating (1) CCR5 and CXCR4 gene expression, (2) CCR5 and CXCR4 protein function, and/or (3) levels of CCR5 and CXCR4 protein.

The presently disclosed subject matter provides for genome editing systems comprising a first gRNA molecule comprising a first targeting domain that is complementary with a target sequence of a CCR5 gene and a second gRNA molecule comprising a second targeting domain that is complementary with a target sequence of a CXCR4 gene.

In certain embodiments, the first targeting domain comprises a nucleotide sequence selected from SEQ ID NOS: 476 to 1569 and 1947 to 3663, and the second targeting domain comprises a nucleotide sequence selected from SEQ ID NOS: 4064 to 5208, and 5921 to 8355.

In certain embodiments, the first targeting domain comprises a nucleotide sequence selected from SEQ ID NOS: 208 to 475, and 1614 to 1946, and the second targeting domain comprises a nucleotide sequence selected from SEQ ID NOS: 3740 to 4063, and 5241 to 5920.

In certain embodiments, the first targeting domain comprises a nucleotide sequence selected from SEQ ID NOS: 476 to 1569 and 1947 to 3663, and the second targeting domain comprises a nucleotide sequence selected from SEQ ID NOS: 3740 to 4063, and 5241 to 5920.

In certain embodiments, the first targeting domain comprises a nucleotide sequence selected from SEQ ID NOS: 208 to 475, and 1614 to 1946, and the second targeting domain comprises a nucleotide sequence selected from SEQ ID NOS: 4064 to 5208, and 5921 to 8355.

In certain embodiments, the first targeting domain comprises a nucleotide sequence selected from SEQ ID NOS: 335, 480, 482, 486, 488, 490, 492, 512, 521, 535, 1000, and 1002, and the second targeting domain comprises a nucleotide sequence selected from SEQ ID NO: 3973, 4118, and 4604. In certain embodiments, the first targeting domain and the second targeting domain are selected from the group consisting of:

(a) a first targeting domain comprising the nucleotide sequence set forth in SEQ ID NO: 335, and a second targeting domain comprising the nucleotide sequence set forth in SEQ ID NO: 3973;

(b) a first targeting domain comprising the nucleotide sequence set forth in SEQ ID NO: 335, and a second targeting domain comprising the nucleotide sequence set forth in SEQ ID NO: 4604;

(c) a first targeting domain comprising the nucleotide sequence set forth in SEQ ID NO: 488, and a second targeting domain comprising the nucleotide sequence set forth in SEQ ID NO: 4604; and

(d) a first targeting domain comprising the nucleotide sequence set forth in SEQ ID NO: 480, and a second targeting domain comprising the nucleotide sequence set forth in SEQ ID NO: 4118.

In certain embodiments, one or both of the first and second gRNA molecules are modified at its 5′ end. In certain embodiments, the modification comprises an inclusion of a 5′ cap. In certain embodiments, the 5′ cap comprises a 3′-O-Me-m⁷ G(5′)ppp(5′)G anti reverse cap analog (ARCA). In certain embodiments, one or both of the first and second gRNA molecules comprise a 3′ polyA tail that is comprised of about 10 to about 30 adenine nucleotides. In certain embodiments, the 3′ polyA tail is comprised of 20 adenine nucleotides.

In certain embodiments, the genome editing system further comprises a first Cas9 molecule and a second Cas9 molecule that are configured to form complexes with the first and second gRNAs. In certain embodiments, at least one of the first and second Cas9 molecules comprises an S. pyogenes Cas9 molecule or an S. aureus Cas9 molecule. In certain embodiments, wherein at least one of the first and second Cas9 molecules comprises a wild-type Cas9 molecule, a mutant Cas9 molecule, or a combination thereof. In certain embodiments, the mutant Cas9 molecule comprises a D10A mutation. In certain embodiments, the genome editing system further comprises an oligonucleotide donor encoding a de132 mutation in the CCR5 gene.

The presently disclosed subject matter further provides for genome editing systems comprising a gRNA molecule comprising a targeting domain that is complementary with a target sequence of a CCR5 gene.

In certain embodiments, the targeting domain comprises a nucleotide sequence selected from SEQ ID NOS: 476 to 1569 and 1947 to 3663. In certain embodiments, the targeting domain comprises a nucleotide sequence selected from SEQ ID NOS: 208 to 475, and 1614 to 1946.

In certain embodiments, the targeting domain comprises a nucleotide sequence selected from SEQ ID NOS: 335, 480, 482, 486, 488, 490, 492, 512, 521,535, 1000, and 1002. In certain embodiments, the genome editing system further comprises an oligonucleotide donor encoding a de132 mutation in the CCR5 gene.

The presently disclosed subject matter further provides for genome editing systems comprising a gRNA molecule comprising a targeting domain that is complementary with a target sequence of a CXCR4 gene.

In certain embodiments, the targeting domain comprises a nucleotide sequence selected from SEQ ID NOS: 4064 to 5208, and 5921 to 8355. In certain embodiments, the targeting domain comprises a nucleotide sequence selected from 3740 to 4063, and 5241 to 5920. In certain embodiments, the targeting domain comprises a nucleotide sequence selected from SEQ ID NOS: 3973, 4118, and 4604.

In certain embodiments, any of the above-described gRNA molecules can be modified at its 5′ end. In certain embodiments, the modification comprises an inclusion of a 5′ cap. In certain embodiments, wherein the 5′ cap comprises a 3′-O-Me-m⁷ G(5′)ppp(5′)G anti reverse cap analog (ARCA). In certain embodiments, the gRNA molecule comprises a 3′ polyA tail that is comprised of about 10 to about 30 adenine nucleotides. In certain embodiments, the 3′ polyA tail is comprised of 20 adenine nucleotides.

The genome editing systems can comprise two, three or four gRNA molecules. In certain embodiments, the genome editing system further comprises at least one Cas9 molecule. In certain embodiments, the at least one Cas9 molecule is an S. pyogenes Cas9 molecule or an S. aureus Cas9 molecule. In certain embodiments, the at least one Cas9 molecule comprises an S. pyogenes Cas9 molecule and an S. aureus Cas9 molecule. In certain embodiments, the at least one Cas9 molecule comprises a wild-type Cas9 molecule, a mutant Cas9 molecule, or a combination thereof. In certain embodiments, the mutant Cas9 molecule comprises a D10A mutation.

The above-described genome editing systems can be used in a medicament, or for therapy. The above-described genome editing systems can be used in altering a CCR5 gene, altering a CXCR4 gene, or altering a CCR5 and a CXCR4 gene in a cell. In certain embodiments, the cell is from a subject suffering from HIV infection or AIDS. The above-described genome editing systems can be used in treating HIV infection or AIDS.

The presently disclosed subject matter provides for compositions comprising a first gRNA molecule comprising a first targeting domain that is complementary with a target sequence of a CCR5 gene, and a second gRNA molecule comprising a second targeting domain that is complementary with a target sequence of a CXCR4 gene.

In certain embodiments, the first targeting domain comprises a nucleotide sequence selected from SEQ ID NOS: 476 to 1569 and 1947 to 3663, and the second targeting domain comprises a nucleotide sequence selected from SEQ ID NOS: 4064 to 5208, and 5921 to 8355. In certain embodiments, the first targeting domain comprises a nucleotide sequence selected from SEQ ID NOS: 208 to 475, and 1614 to 1946, and the second targeting domain comprises a nucleotide sequence selected from SEQ ID NOS: 3740 to 4063, and 5241 to 5920. In certain embodiments, the first targeting domain comprises a nucleotide sequence selected from SEQ ID NOS: 476 to 1569 and 1947 to 3663, and the second targeting domain comprises a nucleotide sequence selected from SEQ ID NOS: 3740 to 4063, and 5241 to 5920. In certain embodiments, the first targeting domain comprises a nucleotide sequence selected from SEQ ID NOS: 208 to 475, and 1614 to 1946, and the second targeting domain comprises a nucleotide sequence selected from SEQ ID NOS: 4064 to 5208, and 5921 to 8355.

In certain embodiments, the composition further comprises a first Cas9 molecule and a second Cas9 molecule that are configured to form complexes with the first and second gRNAs. In certain embodiments, the at least one of the first and second Cas9 molecules comprises an S. pyogenes Cas9 molecule or an S. aureus Cas9 molecule. In certain embodiments, at least one of the first and second Cas9 molecules comprises a wild-type Cas9 molecule, a mutant Cas9 molecule, or a combination thereof. In certain embodiments, the mutant Cas9 molecule comprises a D10A mutation.

In certain embodiments, the composition is a ribonucleoprotein (RNP) composition, wherein at least one of the first and second Cas9 molecules is complexed with at least one of the first and second gRNA molecules.

The presently disclosed subject matter provides for compositions comprising a gRNA molecule comprising a targeting domain that is complementary with a target sequence of a CCR5 gene. In certain embodiments, the targeting domain comprises a nucleotide sequence selected from SEQ ID NOS: 476 to 1569 and 1947 to 3663. In certain embodiments, the targeting domain comprises a nucleotide sequence selected from SEQ ID NOS: 208 to 475, and 1614 to 1946. In certain embodiments, the targeting domain comprises a nucleotide sequence selected from SEQ ID NOS: 335, 480, 482, 486, 488, 490, 492, 512, 521,535, 1000, and 1002. In certain embodiments, the composition further comprises an oligonucleotide donor encoding a de132 mutation in the CCR5 gene.

The presently disclosed subject matter provides for compositions comprising a gRNA molecule comprising a targeting domain that is complementary with a target sequence of a CXCR4 gene. In certain embodiments, the targeting domain comprises a nucleotide sequence selected from SEQ ID NOS: 4064 to 5208, and 5921 to 8355. In certain embodiments, the targeting domain comprises a nucleotide sequence selected from SEQ ID NOS: 3740 to 4063, and 5241 to 5920. In certain embodiments, the targeting domain comprises a nucleotide sequence selected from SEQ ID NOS: 3973, 4118, and 4604.

The composition can comprise one, two, three, or four gRNA molecules. In certain embodiments, the composition further comprises at least one Cas9 molecule. In certain embodiments, the at least one Cas9 molecule is an S. pyogenes Cas9 molecule or an S. aureus Cas9 molecule. In certain embodiments, the at least one Cas9 molecule comprises an S. pyogenes Cas9 molecule and an S. aureus Cas9 molecule. In certain embodiments, the at least one Cas9 molecule comprises a wild-type Cas9 molecule, a mutant Cas9 molecule, or a combination thereof. In certain embodiments, the mutant Cas9 molecule comprises a D10A mutation. In certain embodiments, the composition is a ribonucleoprotein (RNP) composition, wherein the at least Cas9 molecules is complexed with the gRNA molecule.

The above-described compositions can be used in a medicament. The above-described compositions can be used in altering a CCR5 gene, altering a CXCR4 gene, or altering a CCR5 and a CXCR4 gene in a cell. In certain embodiments, the cell is from a subject suffering from HIV infection or AIDS. The above-described compositions can be used in treating HIV infection or AIDS.

The presently disclosed subject matter further provides for vectors comprising a polynucleotide encoding one gRNA molecule comprising a targeting domain that is complementary with a target sequence of a CCR5 gene. In certain embodiments, the targeting domain comprises a nucleotide sequence selected from SEQ ID NOS: 476 to 1569 and 1947 to 3663. In certain embodiments, the targeting domain comprises a nucleotide sequence selected from SEQ ID NOS: 208 to 475, and 1614 to 1946.

The presently disclosed subject matter provides for vectors comprising a gRNA molecule comprising a targeting domain that is complementary with a target sequence of a CXCR4 gene. In certain embodiments, the targeting domain comprises a nucleotide sequence selected from SEQ ID NOS: 4064 to 5208, and 5921 to 8355. In certain embodiments, the targeting domain comprises a nucleotide sequence selected from 3740 to 4063, and 5241 to 5920.

The presently disclosed subject matter provides for vectors comprising a polynucleotide encoding at least one of a first gRNA molecule comprising a first targeting domain that is complementary with a target sequence of a CCR5 gene, and a second gRNA molecule comprising a second targeting domain that is complementary with a target sequence of a CXCR4 gene. In certain embodiments, the first targeting domain comprises a nucleotide sequence selected from SEQ ID NOS: the first targeting domain comprises a nucleotide sequence selected from SEQ ID NOS: 476 to 1569 and 1947 to 3663, and the second targeting domain comprises a nucleotide sequence selected from SEQ ID NOS: 4064 to 5208, and 5921 to 8355. In certain embodiments, the first targeting domain comprises a nucleotide sequence selected from SEQ ID NOS: 208 to 475, and 1614 to 1946, and the second targeting domain comprises a nucleotide sequence selected from SEQ ID NOS: 3740 to 4063, and 5241 to 5920. In certain embodiments, the first targeting domain comprises a nucleotide sequence selected from SEQ ID NOS: 476 to 1569 and 1947 to 3663, and the second targeting domain comprises a nucleotide sequence selected from SEQ ID NOS: 3740 to 4063, and 5241 to 5920. In certain embodiments, the first targeting domain comprises a nucleotide sequence selected from SEQ ID NOS: 208 to 475, and 1614 to 1946, and the second targeting domain comprises a nucleotide sequence selected from SEQ ID NOS: 4064 to 5208, and 5921 to 8355.

In certain embodiments, the vector is a viral vector. In certain embodiments, the vector is an adeno-associated virus (AAV) vector.

The presently disclosed subject matter provides for methods of altering a CCR5 gene in a cell, comprising administering to the cell one of the above-described genome editing systems, or one of the above-described compositions. In certain embodiments, the alteration comprises introducing one or more mutations in the CCR5 gene, knocking out the CCR5 gene, knocking down the CCR5 gene, or combinations thereof. In certain embodiments, the method comprises introducing one or more protective mutations in the CCR5 gene. In certain embodiments, the one or more protective mutations comprise a CCR5 delta 32 mutation. In certain embodiments, the alteration of the CCR5 gene comprise homology-directed repair. In certain embodiments, the method further comprises administering to the cell a donor template. In certain embodiments, the donor template encodes an HIV fusion inhibitor.

The presently disclosed subject matter provides for methods of altering a CXCR4 gene in a cell, comprising administering to the cell one of the above-described genome editing systems, or one of the above-described compositions. In certain embodiments, the alteration comprises knocking out the CXCR4 gene, knocking down the CXCR4 gene, introducing one or more mutations in the CXCR4 gene, or combinations thereof. In certain embodiments, the one or more mutations comprise one or more single base substitutions, one or more two base substitutions, or combinations thereof.

The presently disclosed subject matter provides for methods of altering a CCR5 gene and a CXCR4 gene in a cell, comprising administering to the cell one of the above-described genome editing systems, or one of the above-described compositions. In certain embodiments, the alteration of the CCR5 gene comprises introducing one or more mutations in the CCR5 gene, knocking out the CCR5 gene, knocking down the CCR5 gene, or combinations thereof; and the alteration of the CXCR4 gene comprises knocking out the CXCR4 gene, knocking down the CXCR4 gene, introducing one or more mutations in the CXCR4 gene, or combinations thereof. In certain embodiments, the alteration of the CCR5 gene comprises introducing one or more protective mutation in the CCR5 gene. In certain embodiments, the one or more protective mutations comprise a CCR5 delta 32 mutation. In certain embodiments, the one or more mutations in the CXCR4 gene comprise one or more single base substitutions, one or more two base substitutions, or combinations thereof. In certain embodiments, at least one of the alteration of the CCR5 gene and the alteration of the CXCR4 gene comprise homology-directed repair. In certain embodiments, the method further comprises administering to the cell a donor template. In certain embodiments, the donor template encodes an HIV fusion inhibitor. In certain embodiments, the CCR5 gene and the CXCR4 gene are altered simultaneously or sequentially.

In certain embodiments, the cell is from a subject suffering from HIV infection or AIDS.

The presently disclosed subject matter provides for methods of treating or preventing HIV infection or AIDS, comprising administering to the subject one of the above-described genome editing systems, or one of the above-described compositions.

The presently disclosed subject matter provides forcells comprising at least one edited allele of a CCR5 a gene nd at least one edited allele of a CXCR4 gene. In certain embodiments, the cell is a hematopoietic stem cell, a hematopoietic progenitor cell, a multipotent progenitor cell, a common lymphoid progenitor, a common myeloid progenitor, lymphoid progenitor, a myeloid progenitor, a mature myeloid cell, a T memory stem (TSCM) cell, or a mature lymphoid cell. In the cell, the at least one edited allele of CCR5 optionally includes a transgene expression cassette encoding an anti-HIV transgene or element, or includes a selectable marker. In certain embodiments, the at least one edited allele of the CCR5 gene comprises a transgene expression cassette encoding an anti-HIV transgene or element. In certain embodiments, the edited allele of the CCR5 gene comprises a selectable marker.

The presently disclosed subject matter also provides for compositions comprising a plurality of cells characterized by at least 4% editing of a CCR5 a gene nd at least 4% editing of a CXCR4 gene, for example as measured by quantitative PCR. The plurality of cells optionally includes at least one of a hematopoietic stem cell, a hematopoietic progenitor cell, a multipotent progenitor cell, a common lymphoid progenitor, a common myeloid progenitor, lymphoid progenitor, a myeloid progenitor, a mature myeloid cell, a T memory stem (TSCM) cell, and a mature lymphoid cell, and is, in various embodiments, autologous or allogeneic.

The presently disclosed subject matter provides for methods of preparing a cell for transplantation, comprising contacting the cell with one of the above-described genome editing systems, or one of the above-described compositions.

The presently disclosed subject matter also provides for cells comprising the one of the above-described genome editing systems, one of the above-described compositions, or one of the above-described vectors.

### Alteration of CCR5

In certain embodiments, the methods, genome editing systems, and compositions discussed herein, inhibit or block a critical aspect of the HIV life cycle, i.e., CCR5-mediated entry into T cells, by alteration (e.g., inactivation of the CCR5 gene or truncation of the gene product) of CCR5 expression. Exemplary mechanisms that can be associated with the alteration of the CCR5 gene include, but are not limited to, non-homologous end joining (NHEJ) (e.g., classical or alternative), microhomology-mediated end joining (MMEJ), homology-directed repair (e.g., endogenous donor template mediated), SDSA (synthesis dependent strand annealing), single strand annealing or single strand invasion. Alteration of the CCR5 gene, e.g., mediated by NHEJ, can result in a mutation, which typically comprises a deletion or insertion (indel). The introduced mutation can take place in any region of the CCR5 gene, e.g., a promoter region or other non-coding region, or a coding region, so long as the mutation results in reduced or loss of the ability to mediate HIV entry into the cell.

In certain embodiments, the methods, genome editing systems, and compositions discussed herein are used to alter the CCR5 gene to treat or prevent HIV infection or AIDS by targeting the coding sequence of the CCR5 gene.

In certain embodiments, the gene, e.g., the coding sequence of the CCR5 gene, is targeted to knock out the gene, e.g., to eliminate expression of the gene, e.g., to knock out both alleles of the CCR5 gene, e.g., by introduction of an alteration comprising a mutation (e.g., an insertion or deletion) in the CCR5 gene. This type of alteration is sometimes referred to as “knocking out” the CCR5 gene. In certain embodiments, a targeted knockout approach is mediated by NHEJ using a CRISPR/Cas system comprising a Cas9 molecule, e.g., an enzymatically active Cas9 (eaCas9) molecule, as described herein.

In certain embodiments, the methods, genome editing systems, and compositions discussed herein are used to alter the CCR5 gene to treat or prevent HIV infection or AIDS by targeting a non-coding sequence of the CCR5 gene, e.g., a promoter, an enhancer, an intron, a 3′UTR, and/or a polyadenylation signal.

In certain embodiments, the gene, e.g., the non-coding sequence of the CCR5 gene, is targeted to knock out the gene, e.g., to eliminate expression of the gene, e.g., to knock out both alleles of the CCR5 gene, e.g., by introduction of an alteration comprising a mutation (e.g., an insertion or deletion) in the CCR5 gene. In certain embodiments, the method provides an alteration that comprises an insertion or deletion. This type of alteration is also sometimes referred to as “knocking out” the CCR5 gene. In certain embodiments, a targeted knockout approach is mediated by NHEJ using a CRISPR/Cas system comprising a Cas9 molecule, e.g., an enzymatically active Cas9 (eaCas9) molecule, as described herein.

In certain embodiments, the methods, genome editing systems, and compositions discussed herein, provide for introducing one or more mutations in the CCR5 gene. In certain embodiments, the one or more mutations comprises one or more protective mutations. In certain embodiments, the one or more protective mutations comprise a delta32 mutation in the CCR5 gene.

In certain embodiments, the methods, genome editing systems, and compositions discussed herein, provide for knocking out the CCR5 gene. In certain embodiments, knocking out the CCR5 gene comprises (1) insertion or deletion (e.g., NHEJ-mediated insertion or deletion) of one or more nucleotides of the CCR5 gene (e.g., in close proximity to or within an early coding region or in a non-coding region), and/or (2) deletion (e.g., NHEJ-mediated deletion) of a genomic sequence of the CCR5 gene (e.g., in a coding region or in a non-coding region). Both approaches can give rise to alteration (e.g., knockout) of the CCR5 gene as described herein. In certain embodiments, a CCR5 target knockout position is altered by genome editing using the CRISPR/Cas9 system. The CCR5 target knockout position can be targeted by cleaving with either one or more nucleases, or one or more nickases, or a combination thereof. In certain embodiments, knockout of a CCR5 gene is combined with a concomitant knockin of an anti-HIV gene or genes under expression of endogenous promoter or Pol III promoter. In certain embodiments, knockout of a CCR5 gene is combined with a concomitant knockin of a drug resistance selectable marker for enabling selection of modified HSCs.

“CCR5 target knockout position”, as used herein, refers to a position in the CCR5 gene, which if altered, e.g., disrupted by insertion or deletion of one or more nucleotides, e.g., by NHEJ-mediated alteration, results in alteration of the CCR5 gene. In certain embodiments, the position is in the CCR5 coding region, e.g., an early coding region. In certain embodiments, the position is in a non-coding sequence of the CCR5 gene, e.g., a promoter, an enhancer, an intron, a 3′UTR, and/or a polyadenylation signal.

In certain embodiments, the CCR5 gene is targeted for knocking down, e.g., for reducing or eliminating expression of the CCR5 gene, e.g., knocking down one or both alleles of the CCR5 gene.

In certain embodiments, the coding region of the CCR5 gene, is targeted to alter the expression of the gene. In certain embodiments, a non-coding region (e.g., an enhancer region, a promoter region, an intron, a 5′ UTR, a 3′UTR, or a polyadenylation signal) of the CCR5 gene is targeted to alter the expression of the gene. In certain embodiments, the promoter region of the CCR5 gene is targeted to knock down the expression of the CCR5 gene. This type of alteration is also sometimes referred to as “knocking down” the CCR5 gene. In certain embodiments, a targeted knockdown approach is mediated by a CRISPR/Cas system comprising a Cas9 molecule, e.g., an enzymatically inactive Cas9 (eiCas9) molecule or an eiCas9 fusion protein (e.g., an eiCas9 fused to a transcription repressor domain or chromatin modifying protein), as described herein. In certain embodiments, the CCR5 gene is targeted to alter (e.g., to block, reduce, or decrease) the transcription of the CCR5 gene. In certain embodiments, the CCR5 gene is targeted to alter the chromatin structure (e.g., one or more histone and/or DNA modifications) of the CCR5 gene. In certain embodiments, one or more gRNA molecules comprising a targeting domain are configured to target an enzymatically inactive Cas9 (eiCas9) molecule or an eiCas9 fusion protein (e.g., an eiCas9 fused to a transcription repressor domain), sufficiently close to a CCR5 target knockdown position to reduce, decrease or repress expression of the CCR5 gene.

“CCR5 target knockdown position”, as used herein, refers to a position in the CCR5 gene, which if targeted, e.g., by an eiCas9 molecule or an eiCas9 fusion described herein, results in reduction or elimination of expression of functional CCR5 gene product. In certain embodiments, the transcription of the CCR5 gene is reduced or eliminated. In certain embodiments, the chromatin structure of the CCR5 gene is altered. In certain embodiments, the position is in the CCR5 promoter sequence. In certain embodiments, a position in the promoter sequence of the CCR5 gene is targeted by an enzymatically inactive Cas9 (eiCas9) molecule or an eiCas9 fusion protein, as described herein.

“CCR5 target position”, as used herein, refers to any position that results in alteration of a CCR5 gene. In certain embodiments, a CCR5 target position comprisesa CCR5 target knockout position, a CCR5 target knockdown position, or a position within the CCR5 gene that is targeted for introduction of one or more mutations (e.g., one or more protective mutations, e.g., delta32 mutation).

In certain embodiments, disclosed herein is a gRNA molecule, e.g., an isolated or non-naturally occurring gRNA molecule, comprising a targeting domain which is complementary with a target domain (also referred to as “target sequence”) from the CCR5 gene.

In certain embodiments, the targeting domain of the gRNA molecule is configured to provide a cleavage event, e.g., a double strand break or a single strand break, sufficiently close to a CCR5 target position in the CCR5 gene to allow alteration, e.g., alteration associated with NHEJ, of a CCR5 target position in the CCR5 gene. In certain embodiments, the alteration comprises an insertion or deletion. In certain embodiments, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 300, 400, 450, or 500 nucleotides of a CCR5 target position. The break, e.g., a double strand or single strand break, can be positioned upstream or downstream of a CCR5 target position in the CCR5 gene.

In certain embodiments, a second gRNA molecule comprising a second targeting domain is configured to provide a cleavage event, e.g., a double strand break or a single strand break, sufficiently close to the CCR5 target position in the CCR5 gene, to allow alteration, e.g., alteration associated with NHEJ, of the CCR5 target position in the CCR5 gene, either alone or in combination with the break positioned by said first gRNA molecule. In certain embodiments, the targeting domains of the first and second gRNA molecules are configured such that a cleavage event, e.g., a double strand or single strand break, is positioned, independently for each of the gRNA molecules, within 1, 2, 3, 4, 5, 10, 15, 20, 25, 30, 35, 40, 45, 50, 60, 70, 80, 90, 100, 150, 200, 300, 400, 450, or 500 nucleotides of the target position. In certain embodiments, the breaks, e.g., double strand or single strand breaks, are positioned on both sides of a nucleotide of a CCR5 target position in the CCR5 gene. In certain embodiments, the breaks, e.g., double strand or single strand breaks, are positioned on one side, e.g., upstream or downstream, of a nucleotide of a CCR5 target position in the CCR5 gene.

In certain embodiments, when CCR5 is targeted for knock out, a single strand break is accompanied by an additional single strand break, positioned by a second gRNA molecule, as discussed below. For example, the targeting domains are configured such that a cleavage event, e.g., the two single strand breaks, are positioned within 1, 2, 3, 4, 5, 10, 15, 20, 25, 30, 35, 40, 45, 50, 60, 70, 80, 90, 100, 150, 200, 300, 400, 450, or 500 nucleotides of a CCR5 target position. In certain embodiments, the first and second gRNA molecules are configured such, that when guiding a Cas9 molecule, e.g., a Cas9 nickase, a single strand break can be accompanied by an additional single strand break, positioned by a second gRNA, sufficiently close to one another to result in alteration of a CCR5 target position in the CCR5 gene. In certain embodiments, the first and second gRNA molecules are configured such that a single strand break positioned by said second gRNA is within 1, 2, 3, 4, 5, 10, 20, 30, 40, 50, 60, 70, 80, 90, 100, 200, 300, 400, 500, 600, 700, 800, 900, or 1000 nucleotides of the break positioned by said first gRNA molecule, e.g., when the Cas9 molecule is a nickase. In certain embodiments, the two gRNA molecules are configured to position cuts at the same position, or within a few nucleotides of one another, on different strands, e.g., essentially mimicking a double strand break.

In certain embodiments, when CCR5 is targeted for knock out, a double strand break can be accompanied by an additional double strand break, positioned by a second gRNA molecule, as is discussed below. For example, the targeting domain of a first gRNA molecule is configured such that a double strand break is positioned upstream of a CCR5 target position in the CCR5 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position; and the targeting domain of a second gRNA molecule is configured such that a double strand break is positioned downstream of a CCR5 target position in the CCR5 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position. In certain embodiments, the first and second gRNA molecules are configured such that a double strand break positioned by said second gRNA is within 10, 20, 30, 40, 50, 60, 70, 80, 90, 100, 200, 300, 400, 500, 600, 700, 800, 900, or 1000 nucleotides of the break positioned by said first gRNA molecule.

In certain embodiments, the targeting domains of the first and second gRNA molecules are configured such that a cleavage event, e.g., a single strand break, is positioned, independently for each of the gRNA molecules.

In certain embodiments, when CCR5 is targeted for knock out, a double strand break can be accompanied by two additional single strand breaks, positioned by a second gRNA molecule and a third gRNA molecule. For example, the targeting domain of a first gRNA molecule is configured such that a double strand break is positioned upstream of a CCR5 target position in the CCR5 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position; and the targeting domains of a second and third gRNA molecule are configured such that two single strand breaks are positioned downstream of a CCR5 target position in the CCR5 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position. In certain embodiments, the first, second and third gRNA molecules are configured such that a single strand break positioned by said second or third gRNA molecule is within 10, 20, 30, 40, 50, 60, 70, 80, 90, 100, 200, 300, 400, 500, 600, 700, 800, 900, or 1000 nucleotides of the break positioned by said first gRNA molecule. In certain embodiments, the targeting domains of the first, second and third gRNA molecules are configured such that a cleavage event, e.g., a double strand or single strand break, is positioned, independently for each of the gRNA molecules.

In certain embodiments, when CCR5 is targeted for knock out, a first and second single strand breaks can be accompanied by two additional single strand breaks positioned by a third gRNA molecule and a fourth gRNA molecule. For example, the targeting domain of a first and second gRNA molecule are configured such that two single strand breaks are positioned upstream of a CCR5 target position in the CCR5 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position; and the targeting domains of a third and fourth gRNA molecule are configured such that two single strand breaks are positioned downstream of a CCR5 target position in the CCR5 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position. In certain embodiments, the first, second, third and fourth gRNA molecules are configured such that the single strand break positioned by said third or fourth gRNA molecule is within 10, 20, 30, 40, 50, 60, 70, 80, 90, 100, 200, 300, 400, 500, 600, 700, 800, 900, or 1000 nucleotides of the break positioned by said first or second gRNA molecule, e.g., when the Cas9 molecule is a nickase. In certain embodiments, the targeting domains of the first, second, third and fourth gRNA molecules are configured such that a cleavage event, e.g., a single strand break, is positioned, independently for each of the gRNA molecules.

In certain embodiments, when multiple gRNAs are used to generate (1) two single stranded breaks in close proximity, (2) two double stranded breaks, e.g., flanking a CCR5 target position (e.g., to remove a piece of DNA, e.g., a insertion or deletion mutation) or to create more than one indel in an early coding region, (3) one double stranded break and two paired nicks flanking a CCR5 target position (e.g., to remove a piece of DNA, e.g., a insertion or deletion mutation) or (4) four single stranded breaks, two on each side of a CCR5 target position, that they are targeting the same CCR5 target position. It is further contemplated herein that in certain embodiments multiple gRNAs may be used to target more than one target position in the same gene.

In certain embodiments, the targeting domain of the first gRNA molecule and the targeting domain of the second gRNA molecules are complementary to opposite strands of the target nucleic acid molecule. In certain embodiments, the gRNA molecule and the second gRNA molecule are configured such that the PAMs are oriented outward.

In certain embodiments, the targeting domain of a gRNA molecule is configured to avoid unwanted target chromosome elements, such as repeat elements, e.g., Alu repeats, in the target domain (also referred to as “target sequence”). The gRNA molecule may be a first, second, third and/or fourth gRNA molecule, as described herein.

In certain embodiments, the targeting domain of a gRNA molecule is configured to position a cleavage event sufficiently far from a preselected nucleotide, e.g., the nucleotide of a coding region, such that the nucleotide is not altered. In certain embodiments, the targeting domain of a gRNA molecule is configured to position an intronic cleavage event sufficiently far from an intron/exon border, or naturally occurring splice signal, to avoid alteration of the exonic sequence or unwanted splicing events. The gRNA molecule may be a first, second, third and/or fourth gRNA molecule, as described herein.

In certain embodiments, a CCR5 target position is targeted and the targeting domain of a gRNA molecule comprises a sequence that is the same as, or differs by no more than 1, 2, 3, 4, or 5 nucleotides from, a targeting domain sequence comprising a nucleotide sequence selected from SEQ ID NOS: 208 to 3739. In certain embodiments, the targeting domain comprises a nucleotide sequence selected from SEQ ID NOS: 208 to 3739. In certain embodiments, the targeting domain is independently selected from:

(SEQ ID NO: 208)

ACUAUGCUGCCGCCCAG;

(SEQ ID NO: 209)

UCCUCCUGACAAUCGAU;

(SEQ ID NO: 210)

CUAUGCUGCCGCCCAGU;

(SEQ ID NO: 211)

GCCGCCCAGUGGGACUU;

(SEQ ID NO: 212)

UUGACAGGGCUCUAUUUUAU;
or

(SEQ ID NO: 213)

UCACUAUGCUGCCGCCCAGU.

In certain embodiments, the targeting domain comprises a nucleotide sequence selected from SEQ ID NOS: 208 to 1569 and 1614 to 3663. In certain embodiments, the targeting domain comprises a nucleotide sequence selected from 335, 480, 482, 486, 488, 490, 492, 512, 521, 535, 1000, and 1002.

In certain embodiments, more than one gRNA is used to position breaks, e.g., two single stranded breaks or two double stranded breaks, or a combination of single strand and double strand breaks, e.g., to create one or more indels, in the target nucleic acid sequence. In certain embodiments, two, three or four gRNA molecules are used to position breaks. In certain embodiments, the targeting domain of each gRNA molecules comprises a nucleotide sequence selected from SEQ ID NOS: 208 to 3739. In certain embodiments, the targeting domain of each gRNA molecules comprises a nucleotide sequence selected from SEQ ID NOS: 208 to 1569 and 1614 to 3663. In certain embodiments, the genome editing systems or compositions described herein comprise two gRNA molecules that target a CCR5 gene (a first CCR5 gRNA molecule and a second CCR5 gRNA molecule). In certain embodiments, the first CCR5 gRNA molecule comprises a targeting domain comprising the nucleotide sequence set forth in SEQ ID NO: 480, and the second CCR5 gRNA molecule comprises a targeting domain comprising the nucleotide sequence set forth in SEQ ID NO: 448. In certain embodiments, the first CCR5 gRNA molecule comprises a targeting domain comprising the nucleotide sequence set forth in SEQ ID NO: 480, and the second CCR5 gRNA molecule comprises a targeting domain comprising the nucleotide sequence set forth in SEQ ID NO: 335.

In certain embodiments, the targeting domain of the gRNA molecule is configured to target an enzymatically inactive Cas9 (eiCas9) molecule or an eiCas9 fusion protein (e.g., an eiCas9 fused to a transcription repressor domain), sufficiently close to a CCR5 transcription start site (TSS) to reduce (e.g., block) transcription, e.g., transcription initiation or elongation, binding of one or more transcription enhancers or activators, and/or RNA polymerase. In certain embodiments, the targeting domain is configured to target between 1000 bp upstream and 1000 bp downstream (e.g., between 500 bp upstream and 1000 bp downstream, between 1000 bp upstream and 500 bp downstream, between 500 bp upstream and 500 bp downstream, within 500 bp or 200 bp upstream, or within 500 bp or 200 bp downstream) of the TSS of the CCR5 gene. One or more gRNAs may be used to target an eiCas9 to the promoter region of the CCR5 gene.

In certain embodiments, the targeting domain comprises a nucleotide sequence that is the same as, or differs by no more than 1, 2, 3, 4, or 5 nucleotides from, a nucleotide sequence selected from SEQ ID NO: 208 to 3739. In certain embodiments, the targeting domain comprises a nucleotide sequence selected from SEQ ID NOS: 208 to 3739. In certain embodiments, the targeting domain comprises a nucleotide sequence selected from SEQ ID NOS: 208 to 1569 and 1614 to 3663.

In certain embodiments, the CCR5 gene is targeted for knockout, and the targeting domain of the gRNA molecule can comprise a nucleotide sequence that is the same as, or differs by no more than 1, 2, 3, 4, or 5 nucleotides from, the nucleotide sequence selected from SEQ ID NOS: 208 to 1613. In certain embodiments, the targeting domain comprises a nucleotide sequence selected from SEQ ID NOS: 208 to 1613. In certain embodiments, the targeting domain comprises a nucleotide sequence selected from SEQ ID NOS: 208 to 1569. In certain embodiments, the targeting domain comprises a nucleotide sequence selected from 335, 480, 482, 486, 488, 490, 492, 512, 521, 535, 1000, and 1002.

In certain embodiments, when the CCR5 gene is targeted for knockdown, and the targeting domain of the gRNA molecule can comprise a nucleotide sequence that is the same as, or differs by no more than 1, 2, 3, 4, or 5 nucleotides from, the nucleotide sequence selected from SEQ ID NOS: 1614 to 3739. In certain embodiments, the targeting domain comprises a nucleotide sequence selected from SEQ ID NOS: 1614 to 3739. In certain embodiments, the targeting domain comprises a nucleotide sequence selected from SEQ ID NOS: 1614 to 3663.

In certain embodiments, the promoter region of the CCR5 gene is targeted for knowdown. In certain embodiments, when the CCR5 target knockdown position is the CCR5 promoter region and more than one gRNA molecule is used to position an eiCas9 molecule or an eiCas9-fusion protein (e.g., an eiCas9-transcription repressor domain fusion protein), in the target nucleic acid sequence, the targeting domain for each gRNA molecule comprises a nucleotide sequence selected from SEQ ID NOS: 1614 to 3739. In certain embodiments, the targeting domain comprises a nucleotide sequence selected from SEQ ID NOS: 1614 to 3663.

In certain embodiments, the targeting domain which is complementary with a target domain (also referred to as “target sequence”) from the CCR5 target position in the CCR5 gene is 16 nucleotides or more in length. In certain embodiments, the targeting domain is 16 nucleotides in length. In certain embodiments, the targeting domain is 17 nucleotides in length. In other embodiments, the targeting domain is 18 nucleotides in length. In still other embodiments, the targeting domain is 19 nucleotides in length. In still other embodiments, the targeting domain is 20 nucleotides in length. In certain embodiments, the targeting domain is 21 nucleotides in length. In certain embodiments, the targeting domain is 22 nucleotides in length. In certain embodiments, the targeting domain is 23 nucleotides in length. In certain embodiments, the targeting domain is 24 nucleotides in length. In certain embodiments, the targeting domain is 25 nucleotides in length. In certain embodiments, the targeting domain is 26 nucleotides in length.

In certain embodiments, the targeting domain comprises 16 nucleotides. In certain embodiments, the targeting domain comprises 17 nucleotides. In certain embodiments, the targeting domain comprises 18 nucleotides. In certain embodiments, the targeting domain comprises 19 nucleotides. In certain embodiments, the targeting domain comprises 20 nucleotides. In certain embodiments, the targeting domain comprises 21 nucleotides. In certain embodiments, the targeting domain comprises 22 nucleotides. In certain embodiments, the targeting domain comprises 23 nucleotides. In certain embodiments, the targeting domain comprises 24 nucleotides. In certain embodiments, the targeting domain comprises 25 nucleotides. In certain embodiments, the targeting domain comprises 26 nucleotides.

A gRNA as described herein may comprise from 5′ to 3′: a targeting domain (comprising a “core domain”, and optionally a “secondary domain”); a first complementarity domain; a linking domain; a second complementarity domain; a proximal domain; and a tail domain. In certain embodiments, the proximal domain and tail domain are taken together as a single domain.

In certain embodiments, a gRNA comprises a linking domain of no more than 25 nucleotides in length; a proximal and tail domain, that taken together, are at least 20, at least 25, at least 30, at least 35, or at least 40 nucleotides in length; and a targeting domain equal to or greater than 16, 17, 18, 19, 20, 21, 22, 23, 24, 25 or 26 nucleotides in length.

A cleavage event, e.g., a double strand or single strand break, is generated by a Cas9 molecule. The Cas9 molecule may be an enzymatically active Cas9 (eaCas9) molecule, e.g., an eaCas9 molecule that forms a double strand break in a target nucleic acid or an eaCas9 molecule forms a single strand break in a target nucleic acid (e.g., a nickase molecule).

In certain embodiments, the eaCas9 molecule catalyzes a double strand break.

In certain embodiments, the eaCas9 molecule comprises HNH-like domain cleavage activity but has no, or no significant, N-terminal RuvC-like domain cleavage activity. In this case, the eaCas9 molecule is an HNH-like domain nickase, e.g., the eaCas9 molecule comprises a mutation at D10, e.g., D10A. In other embodiments, the eaCas9 molecule comprises N-terminal RuvC-like domain cleavage activity but has no, or no significant, HNH-like domain cleavage activity. In certain embodiments, the eaCas9 molecule is an N-terminal RuvC-like domain nickase, e.g., the eaCas9 molecule comprises a mutation at H840, e.g., H840A. In certain embodiments, the eaCas9 molecule is an N-terminal RuvC-like domain nickase, e.g., the eaCas9 molecule comprises a mutation at N863, e.g., N863A. In certain embodiments, the eaCas9 molecule is an N-terminal RuvC-like domain nickase, e.g., the eaCas9 molecule comprises a mutation at N580, e.g., N580A.

In certain embodiments, a single strand break is formed in the strand of the target nucleic acid to which the targeting domain of said gRNA is complementary. In certain embodiments, a single strand break is formed in the strand of the target nucleic acid other than the strand to which the targeting domain of said gRNA is complementary.

The presently disclosed subject matter also provides for a nucleic acid composition, e.g., an isolated or non-naturally occurring nucleic acid composition, e.g., DNA, that comprises (a) a first nucleotide sequence that encodes a first gRNA molecule comprising a targeting domain that is complementary with a CCR5 target position in the CCR5 gene as disclosed herein. In certain embodiments, the first gRNA molecule comprises a targeting domain configured to provide a cleavage event, e.g., a double strand break or a single strand break, sufficiently close to a CCR5 target position in the CCR5 gene to allow alteration, e.g., alteration associated with NHEJ, of a CCR5 target position in the CCR5 gene. In certain embodiments, the first gRNA molecule comprises a targeting domain configured to target an enzymatically inactive Cas9 (eiCas9) molecule or an eiCas9 fustion protein (e.g., an eiCas9 fused to a transcription repressor domain or chromatin modifying protein), sufficiently close to a CCR5 knockdown target position to reduce, decrease or repress expression of the CCR5 gene. In certain embodiments, the first gRNA molecule comprises a targeting domain comprising a nucleotide sequence that is the same as, or differs by no more than 1, 2, 3, 4, or 5 nucleotides from, a nucleotide sequence selected from SEQ ID NOS: 208 to 3739, SEQ ID NOS: 208 to 1613, or SEQ ID NOS: 1614 to 3739. In certain embodiments, the first gRNA molecule comprises a targeting domain comprising a nucleotide sequence selected from SEQ ID NOS: 208 to 3739, SEQ ID NOS: 208 to 1613, or SEQ ID NOS: 1614 to 3739.

In certain embodiments, the nucleic acid composition further comprises (b) a second nucleotide sequence that encodes a Cas9 molecule. In certain embodiments, the Cas9 molecule is a nickase molecule, an enzymatically active Cas9 (eaCas9) molecule, e.g., an eaCas9 molecule that forms a double strand break in a target nucleic acid and/or an eaCas9 molecule that forms a single strand break in a target nucleic acid. In certain embodiments, a single strand break is formed in the strand of the target nucleic acid to which the targeting domain of said gRNA is complementary. In certain embodiments, a single strand break is formed in the strand of the target nucleic acid other than the strand to which to which the targeting domain of said gRNA is complementary. In certain embodiments, the eaCas9 molecule catalyzes a double strand break.

In certain embodiments, the eaCas9 molecule comprises HNH-like domain cleavage activity but has no, or no significant, N-terminal RuvC-like domain cleavage activity. In certain embodiments, the said eaCas9 molecule is an HNH-like domain nickase, e.g., the eaCas9 molecule comprises a mutation at D10, e.g., D10A. In certain embodiments, the eaCas9 molecule comprises N-terminal RuvC-like domain cleavage activity but has no, or no significant, HNH-like domain cleavage activity. In certain embodiments, the eaCas9 molecule is an N-terminal RuvC-like domain nickase, e.g., the eaCas9 molecule comprises a mutation at H840, e.g., H840A. In certain embodiments, the eaCas9 molecule is an N-terminal RuvC-like domain nickase, e.g., the eaCas9 molecule comprises a mutation at N863, e.g., N863A. In certain embodiments, the eaCas9 molecule is an N-terminal RuvC-like domain nickase, e.g., the eaCas9 molecule comprises a mutation at N580, e.g., N580A.

In certain embodiments, the Cas9 molecule is an enzymatically inactive Cas9 (eiCas9) molecule or a modified eiCas9 molecule, e.g., the eiCas9 molecule is fused to Krüppel-associated box (KRAB) to generate an eiCas9-KRAB fusion protein molecule.

In certain embodiments, the nucleic acid composition further comprises (c)(i) a third nucleotide sequence that encodes a second gRNA molecule described herein having a targeting domain that is complementary to a second target domain of the CCR5 gene, and optionally, (c)(ii) a fourth nucleotide sequence that encodes a third gRNA molecule described herein having a targeting domain that is complementary to a third target domain of the CCR5 gene; and optionally, (c)(iii) a fifth nucleotide sequence that encodes a fourth gRNA molecule described herein having a targeting domain that is complementary to a fourth target domain of the CCR5 gene.

In certain embodiments, the second gRNA molecule comprises a targeting domain configured to provide a cleavage event, e.g., a double strand break or a single strand break, sufficiently close to a CCR5 target position in the CCR5 gene, to allow alteration, e.g., alteration associated with NHEJ, of a CCR5 target position in the CCR5 gene, either alone or in combination with the break positioned by said first gRNA molecule. In certain embodiments, the second gRNA molecule comprises a targeting domain configured to target an enzymatically inactive Cas9 (eiCas9) molecule or an eiCas9 fustion protein (e.g., an eiCas9 fused to a transcription repressor domain or chromatin modifying protein), sufficiently close to a CCR5 knockdown target position to reduce, decrease or repress expression of the CCR5 gene.

In certain embodiments, the third gRNA molecule comprises a targeting domain configured to provide a cleavage event, e.g., a double strand break or a single strand break, sufficiently close to a CCR5 target position in the CCR5 gene to allow alteration, e.g., alteration associated with NHEJ, of a CCR5 target position in the CCR5 gene, either alone or in combination with the break positioned by the first and/or second gRNA molecule. In certain embodiments, the third gRNA molecule comprises a targeting domain configured to target an enzymatically inactive Cas9 (eiCas9) molecule or an eiCas9 fustion protein (e.g., an eiCas9 fused to a transcription repressor domain or chromatin remodeling protein), sufficiently close to a CCR5 knockdown target position to reduce, decrease or repress expression of the CCR5 gene.

In certain embodiments, the fourth gRNA molecule comprises a targeting domain configured to provide a cleavage event, e.g., a double strand break or a single strand break, sufficiently close to a CCR5 target position in the CCR5 gene to allow alteration, e.g., alteration associated with NHEJ, of a CCR5 target position in the CCR5 gene, either alone or in combination with the break positioned by the first gRNA molecule, the second gRNA molecule and/or the third gRNA molecule.

In certain embodiments, the second gRNA targets the same CCR5 target position as the first gRNA molecule. In certain embodiments, the third gRNA molecule and the fourth gRNA molecule target the same CCR5 target position as the first and second gRNA molecules.

The targeting domain of each of the second, third, and fourth gRNA molecules can comprise a nucleotide sequence that is the same as, or differs by no more than 1, 2, 3, 4, or 5 nucleotides from, a nucleotide sequence selected from SEQ ID NOS: 208 to 3739, SEQ ID NOS: 208 to 1613, or SEQ ID NOS: 1614 to 3739. In certain embodiments, the targeting domain of each of the second, third, and fourth gRNA molecules comprises a nucleotide sequence selected from SEQ ID NOS: 208 to 3739, SEQ ID NOS: 208 to 1613, or SEQ ID NOS: 1614 to 3739.

When multiple gRNAs are used, any combination of modular or chimeric gRNAs may be used.

In certain embodiments, the first gRNA molecule of (a) and the Cas9 molecule of (b) are present on one nucleic acid molecule, e.g., one vector, e.g., one viral vector, e.g., one adeno-associated virus (AAV) vector. In certain embodiments, the nucleic acid molecule is an AAV vector. Exemplary AAV vectors that may be used in any of the described compositions and methods include an AAV1 vector, a modified AAV1 vector, an AAV2 vector, a modified AAV2 vector, an AAV3 vector, an AAV4 vector, a modified AAV4 vector, an AAV5 vector, a modified AAV5 vector, a modified AAV3 vector, an AAV6 vector, a modified AAV6 vector, an AAV8 vector an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector.

In certain embodiments, (a) is present on a first nucleic acid molecule, e.g. a first vector, e.g., a first viral vector, e.g., a first AAV vector; and (b) is present on a second nucleic acid molecule, e.g., a second vector, e.g., a second vector, e.g., a second AAV vector. The first and second nucleic acid molecules may be AAV vectors.

In certain embodiments, the first gRNA molecule of (a) and the second gRNA molecule of (c)(i), optionally, the fourth gRNA molecule of (c)(ii) and the fifth gRNA molecule of (c)(iii) are present on one nucleic acid molecule, e.g., one vector, e.g., one viral vector, e.g., one AAV vector. In certain embodiments, the nucleic acid molecule is an AAV vector.

In certain embodiments, (a) and (c)(i) are present on different vectors. For example, (a) is present on a first nucleic acid molecule, e.g. a first vector, e.g., a first viral vector, e.g., a first AAV vector; and (c)(i) is present on a second nucleic acid molecule, e.g., a second vector, e.g., a second vector, e.g., a second AAV vector. In certain embodiments, the first and second nucleic acid molecules are AAV vectors.

In certain embodiments, each of (a), (b), and (c)(i) are present on one nucleic acid molecule, e.g., one vector, e.g., one viral vector, e.g., an AAV vector. In certain embodiments, the nucleic acid molecule is an AAV vector. In certain embodiment, one of (a), (b), and (c)(i) is encoded on a first nucleic acid molecule, e.g., a first vector, e.g., a first viral vector, e.g., a first AAV vector; and a second and third of (a), (b), and (c)(i) is encoded on a second nucleic acid molecule, e.g., a second vector, e.g., a second vector, e.g., a second AAV vector. The first and second nucleic acid molecule may be AAV vectors.

In certain embodiments, (a) is present on a first nucleic acid molecule, e.g., a first vector, e.g., a first viral vector, a first AAV vector; and (b) and (c)(i) are present on a second nucleic acid molecule, e.g., a second vector, e.g., a second vector, e.g., a second AAV vector. The first and second nucleic acid molecule may be AAV vectors.

In certain embodiments, (b) is present on a first nucleic acid molecule, e.g., a first vector, e.g., a first viral vector, e.g., a first AAV vector; and (a) and (c)(i) are present on a second nucleic acid molecule, e.g., a second vector, e.g., a second vector, e.g., a second AAV vector. The first and second nucleic acid molecule may be AAV vectors.

In certain embodiments, (c)(i) is present on a first nucleic acid molecule, e.g., a first vector, e.g., a first viral vector, e.g., a first AAV vector; and (b) and (a) are present on a second nucleic acid molecule, e.g., a second vector, e.g., a second vector, e.g., a second AAV vector. The first and second nucleic acid molecule may be AAV vectors.

In certain embodiments, (a), (b) and (c)(i), optionally (c)(ii) and (c)(iii) are present together in a genome editing system. In certain embodiments, each of (a), (b) and (c)(i) are present on different nucleic acid molecules, e.g., different vectors, e.g., different viral vectors, e.g., different AAV vector. For example, (a) may be on a first nucleic acid molecule, (b) on a second nucleic acid molecule, and (c)(i) on a third nucleic acid molecule. The first, second and third nucleic acid molecule may be AAV vectors.

In certain embodiments, when a third and/or fourth gRNA molecule are present, each of (a), (b), (c)(i), (c)(ii) and (c)(iii) may be present on one nucleic acid molecule, e.g., one vector, e.g., one viral vector, e.g., an AAV vector. In certain embodiments, the nucleic acid molecule is an AAV vector. In certain embodiments, each of (a), (b), (c)(i), (c)(ii) and (c)(iii) may be present on the different nucleic acid molecules, e.g., different vectors, e.g., the different viral vectors, e.g., different AAV vectors. In a further embodiment, each of (a), (b), (c)(i), (c)(ii) and (c)(iii) may be present on more than one nucleic acid molecule, but fewer than five nucleic acid molecules, e.g., AAV vectors.

The nucleic acid composition described herein may comprise a promoter operably linked to the first nucleotide sequence that encodes the first gRNA molecule of (a), e.g., a promoter described herein. The nucleic acid composition may further comprise a second promoter operably linked to the third nucleotide sequence that encodes the second gRNA molecule of (c)(i), e.g., a promoter described herein. The promoter and second promoter differ from one another. In certain embodiments, the promoter and second promoter are the same.

The nucleic acid composition described herein may further comprise a promoter operably linked to the second nucleotide sequence that encodes the Cas9 molecule of (b), e.g., a promoter described herein.

In certain embodiments, disclosed herein is a composition comprising (a) a gRNA molecule comprising a targeting domain that is complementary with a target domain (also referred to as “target sequence”) in the CCR5 gene, as described herein. The composition of (a) may further comprise (b) a Cas9 molecule, e.g., a Cas9 molecule as described herein. A composition of (a) and (b) may further comprise (c) a second gRNA molecule, optionally a third gRNA molecule and a fourth gRNA molecule, e.g., a second, third and/or fourth gRNA molecule described herein. In certain embodiments, the composition is a pharmaceutical composition, e.g. a composition including a pharmaceutically acceptable carrier or excipient. The compositions described herein, e.g., pharmaceutical compositions described herein, can be used in the treatment or prevention of HIV or AIDS in a subject, e.g., in accordance with a method disclosed herein.

In certain embodiments, disclosed herein is a method of altering a cell, e.g., altering the structure, e.g., altering the sequence, of a target nucleic acid of a cell, comprising contacting said cell with: (a) a gRNA that targets the CCR5 gene, e.g., a gRNA as described herein; (b) a Cas9 molecule, e.g., a Cas9 molecule as described herein; and optionally, (c) a second gRNA molecule that targets the CCR5 gene, as described herein. In certain embodiments, the method comprises contacting the cell with a third gRNA molecule and further with a fourth gRNA molcule, as described herein.

In certain embodiments, the method comprises contacting said cell with (a) and (b). In certain embodiments, the method comprises contacting said cell with (a), (b), and (c).

In certain embodiments, the cell is from a subject suffering from or likely to develop an HIV infection or AIDS. The cell may be from a subject who does not have a mutation at a CCR5 target position.

In certain embodiments, the cell being contacted in the disclosed method is a target cell from a circulating blood cell, a progenitor cell, or a stem cell, e.g., a hematopoietic stem cell (HSC) or a hematopoietic stem/progenitor cell (HSPC). In certain embodiments, the target cell is a T cell (e.g., a CD4⁺ T cell, a CD8⁺ T cell, a helper T cell, a regulatory T cell, a cytotoxic T cell, a memory T cell, a T cell precursor or a natural killer T cell), a B cell (e.g., a progenitor B cell, a Pre B cell, a Pro B cell, a memory B cell, a plasma B cell), a monocyte, a megakaryocyte, a neutrophil, an eosinophil, a basophil, a mast cell, a reticulocyte, a lymphoid progenitor cell, a myeloid progenitor cell, or a hematopoietic stem cell, or a hematopoietic progenitor cell. In certain embodiments, the target cell is a bone marrow cell, (e.g., a lymphoid progenitor cell, a myeloid progenitor cell, an erythroid progenitor cell, a hematopoietic stem cell, a hematopoietic progenitor cell, an endothelial cell, or a mesenchymal stem cell). In certain embodiments, the cell is a CD4 cell, a T cell, a gut associated lymphatic tissue (GALT), a macrophage, a dendritic cell, a myeloid precursor cell, or a microglial cell. The contacting may be performed ex vivo and the contacted cell may be returned to the subject's body after the contacting step. In certain embodiments, the contacting step may be performed in vivo.

In certain embodiments, the method of altering a cell as described herein comprises acquiring knowledge of the presence of a CCR5 target position in said cell, prior to the contacting step. Acquiring knowledge of the presence of a CCR5 target position in the cell may be by sequencing the CCR5 gene, or a portion of the CCR5 gene.

In certain embodiments, the method comprises contacting the cell with a nucleic acid composition, e.g., a vector, e.g., an AAV vector, that expresses at least one of (a), (b), and (c). In certain embodiments, the method comprises contacting the cell with a nucleic acid composition, e.g., a vector, e.g., an AAV vector, that encodes each of (a), (b), and (c). In certain embodiments, the method comprises delivering to the cell the Cas9 molecule of (b) and a nucleic acid composition that encodes a gRNA molecule of (a) and optionally, a second gRNA molecule of (c)(i) (and further optionally, a third gRNA molecule of (c)(ii) and/or fourth gRNA molecule of (c)(iii).

In certain embodiments, the method comprises contacting the cell with a nucleic acid composition, e.g., a vector. In certain embodiments, the vector is an AAV vector, e.g., an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43vector, a modified AAV.rh43vector, an AAV.rh64R1vector, and a modified AAV.rh64R1vector, as described herein. In certain embodiments, the vector is a lentivirus, e.g., an IDLV (integration deficienct lentivirus vector).

In certain embodiments, the method comprises delivering to the cell a Cas9 molecule of (b), as a protein or an mRNA, and a nucleic acid composition that encodes a gRNA molecule of (a) and optionally a second, third and/or fourth gRNA molecule of (c). In certain embodiments, the method comprises delivering to the cell a Cas9 molecule of (b), as a protein or an mRNA, said gRNA molecule of (a), as an RNA, and optionally said second, third and/or fourth gRNA molecule of (c), as an RNA. In certain embodiments, the method comprises delivering to the cell a gRNA molecule of (a) as an RNA, optionally the second, third and/or fourth gRNA molecule of (c) as an RNA, and a nucleic acid that encodes the Cas9 molecule of (b). In certain embodiments, the first gRNA molecule, the Cas 9 molecule, and the second gRNA molecule are present together in a genome editing system.

In certain embodiments, the contacting step further comprises contacting the cell with an HSC self-renewal agonist, e.g., UM171 ((I r,4r)-N1-(2-benzyl-7-(2-methyl-2H-tetrazol-5-yl)-9H-pyrimido[4,5-b]indol-4-yl)cyclohexane-1,4-diamine) or a pyrimidoindole derivative described in Fares et al., Science, 2014, 345(6203): 1509-1512). In certain embodiments, the cell is contacted with the HSC self-renewal agonist before (e.g., at least 1, 2, 4, 8, 12, 24, 36, or 48 hours before, e.g., about 2 hours before) the cell is contacted with a gRNA molecule and/or a Cas9 molecule. In certain embodiments, the cell is contacted with the HSC self-renewal agonist after (e.g., at least 1, 2, 4, 8, 12, 24, 36, or 48 hours after, e.g., about 24 hours after) the cell is contacted with a gRNA molecule and/or a Cas9 molecule. In yet certain embodiments, the cell is contacted with the HSC self-renewal agonist before (e.g., at least 1, 2, 4, 8, 12, 24, 36, or 48 hours before) and after (e.g., at least 1, 2, 4, 8, 12, 24, 36, or 48 hours after) the cell is contacted with a gRNA molecule and/or a Cas9 molecule. In certain embodiments, the cell is contacted with the HSC self-renewal agonist about 2 hours before and about 24 hours after the cell is contacted with a gRNA molecule and/or a Cas9 molecule. In certain embodiments, the cell is contacted with the HSC self-renewal agonist at the same time the cell is contacted with a gRNA molecule and/or a Cas9 molecule. In certain embodiments, the HSC self-renewal agonist, e.g., UM171, is used at a concentration between 5 and 200 nM, e.g., between 10 and 100 nM or between 20 and 50 nM, e.g., about 40 nM.

The presently disclosed subject matter further provides for a cell or a population of cells produced (e.g., altered) by a method described herein.

The presently disclosed subject matter further provides for a method of treating a subject suffering from or likely to develop an HIV infection or AIDS, e.g., altering the structure, e.g., sequence, of a target nucleic acid of the subject, comprising contacting the subject (or a cell from the subject) with:

(a) a gRNA molecule that targets the CCR5 gene, e.g., a gRNA disclosed herein;

(b) a Cas9 molecule, e.g., a Cas9 molecule disclosed herein; and

optionally, (c)(i) a second gRNA molecule that targets the CCR5 gene, e.g., a second gRNA disclosed herein, and

further optionally, (c)(ii) a third gRNA molecule, and still further optionally, (c)(iii) a fourth gRNA molecule that target the CCR5 gene, e.g., a third and fourth gRNA disclosed herein.

In certain embodiments, contacting comprises contacting with (a) and (b). In certain embodiments, contacting comprises contacting with (a), (b), and (c)(i). In certain embodiments, contacting comprises contacting with (a), (b), (c)(i) and (c)(ii). In certain embodiments, contacting comprises contacting with (a), (b), (c)(i), (c)(ii) and (c)(iii). In certain embodiments, the method comprises acquiring knowledge of the presence or absence of a mutation at a CCR5 target position in said subject. In certain embodiments, the method comprises acquiring knowledge of the presence or absence of a mutation at a CCR5 target position in said subject by sequencing the CCR5 gene or a portion of the CCR5 gene. In certain embodiments, the method comprises introducing a mutation at a CCR5 target position. In certain embodiments, the method comprises introducing a mutation at a CCR5 target position, e.g., by NHEJ. When the method comprises introducing a mutation at a CCR5 target position, e.g., by NHEJ, in the coding region or a non-coding region, a Cas9 of (b) and at least one guide RNA (e.g., a guide RNA of (a)) are included in the contacting step.

In certain embodiments, a cell of the subject is contacted ex vivo with (a), (b) and optionally (c)(i), further optionally (c)(ii), and still further optionally (c)(iii). In certain embodiments, said cell is returned to the subject's body.

In certain embodiments, a cell of the subject is contacted is in vivo with (a), (b) and optionally (c)(i), further optionally (c)(ii), and still further optionally (c)(iii). In certain embodiments, the cell of the subject is contacted in vivo by intravenous delivery of (a), (b) and optionally (c)(i), further optionally (c)(ii), and still further optionally (c)(iii).

In certain embodiments, the method comprises contacting the subject with a nucleic acid composition, e.g., a vector (e.g., an AAV vector or an DLV vector), described herein, e.g., a nucleic acid composition that encodes at least one of (a), (b), and optionally (c)(i), further optionally (c)(ii), and still further optionally (c)(iii).

In certain embodiments, the method comprises delivering to said subject said Cas9 molecule of (b), as a protein or mRNA, and a nucleic acid composition that encodes (a) and optionally (c)(i), further optionally (c)(ii), and still further optionally (c)(iii).

In certain embodiments, the method comprises delivering to the subject the Cas9 molecule of (b), as a protein or mRNA, said gRNA molecule of (a), as an RNA, and optionally said second gRNA molecule of (c)(i), further optionally said third gRNA molecule of (c)(ii), and still further optionally said fourth gRNA molecule of (c)(iii), as an RNA.

In certain embodiments, the method comprises delivering to the subject the gRNA molecule of (a), as an RNA, optionally said second gRNA molecule of (c)(i), further optionally said third gRNA molecule of (c)(ii), and still further optionally said fourth gRNA molecule of (c)(iii), as an RNA, and a nucleic acid composition that encodes the Cas9 molecule of (b).

The presently disclosed subject matter also provides for a reaction mixture comprising a gRNA molecule, a nucleic acid composition, or a composition described herein, and a cell, e.g., a cell from a subject having, or likely to develop and HIV infection or AIDS, or a subject having a mutation at a CCR5 target position (e.g., a heterozygous carrier of a CCR5 mutation).

The presently disclosed subject matter also provides for a kit comprising, (a) a gRNA molecule described herein, or a nucleic acid composition that encodes the gRNA, and one or more of the following:

(b) a Cas9 molecule, e.g., a Cas9 molecule described herein, or a nucleic acid composition or mRNA that encodes the Cas9;

(c)(i) a second gRNA molecule, e.g., a second gRNA molecule described herein or a nucleic acid composition that encodes (c)(i);

(c)(ii) a third gRNA molecule, e.g., a third gRNA molecule described herein or a nucleic acid composition that encodes (c)(ii);

(c)(iii) a fourth gRNA molecule, e.g., a fourth gRNA molecule described herein or a nucleic acid composition that encodes (c)(iii).

In certain embodiments, the kit comprises a nucleic acid composition, e.g., an AAV vector, that encodes one or more of (a), (b), (c)(i), (c)(ii), and (c)(iii).

The presently disclosed subject matter further provides for a gRNA molecule, e.g., a gRNA molecule described herein, for use in treating, or delaying the onset or progression of, HIV infection or AIDS in a subject, e.g., in accordance with a method of treating, or delaying the onset or progression of, HIV infection or AIDS as described herein. In certain embodiments, the gRNA molecule in used in combination with a Cas9 molecule, e.g., a Cas9 molecule described herein. Additionaly or alternatively, in certain embodiments, the gRNA molecule is used in combination with a second, third and/or fouth gRNA molecule, e.g., a second, third and/or fouth gRNA molecule described herein.

The presently disclosed subject matter further provides for use of a gRNA molecule, e.g., a gRNA molecule described herein, in the manufacture of a medicament for treating, or delaying the onset or progression of, HIV infection or AIDS in a subject, e.g., in accordance with a method of treating, or delaying the onset or progression of, HIV infection or AIDS as described herein. In certain embodiments, the medicament comprises a Cas9 molecule, e.g., a Cas9 molecule described herein. Additionally or alternatively, in certain embodiments, the medicament comprises a second, third and/or fouth gRNA molecule, e.g., a second, third and/or fouth gRNA molecule described herein.

### Alteration of CXCR4

In certain embodiments, the methods, genome editing systems, and compositions discussed herein, inhibit or block a critical aspect of the HIV life cycle, i.e., CXCR4-mediated entry into T cells, i.e., CXCR4-mediated entry into B cells, by alteration (e.g., inactivation) of the CXCR4 gene. Exemplary mechanisms that can be associated with the alteration of the CXCR4 gene include, but are not limited to, non-homologous end joining (NHEJ) (e.g., classical or alternative), microhomology-mediated end joining (MMEJ), homology-directed repair (e.g., endogenous donor template mediated), SDSA (synthesis dependent strand annealing), single strand annealing or single strand invasion. Alteration of the CXCR4 gene, e.g., mediated by NHEJ, can result in a mutation (e.g. a single point mutation), which can comprise a deletion or insertion (indel). The introduced mutation can take place in any region of the CXCR4 gene, e.g., a promoter region or other non-coding region, or a coding region, so long as the mutation results in reduced or loss of the ability to mediate HIV entry into the cell.

In certain embodiments, the methods, genome editing systems, and compositions discussed herein are used to alter the CXCR4 gene to treat or prevent HIV infection or AIDS by targeting the coding sequence of the CXCR4 gene.

In certain embodiments, the gene, e.g., the coding sequence of the CXCR4 gene, is targeted for knocking out, e.g., to eliminate expression of the gene, e.g., to knock out both alleles of the CXCR4 gene, e.g., by introduction of an alteration comprising a mutation (e.g., a single point mutation, an insertion or a deletion) in the CXCR4 gene. This type of alteration is sometimes referred to as “knocking out” the CXCR4 gene. In certain embodiments, a targeted knockout approach is mediated by NHEJ using a CRISPR/Cas system comprising a Cas9 molecule, e.g., an enzymatically active Cas9 (eaCas9) molecule, as described herein.

In certain embodiments, the methods, genome editing systems, and compositions discussed herein are used to alter the CXCR4 gene to treat or prevent HIV infection or AIDS by targeting a non-coding sequence of the CXCR4 gene, e.g., a promoter, an enhancer, an intron, a 5′ UTR, a 3′UTR, and/or a polyadenylation signal.

In certain embodiments, the non-coding sequence of the CXCR4 gene is targeted for knocking out, e.g., to eliminate expression of the gene, e.g., to knock out both alleles of the CXCR4 gene, e.g., by introduction of an alteration comprising a mutation (e.g., a single point mutation, an insertion or/or a deletion) in the CXCR4 gene.

In certain embodiments, the method provides an alteration that comprises, e.g., a single point mutation, an insertion and/or a deletion. This type of alteration is also sometimes referred to as “knocking out” the CXCR4 gene. In certain embodiments, a targeted knockout approach is mediated by NHEJ using a CRISPR/Cas system comprising a Cas9 molecule, e.g., an enzymatically active Cas9 (eaCas9) molecule, as described herein.

In certain embodiments, the methods, genome editing systems, and compositions discussed herein, provide for knocking out the CXCR4 gene. In certain embodiments, knocking out the CXCR4 gene comprises (1) insertion or deletion (e.g., NHEJ-mediated insertion or deletion) of one or more nucleotides of the CXCR4 gene (e.g., in close proximity to or within an early coding region or in a non-coding region), and/or (2) deletion (e.g., NHEJ-mediated deletion) of a genomic sequence of the CXCR4 gene (e.g., in a coding region or in a non-coding region). Both approaches can give rise to alteration (e.g., knockout) of the CXCR4 gene as described herein. In certain embodiments, a CXCR4 target knockout position is altered by genome editing using the CRISPR/Cas9 system. The CXCR4 target knockout position can be targeted by cleaving with either one or more nucleases, or one or more nickases, or a combination thereof.

“CXCR4 target knockout position”, as used herein, refers to a position in the CXCR4 gene, which if altered, e.g., disrupted by insertion or deletion of one or more nucleotides, e.g., by NHEJ-mediated alteration, results in alteration of the CXCR4 gene. In certain embodiments, the position is in the CXCR4 coding region, e.g., an early coding region. In certain embodiments, the position is in a non-coding sequence of the CXCR4 gene, e.g., a promoter, an enhancer, an intron, a 5′ UTR, a 3′UTR, and/or a polyadenylation signal.

In certain embodiments, the CXCR4 gene is targeted for knocking down, e.g., to reduce or eliminate expression of the CXCR4 gene, e.g., to knock down one or both alleles of the CXCR4 gene.

In certain embodiments, the coding region of the CXCR4 gene is targeted to alter the expression of the gene. In certain embodiments, a non-coding region (e.g., an enhancer region, a promoter region, an intron, a 5′ UTR, a 3′UTR, or a polyadenylation signal) of the CXCR4 gene is targeted to alter the expression of the gene. In certain embodiments, the promoter region of the CXCR4 gene is targeted to knock down the expression of the CXCR4 gene. This type of alteration is also sometimes referred to as “knocking down” the CXCR4 gene. In certain embodiments, a targeted knockdown approach is mediated by a CRISPR/Cas system comprising a Cas9 molecule, e.g., an enzymatically inactive Cas9 (eiCas9) molecule or an eiCas9 fusion protein (e.g., an eiCas9 fused to a transcription repressor domain or chromatin modifying protein), as described herein. In certain embodiments, the CXCR4 gene is targeted to alter (e.g., to block, reduce, or decrease) the transcription of the CXCR4 gene. In certain embodiments, the CXCR4 gene is targeted to alter the chromatin structure (e.g., one or more histone and/or DNA modifications) of the CXCR4 gene. In certain embodiments, one or more gRNA molecules comprising a targeting domain are configured to target an enzymatically inactive Cas9 (eiCas9) molecule or an eiCas9 fusion protein (e.g., an eiCas9 fused to a transcription repressor domain), sufficiently close to a CXCR4 target knockdown position to reduce, decrease or repress expression of the CXCR4 gene.

“CXCR4 target knockdown position”, as used herein, refers to a position in the CXCR4 gene, which if targeted, e.g., by an eiCas9 molecule or an eiCas9 fusion described herein, results in reduction or elimination of expression of functional CXCR4 gene product. In certain embodiments, the transcription of the CXCR4 gene is reduced or eliminated. In certain embodiments, the chromatin structure of the CXCR4 gene is altered. In certain embodiments, the position is in the CXCR4 promoter sequence. In certain embodiments, a position in the promoter sequence of the CXCR4 gene is targeted by an enzymatically inactive Cas9 (eiCas9) molecule or an eiCas9 fusion protein, as described herein.

In certain embodiments, the methods, genome editing systems, and compositions discussed herein, provide for introduction of one or more mutations in the CXCR4 gene. In certain embodiments, the introduction is mediated by HDR. In certain embodiments, the one or more mutations comprise one or more single or two base substitutions. In certain embodiments, the one or more mutations disrupt HIV gp1230 binding to CXCR4.

“CXCR4 target position”, as used herein, refers to any position that results in inactivation of the CXCR4 gene. In certain embodiments, a CXCR4 target position comprises a CXCR4 target knockout position, a CXCR4 target knockdown position,or a position within the CXCR4 gene that is targeted for introduction of one or more mutations.

The presently disclosed subject matter provides for a gRNA molecule, e.g., an isolated or non-naturally occurring gRNA molecule, comprising a targeting domain which is complementary with a target domain (also referred to as “target sequence”) from the CXCR4 gene.

In certain embodiments, the targeting domain of the gRNA molecule is configured to provide a cleavage event, e.g., a double strand break or a single strand break, sufficiently close to a CXCR4 target position in the CXCR4 gene to allow alteration, e.g., alteration associated with NHEJ, of a CXCR4 target position in the CXCR4 gene. In certain embodiments, the alteration comprises an insertion or deletion. In certain embodiments, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 300, 400, 450, or 500 nucleotides of a CXCR4 target position. The break, e.g., a double strand or single strand break, can be positioned upstream or downstream of a CXCR4 target position in the CXCR4 gene.

In certain embodiments, a second gRNA molecule comprising a second targeting domain is configured to provide a cleavage event, e.g., a double strand break or a single strand break, sufficiently close to the CXCR4 target position in the CXCR4 gene, to allow alteration, e.g., alteration associated with NHEJ, of the CXCR4 target position in the CXCR4 gene, either alone or in combination with the break positioned by said first gRNA molecule. In certain embodiments, the targeting domains of the first and second gRNA molecules are configured such that a cleavage event, e.g., a double strand or single strand break, is positioned, independently for each of the gRNA molecules, within 1, 2, 3, 4, 5, 10, 15, 20, 25, 30, 35, 40, 45, 50, 60, 70, 80, 90, 100, 150, 200, 300, 400, 450, or 500 nucleotides of the target position. In certain embodiments, the breaks, e.g., double strand or single strand breaks, are positioned on both sides of a nucleotide of a CXCR4 target position in the CXCR4 gene. In certain embodiments, the breaks, e.g., double strand or single strand breaks, are positioned on one side, e.g., upstream or downstream, of a nucleotide of a CXCR4 target position in the CXCR4 gene.

In certain embodiments, a single strand break is accompanied by an additional single strand break, positioned by a second gRNA molecule, as discussed below. For example, the targeting domains are configured such that a cleavage event, e.g., the two single strand breaks, are positioned within 1, 2, 3, 4, 5, 10, 15, 20, 25, 30, 35, 40, 45, 50, 60, 70, 80, 90, 100, 150, 200, 300, 400, 450, or 500 nucleotides of a CXCR4 target position. In certain embodiments, the first and second gRNA molecules are configured such, that when guiding a Cas9 molecule, e.g., a Cas9 nickase, a single strand break can be accompanied by an additional single strand break, positioned by a second gRNA, sufficiently close to one another to result in alteration of a CXCR4 target position in the CXCR4 gene. In certain embodiments, the first and second gRNA molecules are configured such that a single strand break positioned by said second gRNA is within 1, 2, 3, 4, 5, 10, 20, 30, 40, 50, 60, 70, 80, 90, 100, 200, 300, 400, 500, 600, 700, 800, 900, or 1000 nucleotides of the break positioned by said first gRNA molecule, e.g., when the Cas9 molecule is a nickase. In certain embodiments, the two gRNA molecules are configured to position cuts at the same position, or within a few nucleotides of one another, on different strands, e.g., essentially mimicking a double strand break.

In certain embodiments, a double strand break can be accompanied by an additional double strand break, positioned by a second gRNA molecule, as is discussed below. For example, the targeting domain of a first gRNA molecule is configured such that a double strand break is positioned upstream of a CXCR4 target position in the CXCR4 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position; and the targeting domain of a second gRNA molecule is configured such that a double strand break is positioned downstream of a CXCR4 target position in the CXCR4 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position. In certain embodiments, the first and second gRNA molecules are configured such that a double strand break positioned by said second gRNA is within 10, 20, 30, 40, 50, 60, 70, 80, 90, 100, 200, 300, 400, 500, 600, 700, 800, 900, or 1000 nucleotides of the break positioned by said first gRNA molecule.

In certain embodiments, the targeting domains of the first and second gRNA molecules are configured such that a cleavage event, e.g., a single strand break, is positioned, independently for each of the gRNA molecules.

In certain embodiments, a double strand break can be accompanied by two additional single strand breaks, positioned by a second gRNA molecule and a third gRNA molecule. For example, the targeting domain of a first gRNA molecule is configured such that a double strand break is positioned upstream of a CXCR4 target position in the CXCR4 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position; and the targeting domains of a second and third gRNA molecule are configured such that two single strand breaks are positioned downstream of a CXCR4 target position in the CXCR4 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position. In certain embodiments, the first, second and third gRNA molecules are configured such that a single strand break positioned by said second or third gRNA molecule is within 10, 20, 30, 40, 50, 60, 70, 80, 90, 100, 200, 300, 400, 500, 600, 700, 800, 900, or 1000 nucleotides of the break positioned by said first gRNA molecule. In certain embodiments, the targeting domains of the first, second and third gRNA molecules are configured such that a cleavage event, e.g., a double strand or single strand break, is positioned, independently for each of the gRNA molecules.

In certain embodiments, when CXCR4 is targeted for knock out, a first and second single strand breaks can be accompanied by two additional single strand breaks positioned by a third gRNA molecule and a fourth gRNA molecule. For example, the targeting domain of a first and second gRNA molecule are configured such that two single strand breaks are positioned upstream of a CXCR4 target position in the CXCR4 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position; and the targeting domains of a third and fourth gRNA molecule are configured such that two single strand breaks are positioned downstream of a CXCR4 target position in the CXCR4 gene, e.g., within 1, 2, 3, 4, 5, 10, 15, 20, 25, 30, 35, 40, 45, 50, 60, 70, 80, 90, 100, 150, 200, 300, 400, 450, or 500 nucleotides of the target position. In certain embodiments, the first, second, third and fourth gRNA molecules are configured such that the single strand break positioned by said third or fourth gRNA molecule is within 10, 20, 30, 40, 50, 60, 70, 80, 90, 100, 200, 300, 400, 500, 600, 700, 800, 900, or 1000 nucleotides of the break positioned by said first or second gRNA molecule, e.g., when the Cas9 molecule is a nickase. In certain embodiments, the targeting domains of the first, second, third and fourth gRNA molecules are configured such that a cleavage event, e.g., a single strand break, is positioned, independently for each of the gRNA molecules.

In certain embodiments, when multiple gRNAs are used to generate (1) two single stranded breaks in close proximity, (2) two double stranded breaks, e.g., flanking a CXCR4 target position (e.g., to remove a piece of DNA, e.g., a insertion or deletion mutation) or to create more than one indel in an early coding region, (3) one double stranded break and two paired nicks flanking a CXCR4 target position (e.g., to remove a piece of DNA, e.g., a insertion or deletion mutation) or (4) four single stranded breaks, two on each side of a CXCR4 target position, that they are targeting the same CXCR4 target position. In certain embodiments multiple gRNAs may be used to target more than one target position in the same gene.

In certain embodiments, the targeting domain of the first gRNA molecule and the targeting domain of the second gRNA molecules are complementary to opposite strands of the target nucleic acid molecule. In certain embodiments, the gRNA molecule and the second gRNA molecule are configured such that the PAMs are oriented outward.

In certain embodiments, the targeting domain of a gRNA molecule is configured to avoid unwanted target chromosome elements, such as repeat elements, e.g., Alu repeats, in the target domain (also referred to as “target sequence”). The gRNA molecule may be a first, second, third and/or fourth gRNA molecule, as described herein.

In certain embodiments, the targeting domain of a gRNA molecule is configured to position a cleavage event sufficiently far from a preselected nucleotide, e.g., the nucleotide of a coding region, such that the nucleotide is not altered. In certain embodiments, the targeting domain of a gRNA molecule is configured to position an intronic cleavage event sufficiently far from an intron/exon border, or naturally occurring splice signal, to avoid alteration of the exonic sequence or unwanted splicing events. The gRNA molecule may be a first, second, third and/or fourth gRNA molecule, as described herein.

In certain embodiments, a CXCR4 target position is targeted and the targeting domain of a gRNA molecule comprises a nucleotide sequence that is the same as, or differs by no more than 1, 2, 3, 4, or 5 nucleotides from, a nucleotide sequence selected from SEQ ID NOS: 3740 to 8407. In certain embodiments, the targeting domain comprises a nucleotide sequence selected from SEQ ID NOS: 3740 to 8407. In certain embodiments, the targeting domain comprises a nucleotide sequence selected from SEQ ID NOS: 3740 to 5208 and 5241 to 8355. In certain embodiments, the targeting domain comprises a nucleotide sequence independently selected from:

(SEQ ID NO: 3740)

GUUGGUGGCGUGGACGA;

(SEQ ID NO: 3741)

UUGAUGCCGUGGCAAAC;

(SEQ ID NO: 3742)

GGAGGUCGGCCACUGAC;

(SEQ ID NO: 3743)

CAAUGGAUUGGUCAUCC;

(SEQ ID NO: 3744)

UGGUCUAUGUUGGCGUC;

(SEQ ID NO: 3745)

CGCAUCUGGAGAACCAG;

(SEQ ID NO: 3746)

UGGUUCUCCAGAUGCGG;

(SEQ ID NO: 3747)

ACGGCAUCAACUGCCCAGAA;

(SEQ ID NO: 3748)

CCCAAAGUACCAGUUUGCCA;

(SEQ ID NO: 3749)

UGGAUUGGUCAUCCUGGUCA;

(SEQ ID NO: 3750)

GAACCAGCGGUUACCAUGGA;

(SEQ ID NO: 3751)

GUAGCGGUCCAGACUGAUGA;

(SEQ ID NO: 3752)

CAGUUGAUGCCGUGGCAAAC;

(SEQ ID NO: 3753)

AGAGGAGGUCGGCCACUGAC;

(SEQ ID NO: 3754)

GAAGCAUGACGGACAAGUAC;

(SEQ ID NO: 3755)

UCUUCUGGUAACCCAUGACC;

(SEQ ID NO: 3756)

AUCCCCUCCAUGGUAACCGC;

(SEQ ID NO: 3757)

AGGUGGUCUAUGUUGGCGUC;

(SEQ ID NO: 3758)

UUGUCAUCACGCUUCCCUUC;

(SEQ ID NO: 3759)

CACCGCAUCUGGAGAACCAG;

(SEQ ID NO: 3760)

UCCACGCCACCAACAGUCAG;

(SEQ ID NO: 3761)

CACUUCAGAUAACUACACCG;

(SEQ ID NO: 3762)

CUUCUGGGCAGUUGAUGCCG;

(SEQ ID NO: 3763)

GCCUCUGACUGUUGGUGGCG;

(SEQ ID NO: 3764)

GAAGCGUGAUGACAAAGAGG;

(SEQ ID NO: 3765)

CGCUGGUUCUCCAGAUGCGG;

(SEQ ID NO: 3766)

AGAACCAGCGGUUACCAUGG;

(SEQ ID NO: 3767)

AACCGCUGGUUCUCCAGAUG;

(SEQ ID NO: 3768)

GGAUUGGUCAUCCUGGUCAU;

(SEQ ID NO: 3769)

UGUCAUCACGCUUCCCUUCU;

(SEQ ID NO: 3770)

GCUGAAAAGGUGGUCUAUGU;

(SEQ ID NO: 3771)

GCCGUGGCAAACUGGUACUU;
and

(SEQ ID NO: 3772)

CCGUGGCAAACUGGUACUUU.

In certain embodiments, when CXCR4 is targeted for knock out or knock down, more than one gRNA is used to position breaks, e.g., two single stranded breaks or two double stranded breaks, or a combination of single strand and double strand breaks, e.g., to create one or more indels, in the target nucleic acid sequence. In certain embodiments, two, three or four gRNA molecules are used to knockout or knockdown the CCR5 gene.

In certain embodiments, when CXCR4 is targeted for knock out or knock down, the targeting domain of the gRNA molecule is configured to target an enzymatically inactive Cas9 (eiCas9) molecule or an eiCas9 fusion protein (e.g., an eiCas9 fused to a transcription repressor domain), sufficiently close to a CXCR4 transcription start site (TSS) to reduce (e.g., block) transcription, e.g., transcription initiation or elongation, binding of one or more transcription enhancers or activators, and/or RNA polymerase. In certain embodiments, the targeting domain is configured to target between 1000 bp upstream and 1000 bp downstream (e.g., between 500 bp upstream and 1000 bp downstream, between 1000 bp upstream and 500 bp downstream, between 500 bp upstream and 500 bp downstream, within 500 bp or 200 bp upstream, or within 500 bp or 200 bp downstream) of the TSS of the CXCR4 gene. One or more gRNAs may be used to target an eiCas9 to the promoter region of the CXCR4 gene.

In certain embodiments, the CXCR4 gene is targeted for knockout, the targeting domain of the gRNA molecule can comprise a nucleotide sequence that is the same as, or differs by no more than 1, 2, 3, 4, or 5 nucleotides from, a nucleotide sequence selected from SEQ ID NOS: 3740 to 5240. In certain embodiments, the targeting domain comprises a nucleotide sequence selected from SEQ ID NOS: 3740 to 5240. In certain embodiments, the targeting domain comprises a nucleotide sequence selected from SEQ ID NOS: 3740 to 5208. In certain embodiments, the targeting domain comprises a nucleotide sequence selected from SEQ ID NOS: 3973, 4118, and 4604. In certain embodiments, the targeting domain comprises a nucleotide sequence selected from SEQ ID NOS: 3740 to 3772. In certain embodiments, the targeting domain comprises a nucleotide sequence selected from SEQ ID NOS: 4064 to 4125. In certain embodiments, the targeting domain comprises a nucleotide sequence selected from SEQ ID NOS: 5209 to 5219.

In certain embodiments, the CXCR4 gene is targeted for knockdown, and the targeting domain of the gRNA molecule can comprise a nucleotide sequence that is the same as, or differs by no more than 1, 2, 3, 4, or 5 nucleotides from, a nucleotide sequence selected from SEQ ID NOS: 5241 to 8407. In certain embodiments, the targeting domain comprises a nucleotide sequence selected from SEQ ID NOS: 5241 to 8407. In certain embodiments, the targeting domain comprises a nucleotide sequence selected from SEQ ID NOS: 5241 to 8355. In certain embodiments, the targeting domain comprises a nucleotide sequence selected from SEQ ID NOS: 5241 to 5349. In certain embodiments, the targeting domain comprises a nucleotide sequence selected from SEQ ID NOS: 5921 to 6046. In certain embodiments, the targeting domain comprises a nucleotide sequence selected from SEQ ID NOS: 8356 to 8377.

In certain embodiments, the CXCR4 target knockdown position is the promoter region of the CXCR4 gene. In certain embodiments, when the CXCR4 target knockdown position is the CXCR4 promoter region and more than one gRNA is used to position an eiCas9 molecule or an eiCas9-fusion protein (e.g., an eiCas9-transcription repressor domain fusion protein), in the target nucleic acid sequence, the targeting domain for each guide RNA comprises a nucleotide sequence selected from SEQ ID NOS: 5241 to 8407.

In certain embodiments, the targeting domain which is complementary with a target domain (also referred to as “target sequence”) from the CXCR4 target position in the CXCR4 gene is 16 nucleotides or more in length. In certain embodiments, the targeting domain is 16 nucleotides in length. In certain embodiments, the targeting domain is 17 nucleotides in length. In other embodiments, the targeting domain is 18 nucleotides in length. In still other embodiments, the targeting domain is 19 nucleotides in length. In still other embodiments, the targeting domain is 20 nucleotides in length. In certain embodiments, the targeting domain is 21 nucleotides in length. In certain embodiments, the targeting domain is 22 nucleotides in length. In certain embodiments, the targeting domain is 23 nucleotides in length. In certain embodiments, the targeting domain is 24 nucleotides in length. In certain embodiments, the targeting domain is 25 nucleotides in length. In certain embodiments, the targeting domain is 26 nucleotides in length.

In certain embodiments, the targeting domain comprises 16 nucleotides. In certain embodiments, the targeting domain comprises 17 nucleotides. In certain embodiments, the targeting domain comprises 18 nucleotides. In certain embodiments, the targeting domain comprises 19 nucleotides. In certain embodiments, the targeting domain comprises 20 nucleotides. In certain embodiments, the targeting domain comprises 21 nucleotides. In certain embodiments, the targeting domain comprises 22 nucleotides. In certain embodiments, the targeting domain comprises 23 nucleotides. In certain embodiments, the targeting domain comprises 24 nucleotides. In certain embodiments, the targeting domain comprises 25 nucleotides. In certain embodiments, the targeting domain comprises 26 nucleotides.

A gRNA as described herein may comprise from 5′ to 3′: a targeting domain (comprising a “core domain”, and optionally a “secondary domain”); a first complementarity domain; a linking domain; a second complementarity domain; a proximal domain; and a tail domain. In certain embodiments, the proximal domain and tail domain are taken together as a single domain.

In certain embodiments, a gRNA comprises a linking domain of no more than 25 nucleotides in length; a proximal and tail domain, that taken together, are at least 20, at least 25, at least 30, at least 35, or at least 40 nucleotides in length; and a targeting domain equal to or greater than 16, 17, 18, 19, 20, 21, 22, 23, 24, 25 or 26 nucleotides in length.

A cleavage event, e.g., a double strand or single strand break, is generated by a Cas9 molecule. The Cas9 molecule may be an enzymatically active Cas9 (eaCas9) molecule, e.g., an eaCas9 molecule that forms a double strand break in a target nucleic acid or an eaCas9 molecule forms a single strand break in a target nucleic acid (e.g., a nickase molecule).

In certain embodiments, the eaCas9 molecule catalyzes a double strand break.

In certain embodiments, the eaCas9 molecule comprises HNH-like domain cleavage activity but has no, or no significant, N-terminal RuvC-like domain cleavage activity. In this case, the eaCas9 molecule is an HNH-like domain nickase, e.g., the eaCas9 molecule comprises a mutation at D10, e.g., D10A. In other embodiments, the eaCas9 molecule comprises N-terminal RuvC-like domain cleavage activity but has no, or no significant, HNH-like domain cleavage activity. In certain embodiments, the eaCas9 molecule is an N-terminal RuvC-like domain nickase, e.g., the eaCas9 molecule comprises a mutation at H840, e.g., H840A. In certain embodiments, the eaCas9 molecule is an N-terminal RuvC-like domain nickase, e.g., the eaCas9 molecule comprises a mutation at N863, e.g., N863A. In certain embodiments, the eaCas9 molecule is an N-terminal RuvC-like domain nickase, e.g., the eaCas9 molecule comprises a mutation at N580, e.g., N580A.

In certain embodiments, a single strand break is formed in the strand of the target nucleic acid to which the targeting domain of said gRNA is complementary. In certain embodiments, a single strand break is formed in the strand of the target nucleic acid other than the strand to which the targeting domain of said gRNA is complementary.

The presently disclosed subject matter provides for a nucleic acid composition, e.g., an isolated or non-naturally occurring nucleic acid, e.g., DNA, that comprises (a) a first nucleotide equence that encodes a first gRNA molecule comprising a targeting domain that is complementary with a CXCR4 target position in the CXCR4 gene as disclosed herein.

In certain embodiments, the first gRNA molecule comprises a targeting domain configured to provide a cleavage event, e.g., a double strand break or a single strand break, sufficiently close to a CXCR4 target position in the CXCR4 gene to allow alteration, e.g., alteration associated with NHEJ, of a CXCR4 target position in the CXCR4 gene. In certain embodiments, the first gRNA molecule comprises a targeting domain configured to target an enzymatically inactive Cas9 (eiCas9) molecule or an eiCas9 fustion protein (e.g., an eiCas9 fused to a transcription repressor domain or chromatin modifying protein), sufficiently close to a CXCR4 knockdown target position to reduce, decrease or repress expression of the CXCR4 gene. In certain embodiments, the first gRNA molecule comprises a targeting domain comprising a nucleotide sequence that is the same as, or differs by no more than 1, 2, 3, 4, or 5 nucleotides from, a nucleotide sequence selected from SEQ ID NOS: 3740 to 8407, SEQ ID NOS: 3740 to 5240, or SEQ ID NOS: 5241 to 8407. In certain embodiments, the first gRNA molecule comprises a targeting domain that comprises a nucleotide sequence selected from SEQ ID NOS: 3740 to 8407, SEQ ID NOS: 3740 to 5240, or SEQ ID NOS: 5241 to 8407.

In certain embodiments, the nucleic acid composition further comprises (b) a second nucleotide sequence that encodes a Cas9 molecule.

In certain embodiments, the Cas9 molecule is a nickase molecule, an enzymatically active Cas9 (eaCas9) molecule, e.g., an eaCas9 molecule that forms a double strand break in a target nucleic acid and/or an eaCas9 molecule that forms a single strand break in a target nucleic acid. In certain embodiments, a single strand break is formed in the strand of the target nucleic acid to which the targeting domain of said gRNA is complementary. In certain embodiments, a single strand break is formed in the strand of the target nucleic acid other than the strand to which to which the targeting domain of said gRNA is complementary. In certain embodiments, the eaCas9 molecule catalyzes a double strand break.

In certain embodiments, the eaCas9 molecule comprises HNH-like domain cleavage activity but has no, or no significant, N-terminal RuvC-like domain cleavage activity. In certain embodiments, the said eaCas9 molecule is an HNH-like domain nickase, e.g., the eaCas9 molecule comprises a mutation at D10, e.g., D10A. In certain embodiments, the eaCas9 molecule comprises N-terminal RuvC-like domain cleavage activity but has no, or no significant, HNH-like domain cleavage activity. In certain embodiments, the eaCas9 molecule is an N-terminal RuvC-like domain nickase, e.g., the eaCas9 molecule comprises a mutation at H840, e.g., H840A. In certain embodiments, the eaCas9 molecule is an N-terminal RuvC-like domain nickase, e.g., the eaCas9 molecule comprises a mutation at N863, e.g., N863A. In certain embodiments, the eaCas9 molecule is an N-terminal RuvC-like domain nickase, e.g., the eaCas9 molecule comprises a mutation at N580, e.g., N580A.

In certain embodiments, the Cas9 molecule is an enzymatically active Cas9 (eaCas9) molecule. In certain embodiments, the Cas9 molecule is an enzymatically inactive Cas9 (eiCas9) molecule or a modified eiCas9 molecule, e.g., the eiCas9 molecule is fused to Kruppel-associated box (KRAB) to generate an eiCas9-KRAB fusion protein molecule.

In certain embodiments, the nucleic acid composition further comprises (c)(i) a third nucleotide sequence that encodes a second gRNA molecule described herein having a targeting domain that is complementary to a second target domain of the CXCR4 gene, and optionally, (c)(ii) a fourth nucleotide sequence that encodes a third gRNA molecule described herein having a targeting domain that is complementary to a third target domain of the CXCR4 gene; and optionally, (c)(iii) a fifth nucleotide sequence that encodes a fourth gRNA molecule described herein having a targeting domain that is complementary to a fourth target domain of the CXCR4 gene.

In certain embodiments, the second gRNA molecule comprises a targeting domain configured to provide a cleavage event, e.g., a double strand break or a single strand break, sufficiently close to a CXCR4 target position in the CXCR4 gene, to allow alteration, e.g., alteration associated with NHEJ, of a CXCR4 target position in the CXCR4 gene, either alone or in combination with the break positioned by said first gRNA molecule. In certain embodiments, the second gRNA molecule comprises a targeting domain configured to target an enzymatically inactive Cas9 (eiCas9) molecule or an eiCas9 fustion protein (e.g., an eiCas9 fused to a transcription repressor domain or chromatin modifying protein), sufficiently close to a CXCR4 knockdown target position to reduce, decrease or repress expression of the CXCR4 gene.

In certain embodiments, the third gRNA molecule comprises a targeting domain configured to provide a cleavage event, e.g., a double strand break or a single strand break, sufficiently close to a CXCR4 target position in the CXCR4 gene to allow alteration, e.g., alteration associated with NHEJ, of a CXCR4 target position in the CXCR4 gene, either alone or in combination with the break positioned by the first and/or second gRNA molecule.

In certain embodiments, the third gRNA molecule comprises a targeting domain configured to target an enzymatically inactive Cas9 (eiCas9) molecule or an eiCas9 fustion protein (e.g., an eiCas9 fused to a transcription repressor domain or chromatin remodeling protein), sufficiently close to a CXCR4 knockdown target position to reduce, decrease or repress expression of the CXCR4 gene.

In certain embodiments, the fourth gRNA molecule comprises a targeting domain configured to provide a cleavage event, e.g., a double strand break or a single strand break, sufficiently close to a CXCR4 target position in the CXCR4 gene to allow alteration, e.g., alteration associated with NHEJ, of a CXCR4 target position in the CXCR4 gene, either alone or in combination with the break positioned by the first gRNA molecule, the second gRNA molecule and/or the third gRNA molecule.

In certain embodiments, the second gRNA targets the same CXCR4 target position as the first gRNA molecule. In certain embodiments, the third gRNA molecule and the fourth gRNA molecule target the same CXCR4 target position as the first and second gRNA molecules.

In certain embodiments, the targeting domain of each of the second, third, and fourth gRNA molecules comprise a nucleotide sequence that is the same as, or differs by no more than 1, 2, 3, 4, or 5 nucleotides from, a nucleotide sequence selected from from SEQ ID NOS: 3740 to 8407, SEQ ID NOS: 3740 to 5240, or SEQ ID NOS: 5241 to 8407. In certain embodiments, the targeting domain of each of the second, third, and fourth gRNA molecules comprise a nucleotide sequence selected from from SEQ ID NOS: 3740 to 8407, SEQ ID NOS: 3740 to 5240, or SEQ ID NOS: 5241 to 8407.

When multiple gRNAs are used, any combination of modular or chimeric gRNAs may be used.

In certain embodiments, the first gRNA of (a) and the Cas9 molecule of (b) are present on one nucleic acid molecule, e.g., one vector, e.g., one viral vector, e.g., one AAV vector. In certain embodiments, the nucleic acid molecule is an AAV vector. Exemplary AAV vectors that may be used in any of the described compositions and methods include an AAV1 vector, a modified AAV1 vector, an AAV2 vector, a modified AAV2 vector, an AAV3 vector, an AAV4 vector, a modified AAV4 vector, an AAV5 vector, a modified AAV5 vector, a modified AAV3 vector, an AAV6 vector, a modified AAV6 vector, an AAV8 vector an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector. In certain embodiments, the nucleic acid molecule is a lentiviral vector, e.g., an IDLV vector.

In certain embodiments, (a) is present on a first nucleic acid molecule, e.g. a first vector, e.g., a first viral vector, e.g., a first AAV vector; and (b) is present on a second nucleic acid molecule, e.g., a second vector, e.g., a second vector, e.g., a second AAV vector. The first and second nucleic acid molecules may be AAV vectors.

In certain embodiments, the first gRNA molecule of (a), the Cas9 molecule of (b), the second gRNA molecule of (c)(i), optoinally the third gRNA molecule of (c)(ii) and the fourth gRNA molecule of (c)(iii) are present on one nucleic acid molecule, e.g., one vector, e.g., one viral vector, e.g., one AAV vector. In certain embodiments, the nucleic acid molecule is an AAV vector.

In certain embodiments, (a) and (c)(i) are present on different vectors. For example, (a) may be present on a first nucleic acid molecule, e.g. a first vector, e.g., a first viral vector, e.g., a first AAV vector; and (c)(i) may be present on a second nucleic acid molecule, e.g., a second vector, e.g., a second vector, e.g., a second AAV vector. In certain embodiments, the first and second nucleic acid molecules are AAV vectors.

In certain embodiments, each of (a), (b), and (c)(i) are present on one nucleic acid molecule, e.g., one vector, e.g., one viral vector, e.g., an AAV vector. In certain embodiments, the nucleic acid molecule is an AAV vector. In certain embodiments, one of (a), (b), and (c)(i) is encoded on a first nucleic acid molecule, e.g., a first vector, e.g., a first viral vector, e.g., a first AAV vector; and a second and third of (e), (f), and (g)(i) is encoded on a second nucleic acid molecule, e.g., a second vector, e.g., a second vector, e.g., a second AAV vector. The first and second nucleic acid molecule may be AAV vectors.

In certain embodiments, (a) is present on a first nucleic acid molecule, e.g., a first vector, e.g., a first viral vector, a first AAV vector; and (b) and (c)(i) are present on a second nucleic acid molecule, e.g., a second vector, e.g., a second vector, e.g., a second AAV vector. The first and second nucleic acid molecule may be AAV vectors.

In certain embodiments, (b) is present on a first nucleic acid molecule, e.g., a first vector, e.g., a first viral vector, e.g., a first AAV vector; and (a) and (c)(i) are present on a second nucleic acid molecule, e.g., a second vector, e.g., a second vector, e.g., a second AAV vector. The first and second nucleic acid molecule may be AAV vectors.

In certain embodiments, (c)(i) is present on a first nucleic acid molecule, e.g., a first vector, e.g., a first viral vector, e.g., a first AAV vector; and (a) and (b) are present on a second nucleic acid molecule, e.g., a second vector, e.g., a second vector, e.g., a second AAV vector. The first and second nucleic acid molecule may be AAV vectors.

In certain embodiments, each of (a), (b) and (c)(i) are present on different nucleic acid molecules, e.g., different vectors, e.g., different viral vectors, e.g., different AAV vector. For example, (a) may be on a first nucleic acid molecule, (b) on a second nucleic acid molecule, and (c)(i) on a third nucleic acid molecule. The first, second and third nucleic acid molecule may be AAV vectors.

In certain embodiments, when a third and/or fourth gRNA molecule are present, each of (a), (b), (c)(i), (c)(ii) and (c)(iii) may be present on the same nucleic acid molecule, e.g., the same vector, e.g., the same viral vector, e.g., an AAV vector. In certain embodiments, the nucleic acid molecule is an AAV vector. In an alternate embodiment, each of (a), (b), (c)(i), (c)(ii) and (c)(iii) may be present on the different nucleic acid molecules, e.g., different vectors, e.g., the different viral vectors, e.g., different AAV vectors. In a further embodiment, each of (a), (b), (c)(i), (c)(ii) and (c)(iii) may be present on more than one nucleic acid molecule, but fewer than five nucleic acid molecules, e.g., AAV vectors.

The nucleic acid composition may comprise a promoter operably linked to the first nucleotide sequence that encodes the first gRNA molecule of (a), e.g., a promoter described herein. The nucleic acid composition may further comprise a second promoter operably linked to the third nucleotide sequence that encodes the second gRNA molecule of (c)(i), e.g., a promoter described herein. The promoter and second promoter differ from one another. In certain embodiments, the promoter and second promoter are the same.

The nucleic acid composition described herein may further comprise a promoter operably linked to the second sequence that encodes the Cas9 molecule of (f), e.g., a promoter described herein.

The presently disclosed subject matter also provides for a composition comprising (a) a gRNA molecule comprising a targeting domain that is complementary with a target domain (also referred to as “target sequence”) in the CXCR4 gene, as described herein. The composition may further comprise (b) a Cas9 molecule, e.g., a Cas9 molecule as described herein. The composition may further comprise (c)(i) a second gRNA molecule, as described herein. The composition may further comprise (c)(ii) a third gRNA molecule, and (c)(iii) a fourth gRNA molecule, as described herein. In certain embodiments, the composition is a pharmaceutical composition. The compositions described herein, e.g., pharmaceutical compositions described herein, can be used in the treatment or prevention of HIV or AIDS in a subject, e.g., in accordance with a method disclosed herein.

The presently disclosed subject matter further provides for a method of altering a cell, e.g., altering the structure, e.g., altering the sequence, of a target nucleic acid of a cell, comprising contacting said cell with: (a) a gRNA that targets the CXCR4 gene, e.g., a gRNA as described herein; (b) a Cas9 molecule, e.g., a Cas9 molecule as described herein; and optionally, (c)(i) a second gRNA that targets CXCR4 gene, as described herein. In certain embodiments, the method comprises contacting said cell with (c)(ii) a third gRNA molecule, and (c)(iii) a fourth gRNA molecule, as described herein.

In certain embodiments, the method comprises contacting said cell with (a) and (b). In certain embodiments, the method comprises contacting said cell with (a), (b), and (c)(ii). In certain embodiments, the cell is from a subject suffering from or likely to develop an HIV infection or AIDS. The cell may be from a subject who does not have a mutation at a CXCR4 target position.

In certain embodiments, the cell being contacted in the disclosed method is a target cell from a circulating blood cell, a progenitor cell, or a stem cell, e.g., a hematopoietic stem cell (HSC) or a hematopoietic stem/progenitor cell (HSPC). In certain embodiments, the target cell is a T cell (e.g., a CD4+ T cell, a CD8+ T cell, a helper T cell, a regulatory T cell, a cytotoxic T cell, a memory T cell, a T cell precursor or a natural killer T cell), a B cell (e.g., a progenitor B cell, a Pre B cell, a Pro B cell, a memory B cell, a plasma B cell), a monocyte, a megakaryocyte, a neutrophil, an eosinophil, a basophil, a mast cell, a reticulocyte, a lymphoid progenitor cell, a myeloid progenitor cell, a hematopoietic stem cell, or a hematopoietic progenitor cell. In certain embodiments, the target cell is a bone marrow cell, (e.g., a lymphoid progenitor cell, a myeloid progenitor cell, an erythroid progenitor cell, a hematopoietic stem cell, a hematopoietic progenitor cell, an endothelial cell or a mesenchymal stem cell). In certain embodiments, the cell is a CD4 cell, a T cell, a gut associated lymphatic tissue (GALT), a macrophage, a dendritic cell, a myeloid precursor cell, or a microglial cell. The contacting may be performed ex vivo and the contacted cell may be returned to the subject's body after the contacting step. In certain embodiments, the contacting step may be performed in vivo.

In certain embodiments, the method of altering a cell as described herein comprises acquiring knowledge of the presence of a CXCR4 target position in said cell, prior to the contacting step. Acquiring knowledge of the presence of a CXCR4 target position in the cell may be by sequencing the CXCR4 gene, or a portion of the CXCR4 gene.

In certain embodiments, the method comprises contacting the cell with a nucleic acid composition, e.g., a vector, e.g., an AAV vector, that expresses at least one of (a), (b), and (c)(i). In certain embodiments, the method comprises contacting the cell with a nucleic acid composition, e.g., a vector, e.g., an AAV vector, that encodes each of (a), (b), and (c)(i). In certain embodiments, the method comprises delivering to the cell a Cas9 molecule of (f) and a nucleic acid composition that encodes a gRNA molecule of (a) and optionally, a second gRNA molecule of (c)(i) (and further optionally, a third gRNA molecule of (c)(ii) and/or fourth gRNA molecule of (c)(iii).

In certain embodiments, the method comprises contacting the cell with a nucleic acid composition, e.g., a vector. In certain embodiments, the vector is, an AAV vector, e.g., an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43vector, a modified AAV.rh43vector, an AAV.rh64R1vector, or a modified AAV.rh64R1vector, as described herein. In certain embodiments, the vector is a lentiviral vector, e.g., an IDLV vector.

In certain embodiments, the method comprises delivering to the cell a Cas9 molecule of (b), as a protein or an mRNA, and a nucleic acid composition that encodes a gRNA molecule of (a) and optionally a second, third and/or fourth gRNA molecule of (c)(i), (c)(ii), and/or (c)(iii). In certain embodiments, the method comprises delivering to the cell a Cas9 molecule of (b), as a protein or an mRNA, said gRNA molecule of (a), as an RNA, and optionally said second, third and/or fourth gRNA molecule of(c)(i), (c)(ii), and/or (c)(iii), as an RNA. In certain embodiments, the method comprises delivering to the cell a gRNA molecule of (a) as an RNA, optionally the second, third and/or fourth gRNA molecule of (c)(i), (c)(ii), and/or (c)(iii) as an RNA, and a nucleic acid composition that encodes the Cas9 molecule of (b).

In certain embodiments, the contacting step further comprises contacting the cell with an HSC self-renewal agonist, e.g., UM171 (1r,4r)-N1-)2-benzyl-7-(2-methyl-2H-tetrazol-5-yl)-9H-pyrimido[4,5-b]indol-4-yl)cyclohexane-1,4-diamine) or a pyrimidoindole derivative described in Fares et at, Science, 2014. 345(6203): 1509-1512). In certain embodiments, the cell is contacted with the HSC self-renewal agonist before (e.g., at least 1, 2, 4, 8, 12, 24, 36, or 48 hours before, e.g., about 2 hours before) the cell is contacted with a gRNA molecule and/or a Cas9 molecule. In certain embodiments, the cell is contacted with the HSC self-renewal agonist after (e.g., at least 1, 2, 4, 8, 12, 24, 36, or 48 hours after, e.g., about 24 hours after) the cell is contacted with a gRNA molecule and/or a Cas9 molecule. In yet certain embodiments, the cell is contacted with the HSC self-renewal agonist before (e.g., at least 1, 2, 4, 8, 12, 24, 36, or 48 hours before) and after (e.g., at least 1, 2, 4, 8, 12, 24, 36, or 48 hours after) the cell is contacted with a gRNA molecule and/or a Cas9 molecule. In certain embodiments, the cell is contacted with the HSC self-renewal agonist about 2 hours before and about 24 hours after the cell is contacted with a gRNA molecule and/or a Cas9 molecule. In certain embodiments, the cell is contacted with the HSC self-renewal agonist at the same time the cell is contacted with a gRNA molecule and/or a Cas9 molecule. In certain embodiments, the HSC self-renewal agonist, e.g., UM171, is used at a concentration between 5 and 200 nM, e.g., between 10 and 100 nM or between 20 and 50 nM, e.g., about 40 nM.

The presently disclosed subject matter further provides for a cell or a population of cells produced (e.g., altered) by a method described herein.

The presently disclosed subject matter further provides for a method of treating a subject suffering from or likely to develop an HIV infection or AIDS, e.g., altering the structure, e.g., sequence, of a target nucleic acid of the subject, comprising contacting the subject (or a cell from the subject) with:

(a) a gRNA molecule that targets the CXCR4 gene, e.g., a gRNA disclosed herein;

(b) a Cas9 molecule, e.g., a Cas9 molecule disclosed herein; and

optionally, (c)(i) a second gRNA molecule that targets the CXCR4 gene, e.g., a second gRNA disclosed herein, and

further optionally, (c)(ii) a third gRNA, and still further optionally, (c)(iii) a fourth gRNA that target the CXCR4 gene, e.g., a third and fourth gRNA disclosed herein.

In certain embodiments, contacting comprises contacting with (a) and (b). In certain embodiments, contacting comprises contacting with (a), (b), and (c)(i). In certain embodiments, contacting comprises contacting with (a), (b), and (c)(i) and (c)(ii). In certain embodiments, contacting comprises contacting with (a), (b), and (c)(i), (c)(ii) and (c)(iii).

In certain embodiments, the method comprises acquiring knowledge of the presence or absence of a mutation at a CXCR4 target position in said subject. In certain embodiments, the method comprises acquiring knowledge of the presence or absence of a mutation at a CXCR4 target position in said subject by sequencing the CXCR4 gene or a portion of the CXCR4 gene. In certain embodiments, the method comprises introducing a mutation at a CXCR4 target position. In certain embodiments, the method comprises introducing a mutation at a CXCR4 target position by NHEJ. When the method comprises introducing a mutation at a CXCR4 target position, e.g., by NHEJ in the coding region or a non-coding region, a Cas9 of (b) and at least one guide RNA (e.g., a guide RNA of (a)) are included in the contacting step.

In certain embodiments, a cell of the subject is contacted ex vivo with (a), (b) and optionally (c)(i), further optionally (c)(ii), and still further optionally (c)(iii). In certain embodiments, said cell is returned to the subject's body.

In certain embodiments, a cell of the subject is contacted is in vivo with (a), (b) and optionally (c)(i), further optionally (c)(ii), and still further optionally (c)(iii). In certain embodiments, the cell of the subject is contacted in vivo by intravenous delivery of (e), (f) and optionally (g)(i), further optionally (c)(ii), and still further optionally (c)(iii).

In certain embodiments, the contacting step comprises contacting the subject with a nucleic acid, e.g., a vector, e.g., an AAV vector, described herein, e.g., a nucleic acid that encodes at least one of (a), (b) and optionally (c)(i), further optionally (c)(ii), and still further optionally (c)(iii).

In certain embodiments, the contacting step comprises delivering to said subject said Cas9 molecule of (b), as a protein or mRNA, and a nucleic acid which encodes (a) and optionally (c)(i), further optionally (g)(ii), and still further optionally (c)(iii).

In certain embodiments, the contacting step comprises delivering to the subject the Cas9 molecule of (b), as a protein or mRNA, said gRNA molecule of (a), as an RNA, and optionally said second gRNA molecule of (c)(i), further optionally said third gRNA molecule of (c)(ii), and still further optionally said fourth gRNA molecule of (c)(iii), as an RNA.

In certain embodiments, the contacting step comprises delivering to the subject the gRNA molecule of (a), as an RNA, optionally said second gRNA molecule of (c)(i), further optionally said third gRNA molecule of (c)(ii), and still further optionally said fourth gRNA molecule of (c)(iii), as an RNA, and a nucleic acid that encodes the Cas9 molecule of (b).

The presently disclosed subject matter further provides for a reaction mixture comprising a gRNA molecule, a nucleic acid, or a composition described herein, and a cell, e.g., a cell from a subject having, or likely to develop and HIV infection or AIDS, or a subject having a mutation at a CXCR4 target position (e.g., a heterozygous carrier of a CXCR4 mutation).

The presently disclosed subject matter further provides for a kit comprising, (a) a gRNA molecule described herein, or a nucleic acid that encodes the gRNA, and one or more of the following:

(b) a Cas9 molecule, e.g., a Cas9 molecule described herein, or a nucleic acid or mRNA that encodes the Cas9;

(c)(i) a second gRNA molecule, e.g., a second gRNA molecule described herein or a nucleic acid that encodes (c)(i);

(c)(ii) a third gRNA molecule, e.g., a third gRNA molecule described herein or a nucleic acid that encodes (c)(ii);

(c)(iii) a fourth gRNA molecule, e.g., a fourth gRNA molecule described herein or a nucleic acid that encodes (c)(iii).

In certain embodiments, the kit comprises a nucleic acid, e.g., an AAV vector, that encodes one or more of (a), (b), (c)(i), (c)(ii), and (c)(iii).

The presently disclosed subject matter further provides for a gRNA molecule, e.g., a gRNA molecule described herein, for use in treating, or delaying the onset or progression of, HIV infection or AIDS in a subject, e.g., in accordance with a method of treating, or delaying the onset or progression of, HIV infection or AIDS as described herein. In certain embodiments, the gRNA molecule in used in combination with a Cas9 molecule, e.g., a Cas9 molecule described herein. Additionaly or alternatively, in certain embodiments, the gRNA molecule is used in combination with a second, third and/or fouth gRNA molecule, e.g., a second, third and/or fouth gRNA molecule described herein.

The presently disclosed subject matter further provides for use of a gRNA molecule, e.g., a gRNA molecule described herein, in the manufacture of a medicament for treating, or delaying the onset or progression of, HIV infection or AIDS in a subject, e.g., in accordance with a method of treating, or delaying the onset or progression of, HIV infection or AIDS as described herein. In certain embodiments, the medicament comprises a Cas9 molecule, e.g., a Cas9 molecule described herein. Additionally or alternatively, in certain embodiments, the medicament comprises a second, third and/or fouth gRNA molecule, e.g., a second, third and/or fouth gRNA molecule described herein.

### Alteration of CCR5 and CXCR4

In certain embodiments, the methods, genome editing systems, and compositions discussed herein, inhibit or block critical aspects of the HIV life cycle, i.e., CCR5 and CXCR4-mediated entry into T cells, i.e., CCR5 and CXCR4-mediated entry into B cells, by alteringboth CCR5 gene and the CXCR4 gene. Exemplary mechanisms that can be associated with the alteration of the CCR5 gene and the CXCR4 gene include, but are not limited to, non-homologous end joining (NHEJ) (e.g., classical or alternative), microhomology-mediated end joining (MMEJ), homology-directed repair (e.g., endogenous donor template mediated), SDSA (synthesis dependent strand annealing), single strand annealing or single strand invasion. Alteration of both the CCR5 gene and the CXCR4 gene, e.g., mediated by NHEJ, can result in mutations, which typically comprise a deletion or insertion (indel). The introduced mutations can take place in any region of the CCR5 gene and in any region of the CXCR4 gene, e.g., a non-coding region (e.g., a promoter region, an enhancer region, a promoter region, an intron, a 5′ UTR, a 3′UTR, or a polyadenylation signal), or a coding region. In certain embodiments, the mutations result in reduced or loss of the ability to mediate HIV entry into the cell.

In certain embodiments, the methods, genome editing systems, and compositions discussed herein may be used to alter both the CCR5 gene and the CXCR4 gene to treat or prevent HIV infection or AIDS by targeting the coding sequences of both the CCR5 gene and the CXCR4 gene.

The methods, genome editing systems, and compositions described herein that alter the CCR5 gene, e.g., knock out, knock down or introduce one or more mutations (e.g., one or more protective mutations) in the CCR5 gene can be combined with the methods, genome editing systems, and compositions described herein that alter the CXCR4 gene, e.g., knock out, knock down or introduce one or more mutations (e.g., one or more single or two base substitutions) in the CXCR4 gene. In certain embodiments, both the CCR5 gene and the CXCR4 gene are knocked out. In certain embodiments, both the CCR5 gene and the CXCR4 gene are knocked down. In certain embodiments, the CCR5 gene is knocked down and the CXCR4 gene is knocked out. In certain embodiments, the CCR5 gene is knocked out and the CXCR4 gene is knocked down. In certain embodiments, one or more mutations (e.g., one or more protective mutations) are introduced in the CCR5 gene and the CXCR4 gene is knocked out. In certain embodiments, one or more mutations (e.g., one or more protective mutations) are introduced in the CCR5 gene and the CXCR4 gene is knocked down. In certain embodiments, one or more mutations (e.g., one or more single or two base substitutions) are introduced in the CXCR4 gene and the CCR5 gene is knocked out. In certain embodiments, one or more mutations (e.g., one or more single or two base substitutions) are introduced in the CXCR4 gene and the CCR5 gene is knocked down. In certain embodiments, one or more mutations (e.g., one or more protective mutations) are induced in the CCR5 gene and one or more mutations (e.g., one or more single or two base substitutions) are introduced in the CXCR4 gene.

In certain embodiments, knock out of both CCR5 and CXCR4 prevents and/or treats HIV infection or AIDS. In certain embodiments, knockdown of both CCR5 and CXCR4 prevents and/or treats HIV infection or AIDS. In certain embodiments, knockout of CCR5 and knockdown of CXCR4 prevent and/or treat HIV infection or AIDS. In certain embodiments, knockdown of CCR5 and knock out of CXCR4 prevent and/or treat HIV infection or AIDS. In certain embodiments, introduction of one or more mutations (e.g., one or more protective mutations) in the CCR5 gene and knockout of CXCR4 prevent and/or treat HIV infection or AIDS. In certain embodiments, introduction of one or more mutations (e.g., one or more protective mutations) in the CCR5 gene and knockdown of CXCR4 prevent and/or treat HIV infection or AIDS. In certain embodiments, introduction of one or more mutations (e.g., one or more single or two base substitutions) in the CXCR4 gene and knockout of CCR5 prevent and/or treat HIV infection or AIDS. In certain embodiments, introduction of one or more mutations (e.g., one or more single or two base substitutions) in the CXCR4 gene and knockdown of CCR5 prevent and/or treat HIV infection or AIDS. In certain embodiments, introduction of one or more mutations (e.g., one or more single or two base substitutions) in the CXCR4 gene and introduction of one or more mutations (e.g., one or more protective mutations) in the CCR5 gene prevent and/or treat HIV infection or AIDS. Introduction of the one or more mutations in the CCR5 gene and/or the CXCR4 gene can be done by co-delivery of an oligonucleotide donor (e.g., a donor DNA repair template) that encodes regions of homology proximal to the targeted mutation site(s) and encodes the specific mutation(s). The donor DNA repair template can be delivered in the context of a single strand deoxynucleotide donor (ssODN), a double strand deoxynucletide donor, or a viral vector (e.g., AAV or IDLV).

In certain embodiments, the genes, e.g., the coding sequence of the CCR5 gene and the coding sequence of the CXCR4 gene, are targeted to knock out the genes, e.g., to reduce or eliminate expression of the genes, e.g., to knock out both alleles of the CCR5 gene and the CXCR4 gene, e.g., by introducing an alteration comprising a mutation (e.g., a single point mutation, an insertion and/or a deletion) in both the CCR5 gene and the CXCR4 gene. This type of alteration is sometimes referred to as “knocking out” both the CCR5 gene and the CXCR4 gene. In certain embodiments, a targeted knockout approach is mediated by NHEJ using a CRISPR/Cas system comprising a Cas9 molecule, e.g., an enzymatically active Cas9 (eaCas9) molecule, as described herein.

When two or more genes (e.g., CCR5 and CXCR4) are targeted for alteration, the two or more genes (e.g., CCR5 and CXCR4) can be altered sequentially or simultaneously. In certain embodiments, the CCR5 gene and the CXCR4 gene are altered simultaneously. In certain embodiments, the CCR5 gene and the CXCR4 gene are altered sequentially. In certain embodiments, the alteration of the CXCR4 gene is prior to the alteration of the CCR5 gene. In certain embodiments, the alteration of the CXCR4 gene is concurrent with the alteration of the CCR5 gene. In certain embodiments, the alteration of the CXCR4 gene is subsequent to the alteration of the CCR5 gene. In certain embodiments, the effect of the alterations is synergistic. In certain embodiments, the two or more genes (e.g., CCR5 and CXCR4) are altered sequentially in order to reduce the probability of introducing genomic rearrangements (e.g., translocations) involving the two target positions.

In another aspect, the methods, genome editing systems, and compositions discussed herein are used to alter both the CCR5 gene and the CXCR4 gene to treat or prevent HIV infection or AIDS by targeting a non-coding sequence of the CCR5 gene and by targeting a non-coding sequence of the CXCR4 gene, e.g., a promoter, an enhancer, an intron, a 3′UTR, and/or a polyadenylation signal.

In certain embodiments, two distinct gRNA molecules are used to target two target positions, e.g., a CCR5 target position and a CXCR4 target position in two genes, e.g., the CCR5 gene and the CXCR4 gene. In certain embodiments, three or more distinct gRNA molecules are used to target two target positions, e.g., a CCR5 target position and a CXCR4 target position in two genes, e.g., the CCR5 gene and the CXCR4 gene. In certain embodiments, three or more distinct gRNA molecules are used to target three or more distinct target positions in two genes, e.g., the CCR5 gene and the CXCR4 gene.

In certain embodiments, the genome editing systems or compositions described herein comprise a first gRNA molecule comprising a first targeting domain that is complementary with a target domain (also referred to as “target sequence”) of a CCR5 gene, wherein the first targeting domain comprises a nucleotide sequence selected from SEQ ID NOS: 208 to 3739 and a second gRNA molecule comprising a second targeting domain that is complementary with a target domain (also referred to as “target sequence”) of a CXCR4 gene, wherein the second targeting domain comprises a nucleotide sequence selected from SEQ ID NOS: 3740 to 8407.

In certain embodiments, the first targeting domain comprises a nucleotide sequence selected from SEQ ID NOS: 208 to 1569, and 1614 to 3663, and the second targeting domain comprises a nucleotide sequence selected from SEQ ID NO: SEQ ID NOS: 3740 to 5208, and 5241 to 8355.

In certain embodiments, the first targeting domain comprises a nucleotide sequence selected from SEQ ID NOS: 335, 480, 482, 486, 488, 490, 492, 512, 521, 535, 1000, and 1002, and the second targeting domain comprises a nucleotide sequence selected from SEQ ID NO: 3973, 4118, and 4604.

In Certain Embodiments, the First Targeting Domain and the Second Targeting Domain are Selected from the Group Consisting of:

(a) a first targeting domain comprising the nucleotide sequence set forth in SEQ ID NO: 335, and a second targeting domain comprising the nucleotide sequence set forth in SEQ ID NO: 3973;

(b) a first targeting domain comprising the nucleotide sequence set forth in SEQ ID NO: 335, and a second targeting domain comprising the nucleotide sequence set forth in SEQ ID NO: 4604;

(c) a first targeting domain comprising the nucleotide sequence set forth in SEQ ID NO: 488, and a second targeting domain comprising the nucleotide sequence set forth in SEQ ID NO: 4604; and

(d) a first targeting domain comprising the nucleotide sequence set forth in SEQ ID NO: 480, and a second targeting domain comprising the nucleotide sequence set forth in SEQ ID NO: 4118.

In certain embodiments, a nucleic acid composition comprises (a) a nucleotide sequence that encodes a gRNA molecule e.g., the first gRNA molecule, comprising a targeting domain that is complementary with a target domain (also referred to as “target sequence”) in the CCR5 gene as disclosed herein, and further comprising (e) a nucleotide sequence that encodes a gRNA molecule e.g., the second gRNA molecule, comprising a targeting domain that is complementary with a target domain (also referred to as “target sequence”) in the CXCR4 gene as disclosed herein, and further comprising (b) a nucleotide sequence that encodes a Cas9 molecule.

In certain embodiments, a nucleic acid composition comprises (a) a nucleotide sequence that encodes a gRNA molecule e.g., the first gRNA molecule, comprising a targeting domain that is complementary with a target domain (also referred to as “target sequence”) in the CCR5 gene as disclosed herein, and further comprising (e) a nucleotide sequence that encodes a gRNA molecule e.g., the second gRNA molecule, comprising a targeting domain that is complementary with a target domain (also referred to as “target sequence”) in the CXCR4 gene as disclosed herein, and further comprising (b) a nucleotide sequence that encodes a Cas9 molecule specific for the CCR5 target position, and further comprising (f) a nucleotide sequence that encodes a second Cas9 molecule specific for the CXCR4 target position.

In certain embodiments, the at least one Cas9 molecule is an S. pyogenes Cas9 molecule or an S. aureus Cas9 molecule. In certain embodiments, the at least one Cas9 molecule comprises an S. pyogenes Cas9 molecule and an S. aureus Cas9 molecule. In certain embodiments, the at least one Cas9 molecule comprises a wild-type Cas9 molecule, a mutant Cas9 molecule, or a combination thereof. In certain embodiments, the mutant Cas9 molecule comprises a D10A mutation.

A nucleic acid composition disclosed herein may comprise (a) a sequence that encodes a first gRNA molecule comprising a targeting domain that is complementary with a target domain in the CCR5 gene as disclosed herein; (e) a sequence that encodes a second gRNA molecule e.g., the second gRNA molecule, comprising a targeting domain that is complementary with a target domain in the CXCR4 gene as disclosed herein; (b) a sequence that encodes a Cas9 molecule; and further may comprise (c)(i) a sequence that encodes a third gRNA molecule described herein having a targeting domain that is complementary to a second target domain of the CCR5 gene, and optionally, (g)(i) a sequence that encodes a fourth gRNA molecule described herein having a targeting domain that is complementary to a second target domain of the CXCR4 gene, and optionally, (c)(ii) a sequence that encodes a fifth gRNA molecule described herein having a targeting domain that is complementary to a third target domain of the CCR5 gene, and optionally, (g)(ii) a sequence that encodes a sixth gRNA molecule described herein having a targeting domain that is complementary to a third target domain of the CXCR4 gene; and optionally, (c)(iii) a sequence that encodes a seventh gRNA molecule described herein having a targeting domain that is complementary to a fourth target domain of the CCR5 gene, and optionally, (g)(iii) a sequence that encodes an eighth gRNA molecule described herein having a targeting domain that is complementary to a fourth target domain of the CXCR4 gene.

In certain embodiments, the first, third, fifth and seventh gRNA molecules comprising a CCR5 targeting domain correspond to the first, second, third and fourth gRNAs, respectively, described herein, e.g., described in the section “Alteration of CCR5”. In certain embodiments, the second, fourth, sixth and eighth gRNA molecules comprising a CXCR4 targeting domain correspond to the first, second, third and fourth gRNAs, respectively, described herein, e.g., described in the section “Alteration of CXCR4”.

In certain embodiments, a nucleic acid composition encodes (a) a first nucleotide sequence that encodes a first gRNA molecule comprising a targeting domain that is complementary with a target domain in the CCR5 gene as disclosed herein, and (b) a second nucleotide sequence that encodes a second gRNA molecule comprising a targeting domain that is complementary with a target domain in the CXCR4 gene as disclosed herein, and (c) a third nucleotide sequence that encodes a Cas9 molecule or molecules, e.g., a Cas9 molecule described herein. In certain embodiments, (a), (b) and (c) are present on one nucleic acid molecule, e.g., one vector, e.g., one viral vector, e.g., one AAV vector. In certain embodiments, the nucleic acid molecule is an AAV vector. Exemplary AAV vectors that may be used in any of the described compositions and methods include an AAV1 vector, a modified AAV1 vector, an AAV2 vector, a modified AAV2 vector, an AAV3 vector, an AAV4 vector, a modified AAV4 vector, an AAV5 vector, a modified AAV5 vector, a modified AAV3 vector, an AAV6 vector, a modified AAV6 vector, an AAV8 vector an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector. In certain embodiments, the nucleic acid molecule is a lentiviral vector, e.g., an DLV (integration deficienct lentivirus vector).

In certain embodiments, (a) and (b) are present on a first nucleic acid molecule, e.g. a first vector, e.g., a first viral vector, e.g., a first AAV vector; and (c) is present on a second nucleic acid molecule, e.g., a second vector, e.g., a second vector, e.g., a second AAV vector. The first and second nucleic acid molecules may be AAV vectors.

In certain embodiments, (a) is present on a first nucleic acid molecule, e.g. a first vector, e.g., a first viral vector, e.g., a first AAV vector; and (b) is present on a second nucleic acid molecule, e.g., a second vector, e.g., a second vector, e.g., a second AAV vector; and (c) is present on a third nucleic acid molecule, e.g., a third vector, e.g., a third vector, e.g., a third AAV vector. The first and second and third nucleic acid molecules may be AAV vectors.

In certain embodiments, the nucleic acid composition further comprises (d) a fourth nucleotide sequence that encodes a third gRNA molecule comprising a targeting domain that is complementary to a second target domain of the CCR5 gene. In certain embodiments, the nucleic acid composition further comprises (e) a fifth nucleotide sequence that encodes a fourth gRNA molecule comprising a targeting domain that is complementary to a third target domain of the CCR5 gene. In certain embodiments, the nucleic acid composition further comprises (f) a sixth nucleotide sequence that encodes a fifth gRNA molecule comprising a targeting domain that is complementary to a fourth target domain of the CCR5 gene.

In certain embodiments, the nucleic acid composition further comprises (g) a seventh nucleotide sequence that encodes a sixth gRNA molecule comprising a targeting domain that is complementary to a second target domain of the CXCR4 gene. In certain embodiments, the nucleic acid composition further comprises (h) an eighth nucleotide sequence that encodes a seventh gRNA molecule comprising a targeting domain that is complementary to a third target domain of the CXCR4 gene. In certain embodiments, the nucleic acid composition further comprises (i) a ninth nucleotide sequence that encodes an eighth gRNA molecule comprising a targeting domain that is complementary to a fourth target domain of the CXCR4 gene.

Each of (a) to (i) may be present on the same or different nucleic acid molecule(s), e.g., vector (s), e.g., viral vector(s), e.g., AAV vector(s).

The presently disclosed subject matter further provides for a composition comprising (a) a first gRNA molecule comprising a targeting domain that is complementary with a target domain in the CCR5 gene, and (b) a second gRNA molecule comprising a targeting domain that is complementary with a target domain in the CXCR4 gene, as described herein. The composition may further comprise (c) a Cas9 molecule or molecules, e.g., a Cas9 molecule as described herein. The composition may further comprise a third, fourth, fifth, sixth, seventh, and/or eighth gRNA molecules. The compositions described herein, e.g., pharmaceutical compositions described herein, can be used in the treatment or prevention of HIV or AIDS in a subject, e.g., in accordance with a method disclosed herein.

The presently disclosed subject matter further provides for a method of altering a cell, e.g., altering the structure, e.g., altering the sequence, of a target nucleic acid of a cell, comprising contacting said cell with: (a) a first gRNA molecule that targets the CCR5 gene, e.g., a gRNA molecule as described herein; (b) a second gRNA molecule that targets the CXCR4 gene, e.g., a gRNA molecule as described herein; (c) a Cas9 molecule or molecules, e.g., a Cas9 molecule as described herein. In certain embodiments, the method comprises contacting the cell with a third gRNA molecule, optionally a fourth gRNA molecule and/or a fifth gRNA molecule, each of which targets the CCR5 gene. In certain embodiments, the method comprises contacting the cell with a sixth gRNA molecule, optionally a seventh gRNA molecule and/or an eighth gRNA molecule, each of which targets the CXCR4 gene.

In certain embodiments, the method comprises contacting a cell from a subject suffering from or likely to develop an HIV infection or AIDS. The cell may be from a subject who does not have a mutation at a CCR5 target position.

In certain embodiments, the cell being contacted in the disclosed method is a target cell from a circulating blood cell, a progenitor cell, or a stem cell, e.g., a hematopoietic stem cell (HSC) or a hematopoietic stem/progenitor cell (HSPC). In certain embodiments, the target cell is a T cell (e.g., a CD4+ T cell, a CD8+ T cell, a helper T cell, a regulatory T cell, a cytotoxic T cell, a memory T cell, a T cell precursor or a natural killer T cell), a B cell (e.g., a progenitor B cell, a Pre B cell, a Pro B cell, a memory B cell, a plasma B cell), a monocyte, a megakaryocyte, a neutrophil, an eosinophil, a basophil, a mast cell, a reticulocyte, a lymphoid progenitor cell, a myeloid progenitor cell, or a hematopoietic stem cell. In certain embodiments, the target cell is a bone marrow cell, (e.g., a lymphoid progenitor cell, a myeloid progenitor cell, an erythroid progenitor cell, a hematopoietic stem cell, or a mesenchymal stem cell). In certain embodiments, the cell is a CD4 cell, a T cell, a gut associated lymphatic tissue (GALT), a macrophage, a dendritic cell, a myeloid precursor cell, or a microglial cell. The contacting may be performed ex vivo and the contacted cell may be returned to the subject's body after the contacting step. In certain embodiments, the contacting step may be performed in vivo.

In certain embodiments, the method of altering a cell as described herein comprises acquiring knowledge of the presence of a CCR5 target position in said cell, prior to the contacting step. Acquiring knowledge of the presence of a CCR5 target position in the cell may be by sequencing the CCR5 gene, or a portion of the CCR5 gene. In certain embodiments, the method of altering a cell as described herein comprises acquiring knowledge of the presence of a CXCR4 target position in said cell, prior to the contacting step. Acquiring knowledge of the presence of a CXCR4 target position in the cell may be by sequencing the CXCR4 gene, or a portion of the CXCR4 gene.

In certain embodiments, the method comprises delivering to the cell a Cas9 molecule or molecules of (c), as a protein or an mRNA, and a nucleic acid composition that encodes a first gRNA molecule of (a) and a second gRNA molecule of (b) and optionally a third, fourth, and/or fifth gRNA molecule and optionally a sixth, seventh, and/or eighth gRNA molecule.

In certain embodiments, the method delivering to the cell a Cas9 molecule or molecules of (c), as a protein or an mRNA, said gRNAs of (a) and (b), as an RNA, and optionally said third, fourth, and/or fifth gRNA molecule, as an RNA, and optionally said sixth, seventh, and/or eighth gRNA molecule, as an RNA.

In certain embodiments, the method comprises delivering to the cell a first gRNA molecule of (a) as an RNA, a second gRNA molecule of (b) as an RNA, and optionally the third, fourth, and/or fifth gRNA molecule as an RNA, and optionally the sixth, seventh, and/or eighth gRNA molecule, as an RNA, and a nucleic acid composition that encodes the Cas9 molecule or molecules of (c).

In certain embodiments, the method further comprises contacting the cell with an HSC self-renewal agonist, e.g., UM171 ((1r,4r)-N1-(2-benzyl-7-(2-methyl-2H-tetrazol-5-yl)-9H-pyrimido[4,5-b]indol-4-yl)cyclohexane-1,4-diamine) or a pyrimidoindole derivative described in Fares et al., Science, 2014, 345(6203): 1509-1512). In certain embodiments, the cell is contacted with the HSC self-renewal agonist before (e.g., at least 1, 2, 4, 8, 12, 24, 36, or 48 hours before, e.g., about 2 hours before) the cell is contacted with a gRNA molecule and/or a Cas9 molecule. In certain embodiments, the cell is contacted with the HSC self-renewal agonist after (e.g., at least 1, 2, 4, 8, 12, 24, 36, or 48 hours after, e.g., about 24 hours after) the cell is contacted with a gRNA molecule and/or a Cas9 molecule. In yet certain embodiments, the cell is contacted with the HSC self-renewal agonist before (e.g., at least 1, 2, 4, 8, 12, 24, 36, or 48 hours before) and after (e.g., at least 1, 2, 4, 8, 12, 24, 36, or 48 hours after) the cell is contacted with a gRNA molecule and/or a Cas9 molecule. In certain embodiments, the cell is contacted with the HSC self-renewal agonist about 2 hours before and about 24 hours after the cell is contacted with a gRNA molecule and/or a Cas9 molecule. In certain embodiments, the cell is contacted with the HSC self-renewal agonist at the same time the cell is contacted with a gRNA molecule and/or a Cas9 molecule. In certain embodiments, the HSC self-renewal agonist, e.g., UM171, is used at a concentration between 5 and 200 nM, e.g., between 10 and 100 nM or between 20 and 50 nM, e.g., about 40 nM.

The presently disclosed subject matter further provides for a cell or a population of cells produced (e.g., altered) by a method described herein.

The presently disclosed subject matter further provides for a method of treating a subject suffering from or likely to develop an HIV infection or AIDS, e.g., altering the structure, e.g., sequence, of a target nucleic acid of the subject, comprising contacting the subject (or a cell from the subject) with:

(a) a first gRNA molecule that targets the CCR5 gene, e.g., a gRNA molecule disclosed herein;

(b) a second gRNA molecule that targets the CXCR4 gene, e.g., a gRNA molecule disclosed herein;

(c) a Cas9 molecule or molecules, e.g., a Cas9 molecule disclosed herein; and

optionally, (d) a third gRNA molecule that targets the CCR5 gene, and optionally, (e) a fourth gRNA molecule that target the CCR5 gene, and still further optionally, (f) a fifth gRNA molecule that target the CCR5 gene, and optionally (g) a sixth gRNA molecule that targets the CXCR4 gene, and optionally, (h) a seventh gRNA molecule that target the CXCR4 gene, and still further optionally, (i) an eighth gRNA molecule that target the CXCR4 gene.

In certain embodiments, the method comprises contacting with (a), (b) and (c). In certain embodiments, the method comprises contacting the cell with (a), (b), (c), and (d). In certain embodiments, the method comprises contacting the cell with (a), (b), (c), (d), and (g).

The gRNA molecules that target the CCR5 gene (the gRNA molecules of (a), (d), (e) and (f)) may comprise a targeting domain that comprises a nucleotide sequence selected from SEQ ID NOS: 208 to 3739, or comprise a targeting domain that comprises a nucleotide sequence that differs by no more than 1, 2, 3, 4, or 5 nucleotides from, a nucleotide sequence selected from SEQ ID NOS: 208 to 3739.

The gRNA molecule that target the CXCR4 gene (the gRNA molecules of (b), (g), (h) and (i)) may comprise a targeting domain that comprises a nucleotide sequence selected from SEQ ID NOS: 3740 to 8407, or comprise a targeting domain that comprises a nucleotide sequence that differs by no more than 1, 2, 3, 4, or 5 nucleotides from, a nucleotide sequence selected from SEQ ID NOS: 3740 to 8407.

In certain embodiments, the method comprises acquiring knowledge of the presence or absence of a mutation at a CCR5 target position in said subject. In certain embodiments, the method comprises acquiring knowledge of the presence or absence of a mutation at a CCR5 target position in said subject by sequencing the CCR5 gene or a portion of the CCR5 gene. In certain embodiments, the method comprises acquiring knowledge of the presence or absence of a mutation at a CXCR4 target position in said subject. In certain embodiments, the method comprises acquiring knowledge of the presence or absence of a mutation at a CXCR4 target position in said subject by sequencing the CXCR4 gene or a portion of the CXCR4 gene. In certain embodiments, the method comprises introducing a mutation at a CCR5 target position and introducing a mutation at a CXCR4 target position. In certain embodiments, the method comprises introducing a mutation at a CCR5 target position, e.g., by NHEJ, and introducing a mutation at a CXCR4 target position, e.g., by NHEJ.

When the method comprises introducing a mutation at a CCR5 target position and introducing a mutation at a CXCR4 target position, e.g., by NHEJ in the coding region or a non-coding region of CCR5 gene, e.g., by NHEJ in the coding region or a non-coding region of CXCR4 gene, a Cas9 of (b) and at least two guide RNAs (e.g., a guide RNA of (a) and a guide RNA of (e)) are included in the contacting step.

In certain embodiments, a cell of the subject is contacted ex vivo with (a), (b), (c) and optionally (d), further optionally (g), further optionally one or more of (e), (f), (h) and (i). In certain embodiments, said cell is returned to the subject's body. In certain embodiments, a cell of the subject is contacted is in vivo with (a), (b), (c) and optionally (d), further optionally (g), further optionally one or more of (e), (f), (h) and (i). In certain embodiments, the method comprises contacting the subject with a nucleic acid composition, e.g., a vector, e.g., an AAV vector, described herein, e.g., a nucleic acid that encodes at least one of (a), (b), (c), and optionally (d), further optionally (g), further optionally one or more of (e), (f), (h) and (i).

In certain embodiments, the method comprises delivering to said subject said Cas9 molecule or molecules of (c), as a protein or mRNA, and a nucleic acid composition that encodes (a) and (b) and optionally (d), further optionally (g), further optionally one or more of (e), (f), (h) and (i).

In certain embodiments, the method comprises delivering to the subject the Cas9 molecule or molecules of (c), as a protein or mRNA, said first and second gRNAs of (a) and of (b), as an RNA, and optionally said third gRNA molecule of (d), further optionally further optionally (g), further optionally one or more of (e), (f), (h) and (i) as an RNA.

In certain embodiments, the method comprises delivering to the subject the first and second gRNAs of (a) and (b), as an RNA, optionally said third gRNA molecule of (d), further optionally (g), further optionally one or more of (e), (f), (h) and (i) as an RNA, and a nucleic acid composition that encodes the Cas9 molecule or molecules of (c).

The presently disclosed subject matter further provides for a reaction mixture comprising two or more gRNA molecules, a nucleic acid composition, or a composition described herein, and a cell, e.g., a cell from a subject having, or likely to develop and HIV infection or AIDS, a subject having a mutation at a CCR5 target position (e.g., a heterozygous carrier of a CCR5 mutation), or a subject having a mutation at a CXCR4 target position (e.g., a heterozygous carrier of a CXCR4 mutation).

The presently disclosed subject matter further provides for a kit comprising, (a) a first gRNA molecule that targets the CCR5 gene, as described herein or a nucleic acid that encodes thereof, (b) a second gRNA molecule that targets the CXCR4 gene, as described herein or a nucleic acid that encodes thereof, and one or more of the following:

(c) a Cas9 molecule or molecules, e.g., a Cas9 molecule described herein, or a nucleic acid or mRNA that encodes the Cas9 molecule; and optionally,

(d), (e), and/or (f) a third, fourth, and/or fifth gRNA molecule, each of which targets the CCR5 gene, e.g., a third gRNA molecule described herein or a nucleic acid that encodes (c)(i); further optionally,

(g), (h), and/or (i) a sixth, seventh, and/or eight gRNA molecule, each of which targets the CXCR4 gene.

The presently disclosed subject matter further provides for two or more (e.g., 3, 4, 5, 6, 7, or 8) of the gRNA molecules described herein, for use in treating, or delaying the onset or progression of, HIV infection or AIDS in a subject, e.g., in accordance with a method of treating, or delaying the onset or progression of, HIV infection or AIDS as described herein. In certain embodiments, the gRNA molecules used in combination with a Cas9 molecule, e.g., a Cas9 molecule described herein.

The presently disclosed subject matter further provides for use of two or more (e.g., 3, 4, 5, 6, 7, or 8) of the gRNA molecules described herein, in the manufacture of a medicament for treating, or delaying the onset or progression of, HIV infection or AIDS in a subject, e.g., in accordance with a method of treating, or delaying the onset or progression of, HIV infection or AIDS as described herein. In certain embodiments, the medicament comprises a Cas9 molecule, e.g., a Cas9 molecule described herein.

The gRNA molecules and methods, as disclosed herein, can be used in combination with a governing gRNA molecule. As used herein, a governing gRNA molecule refers to a gRNA molecule comprising a targeting domain which is complementary to a target domain on a nucleic acid that encodes a component of the CRISPR/Cas system introduced into a cell or subject. For example, the methods described herein can further include contacting a cell or subject with a governing gRNA molecule or a nucleic acid encoding a governing molecule. In certain embodiments, the governing gRNA molecule targets a nucleic acid that encodes a Cas9 molecule or a nucleic acid that encodes a target gene gRNA molecule. In certain embodiments, the governing gRNA comprises a targeting domain that is complementary to a target domain in a sequence that encodes a Cas9 component, e.g., a Cas9 molecule or target gene gRNA molecule. In certain embodiments, the target domain is designed with, or has, minimal homology to other nucleic acid sequences in the cell, e.g., to minimize off-target cleavage. For example, the targeting domain on the governing gRNA can be selected to reduce or minimize off-target effects. In certain embodiments, a target domain for a governing gRNA can be disposed in the control or coding region of a Cas9 molecule or disposed between a control region and a transcribed region. In certain embodiments, a target domain for a governing gRNA can be disposed in the control or coding region of a target gene gRNA molecule or disposed between a control region and a transcribed region for a target gene gRNA. In certain embodiments, altering, e.g., inactivating, a nucleic acid that encodes a Cas9 molecule or a nucleic acid that encodes a target gene gRNA molecule can be effected by cleavage of the targeted nucleic acid sequence or by binding of a Cas9 molecule/governing gRNA molecule complex to the targeted nucleic acid sequence.

The compositions, reaction mixtures and kits, as disclosed herein, can also include a governing gRNA molecule, e.g., a governing gRNA molecule disclosed herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Headings, including numeric and alphabetical headings and subheadings, are for organization and presentation and are not intended to be limiting.

Other features and advantages of the invention can be apparent from the detailed description, drawings, and from the claims.

### BRIEF DESCRIPTION OF THE DRAWINGS

FIGS. 1A-1I are representations of several exemplary gRNAs. FIG. 1A depicts a modular gRNA molecule derived in part (or modeled on a sequence in part) from Streptococcus pyogenes (S. pyogenes) as a duplexed structure (SEQ ID NOs:39 and 40, respectively, in order of appearance); FIG. 1B depicts a unimolecular gRNA molecule derived in part from S. pyogenes as a duplexed structure (SEQ ID NO:41); FIG. 1C depicts a unimolecular gRNA molecule derived in part from S. pyogenes as a duplexed structure (SEQ ID NO:42); FIG. 1D depicts a unimolecular gRNA molecule derived in part from S. pyogenes as a duplexed structure (SEQ ID NO:43); FIG. 1E depicts a unimolecular gRNA molecule derived in part from S. pyogenes as a duplexed structure (SEQ ID NO:44); FIG. 1F depicts a modular gRNA molecule derived in part from Streptococcus thermophilus (S. thermophilus) as a duplexed structure (SEQ ID NOs:45 and 46, respectively, in order of appearance); and FIG. 1G depicts an alignment of modular gRNA molecules of S. pyogenes and S. thermophilus (SEQ ID NOs:39, 45, 47, and 46, respectively, in order of appearance). FIGS. 1H-1I depicts additional exemplary structures of unimolecular gRNA molecules. FIG. 1H shows an exemplary structure of a unimolecular gRNA molecule derived in part from S. pyogenes as a duplexed structure (SEQ ID NO:42). FIG. 1I shows an exemplary structure of a unimolecular gRNA molecule derived in part from S. aureus as a duplexed structure (SEQ ID NO:38).

FIGS. 2A-2G depict an alignment of Cas9 sequences (Chylinski 2013). The N-terminal RuvC-like domain is boxed and indicated with a “Y.” The other two RuvC-like domains are boxed and indicated with a “B.” The HNH-like domain is boxed and indicated by a “G.” Sm: S. mutans (SEQ ID NO:1); Sp: S. pyogenes (SEQ ID NO:2); St: S. thermophilus (SEQ ID NO:4); and Li: L. innocua (SEQ ID NO:5). “Motif” (SEQ ID NO:14) is a consensus sequence based on the four sequences. Residues conserved in all four sequences are indicated by single letter amino acid abbreviation; “*” indicates any amino acid found in the corresponding position of any of the four sequences; and “-” indicates absent.

FIGS. 3A-3B show an alignment of the N-terminal RuvC-like domain from the Cas9 molecules disclosed in Chylinski 2013 (SEQ ID NOs:52-95, 120-123). The last line of FIG. 3B identifies 4 highly conserved residues.

FIGS. 4A-4B show an alignment of the N-terminal RuvC-like domain from the Cas9 molecules disclosed in Chylinski 2013 with sequence outliers removed (SEQ ID NOs:52-123). The last line of FIG. 4B identifies 3 highly conserved residues.

FIGS. 5A-5C show an alignment of the HNH-like domain from the Cas9 molecules disclosed in Chylinski 2013 (SEQ ID NOs:124-198). The last line of FIG. 5C identifies conserved residues.

FIGS. 6A-6B show an alignment of the HNH-like domain from the Cas9 molecules disclosed in Chylinski 2013 with sequence outliers removed (SEQ ID NOs:124-141, 148, 149, 151-153, 162, 163, 166-174, 177-187, 194-198). The last line of FIG. 6B identifies 3 highly conserved residues.

FIG. 7 illustrates gRNA domain nomenclature using an exemplary gRNA sequence (SEQ ID NO:42).

FIGS. 8A and 8B provide schematic representations of the domain organization of S. pyogenes Cas9. FIG. 8A shows the organization of the Cas9 domains, including amino acid positions, in reference to the two lobes of Cas9 (recognition (REC) and nuclease (NUC) lobes). FIG. 8B shows the percent homology of each domain across 83 Cas9 orthologs.

FIG. 9 depicts the efficiency of NHEJ mediated by a Cas9 molecule and exemplary gRNA molecules targeting the CCR5 locus.

FIG. 10 depicts flow cytometry analysis of genome edited HSCs to determine co-expression of stem cell phenotypic markers CD34 and CD90 and for viability (7-AAD-AnnexinV-cells). CD34+ HSCs maintain phenotype and viability after Nucleofection™ with Cas9 and CCR5 gRNA plasmid DNA (96 hours).

FIGS. 11A-11B depict exemplary results illustrating UM171 pre-treated CD34⁺ HSCs maintain proliferation potential and exhibit increased genome editing at the CXCR4 locus after Nucleofection™ with plasmids expressing S. aureus (Sa) or S. pyogenes (Spy) Cas9 paired with CXCR4-836 and CXCR4-231 gRNAs, respectively. FIG. 11A depicts an exemplary result of the fold expansion of Nucleofected™ CD34⁺ cells 96 hours after delivery of the indicated Cas9 variant paired with CXCR4 gRNA or GFP-expressing plasmid alone (pmax GFP). FIG. 11B depicts an exemplary result of the percentage of indels as detected by T7E1 assays in CD34⁺ HSC after the indicated Nucleofections™. The plus and minus signs under the x-axes indicate treatment +/−40 nM UM171 is indicated.

FIGS. 12A-12B depict exemplary results illustrating effective multiplex genome editing of CD34⁺ HSCs after Nucleofection™ based co-delivery of plasmids expressing S. pyogenes (Spy) Cas9, one CXCR4 gRNA, and one CCR5 gRNA. FIG. 12A depicts an exemplary result of the fold expansion of Nucleofected™ CD34⁺ cells 96 hours after co-delivery of Cas9 paired with CXCR4 gRNA (CXCR4-231) and CCR5 gRNA (CCR5-U43) plasmids. FIG. 12B depicts an exemplary result of the percentage of indels detected by T7E1 assays in CD34⁺ HSCs at CCR5 and CXCR4 genomic loci.

FIGS. 13A-13C depicts electroporation of capped and tailed gRNAs increases human CD34⁻ cell survival and viability. CD34⁺ cells were electroporated with the indicated uncapped/untailed gRNAs or capped/tailed gRNAs with paired Cas9 mRNA (either S. pyogenes (Sp)or S. aureus Sa Cas9). Control samples include: cells that were electroporated with GFP mRNA alone or were not electroporated but were cultured for the indicated time frame. FIG. 13A shows the kinetics of CD34⁺ cell expansion after electroporation. FIG. 13B shows the fold change in total live CD34⁺ cells 72 hours after electroporation. FIG. 13C depicts representative flow cytometry data showing maintenance of viable (propidium iodide negative) human CD34⁺ cells after electroporation with capped and tailed AAVS1 gRNA and Cas9 mRNA.

FIGS. 14A-14G depicts electroporation of Cas9 mRNA and capped and tailed gRNA supports efficient editing in human CD34⁺ cells and their progeny. FIG. 14A shows the percentage of insertions/deletions (indels) detected in CD34⁺ cells and their hematopoietic colony forming cell (CFC) progeny at the targeted AAVS1 locus after delivery of Cas9 mRNA with capped and tailed AAVS1 gRNA compared to uncapped and untailed AAVS1 gRNA. FIG. 14B is an exemplary result demonstrating that hematopoietic colony forming potential (CFCs) is maintained in CD34+ cells after editing with capped/tailed AAVS1 gRNA. Note loss of CFC potential for cells electroporated with uncapped/untailed AAVS1 gRNA. FIG. 14C is an exemplary result demonstrating that delivery of capped and tailed HBB gRNA with S. pyogenes Cas9 mRNA or ribonucleoprotein (RNP) supports efficient targeted locus editing (% indels) in the K562 erythroleukemia cell line, a human erythroleukemia cell line has similar properties to HSCs. FIG. 14D depicts an exemplary result showing that Cas9-mediated/capped and tailed gRNA mediated editing (% indels) at the indicated target genetic loci (AAVS1, HBB, CXCR4) in human cord blood CD34⁺ cells. Right: CFC potential of cord blood CD34⁺ cells after electroporation with Cas9 mRNA and capped and tailed HBB_Sp8 gRNA (unelectroporated control or cells electroporated with 2 or 10 μg HBB gRNAs). Cells were electroporated with Cas9 mRNA and 2 or 10 μg of gRNA. FIG. 14E shows CFC assays for cells electroporated with 2 μg or 10 μg of capped/tailed HBB gRNA. CFCs: colony forming cells, GEMM: mixed hematopoietic colony granulocyte-erythrocyte-macrophage-monocyte, E: erythrocyte colony, GM: granulocyte-macrophage colong, G: granulocyte colony. FIG. 14F depicts a representative gel image showing cleavage at the indicated loci (T7E1 analysis) in cord blood CD34⁺ cells at 72 hours after delivery of capped and tailed AAVS1, HBB, or CXCR4 gRNA and S. pyogenes Cas9 mRNA. The example gel corresponds to the summary data shown in FIG. 14D. FIG. 14G depicts cell viability in CB CD34⁺ cells 48 hours after delivery of Cas9 mRNA and indicated gRNAs as determined by co-staining with 7-AAD and Annexin V and flow cyotometry analysis.

FIG. 15 depicts gene editing in genomic DNA from K562 cells after electroporation of plasmid DNA encoding S. aureus Cas9 and DNA encoding each gRNA regulated by U6 promoter as determined by T7E1 endonuclease assay.

### DETAILED DESCRIPTION

For purposes of clarity of disclosure and not by way of limitation, the detailed description is divided into the following subsections:

1. Definitions

2. Human Immunodeficiency Virus (HIV)

3. Methods to Treat or Prevent HIV Infection or AIDS;

4. Methods of Targeting CCR5

5. Methods of Targeting CXCR4

6. Methods of Multiplexed Targeting of Both CCR5 and CXCR4

7. Guide RNA (gRNA) Molecules

8. Methods for Designing gRNAs

9. Cas9 Molecules

10. Functional Analysis of Candidate Molecules

11. Genome Editing Approaches

12. Target Cells

13. Delivery, Formulations and Routes of Administration

14. Modified Nucleosides, Nucleotides, and Nucleic Acids

### 1. Definitions

As used herein, the term “about” or “approximately” means within an acceptable error range for the particular value as determined by one of ordinary skill in the art, which can depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, a “genome editing system” refers to a system that is capable of editing (e.g., modifying or altering) one or more target genes in a cell, for example by means of Cas9-mediated single or double strand breaks. Genome editing systems may comprise, in various embodiments, (a) one or more Cas9/gRNA complexes, and (b) separate Cas9 molecules and gRNAs that are capable of associating in a cell to form one or more Cas9/gRNA complexes. A genome editing system according to the present disclosure may be encoded by one or more nucleotides (e.g. RNA, DNA) comprising coding sequences for Cas9 and/or gRNAs that can associate to form a Cas9/gRNA complex, and the one or more nucleotides encoding the gene editing system may be carried by a vector as described herein.

In certain embodiments, the genome editing system targets a CCR5 gene. In certain embodiments, the CCR5 gene is a human CCR5 gene. In certain embodiments, the genome editing system targets a CXCR4 gene. In certain embodiments, the CXCR4 gene is a human CXCR4 gene. In certain embodiments, the genome editing system targets a CCR5 gene (e.g., a human CCR5 gene) and a CXCR4 gene (e.g., a human CXCR4 gene).

In certain embodiments, the genome editing system that targets a CCR5 gene comprises a first gRNA molecule comprising a targeting domain complementary to a target domain (also referred to as “target sequence”) in the CCR5 gene, or a polynucleotide encoding thereof, and at least one Cas9 molecule or polynucleotide(s) encoding thereof. In certain embodiments, the genome editing system that targets a CCR5 gene further comprises a second gRNA molecule comprising a targeting domain complementary to a second target domain in the CCR5 gene, or a polynucleotide encoding thereof. The the genome editing system that targets a CCR5 gene may further comprise a third and a fourth gRNA molecules that target the CCR5 gene.

In certain embodiments, the genome editing system that targets a CXCR4 gene comprises a first gRNA molecule comprising a targeting domain complementary to a target domain in the CXCR4 gene, or a polynucleotide encoding thereof, and at least one Cas9 molecule or polynucleotide(s) encoding thereof. In certain embodiments, the genome editing system that targets a CXCR4 gene further comprises a second gRNA molecule comprising a targeting domain complementary to a second target domain in the CXCR4gene, or a polynucleotide encoding thereof. The the genome editing system that targets a CXCR4 gene may further comprise a third and a fourth gRNA molecules that target the CXCR4 gene.

In certain embodiments, the genome editing system that targets a CCR5 gene and a CXCR4 gene comprises a first gRNA molecule comprising a targeting domain complementary to a target domain in the CCR5 gene, or a polynucleotide encoding thereof, a second gRNA molecule comprising a targeting domain complementary to a target domain in the CXCR4 gene, or a polynucleotide encoding thereof, and at least one Cas9 molecule or polynucleotide(s) encoding thereof. In certain embodiments, the genome editing system that targets a CCR5 gene and a CXCR4 gene further comprises a third gRNA molecule comprising a targeting domain complementary to a second target domain in the CCR5 gene, or a polynucleotide encoding thereof. In certain embodiments, the genome editing system that targets a CCR5 gene and a CXCR4 gene further comprises a fourth gRNA molecule comprising a targeting domain complementary to a second target domain in the CXCR4 gene, or a polynucleotide encoding thereof. The the genome editing system that targets a CCR5 gene and a CXCR4 may further comprise a fifth and a sixth gRNA molecules that target the CCR5gene, and further a seventh and an eight gRNA molecules that target the CXCR4gene.

In certain embodiments, the genome editing system is implemented in a cell or in an in vitro contact. In certain embodiments, the genome editing system is used in a medicament, e.g., a medicament for modifying one or more target genes (e.g., CCR5 and/or CXCR4 genes), or a medicament for treating HIV infection and AIDS. In certain embodiments, the genome editing system is used in therapy.

“CCR5 target position”, as used herein, refers to any position that results in inactivation of the CCR5 gene. In certain embodiments, a CCR5 target position refers to any of a CCR5 target knockout position or a CCR5 target knockdown position, as described herein.

“CXCR4 target position”, as used herein, refers to any position that results in inactivation of the CXCR4 gene. In certain embodiments, a CXCR4 target position refers to any of a CXCR4 target knockout position or a CXCR4 target knockdown position, as described herein.

“Domain”, as used herein, is used to describe segments of a protein or nucleic acid. Unless otherwise indicated, a domain is not required to have any specific functional property.

Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

“Governing gRNA molecule”, as used herein, refers to a gRNA molecule that comprises a targeting domain that is complementary to a target domain on a nucleic acid that comprises a sequence that encodes a component of the CRISPR/Cas system that is introduced into a cell or subject. A governing gRNA does not target an endogenous cell or subject sequence. In certain embodiments, a governing gRNA molecule comprises a targeting domain that is complementary with a target sequence on: (a) a nucleic acid that encodes a Cas9 molecule; (b) a nucleic acid that encodes a gRNA which comprises a targeting domain that targets the CCR5 gene (a target gene gRNA); or on more than one nucleic acid that encodes a CRISPR/Cas component, e.g., both (a) and (b). In certain embodiments, a nucleic acid molecule that encodes a CRISPR/Cas component, e.g., that encodes a Cas9 molecule or a target gene gRNA, comprises more than one target domain that is complementary with a governing gRNA targeting domain. In certain embodiments, a governing gRNA molecule complexes with a Cas9 molecule and results in Cas9 mediated inactivation of the targeted nucleic acid, e.g., by cleavage or by binding to the nucleic acid, and results in cessation or reduction of the production of a CRISPR/Cas system component. In certain embodiments, the Cas9 molecule forms two complexes: a complex comprising a Cas9 molecule with a target gene gRNA, which complex can alter the CCR5 gene; and a complex comprising a Cas9 molecule with a governing gRNA molecule, which complex can act to prevent further production of a CRISPR/Cas system component, e.g., a Cas9 molecule or a target gene gRNA molecule. In certain embodiments, a governing gRNA molecule/Cas9 molecule complex binds to or promotes cleavage of a control region sequence, e.g., a promoter, operably linked to a sequence that encodes a Cas9 molecule, a sequence that encodes a transcribed region, an exon, or an intron, for the Cas9 molecule. In certain embodiments, a governing gRNA molecule/Cas9 molecule complex binds to or promotes cleavage of a control region sequence, e.g., a promoter, operably linked to a gRNA molecule, or a sequence that encodes the gRNA molecule. In certain embodiments, the governing gRNA, e.g., a Cas9-targeting governing gRNA molecule, or a target gene gRNA-targeting governing gRNA molecule, limits the effect of the Cas9 molecule/target gene gRNA molecule complex-mediated gene targeting. In certain embodiments, a governing gRNA places temporal, level of expression, or other limits, on activity of the Cas9 molecule/target gene gRNA molecule complex. In certain embodiments, a governing gRNA reduces off-target or other unwanted activity. In certain embodiments, a governing gRNA molecule inhibits, e.g., entirely or substantially entirely inhibits, the production of a component of the Cas9 system and thereby limits, or governs, its activity.

“Modulator”, as used herein, refers to an entity, e.g., a drug, that can alter the activity (e.g., enzymatic activity, transcriptional activity, or translational activity), amount, distribution, or structure of a subject molecule or genetic sequence. In certain embodiments, modulation comprises cleavage, e.g., breaking of a covalent or non-covalent bond, or the forming of a covalent or non-covalent bond, e.g., the attachment of a moiety, to the subject molecule. In certain embodiments, a modulator alters the, three dimensional, secondary, tertiary, or quaternary structure, of a subject molecule. A modulator can increase, decrease, initiate, or eliminate a subject activity.

“Large molecule”, as used herein, refers to a molecule having a molecular weight of at least 2, 3, 5, 10, 20, 30, 40, 50, 60, 70, 80, 90, or 100 kD. Large molecules include proteins, polypeptides, nucleic acids, biologics, and carbohydrates.

“Polypeptide”, as used herein, refers to a polymer of amino acids having less than 100 amino acid residues. In certain embodiments, it has less than 50, 20, or 10 amino acid residues.

A “Cas9 molecule” or “Cas9 polypeptide” as used herein refers to a molecule or polypeptide, respectively, that can interact with a gRNA molecule and, in concert with the gRNA molecule, localize to a site comprising a target domain (also referred to as “target sequence”) and, in certain embodiments, a PAM sequence. Cas9 molecules and Cas9 polypeptides include both naturally occurring Cas9 molecules and Cas9 polypeptides and engineered, altered, or modified Cas9 molecules or Cas9 polypeptides that differ, e.g., by at least one amino acid residue, from a reference sequence, e.g., the most similar naturally occurring Cas9 molecule.

A “reference molecule” as used herein refers to a molecule to which a modified or candidate molecule is compared. For example, a reference Cas9 molecule refers to a Cas9 molecule to which a modified or candidate Cas9 molecule is compared. Likewise, a reference gRNA refers to a gRNA molecule to which a modified or candidate gRNA molecule is compared. The modified or candidate molecule may be compared to the reference molecule on the basis of sequence (e.g., the modified or candidate molecule may have X % sequence identity or homology with the reference molecule) or activity (e.g., the modified or candidate molecule may have X % of the activity of the reference molecule). For example, where the reference molecule is a Cas9 molecule, a modified or candidate molecule may be characterized as having no more than 10% of the nuclease activity of the reference Cas9 molecule. Examples of reference Cas9 molecules include naturally occurring unmodified Cas9 molecules, e.g., a naturally occurring Cas9 molecule from S. pyogenes, S. aureus, or N. meningitidis. In certain embodiments, the reference Cas9 molecule is the naturally occurring Cas9 molecule having the closest sequence identity or homology with the modified or candidate Cas9 molecule to which it is being compared. In certain embodiments, the reference Cas9 molecule is a parental molecule having a naturally occurring or known sequence on which a mutation has been made to arrive at the modified or candidate Cas9 molecule.

“Replacement”, or “replaced”, as used herein with reference to a modification of a molecule does not require a process limitation but merely indicates that the replacement entity is present.

“Small molecule”, as used herein, refers to a compound having a molecular weight less than about 2 kD, e.g., less than about 2 kD, less than about 1.5 kD, less than about 1 kD, or less than about 0.75 kD.

“Subject”, as used herein, may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In certain embodiments, the subject is a human. In other embodiments, the subject is poultry.

“Treat”, “treating” and “treatment”, as used herein, mean the treatment of a disease in a mammal, e.g., in a human, including (a) inhibiting the disease, i.e., arresting or preventing its development or progression; (b) relieving the disease, i.e., causing regression of the disease state; (c) relieving one or more symptoms of the disease; and (d) curing the disease.

“Prevent,” “preventing,” and “prevention” as used herein means the prevention of a disease in a mammal, e.g., in a human, including (a) avoiding or precluding the disease; (b) affecting the predisposition toward the disease; (c) preventing or delaying the onset of at least one symptom of the disease.

“X” as used herein in the context of an amino acid sequence, refers to any amino acid (e.g., any of the twenty natural amino acids) unless otherwise specified.

### 2. Human Immunodeficiency Virus

Human Immunodeficiency Virus (HIV) is a virus that causes severe immunodeficiency. In the United States, more than 1 million people are infected with the virus. Worldwide, approximately 30-40 million people are infected.

HIV is a single-stranded RNA virus that preferentially infects CD4 cells. The virus binds to receptors on the surface of CD4⁺ cells to enter and infect these cells. This binding and infection step is vital to the pathogenesis of HIV. The virus attaches to the CD4 receptor on the cell surface via its own surface glycoproteins, gp120 and gp41. These proteins are made from the cleavage product of gp160. Gp120 binds to a CD4 receptor and must also bind to another coreceptor in order for the virus to enter the host cell. In macrophage-(M-tropic) viruses, the coreceptor is CCR5 occassionaly referred to as the CCR5 receptor. M-tropic virus is found most commonly in the early stages of HIV infection.

There are two types of HIV-HIV-1 and HIV-2. HIV-1 is the predominant global form and is a more virulent strain of the virus. HIV-2 has lower rates of infection and, at present, predominantly affects populations in West Africa. HIV is transmitted primarily through sexual exposure, although the sharing of needles in intravenous drug use is another mode of transmission.

As HIV infection progresses, the virus infects CD4 cells and a subject's CD4 counts fall. With declining CD4 counts, a subject is subject to increasing risk of opportunistic infections (OI). Severely declining CD4 counts are associated with a very high likelihood of OIs, specific cancers (such as Kaposi's sarcoma, Burkitt's lymphoma) and wasting syndrome. Normal CD4 counts are between 600-1200 cells/microliter.

Untreated HIV infection is a chronic, progressive disease that leads to acquired immunodeficiency syndrome (AIDS) and death in the vast majority of subjects. Diagnosis of AIDS is made based on infection with a variety of opportunistic pathogens, presence of certain cancers and/or CD4 counts below 200 cells/μL.

HIV was untreatable and invariably led to death until the late 1980's. Since then, antiretroviral therapy (ART) has dramatically slowed the course of HIV infection. Highly active antiretroviral therapy (HAART) is the use of three or more agents in combination to slow HIV. Antiretroviral therapy (ART) is indicated in a subject whose CD4 counts has dropped below 500 cells/μL. Viral load is the most common measurement of the efficacy of HIV treatment and disease progression. Viral load measures the amount of HIV RNA present in the blood.

Treatment with HAART has significantly altered the life expectancy of those infected with HIV. A subject in the developed world who maintains their HAART regimen can expect to live into their 60's and possibly 70's. However, HAART regimens are associated with significant, long term side effects. First, the dosing regimens are complex and associated with strict food requirements. Compliance rates with dosing can be lower than 50% in some populations in the United States. In addition, there are significant toxicities associated with HAART treatment, including diabetes, nausea, malaise, sleep disturbances. A subject who does not adhere to dosing requirements of HAART therapy may have return of viral load in their blood and are at risk for progression to disease and its associated complications.

### 3. Methods to Treat or Prevent HIV Infection or AIDS

Methods and compositions described herein provide for a therapy, e.g., a one-time therapy, or a multi-dose therapy, that prevents or treats HIV infection and/or AIDS. In certain embodiments, a disclosed therapy prevents, inhibits, or reduces the entry of HIV into CD4 cells of a subject who is already infected. In certain embodiments, methods and compositions described herein prevent, inhibit, and/or reduce the entry of HIV into CD4 cells, CD8 cells, T cells, B cells, neutrophils, eosinophils, GALT, dendritic cells, microglia cells, myeloid progenitor cells, and/or lymphoid progenitor cells of a subject who is already infected. In certain embodiments, knocking out CCR5 on CD4 cells, T cells, GALT, macrophages, dendritic cells, and microglia cells, renders the HIV virus unable to enter host immune cells. In certain embodiments, knocking out CXCR4 on CD4 cells, CD8 cells, T cells, B cells, neutrophils and eosinophils renders the HIV virus unable to enter host immune cells. In certain embodiments, knocking out both CCR5 and CXCR4 on CD4 cells, CD8 cells, T cells, B cells, neutrophils, eosinophils, GALT, dendritic cells, microglia cells, myeloid progenitor cells, lymphoid progenitor cells, hematopoietic stem cells and/or hematopoietic progenitor cells renders the HIV virus unable to enter host immune cells.

Viral entry into CD4 cells, CD8 cells, T cells, B cells, neutrophils, eosinophils, GALT, dendritic cells, microglia cells, myeloid progenitor cells, and/or lymphoid progenitor cells requires interaction of the viral glycoproteins gp41 and gp120 with both the CD4 receptor and a coreceptor, e.g., CCR5, e.g., CXCR4. Once a functional coreceptor such as CCR5 and/or CXCR4 has been eliminated from the surface of the CD4 cells, CD8 cells, T cells, B cells, neutrophils, eosinophils, GALT, dendritic cells, microglia cells, myeloid progenitor cells, lymphoid progenitor cells, hematopoietic stem cells, and/or hematopoietic progenitor cells, the virus is prevented from binding and entering the host cells. In certain embodiments, the disease does not progress or has delayed progression compared to a subject who has not received the therapy.

In certain embodiments, subjects with naturally occurring CCR5 receptor mutations who have delayed HIV progression may confer protection by the mechanism of action described herein. Subjects with a specific deletion in the CCR5 gene (e.g., the delta 32 deletion) have been shown to have much higher likelihood of being long-term non-progressors (meaning they did not require HAART and their HIV infection did not progress). See, e.g., Stewart G J et al., 1997 The Australian Long-Term Non-Progressor Study Group. Aids.11:1833-1838. In addition, a subject who was CCR5+ (had a wild type CCR5 receptor) and infected with HIV underwent a bone marrow transplant for acute myeloid lymphoma. See, e.g., Hutter Get al., 2009N ENGL J MED.360:692-698. The bone marrow transplant (BMT) was from a subject homozygous for a CCR5 delta 32 deletion. Following BMT, the subject did not have progression of HIV and did not require treatment with ART. These subjects offer evidence for the fact that alteration of a CCR5 gene (e.g., introduction of one or more mutations (e.g., one or more protective mutations, such as a delta32 mutation), knockout, or knockdown of the CCR5 gene as described in Section 4 below), prevents, delays or diminishes the ability of HIV to infect the subject. Mutation or deletion of the CCR5 gene, or reduced CCR5 gene expression, can therefore reduce the progression, virulence and pathology of HIV.

In certain embodiments, alteration of a CXCR4 gene (e.g., knockout, knockdown, or introduction one or more mutations (e.g., one more single or two base substitutions) of the CXCR4 gene, e.g., as decribed in Section 5 below) eliminates or reduces CXCR4 gene expression. Decreased expression of coreceptor CXCR4 on the surface of CD4 cells, CD8 cells, T cells, B cells, neutrophils and eosinophils can prevent, delay or diminish the ability of T-trophic HIV to infect the subject. Mutation or deletion of the CXCR4 gene, or reduced CXCR4 gene expression, can therefore reduce the progression, virulence and pathology of HIV.

In certain embodiments, alteration of both the CCR5 and CXCR4 gene (e.g., as described in Section 6 below) eliminates or reduces CCR5 and CXCR4 gene expression. Decreased expression of co-receptors CCR5 and CXCR4 on the surface of CD4 cells, CD8 cells, T cells, B cells, neutrophils, eosinophils, GALT, dendritic cells, microglia cells, myeloid progenitor cells, and/or lymphoid progenitor cells can prevent, delay or diminish the ability of both M-trophic and T-trophic HIV to infect the subject. Mutation or deletion of both the CCR5 and the CXCR4 genes, or reduced CCR5 and CXCR4 gene expression, can therefore reduce the progression, virulence and pathology of HIV.

In certain embodiments, a method described herein is used to treat a subject suffering from HIV.

In certain embodiments, a method described herein is used to treat a subject suffering from AIDS.

In certain embodiments, a method described herein is used to prevent, or delay the onset or progression of, HIV infection and AIDS in a subject at high risk for HIV infection.

In certain embodiments, a method described herein results in a selective advantage to survival of treated CD4 cells. In certain embodiments, a method described herein results in a selective advantage to survival of treated CD8 cells, T cells, B cells, neutrophils, eosinophils, GALT, dendritic cells, microglia cells, myeloid progenitor cells, and/or lymphoid progenitor cells. In certain embodiments, some proportion of CD4 cells, T cells, GALT, macrophages, dendritic cells, microglia cells, myeloid progenitor cells, lymphoid progenitor cells, and/or hematopoietic stem cells can be modified and have a CCR5 protective mutation. In certain embodiments, some proportion of CD4 cells, T cells, GALT, macrophages, dendritic cells, microglia cells, myeloid progenitor cells, lymphoid progenitor cells, and/or hematopoietic stem cells can be modified and have a CCR5 deletion mutation. In certain embodiments, some proportion of CD4 cells, T cells, GALT, macrophages, dendritic cells, microglia cells, myeloid progenitor cells, lymphoid progenitor cells, and/or hematopoietic stem cells can be modified and have a CCR5 mutation that decreases CCR5 gene expression. In certain embodiments, some proportion of CD4 cells, CD8 cells, T cells, B cells, neutrophils, eosinophils, myeloid progenitor cells, lymphoid progenitor cells, and/or hematopoietic stem cells can be modified and have a CXCR4 deletion mutation. In certain embodiments, some proportion of CD4 cells, CD8 cells, T cells, B cells, neutrophils, eosinophils, myeloid progenitor cells, lymphoid progenitor cells, and/or hematopoietic stem cells can be modified and have a CXCR4 mutation that decreases CXCR4 gene expression.

In certain embodiments, some proportion of CD4 cells, CD8 cells, T cells, B cells, neutrophils, eosinophils, GALT, dendritic cells, microglia cells, myeloid progenitor cells, lymphoid progenitor cells, and/or hematopoietic stem cells can be modified and have both a CCR5 protective mutation and a CXCR4 deletion mutation. In certain embodiments, some proportion of CD4 cells, CD8 cells, T cells, B cells, neutrophils, eosinophils, GALT, dendritic cells, microglia cells, myeloid progenitor cells, lymphoid progenitor cells, and/or hematopoietic stem cells can be modified and have both a CCR5 protective mutation and a mutation that decreases CXCR4 gene expression.

In certain embodiments, some proportion of CD4 cells, CD8 cells, T cells, B cells, neutrophils, eosinophils, GALT, dendritic cells, microglia cells, myeloid progenitor cells, lymphoid progenitor cells, and/or hematopoietic stem cells can be modified and have both a CCR5 deletion mutation and a CXCR4 deletion mutation. In certain embodiments, some proportion of CD4 cells, CD8 cells, T cells, B cells, neutrophils, eosinophils, GALT, dendritic cells, microglia cells, myeloid progenitor cells, lymphoid progenitor cells, and/or hematopoietic stem cells can be modified and have both a CCR5 deletion mutation and a mutation that decreases CXCR4 gene expression.

In certain embodiments, some proportion of CD4 cells, CD8 cells, T cells, B cells, neutrophils, eosinophils, GALT, dendritic cells, microglia cells, myeloid progenitor cells, lymphoid progenitor cells, and/or hematopoietic stem cells can be modified and have both a mutation that decreases CCR5 gene expression and a CXCR4 deletion mutation. In certain embodiments, some proportion of CD4 cells, CD8 cells, T cells, B cells, neutrophils, eosinophils, GALT, dendritic cells, microglia cells, myeloid progenitor cells, lymphoid progenitor cells, and/or hematopoietic stem cells can be modified and have both a mutation that decreases CCR5 gene expression and a mutation that decreases CXCR4 gene expression. In certain embodiments, these cells are not subject to infection with HIV. Cells that are not modified may be infected with HIV and are expected to undergo cell death. In certain embodiments, after the treatment described herein, treated cells survive, while untreated cells die. In certain embodiments, this selective advantage drives eventual colonization in all body compartments with 100% CCR5-negative CD4 cells, T cells, GALT, macrophages, dendritic cells, microglia cells, myeloid progenitor cells, lymphoid progenitor cells, and hematopoietic stem cells derived from treated cells, conferring complete protection in treated subjects against infection with M tropic HIV. In certain embodiments, this selective advantage drives eventual colonization in all body compartments with 100% CXCR4-negative CD4 cells, CD8 cells, T cells, B cells, neutrophils, eosinophils, myeloid progenitor cells, lymphoid progenitor cells, and hematopoietic stem cells derived from treated cells, conferring complete protection in treated subjects against infection with T tropic HIV. In certain embodiments, this selective advantage drives eventual colonization in all body compartments with 100% CCR5-negative and 100% CXCR4-negative CD4 cells, CD8 cells, T cells, B cells, neutrophils, eosinophils, GALT, dendritic cells, microglia cells, myeloid progenitor cells, lymphoid progenitor cells, and hematopoietic stem cells derived from treated cells, conferring complete protection in treated subjects against infection with both M tropic and T tropic HIV.

In certain embodiments, the method comprises initiating treatment of a subject prior to disease onset.

In certain embodiments, the method comprises initiating treatment of a subject after disease onset.

In certain embodiments, the method comprises initiating treatment of a subject after disease onset, e.g., 1, 2, 3, 4, 5, 6, 7, 8, 9, 10, 12, 16, 24, 36, 48 or more months after onset of HIV infection or AIDS. In certain embodiments, this may be effective as disease progression is slow in some cases and a subject may present well into the course of illness.

In certain embodiments, the method comprises initiating treatment of a subject in an advanced stage of disease, e.g., to slow viral replication and viral load.

Overall, initiation of treatment for a subject at all stages of disease is expected to prevent or reduce disease progression and benefit a subject.

In certain embodiments, the method comprises initiating treatment of a subject prior to disease onset and prior to infection with HIV.

In certain embodiments, the method comprises initiating treatment of a subject in an early stage of disease, e.g., when when a subject has tested positive for HIV infection but has no signs or symptoms associated with HIV.

In certain embodiments, the method comprises initiating treatment of a patient at the appearance of a reduced CD4 count or a positive HIV test.

In certain embodiments, the method comprises treating a subject considered at risk for developing HIV infection.

In certain embodiments, the method comprises treating a subject who is the spouse, partner, sexual partner, newborn, infant, or child of a subject with HIV.

In certain embodiments, the method comprises treating a subject for the prevention or reduction of HIV infection.

In certain embodiments, the method comprises treating a subject at the appearance of any of the following findings consistent with HIV: low CD4 count; opportunistic infections associated with HIV, including but not limited to: candidiasis, mycobacterium tuberculosis, cryptococcosis, cryptosporidiosis, cytomegalovirus; and/or malignancy associated with HIV, including but not limited to: lymphoma, Burkitt's lymphoma, or Kaposi's sarcoma.

In certain embodiments, the method comprises treating a subject who is undergoing a heterologous hematopoietic stem cell transplant, including an umbilical cord blood transplant, e.g., in a subject with or without HIV.

In certain embodiments, a cell is treated ex vivo and returned to a patient.

In certain embodiments, an autologous CD4 cell can be treated ex vivo and returned to the subject. In certain embodiments, an autologous CD8 cell, T cell, B cell, neutrophil, eosinophil, GALT, dendritic cell, microglia cell, myeloid progenitor cell, and/or lymphoid progenitor cell cell can be treated ex vivo and returned to the subject.

In certain embodiments, a heterologous CD4 cell can be treated ex vivo and transplanted into the subject. In certain embodiments, a heterologous CD8 cell, T cell, B cell, neutrophil, eosinophil, GALT, dendritic cell, microglia cell, myeloid progenitor cell, and/or lymphoid progenitor cell cell can be treated ex vivo and returned to the subject.

In certain embodiments, an autologous stem cell, e.g., an autologous hematopoietic stem cell, e.g., an autologous umbilical cord blood transplant cell, can be treated ex vivo and returned to the subject.

In certain embodiments, a heterologous stem cell, e.g., a heterologous hematopoietic stem cell, e.g., an autologous umbilical cord blood transplant cell, can be treated ex vivo and transplanted into the subject.

In certain embodiments, the treatment comprises delivery of a gRNA molecule by intravenous injection, intramuscular injection; subcutaneous injection; intra bone marrow injection; intrathecal injection; or intraventricular injection.

In certain embodiments, the treatment comprises delivery of a gRNA molecule by an AAV.

In certain embodiments, the treatment comprises delivery of a gRNA molecule by a lentivirus.

In certain embodiments, the treatment comprises delivery of a gRNA molecule by a nanoparticle.

In certain embodiments, the treatment comprises delivery of a gRNA molecule by a parvovirus, e.g., a specifically a modified parvovirus designed to target bone marrow cells and/or CD4 cells, CD8 cells, T cells, B cells, neutrophils, eosinophils, GALT, dendritic cells, microglia cells, myeloid progenitor cells, lymphoid progenitor cells, and/or hematopoietic stem cells.

In certain embodiments, the treatment is initiated after a subject is determined to not have a mutation (e.g., an inactivating mutation, e.g., an inactivating mutation in either or both alleles) in CCR5 by genetic screening, e.g., genotyping, wherein the genetic testing was performed prior to or after disease onset.

In certain embodiments, treatment to eliminate or decrease CXCR4 gene expression is initiated after a subject is determined to have a mutation (e.g., an inactivating mutation, e.g., an inactivating mutation in either or both alleles) in CCR5 by genetic screening, e.g., genotyping, wherein the genetic testing was performed prior to or after disease onset.

3.1. Modified HSC Transplantation for the Treatment of HIV/AIDS

Transplantation of HSCs into a subject suffering from HIV is curative if the cells are genetically modified to resist HIV infection (e.g., reduced expression of CXCR4 and/or CCR5 HIV co-receptor). For treatment, the patient is transplanted with either autologous or HLA-matched/HLA-identical HSCs that are genome-edited such that all blood progeny from the modified HSCs are resistant to HIV infection. The HSCs are collected from the donor (either autologous or allogeneic HLA-matched/HLA identical), genome-edited ex vivo to confer resistance to HIV infection, and then infused the patient. After the HSCs engraft, the HSCs can reconstitute the blood lineages such that the HSC progeny (e.g., blood lineages, e.g., myeloid cells, lymphoid cells, microglia) can have altered expression of CCR5 and CXCR4, and thus, the HIV virus is unable to enter the genome-edited blood cells (i.e., the progeny of the genome-edited HSCs). Without wishing to be bound by any theory, it is thought that, insofar as the only cells to survive HIV infection are the cells that are genome-edited to be resistant to HIV infection, the genome-edited lymphoid and myeloid cells will have a selective advantage over the unedited cells. The absence of T cells due to HIV infection provides selective pressure on genome editing HScs to produce HIV resistant blood cells beause there are not enough cells present for immune function. This selective advantage suggests that (while not wishing to be bound by theory) even comparatively low levels of gene editing (<10%, e.g. 4% or 5%) in the HSCs before transplant could be sufficient to support repopulation of the blood in vivo after transplant with genome-edited HIV resistant myeloid and lymphoid progeny. Transplantation of CCR5 and/or CXCR4 genome-edited autologous or allogeneic HLA-matched/HLA-identical HSCs provides an HIV resistant immune system after transplantation.

3.2. Modified T Cell Add-Back in the Case of Allogeneic HSC Transplantation

A subject suffering from HIV who is undergoing allogeneic HSC transplantation is at risk for opportunistic infections in the period immediately following transplantation. A subject suffering from HIV commonly suffers from low T cell counts due to virus induced destruction of T cells; the subject can be T cell depleted prior to HSC transplantation. In addition, the subject receives a myeloablative conditioning regimen to prepare for the HSC transplantation, which further depletes T cells that help prevent infection. Immune reconstitution can take several months in the subject. During this time, HSCs from the donor differentiate into T cells, travel to the thymus and are exposed to antigens and begin to reconstitute adaptive immunity.

In a subject suffering from HIV who is undergoing allogeneic HSC transplantation, the use of modified T cell add-back in the period immediately following the transplant can provide an adaptive immunity lymphoid bridge. HSCs derived from the bone marrow or peripheral blood of the donor are modified according to the methods, e.g., undergo CRISPR/Cas9-mediated modifications at the CXCR4 and/or CCR5 locus, and are differentiated into lymphoid progenitor cells ex vivo. Modification, e.g., CRISPR/Cas9 mediated modifications at the CXCR4 and/or CCR5 locus, renders the cells HIV-resistant. The differentiated, HIV-resistant lymphoid progenitor cells or lymphoid cells are dosed in a subject immediately following myeloablative conditioning and prior to allogeneic HSC transplant, or co-infused with HSC transplant, or dosed following HSC transplant. In certain embodiments, administration of HIV resistant, differentiated lymphoid cells in a subject undergoing HSC transplantation provides a short term lymphoid bridge of HIV resistant cells. These cells provide short term immunity against opportunistic infection. The modified T cells used in lymphoid or T cell add-back may have a limited life span (approximately 2 weeks to 60 days to one year) (Westera et al., Blood 2013; 122(13):2205-2212). In the immediate post-transplantation period, these cells can provide protective immunity in a subject. The dose of such cells can be modified to balance immune protection (conferred by dosing with HIV resistant, differentiated lymphoid cells), Graft vs. Leukemia effect (GVL) in the case where the HIV patient also has concominant blood cancer (e.g., lymphoma), and graft versus host disease (a higher risk of GVHD is associated with higher T cell doses) (Montero et al., Biol Blood Marrow Transplant. 2006 Dec.; 12(12):1318-25). The methods described herein can be dosed one, two, three or multiple times, to maintain T cell counts and immunity until the donor HSC cells have reconstituted the lymphoid lineage.

In a subject suffering from HIV who is undergoing allogeneic HSC transplantation, the use of myeloid and T cell add-back in the period immediately following the transplant can provide a myeloid and adaptive immunity lymphoid bridge. Donor HSCs are modified according to the methods described herein and differentiated into myeloid and lymphoid progenitor cells ex vivo. The differentiated, HIV-resistant myeloid and lymphoid progenitor cells are dosed in a subject immediately following myeloablative conditioning and prior to allogeneic HSC transplant, or co-infused with HSC transplant, or dosed following HSC transplant. The differentiated, HIV-resistant myeloid and lymphoid progenitor cells are dosed together, or are dosed separately, e.g., modified, HIV resistant myeloid progenitor cells are dosed in one dosing regimen and modified, HIV resistant lymphoid progenitor cells are dosed in an alternative dosing regimen. Administration of HIV resistant, differentiated myeloid and lymphoid cells in a subject undergoing HSC transplantation provides a short term myeloid and lymphoid bridge of HIV resistant cells. These cells provide short term protection against anemia and short term immunity against opportunistic infection. These cells can have a limited life span. In the immediate post-transplantation period, these cells can improve anemia and provide protective immunity in a subject. The dose of such cells can be modified to balance immune protection (conferred by dosing with HIV resistant, differentiated myeloid and lymphoid cells) and graft versus host disease (a higher risk of GVHD is associated with higher T cell doses) (Montero et al., Biol Blood Marrow Transplant. 2006 Dec.; 12(12):1318-25). The methods described herein can be dosed one, two, three or multiple times, to maintain myeloid and lymphoid cell counts and until the donor HSC cells have reconstituted the myeloid and lymphoid lineage.

In certain embodiments, the method is used to treat a subject with late-stage HIV who is at risk for opportunistic infection due to very low and/or declining T cell counts. In certain embodiments, the method of T cell add-back is used to treat a subject with late-stage HIV who is undergoing allogeneic HSCT for the treatment of HIV. In certain embodiments, the method of T cell add-back is used to treat a subject with any stage of HIV who is undergoing allogeneic HSCT for the treatment of HIV.

3.3. Modified T Cell Add-Back in the Case of Autologous HSC Transplantation

A subject suffering from HIV who is undergoing autologous HSC transplantation is at risk for opportunistic infections in the period immediately following transplantation. A subject suffering from HIV commonly suffers from low T cell counts due to virus induced destruction of T cells. The HIV-positive subject who is a candidate for HSC transplantation receives a myeloablative conditioning regimen to prepare for the HSC transplantation. Myeloablation further depletes HIV-infected and HIV-uninfected T cells that help prevent infection. Immune reconstitution can take 2-3 months in the subject. During this time, HSCs from the transplant differentiate into T-cells, travel to the thymus and are exposed to antigens and begin to reconstitute adaptive immunity.

In a subject suffering from HIV who is undergoing autologous HSC transplantation, the use of modified T cell add-back in the period immediately following the transplant can provide an adaptive immunity lymphoid bridge. HSCs or PBSCs derived from the bone marrow or peripheral blood of the subject are modified according to the methods, e.g., undergo CRISPR/Cas9-mediated modifications at the CXCR4 and/or CCR5 locus, and are differentiated into lymphoid progenitor cells ex vivo. Modification, e.g., CRISPR/Cas9 mediated modifications at the CXCR4 and/or CCR5 locus, renders the cells HIV-resistant.

An advantage of modifying HSCs or lymphoid progenitor cells (as opposed to modifying T cells) is that these cells are not infected with HIV (HSCs and progenitors do not express both HIV co-receptors that are required for viral entry). T cells that have been modified by the methods, e.g., autologous T cells that have been differentiated from HIV-negative HSC or progenitors and have been edited by the methods described herein, can be HIV resistant when re-infused back to the subject.

Autologous, differentiated, HIV-resistant lymphoid progenitor cells or T cells can be dosed in a subject immediately following myeloablative conditioning and prior to autologous HSC transplant, or co-infused with HSC transplant, or dosed following HSC transplant. In certain embodiments, administration of HIV resistant, differentiated lymphoid cells or T cells in a subject undergoing autologous HSC transplantation provides a short term lymphoid bridge of HIV resistant cells. These cells provide short term immunity against opportunistic infection. The modified T cells used in lymphoid or T cell add-back can have a limited life span (approximately 2 weeks to 60 days to 1 year) (Westera et al., Blood 2013; 122(13):2205-2212). In the immediate post-transplantation period, these cells can provide protective immunity in a subject. The dose of such cells can be modified to balance immune protection (conferred by dosing with HIV resistant, differentiated myeloid and lymphoid cells) and graft versus host disease (a higher risk of GVHD is associated with higher T cell doses) (Montero et al., Biol Blood Marrow Transplant. 2006 Dec.; 12(12):1318-25). The methods described herein can be dosed one, two, three or multiple times, to maintain T cell counts and immunity until the autologous HSC cells have reconstituted the lymphoid lineage.

In a subject suffering from HIV who is undergoing autologous HSC transplantation, the use of myeloid and T cell add-back in the period immediately following the transplant can provide a myeloid and adaptive immunity lymphoid bridge. HSCs derived from the bone marrow or mobilized peripheral blood of the subject are modified according to the methods described herein and differentiated into myeloid and lymphoid progenitor cells ex vivo. An advantage of modifying HSCs mobilized peripheral blood (as opposed to modifying T-cells) is that these cells are not infected with HIV (stem cells are HIV resistant as they do not express both HIV co-receptors) and when added back to the subject can be HIV naïve (as well as HIV resistant). The differentiated, HIV-resistant myeloid and lymphoid progenitor cells are dosed in a subject immediately following myeloablative conditioning and prior to autologous HSC transplant, or co-infused with HSC transplant, or dosed following HSC transplant. The differentiated, HIV-resistant myeloid and lymphoid progenitor cells are dosed together, or are dosed separately, e.g., modified, HIV resistant myeloid progenitor cells are dosed in one dosing regimen and modified, HIV resistant lymphoid progenitor cells are dosed in an alternative dosing regimen. In certain embodiments, administration of HIV resistant, differentiated myeloid and lymphoid cells in a subject undergoing HSC transplantation provides a short term myeloid and lymphoid bridge of HIV resistant cells. These cells provide short term protection against anemia and short term immunity against opportunistic infection. These cells can have a limited life span. In the immediate post-transplantation period, these cells can improve anemia and provide protective immunity in a subject. The dose of such cells can be modified to balance reduced anemia and immune protection (conferred by dosing with HIV resistant, differentiated myeloid and lymphoid cells) and graft versus host disease (a higher risk of GVHD is associated with higher T-cell doses) (Montero et al., Biol Blood Marrow Transplant. 2006 Dec.; 12(12):1318-25). The methods described herein can be dosed one, two, three or multiple times, to maintain myeloid and lymphoid cell counts and until the autologous HSC cells have reconstituted the myeloid and lymphoid lineage.

In certain embodiments, the method is used to treat a subject with late-stage HIV who is at risk for opportunistic infection due to very low and/or declining T-cell counts. In certain embodiments, the method of T-cell add-back is used to treat a subject with late-stage HIV who is undergoing autologous HSCT for the treatment of HIV. In certain embodiments, the method of T-cell add-back is used to treat a subject with any stage of HIV who is undergoing autologous HSCT for the treatment of HIV.

3.4 Stand-Alone T Cell Therapy for HIV—Ex Vivo Modification of Lymphoid Cells and/or T-Cells in Acute or Sub-Acute Setting in a Subject with Opportunistic Infection, Severe HIV and/or Refractory HIV for Short-Term Restoration of T-Cell Mediated Immunity

Autologous or allogeneic HLA-matched or HLA-identical lymphoid cells and/or T-cells can be modified by the methods, e.g., CRISPR/Cas9-mediated modifications at the CXCR4 gene and/or CCR5 gene, and dosed to subjects with HIV, providing short-term adaptive immunity in subjects with HIV.

(a) HSCs derived from the bone marrow or mobilized peripheral blood of the subject are modified according to the methods, e.g., CRISPR/Cas9-mediated modifications at the CXCR4 gene and/or CCR5 gene, and differentiated into lymphoid progenitor cells and/or T-cells ex vivo. An advantage of modifying HSCs (as opposed to modifying lymphoid cells or T-cells) is that HSCs are not infected with HIV. Stem cells are HIV resistant as they do not express both HIV co-receptors. When added back to the subject, after differentiation into T-cells, the T-cells can be HIV naive as well as HIV resistant. These modified cells are also self-derived (autologous) so have no risk of generating a graft vs. host immune reaction in the subject.

(b) HSCs derived from the bone marrow or mobilized peripheral blood of an HLA matched or HLA identical donor are modified ex vivo according to the methods, e.g., CRISPR/Cas9-mediated modifications at the CXCR4 gene and/or CCR5 gene, and differentiated into lymphoid progenitor cells and/or T cells. When added back to the subject, the allogeneic, modified lymphoid cells and/or T cells can be HIV naive as well as HIV resistant.

(c) T-cells derived from the peripheral blood of a donor are modified ex vivo according to the methods, e.g., CRISPR/Cas9-mediated modifications at the CXCR4 gene and/or CCR5 gene s. When added back to the subject, the modified, allogeneic lymphoid cells and/or T cells can be HIV naive as well as HIV resistant. (See Example 9 for data demonstrating T cell modification.)

Modified, HIV-resistant T cells (autologous or allogeneic) are dosed in a subject suffering from HIV, including, but not limited to: a subject having an opportunistic infection, a subject hospitalized for a suspected or known opportunistic infection, a subject having rapidly declining T cell counts, a subject having very low T cell counts and being at risk for opportunistic infection, and a subject preparing for surgery or HSC transplantation and requiring additional T cell immunity. The modified lymphoid progenitor cells or T-cells can be used in the setting of severe, HIV, refractory HIV, end-stage HIV (e.g., AIDS), treatment-resistant HIV. The treatment is given in an acute or sub-acute setting in a subject with severe and/or refractory HIV for short-term or intermediate-term restoration of T cell counts, lymphoid activity and/or recovery from opportunistic infection. The goal of treatment is to provide short or intermediate term lymphoid immunity in the case of low T counts or severe opportunistic infection.

### 4. Methods of Altering CCR5

As disclosed herein, the CCR5 gene can be altered by gene editing, e.g., using CRISPR-Cas9 mediated methods as described herein.

Methods, genome editing systems, and compositions discussed herein, provide for altering a CCR5 target position in the CCR5 gene. A CCR5 target position can be altered by gene editing, e.g., using CRISPR-Cas9-mediated methods, genome editing systems, and compositions described herein.

Altering a CCR5 gene can be achieved by one or more of the following approaches:

(4.1) knocking out the CCR5 gene:
(4.1a) insertion or deletion (e.g., NHEJ-mediated insertion or deletion) of one or more nucleotides in close proximity to or within the early coding region of the CCR5 gene,
(4.1b) deletion (e.g., NHEJ-mediated deletion) of a genomic sequence including at least a portion of the CCR5 gene,
(4.1c) knockout of CCR5 with concomitant knock-in of anti-HIV gene or genes under expression of endogenous promoter or Pol III promoter; and
(4.1d) knockout of CCR5 with concomitant knock-in of drug resistance selectable marker for enabling selection of modified HSCs;

(4.2) knocking down the CCR5 gene mediated by enzymatically inactive Cas9 (eiCas9) molecule or an eiCas9-fusion protein; or

(4.3) Introducing one ore more mutations in the CCR5 gene
(4.3a) NHEJ-mediated creation of naturally occurring delta 32 mutation in CCR5 gene; and(4.3b) HDR-mediated introduction of delta 32 mutation to CCR5

Exemplary mechanisms that can be associated with the alteration of a CCR5 gene include, but are not limited to, non-homologous end joining (“NHEJ”; e.g., classical or alternative), microhomology-mediated end joining (“MMEJ”), homology-directed repair (“HDR”; e.g., endogenous donor template mediated), synthesis dependent strand annealing (“SDSA”), single strand annealing or single strand invasion.

In certain embodiments, the methods, genome editing systems, and compositions described herein introduce one or more breaks near the early coding region in at least one allele of the CCR5 gene. In certain embodiments, methods, genome editing systems, and compositions described herein introduce two or more breaks to flank at least a portion of the CCR5 gene . The two or more breaks remove (e.g., delete) a genomic sequence including at least a portion of the CCR5 gene. In certain embodiments methods described herein comprises creation of naturally occurring delta 32 mutation in the CCR5 gene. In certain embodiments, methods described herein comprise knocking down the CCR5 gene mediated by enzymatically inactive Cas9 (eiCas9) molecule or an eiCas9-fusion protein by targeting the promoter region of CCR5 target knockdown position. In certain embodiments, methods described herein comprises concomitantly knock down the CCR5 gene and knock-in of anti-HIV gene or genes under expression of endogenous promoter or Pol III promoter. In certain embodiments, methods described herein comprises concomitantly knockout of CCR5 gene and knock-in of drug resistance selectable marker for enabling selection of modified HSCs. In certain embodiments, methods described herein comprises HDR-mediated introduction of delta 32 mutation to CCR5. Methods, e.g., approaches 4.1a, 4.1b, 4.2, 4.3a, 4.3b, and 4.4described herein result in targeting (e.g., alteration) of the CCR5 gene.

### (4.1a) Knocking Out CCR5 by Introducing an Indel in the CCR5 Gene

In certain embodiments, the method comprises introducing an insertion or deletion of one more nucleotides in close proximity to the CCR5 target knockout position (e.g., the early coding region) of the CCR5 gene. As described herein, in certain embodiments, the method comprises the introduction of one or more breaks (e.g., single strand breaks or double strand breaks) sufficiently close to (e.g., either 5′ or 3′ to) the early coding region of the CCR5 target knockout position, such that the break-induced indel could be reasonably expected to span the CCR5 target knockout position (e.g., the early coding region). In certain embodiments, NHEJ-mediated repair of the break(s) allows for the NHEJ-mediated introduction of an indel in close proximity to within the early coding region of the CCR5 target knockout position.

In certain embodiments, the method comprises introducing a deletion of a genomic sequence comprising at least a portion of the CCR5 gene. As described herein, in certain embodiments, the method comprises the introduction of two double stand breaks—one 5′ and the other 3′ to (i.e., flanking) the CCR5 target position. In certain embodiments, two gRNAs, e.g., unimolecular (or chimeric) or modular gRNA molecules, are configured to position the two double strand breaks on opposite sides of the CCR5 target knockout position in the CCR5 gene.

In certain embodiments, a single strand break is introduced (e.g., positioned by one gRNA molecule) at or in close proximity to a CCR5 target position in the CCR5 gene. In certain embodiments, a single gRNA molecule (e.g., with a Cas9 nickase) is used to create a single strand break at or in close proximity to the CCR5 target position, e.g., the gRNA is configured such that the single strand break is positioned either upstream (e.g., within 500 bp upstream, e.g., within 200 bp upstream) or downstream (e.g., within 500 bp downstream, e.g., within 200 bp downstream) of the CCR5 target position. In certain embodiments, the break is positioned to avoid unwanted target chromosome elements, such as repeat elements, e.g., an Alu repeat.

In certain embodiments, a double strand break is introduced (e.g., positioned by one gRNA molecule) at or in close proximity to a CCR5 target position in the CCR5 gene. In certain embodiments, a single gRNA molecule (e.g., with a Cas9 nuclease other than a Cas9 nickase) is used to create a double strand break at or in close proximity to the CCR5 target position, e.g., the gRNA molecule is configured such that the double strand break is positioned either upstream (e.g., within 500 bp upstream, e.g., within 200 bp upstream) or downstream of (e.g., within 500 bp downstream, e.g., within 200 bp downstream) of a CCR5 target position. In certain embodiments, the break is positioned to avoid unwanted target chromosome elements, such as repeat elements, e.g., an Alu repeat.

In certain embodiments, two single strand breaks are introduced (e.g., positioned by two gRNA molecules) at or in close proximity to a CCR5 target position in the CCR5 gene. In certain embodiments, two gRNA molecules (e.g., with one or two Cas9 nickcases) are used to create two single strand breaks at or in close proximity to the CCR5 target position, e.g., the gRNAs molecules are configured such that both of the single strand breaks are positioned e.g., within 500 bp upstream, e.g., within 200 bp upstream) or downstream (e.g., within 500 bp downstream, e.g., within 200 bp downstream) of the CCR5 target position. In certain embodiments, two gRNA molecules (e.g., with two Cas9 nickcases) are used to create two single strand breaks at or in close proximity to the CCR5 target position, e.g., the gRNAs molecules are configured such that one single strand break is positioned upstream (e.g., within 200 bp upstream) and a second single strand break is positioned downstream (e.g., within 200 bp downstream) of the CCR5 target position. In certain embodiments, the breaks are positioned to avoid unwanted target chromosome elements, such as repeat elements, e.g., an Alu repeat.

In certain embodiments, two double strand breaks are introduced (e.g., positioned by two gRNA molecules) at or in close proximity to a CCR5 target position in the CCR5 gene. In certain embodiments, two gRNA molecules (e.g., with one or two Cas9 nucleases that are not Cas9 nickases) are used to create two double strand breaks to flank a CCR5 target position, e.g., the gRNA molecules are configured such that one double strand break is positioned upstream (e.g., within 500 bp upstream, e.g., within 200 bp upstream) and a second double strand break is positioned downstream (e.g., within 500 bp downstream, e.g., within 200 bp downstream) of the CCR5 target position. In certain embodiments, the breaks are positioned to avoid unwanted target chromosome elements, such as repeat elements, e.g., an Alu repeat.

In certain embodiments, one double strand break and two single strand breaks are introduced (e.g., positioned by three gRNA molecules) at or in close proximity to a CCR5 target position in the CCR5 gene. In certain embodiments, three gRNA molecules (e.g., with a Cas9 nuclease other than a Cas9 nickase and one or two Cas9 nickases) to create one double strand break and two single strand breaks to flank a CCR5 target position, e.g., the gRNA molecules are configured such that the double strand break is positioned upstream or downstream of (e.g., within 500 bp, e.g., within 200 bp upstreamor downstream) of the CCR5 target position, and the two single strand breaks are positioned at the opposite site, e.g., downstream or upstrea m (e.g., within 500 bp, e.g., within 200 bp downstream or upstream), of the CCR5 target position. In certain embodiments, the breaks are positioned to avoid unwanted target chromosome elements, such as repeat elements, e.g., an Alu repeat.

In certain embodiments, four single strand breaks are introduced (e.g., positioned by four gRNA molecules) at or in close proximity to a CCR5 target position in the CCR5 gene. In certain embodiments, four gRNA molecule (e.g., with one or more Cas9 nickases are used to create four single strand breaks to flank a CCR5 target position in the CCR5 gene, e.g., the gRNA molecules are configured such that a first and second single strand breaks are positioned upstream (e.g., within 500 bp upstream, e.g., within 200 bp upstream) of the CCR5 target position, and a third and a fourth single stranded breaks are positioned downstream (e.g., within 500 bp downstream, e.g., within 200 bp downstream) of the CCR5 target position. In certain embodiments, the breaks are positioned to avoid unwanted target chromosome elements, such as repeat elements, e.g., an Alu repeat.

In certain embodiments, two or more (e.g., three or four) gRNA molecules are used with one Cas9 molecule. In certain embodiments, when two ore more (e.g., three or four) gRNAs are used with two or more Cas9 molecules, at least one Cas9 molecule is from a different species than the other Cas9 molecule(s). For example, when two gRNA molecules are used with two Cas9 molecules, one Cas9 molecule can be from one species and the other Cas9 molecule can be from a different species. Both Cas9 species are used to generate a single or double-strand break, as desired.

### (4.1b) Knocking Out CCR5 by Deleting a Genomic Sequence Including at Least a Portion of the CCR5 Gene

In certain embodiments, the method comprises deleting (e.g., NHEJ-mediated deletion) a genomic sequence including at least a portion of the CCR5 gene. As described herein, in certain embodiments, the method comprises the introduction two sets of breaks (e.g., a pair of double strand breaks, one double strand break or a pair of single strand breaks, or two pairs of single strand breaks) to flank a region of the CCR5 gene (e.g., a coding region, e.g., an early coding region, or a non-coding region, e.g., a non-coding sequence of the CCR5 gene, e.g., a promoter, an enhancer, an intron, a 3′UTR, and/or a polyadenylation signal). In certain embodiments, NHEJ-mediated repair of the break(s) allows for alteration of the CCR5 gene as described herein, which reduces or eliminates expression of the gene, e.g., to knock out one or both alleles of the CCR5 gene.

In certain embodiments, two double strand breaks are introduced (e.g., positioned by two gRNA molecules) at or in close proximity to a CCR5 target position in the CCR5 gene. In certain embodiments, two gRNA molecules (e.g., with one or two Cas9 nucleases that are not Cas9 nickases) are used to create two double strand breaks to flank a CCR5 target position, e.g., the gRNA molecules are configured such that one double strand break is positioned upstream (e.g., within 500 bp upstream, e.g., within 200 bp upstream) and a second double strand break is positioned downstream (e.g., within 500 bp downstream, e.g., within 200 bp downstream) of the CCR5 target position. In certain embodiments, the breaks are positioned to avoid unwanted target chromosome elements, such as repeat elements, e.g., an Alu repeat.

In certain embodiments, one double strand break and two single strand breaks are introduced (e.g., positioned by three gRNA molecules) at or in close proximity to a CCR5 target position in the CCR5 gene. In certain embodiments, three gRNA molecules (e.g., with a Cas9 nuclease other than a Cas9 nickase and one or two Cas9 nickases) to create one double strand break and two single strand breaks to flank a CCR5 target position, e.g., the gRNA molecules are configured such that the double strand break is positioned upstream or downstream of (e.g., within 500 bp, e.g., within 200 bp upstreamor downstream) of the CCR5 target position, and the two single strand breaks are positioned at the opposite site, e.g., downstream or upstrea m (e.g., within 500 bp, e.g., within 200 bp downstream or upstream), of the CCR5 target position. In certain embodiments, the breaks are positioned to avoid unwanted target chromosome elements, such as repeat elements, e.g., an Alu repeat.

In certain embodiments, four single strand breaks are introduced (e.g., positioned by four gRNA molecules) at or in close proximity to a CCR5 target position in the CCR5 gene. In certain embodiments, four gRNA molecule (e.g., with one or more Cas9 nickases are used to create four single strand breaks to flank a CCR5 target position in the CCR5 gene, e.g., the gRNA molecules are configured such that a first and second single strand breaks are positioned upstream (e.g., within 500 bp upstream, e.g., within 200 bp upstream) of the CCR5 target position, and a third and a fourth single stranded breaks are positioned downstream (e.g., within 500 bp downstream, e.g., within 200 bp downstream) of the CCR5 target position. In certain embodiments, the breaks are positioned to avoid unwanted target chromosome elements, such as repeat elements, e.g., an Alu repeat.

In certain embodiments, two or more (e.g., three or four) gRNA molecules are used with one Cas9 molecule. In certain embodiments, when two ore more (e.g., three or four) gRNAs are used with two or more Cas9 molecules, at least one Cas9 molecule is from a different species than the other Cas9 molecule(s). For example, when two gRNA molecules are used with two Cas9 molecules, one Cas9 molecule can be from one species and the other Cas9 molecule can be from a different species. Both Cas9 species are used to generate a single or double-strand break, as desired.

(4.1c) CCR5 Knock Out with Concomitant Knock-In of Anti-HIV Gene or Genes Under Expression of Endogenous Promoter or Pol III Promoter

The method modifies autologous or allogeneic HSCs ex vivo to increase resistance to HIV. In certain embodiments, the CCR5 gene is knocked out in HSCs or lymphoid progenitors or T lymphocytes ex vivo using the methods described herein, e.g., NHEJ-mediated knock-out, and an anti-HIV gene encoded in a transgene expression cassette is inserted using the methods described herein, e.g., homology directed repair. In certain embodiments, in HSCs or lymphoid progenitors or T lymphocytes ex vivo, the CCR5 gene is knocked down using the methods described herein, e.g., dCas9-mediated knock-down, and CCR5 is knocked out using the methods described herein, e.g., NHEJ-mediated knock-out, and an anti-HIV gene, e.g., an anti-HIV peptide encoded in a transgene expression cassette driven by a Pol III promoter, is inserted using the methods described herein, e.g., homology directed repair.

The cassette expressing an anti-HIV gene is inserted in the CCR5 gene locus, which is considered to be a putative safe harbor locus (Papapetrou et al., Molecular Therapy (12 Feb. 2016)1 doi:10.1038/mt.2016.38). The cassette expressing an anti-HIV gene is inserted in a safe harbor locus. In certain embodiments, a cassette expressing multiple anti-HIV genes are inserted, each with separate promoters, into the CCR5 safe harbor region. In certain embodiments, a cassette expressing multiple anti-HIV genes are inserted, each with separate promoters, into a safe harbor locus. In certain embodiments, the CCR5 coding sequence is disrupted and, simultaneously, another safe harbor site AAVS1 is used for HDR for targeted insertion of an anti-HIV encoding transgene expression cassette.

In certain embodiments, the anti-HIV gene is under the expression of endogenous CCR5 promoter. In certain embodiments, the anti-HIV gene is under the expression of a Pol III promoter that is delivered as an element of the transgene expression cassette.

In certain embodiments, the anti-HIV gene is the coding sequence of any of the molecules listed in Table 17.

In certain embodiments, the anti-HIV gene encodes a siRNA molecule, e.g., shRNA, e-shRNA, hRNA, AgoshRNA.

In certain embodiments, the anti-HIV gene encodes a ribozyme which targets HIV, e.g., a ribozyme targeting tat/vpr, a ribozyme targeting rev/tat, or a ribozyme targeting U5 leader sequence.

In certain embodiments, the anti-HIV gene encodes fusion inhibitor, e.g., N36, T21, CP621-652, CP628-654, C34, DP107, IZN36, N36ccg, SFT, SC22EK, MTSC22, MTSC21, MTSC19, HP23, HP22, HP23E, T-1249, IQN17, IQN23, IQN36, IIN17, IQ22N17, II22N17, II15N17, IZN17, IZN23, IZN36, C46, C46-EHO, C37H6, or CP32M.

In certain embodiments, the anti-HIV gene encodes an HIV-1 trans activation response element (TAR), e.g., TAR decoy or TAR aptamer.

In certain embodiments, the modified HSCs do not express CCR5 and do express an anti-HIV gene, e.g., CCR5−/−/shRNA knock-in+/+, e.g., CCR5−/−/ribozyme knock-in+/+, e.g., CCR5−/−/fusion inhibitor knock-in+/+, e.g., CCR5−/−/C46 fusion inhibitor knock-in+/+, e.g., CCR5−/−/TAR knock-in+/+. In certain embodiments, the method confers resistance to HIV entry into T-cells, e.g., by CCR5 gene knock-down and/or knock-out, and drives expression of an anti-HIV element. The method confers resistance to HIV infection multiple mechanisms, e.g., by CCR5 knock out and siRNA targeting tat/rev, by CCR5 knock out and expression of a ribozyme targeting tat/vpr, by CCR5 knock out and expression of a ribozyme targeting rev/tat, by CCR5 knock out and expression of a ribozyme targeting U5 leader sequence, by CCR5 knock out and expression of a fusion inhibitor, e.g., C46 fusion inhibitor, T20 fusion inhibitor, by CCR5 knock out and expression of an anti-HIV element listed in Table 17. The aim is to target multiple viral pathways to increase resistance of cells to HIV. In subjects suffering from HIV, single use of fusion inhibitors, such as T20 (enfuvirtide), has led to HIV resistance (Greenberg et al., J Antimicrob Chemother 54:333-340). Targeting multiple pathways concomitantly is a well accepted approach to reducing the likelihood of developing therapy-resistant HIV.

TABLE 17
Anti-HIV Transgenes

| | | Citation | | |
|---|---|---|---|---|
| | | demonstrating | HIV Binding | |
| Agent | Class | anti-HIV activity | region | Sequence |
| T1144 | Fusion | Dwyer, Proc Natl | | |
| | inhibitor | Acad Sci USA. | | |
| | | 2007 Jul 31; | | |
| | | 104(31): 12772-7. | | |
| T1249, | Fusion | | | |
| T1144, | inhibitors | | | |
| T267227, | | | | |
| C38, | | | | |
| and N46 | | | | |
| T20 | Fusion | Wild et al., Proc | Targets C- | YTSLIHSLIEESQN |
| (also | inhibitor | Natl Acad Sci USA. | terminal | QQEKNEQELLELD |
| known | | 1994 Oct 11; | heptad repeat | KWASLWNWF |
| as DP- | | 91(21): 9770-4. | region of HIV | (SEQ ID NO: 8412) |
| 178, | | Greenberg et al., | gp41 region | |
| Enfuvirtide, | | J Antimicrob | | |
| and | | Chemother. 2004 | | |
| Fuzeon) | | Aug; 54(2): 333-40. | | |
| | | Gochin et al., Curr | | |
| | | Top Med Chem. | | |
| | | 2011 Dec 1; | | |
| | | 11(24): 3022-3032. | | |
| C37H6 | Fusion | | | |
| | inhibitor | | | |
| CP32M | Fusion | | | |
| | inhibitor | | | |
| sifuvirtide | | Yao et al., J Biol | | |
| | | Chem. | | |
| | | 2012; 287: 6788-6796. | | |
| albuvirtide | | | | |
| 2DLT | | | | |
| AMD3100 | | | | |
| and | | | | |
| AMD070 | | | | |
| SCH-C | | | | |
| and | | | | |
| SCH-D | | | | |
| UK- | | | | |
| 427,857 | | | | |
| N36 | Fusion | Gochin et al., Curr | Targets N- | SGIVQQQNNLLRA |
| | inhibitor | Top Med Chem. | terminal | IEAQQHLLQLTVW |
| | | 2011 Dec 1; | heptad repeat | GIKQLQARIL (SEQ |
| | | 11(24): 3022-3032. | region of HIV | ID NO: 8413) |
| | | | gp41 region | |
| T21 | Fusion | | Targets N- | |
| | inhibitor | | terminal | |
| | | | heptad repeat | |
| | | | region of HIV | |
| | | | gp41 region | |
| CP621- | Fusion | | Target CHR | |
| 652 | inhibitor | | region of HIV | |
| | | | gp41 region | |
| CP628- | Fusion | | Target CHR | |
| 654 | inhibitor | | region of HIV | |
| | | | gp41 region | |
| C34 | Fusion | Gochin et al., Curr | Targets HR2 | WMEWDREINNYT |
| | inhibitor | Top Med Chem. | region of HIV | SLIHSLIEESQNQQ |
| | | 2011 Dec 1; | gp41 region | EKNEQELL (SEQ |
| | | 11(24): 3022-3032. | | ID NO: 8414) |
| DP | | | | YTSLIHSLIEESQN |
| | | | | QQEKNEQELLE |
| | | | | (SEQ ID NO: 8415) |
| DP107 | Fusion | | Targets c- | |
| | inhibitor | | terminal region | |
| | | | of HIV gp41- | |
| | | | HR1 inhibitor | |
| IZN36 | Fusion | | Traps pre- | |
| | inhibitor | | hairpin | |
| | | | intermediate | |
| N36ccg | Fusion | Su et al., J Virol | Traps pre- | |
| | inhibitor | 2015; 89: 5801-5811. | hairpin | |
| | | | intermediate | |
| SFT | Fusion | Su et al., J Virol | Target CHR | |
| | inhibitor | 2015; 89: 5801-5811. | region of HIV | |
| | | | gp41 region | |
| SC22EK | Fusion | Su et al., J Virol | Target CHR | |
| | inhibitor | 2015; 89: 5801-5811. | region of HIV | |
| | | | gp41 region | |
| MTSC22 | Fusion | Su et al., J Virol | Target CHR | |
| | inhibitor | 2015; 89: 5801-5811. | region of HIV | |
| | | | gp41 region | |
| MTSC21 | Fusion | Su et al., J Virol | Target CHR | |
| | inhibitor | 2015; 89: 5801-5811. | region of HIV | |
| | | | gp41 region | |
| MTSC19 | Fusion | Su et al., J Virol | Target CHR | |
| | inhibitor | 2015; 89: 5801-5811. | region of HIV | |
| | | | gp41 region | |
| HP23 | Fusion | Su et al., J Virol | Target CHR | |
| | inhibitor | 2015; 89: 5801-5811. | region of HIV | |
| | | | gp41 region | |
| HP22 | Fusion | Su et al., J Virol | Target CHR | |
| | inhibitor | 2015; 89: 5801-5811. | region of HIV | |
| | | | gp41 region | |
| HP23E | Fusion | Su et al., J Virol | Target CHR | |
| | inhibitor | 2015; 89: 5801-5811. | region of HIV | |
| | | | gp41 region | |
| T-1249 | Fusion | Gochin et al., Curr | | WQEWEQKI------------ |
| | inhibitor | Top Med Chem. | | TALLEQAQIQQEK |
| | | 2011 Dec 1; | | NEYELQKLDKWA |
| | | 11(24): 3022-3032. | | SLWEWF (SEQ ID |
| | | | | NO: 8416) |
| IQN17 | Fusion | Eckert et al., Proc | Targets N- | |
| | inhibitor | Natl Acad Sci USA. | terminal | |
| | | 2001 Sep 25; | heptad repeat | |
| | | 98(20): 11187-11192. | region of HIV | |
| | | | gp41 region | |
| IQN23 | Fusion | Eckert et al., Proc | Targets N- | |
| | inhibitor | Natl Acad Sci USA. | terminal | |
| | | 2001 Sep 25; | heptad repeat | |
| | | 98(20): 11187-11192. | region of HIV | |
| | | | gp41 region | |
| IQN36 | Fusion | Eckert et al., Proc | Targets N- | |
| | inhibitor | Natl Acad Sci USA. | terminal | |
| | | 2001 Sep 25; | heptad repeat | |
| | | 98(20): 11187-11192. | region of HIV | |
| | | | gp41 region | |
| IIN17 | Fusion | Eckert et al., Proc | Targets N- | |
| | inhibitor | Natl Acad Sci USA. | terminal | |
| | | 2001 Sep 25; | heptad repeat | |
| | | 98(20): 11187-11192. | region of HIV | |
| | | | gp41 region | |
| IQ22N17 | Fusion | Eckert et al., Proc | Targets N- | |
| | inhibitor | Natl Acad Sci USA. | terminal | |
| | | 2001 Sep 25; | heptad repeat | |
| | | 98(20): 11187-11192. | region of HIV | |
| | | | gp41 region | |
| II22N17 | Fusion | Eckert et al., Proc | Targets N- | |
| | inhibitor | Natl Acad Sci USA. | terminal | |
| | | 2001 Sep 25; | heptad repeat | |
| | | 98(20): 11187-11192. | region of HIV | |
| | | | gp41 region | |
| II15N17 | Fusion | Eckert et al., Proc | Targets N- | |
| | inhibitor | Natl Acad Sci USA. | terminal | |
| | | 2001 Sep 25; | heptad repeat | |
| | | 98(20): 11187-11192. | region of HIV | |
| | | | gp41 region | |
| IZN17 | Fusion | Eckert et al., Proc | Targets N- | |
| | inhibitor | Natl Acad Sci USA. | terminal | |
| | | 2001 Sep 25; | heptad repeat | |
| | | 98(20): 11187-11192. | region of HIV | |
| | | | gp41 region | |
| IZN23 | Fusion | Eckert et al., Proc | Targets N- | |
| | inhibitor | Natl Acad Sci USA. | terminal | |
| | | 2001 Sep 25; | heptad repeat | |
| | | 98(20): 11187-11192. | region of HIV | |
| | | | gp41 region | |
| IZN36 | Fusion | Eckert et al., Proc | Targets N- | |
| | inhibitor | Natl Acad Sci USA. | terminal | |
| | | 2001 Sep 25; | heptad repeat | |
| | | 98(20): 11187-11192. | region of HIV | |
| | | | gp41 region | |
| C46 and | Fusion | Brauer et al., | Target CHR | |
| C46- | inhibitor | Antimicrob. | region of HIV | |
| EHO | | Agents | gp41 region | |
| | | Chemother. | | |
| | | February 2013 vol. | | |
| | | 57 no. 2 679-688. | | |
| C37H6 | Fusion | Xiao et al., Bioorg | Binds HR1 | |
| | inhibitor | Med Chem Lett. | region of gp41 | |
| | | 2013 Nov 15; | and stabilizes | |
| | | 23(22): 10.1016. | pre-hairpin | |
| | | | structure to | |
| | | | inhibit | |
| | | | membrane | |
| | | | fusion | |
| CP32M | Fusion | Xiao et al., Bioorg | Binds HR1 | |
| | inhibitor | Med Chem Lett. | region of gp41 | |
| | | 2013 Nov 15; | and stabilizes | |
| | | 23(22): 10.1016. | pre-hairpin | |
| | | | structure to | |
| | | | inhibit | |
| | | | membrane | |
| | | | fusion | |
| tat-rev | siRNA | Anderson et al., | | |
| shRNA | | Mol Ther. 2007 | | |
| | | Jun; 15(6): 1182-8. | | |
| | e-shRNA | | | |
| | hRNA | | | |
| | shRNA | | | |
| | AgoshRNA | | | |
| Ribozyme | Ribozyme | | | |
| vs. | | | | |
| tat/vpr | | | | |
| Ribozyme | | | | |
| vs. | | | | |
| rev/tat | | | | |
| Ribozyme | | | | |
| vs. | | | | |
| U5 | | | | |
| leader | | | | |
| sequence | | | | |
| neutralizing | Anti- | Sullenger BA, | | |
| the | HIV-1 | Gallardo HF, | | |
| action of | aptamers- | Ungers GE, Gilboa E | | |
| the HIV-1 | HIV-1 | Cell. 1990 Nov 2; | | |
| proteins | trans- | 63(3): 601-8. | | |
| Tat | activation | | | |
| | response | | | |
| | element | | | |
| | (TAR) | | | |
| neutralizing | Anti- | Lee TC, Sullenger | | |
| the | HIV-1 | BA, Gallardo HF, | | |
| action of | aptamers | Ungers GE, Gilboa E | | |
| the HIV-1 | | New Biol. 1992 | | |
| proteins | | Jan; 4(1): 66-74. | | |
| Rev | | | | |
| | | Michienzi A, Li S, | | |
| | | Zaia JA, Rossi JJ | | |
| | | Proc Natl Acad Sci | | |
| | | USA. 2002 Oct | | |
| | | 29; 99(22): 14047-52. | | |
| | | Bai J, Banda N, | | |
| | | Lee NS, Rossi J, | | |
| | | Akkina R | | |
| | | Mol Ther. 2002 | | |
| | | Dec; 6(6): 770-82. | | |
| Tar | | Banerjea A, Li MJ, | | |
| Decoy | | Remling L, Rossi | | |
| | | J, Akkina R | | |
| | | AIDS Res Ther. | | |
| | | 2004 Dec 17; | | |
| | | 1(1): 2. | | |
| TAR | | | | |
| aptamer | | | | |
| TRIM5a | | | | |
| Multiplex | | Walker et al., J | | |
| | | Virol. 2012 May; | | |
| | | 86(10): 5719-29. | | |
| Not | | | | |
| peptides: | | | | |
| PRO | Block | | | |
| 542 | CD4 | | | |
| | binding | | | |
| BMS- | | | | |
| 806 | | | | |
| TNX- | | | | |
| 355 | | | | |

In the case of autologous HSC modification, modified cells are infused into the subject and are resistant to HIV. In the case of allogeneic HSC modification, modified cells are reinfused into the subject and are resistant to HIV. The aim is to ameliorate or cure HIV in a subject.

### (4.1d) CCR5 Knock Out With Concomitant Knock-In of Drug Resistance Selectable Marker for Enabling Selection of Modified HSCs:

In certain embodiments, in HSCs or lymphoid progenitors or T lymphocytes ex vivo, the CCR5 gene is knocked out using the methods described herein, e.g., NHEJ-mediated knock-out, and a drug resistance selectable marker, encoded in a transgene expression set, e.g., chemotherapy resistance gene P140K driven by a EFS promoter, is inserted at the CCR5 gene locus using homology directed repair. In certain embodiments, in HSCs or lymphoid progenitors or T lymphocytes ex vivo, the CCR5 gene is knocked down using the methods described herein, e.g., dCas9-mediated knock-down, and a drug resistance selectable marker encoded in a transgene expression set, e.g., chemotherapy resistance gene P140K driven by a EFS promoter, is inserted at the CCR5 gene locus using homology directed repair.

The cassette expressing a drug resistance selectable marker is inserted in the CCR5 gene locus which is a safe harbor locus. The cassette expressing a resistance selectable marker is inserted in a safe harbor locus.

In certain embodiments, the drug resistance selectable marker is under the expression of endogenous CCR5 promoter. In certain embodiments, the drug resistance selectable marker is under the expression of a EFS promoter that is an element of the transgene expression cassette.

HSCs are modified ex vivo with the method, knocking out the CCR5 gene and knocking in a gene encoding a drug resistance selectable marker, e.g., chemotherapy resistance gene P140K.

(a) Modified HSCs (e.g., CCR5−/−/P140K knock-in+/+) are exposed to chemotherapy ex vivo. Chemotherapy exposure can destroy unedited cells and only edited cells can be preserved. Only HSCs that have been modified can survive. Selected, modified HSCs can have all have CCR5 gene knock out and can be administered to the subject.

(b) Modified HSCs (e.g., CCR5−/−/P140K knock-in+/+) are transplanted into subject. HSCs are exposed to chemotherapy in vivo. HSCs that have been modified can survive, as chemotherapy exposure can destroy unedited cells. Modified HSCs can have CCR5 gene knock out.

Modified HSCs (e.g., CCR5−/−/P140K knock-in+/+) are HIV resistant. In the case of autologous HSC modification, modified cells are re-infused into the subject and can be resistant to HIV. In the case of allogeneic HSC modification, modified cells are infused into the subject and can be resistant to HIV. The aim is to ameliorate or cure HIV in a subject.

(4.2) Knocking Down CCR5 Mediated by an Enzymatically Inactive Cas9 (eiCas9) Molecule

A targeted knockdown approach reduces or eliminates expression of functional CCR5 gene product. As described herein, in certain embodiments, a targeted knockdown is mediated by targeting an enzymatically inactive Cas9 (eiCas9) molecule or an eiCas9 fused to a transcription repressor domain or chromatin modifying protein to alter transcription, e.g., to block, reduce, or decrease transcription, of the CCR5 gene.

Methods and compositions discussed herein may be used to alter the expression of the CCR5 gene to treat or prevent HIV infection or AIDS by targeting a promoter region of the CCR5 gene. In certain embodiments, the promoter region is targeted to knock down expression of the CCR5 gene. A targeted knockdown approach reduces or eliminates expression of functional CCR5 gene product. As described herein, in certain embodiments, a targeted knockdown is mediated by targeting an enzymatically inactive Cas9 (eiCas9) or an eiCas9 fused to a transcription repressor domain or chromatin modifying protein to alter transcription, e.g., to block, reduce, or decrease transcription, of the CCR5 gene.

In certain embodiments, one or more eiCas9s are used to block binding of one or more endogenous transcription factors. In certain embodiments, an eiCas9 can be fused to a chromatin modifying protein. Altering chromatin status can result in decreased expression of the target gene. One or more eiCas9s fused to one or more chromatin modifying proteins can be used to alter chromatin status.

### (4.3) Introduction of One or More Mutations in CCR5 Gene

In certain embodiments, the method comprises introducing one or more mutations in the CCR5 gene. In cetain embodiments, the one or more mutations comprise one or more protective mutations. In cetain embodiments, the one or more protective mutations comprise a delta32 mutation.

### (4.3a) NHEJ-Mediated Creation of Naturally Occurring Delta 32 Mutation in CCR5 Gene

In certain embodiments, the method comprises deleting (e.g., NHEJ-mediated deletion) a genomic sequence within the coding sequence of the CCR5 gene, e.g., a NHEJ-mediated 32-base pair deletion at cDNA position 794-825 (deletion of codons 175-185). As described herein, in certain embodiments, the method comprises introduction of two sets of breaks (e.g., a pair of double strand breaks, one double strand break or a pair of single strand breaks, or two pairs of single strand breaks) to flank a region of the CCR5 gene (e.g., a coding region). In certain embodiments, NHEJ-mediated repair of the break(s) alters the CCR5 gene to generate a naturally occurring mutation, the delta32 mutation. The delta32 mutation is a 32-base pair deletion that, during translation, leads to a frameshift after codon 174, inclusion of 31 novel amino acids, and premature truncation of the CCR5 protein. The truncated CCR5 receptor does not traffic to the cell membrane and cannot act as a co-receptor for HIV. The delta 32 mutation in CCR5 confers resistance to HIV (Samson et al., Nature 382: 722-725, 1996). The method of deletion (e.g., NHEJ-mediated deletion) of base pairs 794-825 in the CCR5 gene can recreate a naturally occurring mutation and confer resistance to HIV. The method can create a delta 32 mutation in a single allele of CCR5 (CCR5^{+/Δ32}) or a mutation in both alleles of CCR5 (CCR5^{Δ32/Δ32}). The method can be used in a subject suffering from HIV, to ameliorate or cure disease. The method can be used in a subject who is not suffering from HIV, to prevent the disease.

The CCR5 delta32 protective eletion has been found to be associated with a slower progression of disease in certain autoimmune and infectious diseases, including Multiple Sclerosis, transplant rejection and Hepatitis C (Barcellos et al., Immunogenetics 51: 281-288, 2000. Fischereder et al., Neurology 61: 238-240, 2003. Goulding et al., Gut 54: 1157-1161, 2005.). The methods described herein can be used to create a protective delta32 deletion in CCR5 gene to ameliorate Multiple Sclerosis, ameliorate Hepatitis C, slow the progression of transplant loss, or slow progression of other autoimmune and/or infectious diseases.

In certain embodiments, two double strand breaks are introduced (e.g., positioned by two gRNA molecules) at or in close proximity to a CCR5 target position in the CCR5 gene. In certain embodiment, the CCR5 target position comprise a 32 base pair region at c. 794-825. In certain embodiments, two gRNA molecules (e.g., with one or two Cas9 nucleases that are not Cas9 nickases) are used to create two double strand breaks to flank a CCR5 target position, e.g., the gRNA molecules are configured such that one double strand break is positioned upstream (e.g., within 500 bp upstream, e.g., within 200 bp upstream) and a second double strand break is positioned downstream (e.g., within 500 bp downstream, e.g., within 200 bp downstream) of the CCR5 target position. In certain embodiments, the breaks are positioned to avoid unwanted target chromosome elements, such as repeat elements, e.g., an Alu repeat.

In certain embodiments, one double strand break and two single strand breaks are introduced (e.g., positioned by three gRNA molecules) at or in close proximity to a CCR5 target position in the CCR5 gene. In certain embodiments, the CCR5 target position comprises a32 base pair region at c. 794-825. In certain embodiments, three gRNA molecules (e.g., with a Cas9 nuclease other than a Cas9 nickase and one or two Cas9 nickases) to create one double strand break and two single strand breaks to flank a CCR5 target position, e.g., the gRNA molecules are configured such that the double strand break is positioned upstream or downstream of (e.g., within 500 bp, e.g., within 200 bp upstreamor downstream) of the CCR5 target position, and the two single strand breaks are positioned at the opposite site, e.g., downstream or upstream (e.g., within 500 bp, e.g., within 200 bp downstream or upstream), of the CCR5 target position. In certain embodiments, the breaks are positioned to avoid unwanted target chromosome elements, such as repeat elements, e.g., an Alu repeat.

In certain embodiments, four single strand breaks are introduced (e.g., positioned by four gRNA molecules) at or in close proximity to a CCR5 target position in the CCR5 gene. In certain embodiments, the CCR5 target position comprises a 32 base pair region at c. 794-825. In certain embodiments, four gRNA molecule (e.g., with one or more Cas9 nickases are used to create four single strand breaks to flank a CCR5 target position in the CCR5 gene, e.g., the gRNA molecules are configured such that a first and second single strand breaks are positioned upstream (e.g., within 500 bp upstream, e.g., within 200 bp upstream) of the CCR5 target position, and a third and a fourth single stranded breaks are positioned downstream (e.g., within 500 bp downstream, e.g., within 200 bp downstream) of the CCR5 target position. In certain embodiments, the breaks are positioned to avoid unwanted target chromosome elements, such as repeat elements, e.g., an Alu repeat.

In certain embodiments, two or more (e.g., three or four) gRNA molecules are used with one Cas9 molecule. In certain embodiments, when two ore more (e.g., three or four) gRNAs are used with two or more Cas9 molecules, at least one Cas9 molecule is from a different species than the other Cas9 molecule(s). For example, when two gRNA molecules are used with two Cas9 molecules, one Cas9 molecule can be from one species and the other Cas9 molecule can be from a different species. Both Cas9 species are used to generate a single or double-strand break, as desired.

### (4.3b) HDR-Mediated Introduction of Delta 32 Mutation to CCR5

Subjects who are homozygous for the CCR5 Δ32 (CCR5 Δ32/Δ32) mutation are immune to HIV-1 (Samson et al., Nature. 1996 Aug. 22; 382(6593):722-5). The CCR5 delta32 mutation is a naturally occurring 32-base pair deletion that, during translation, leads to a frameshift after codon 174, inclusion of 31 novel amino acids, and premature truncation of the CCR5 protein. The CCR5 receptor does not traffic to T-cell membrane. The CCR5 Δ32 mutation confers resistance to HIV because HIV cannot use the CCR5-coreceptor for viral entry into T-cells. An individual with late stage HIV received a HSC transplantation (to treat leukemia related to HIV) from a subject who was homozygous for the CCR5 Δ32 mutation. Following the transplant, the individual appears to have controlled HIV, with no evidence of HIV and no need for antiretroviral therapy for several years (Hutter, et al., N Engl J Med. 2009 Feb. 12; 360(7):692-8. Allers et al., Blood. 2011 Mar. 10; 117(10):2791-9). The methods can recreate the naturally occurring CCR5 Δ32 mutation in a subject to confer resistance to HIV and/or to cure HIV infection.

The method of deletion, e.g., HDR-mediated deletion of base pairs c.794-825 in the CCR5 gene recreates a naturally occurring mutation and confers resistance to HIV. The method can create a delta 32 mutation in a single allele of CCR5 (CCR5+/Δ32) or a mutation in both alleles of CCR5 (CCR5 Δ32/Δ32). The method can be used in a subject with HIV, to ameliorate or cure disease. The method can be used in a subject who is not suffering from HIV, to prevent disease.

In certain embodiments, the method uses homology directed repair to target the coding region of the CCR5 gene with the aim to produce a truncated CCR5 protein product. In certain embodiments, the coding region of the CCR5 gene is targeted to create a mutation, e.g., a deletion that is a Δ32 mutation at position c.794-825 (deletion of codons 175-185), by homology directed repair. The method recreates a naturally occurring mutation in CCR5 known as the Δ32 mutation. The method can disrupt a CCR5 gene so that the truncated protein product, e.g., the truncated CCR5 receptor, does not traffic to the cell membrane. T-cells lacking a CCR5 receptor can be resistant to HIV, as HIV utilizes the CCR5 receptor as a co-receptor, along with CD4, for viral entry into T-cells. The method ameliorates or cures HIV.

In certain embodiments, the targeting domain of the gRNA molecule is configured to provide a cleavage event, e.g., a double strand break or a single strand break, sufficiently close to (e.g., either 5′ or 3′ to) the target the CCR5 gene for introduction of the Δ32 mutation in the CCR5 gene. In certain embodiments, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position in the CCR5 gene. The break, e.g., a double strand or single strand break, can be positioned upstream or downstream of the target position in the CCR5 gene.

In certain embodiments, a second, third and/or fourth gRNA molecule is configured to provide a cleavage event, e.g., a double strand break or a single strand break, sufficiently close to (e.g., either 5′ or 3′ to) the target position in the CCR5 gene for the introduction of the Δ32 mutation. In certain embodiments, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position in the CCR5 gene. The break, e.g., a double strand or single strand break, can be positioned upstream or downstream of the target position in the CCR5 gene.

In certain embodiments, a single strand break is accompanied by an additional single strand break, positioned by a second, third and/or fourth gRNA molecule, as discussed below. For example, the targeting domains bind configured such that a cleavage event, e.g., the two single strand breaks, are positioned within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position in the CCR5 gene for the introduction of the Δ32 mutation. In certain embodiments, the first and second gRNA molecules are configured such, that when guiding a Cas9 nickase, a single strand break can be accompanied by an additional single strand break, positioned by a second gRNA, sufficiently close to one another to result in an alteration of the target position in the CCR5 gene. In certain embodiments, the first and second gRNA molecules are configured such that a single strand break positioned by said second gRNA is within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the break positioned by said first gRNA molecule, e.g., when the Cas9 is a nickase. In certain embodiments, the two gRNA molecules are configured to position cuts at the same position, or within a few nucleotides of one another, on different strands, e.g., essentially mimicking a double strand break.

In certain embodiments, a double strand break can be accompanied by an additional double strand break, positioned by a second, third and/or fourth gRNA molecule, as is discussed below. For example, the targeting domain of a first gRNA molecule is configured such that a double strand break is positioned upstream of the target position in the CCR5 gene within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position; and the targeting domain of a second gRNA molecule is configured such that a double strand break is positioned downstream the target position in the CCR5 gene, within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position.

In certain embodiments, a double strand break can be accompanied by two additional single strand breaks, positioned by a second gRNA molecule and a third gRNA molecule. For example, the targeting domain of a first gRNA molecule is configured such that a double strand break is positioned upstream of the target position in the CCR5 gene, e.g., within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position; and the targeting domains of a second and third gRNA molecule are configured such that two single strand breaks are positioned downstream of the target position in the CCR5 gene, within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position. In certain embodiments, the targeting domain of the first, second and third gRNA molecules are configured such that a cleavage event, e.g., a double strand or single strand break, is positioned, independently for each of the gRNA molecules.

In certain embodiments, a first and second single strand breaks can be accompanied by two additional single strand breaks positioned by a third gRNA molecule and a fourth gRNA molecule. For example, the targeting domain of a first and second gRNA molecule are configured such that two single strand breaks are positioned upstream of the target position in the CCR5 gene, e.g., within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position in the CCR5 gene; and the targeting domains of a third and fourth gRNA molecule are configured such that two single strand breaks are positioned downstream of the target position in the CCR5 gene, e.g., within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position in the CCR5 gene.

In certain embodiments, a mutation in the CCR5 gene, e.g., Δ32 mutation, is introduced using an exogenously provided template nucleic acid, e.g., by HDR. In certain embodiments, the template nucleic acid is a single strand oligonucleotide.

In certain embodiments, an eaCas9 molecule, e.g., an eaCas9 molecule described herein, is used. In certain embodiments, the eaCas9 molecule comprises HNH-like domain cleavage activity but has no, or no significant, N-terminal RuvC-like domain cleavage activity. In certain embodiments, the eaCas9 molecule is an HNH-like domain nickase. In certain embodiments, the eaCas9 molecule comprises a mutation at D10 (e.g., D10A). In certain embodiments, the eaCas9 molecule comprises N-terminal RuvC-like domain cleavage activity but has no, or no significant, HNH-like domain cleavage activity. In certain embodiments, the eaCas9 molecule is an N-terminal RuvC-like domain nickase. In certain embodiments, the eaCas9 molecule comprises a mutation at H840 (e.g., H840A) or N863 (e.g., N863A).

### 5. Methods of Targeting CXCR4

As disclosed herein, the CXCR4 gene can be altered by gene editing, e.g., using CRISPR-Cas9-mediated methods as described herein.

Methods, genome editing systems, and compositions discussed herein, provide for altering a CXCR4 target position in the CXCR4 gene. A CXCR4 target position can be targeted (e.g., altered) by gene editing, e.g., using CRISPR-Cas9 mediated methods, genome editing systems, and compositions described herein.

Disclosed herein are methods for targeting (e.g., altering) a CXCR4 target position in the CXCR4 gene. Targeting (e.g., aAltering a CXCR4 target position can be achieved by one or more the following approaches:

(5.1) knocking out the CXCR4 gene:
(5.1a) insertion or deletion (e.g., NHEJ-mediated insertion or deletion) of one or more nucleotides in close proximity to or within the early coding region of the CXCR4 gene,
(5.1b) deletion (e.g., NHEJ-mediated deletion) of a genomic sequence including at least a portion of the CXCR4 gene, and
(5.1c) deletion (e.g., NHEJ-mediated deletion) of amino acids in N-terminus in the CXCR4 gene,

(5.2) knocking down the CXCR4 gene mediated by enzymatically inactive Cas9 (eiCas9) molecule or an eiCas9-fusion, and

(5.3) introduction of one or more mutations in the CXCR4 gene.

In certain embodiments, methods described herein introduce one or more breaks near the early coding region in at least one allele of the CXCR4 gene. In certain embodiments, methods described herein introduce two or more breaks to flank at least a portion of the CXCR4 gene. The two or more breaks remove (e.g., delete) a genomic sequence including at least a portion of the CXCR4 gene. In certain embodiments, methods described herein comprise knocking down the CXCR4 gene mediated by enzymatically inactive Cas9 (eiCas9) molecule or an eiCas9-fusion protein by targeting the promoter region of CXCR4 target knockdown position. Methods 3a, 3b and 4 described herein result in targeting (e.g., alteration) of the CXCR4 gene.

The targeting (e.g., alteration) of the CXCR4 gene can be mediated by any mechanism. Exemplary mechanisms that can be associated with the alteration of the CXCR4 gene include, but are not limited to, NHEJ (e.g., classical or alternative), MMEJ, HDR (e.g., endogenous donor template mediated), SDSA, single strand annealing or single strand invasion.

### (5.1a) Knocking Out CXCR4 by Introducing an Indel in the CXCR4 Gene

In certain embodiments, the method comprises introducing an insertion of one more nucleotides in close proximity to the CXCR4 target knockout position (e.g., the early coding region) of the CXCR4 gene. As described herein, in certain embodiments, the method comprises the introduction of one or more breaks (e.g., single strand breaks or double strand breaks) sufficiently close to (e.g., either 5′ or 3′ to) the early coding region of the CXCR4 target knockout position, such that the break-induced indel could be reasonably expected to span the CXCR4 target knockout position (e.g., the early coding region). In certain embodiments, NHEJ-mediated repair of the break(s) allows for the NHEJ-mediated introduction of an indel in close proximity to within the early coding region of the CXCR4 target knockout position.

In certain embodiments, the method comprises introducing a deletion of a genomic sequence comprising at least a portion of the CXCR4 gene. As described herein, in certain embodiments, the method comprises the introduction of two double stand breaks—one 5′ and the other 3′ to (i.e., flanking) the CXCR4 target position. In certain embodiments, two gRNAs, e.g., unimolecular (or chimeric) or modular gRNA molecules, are configured to position the two double strand breaks on opposite sides of the CXCR4 target knockout position in the CXCR4 gene.

In certain embodiments, a single strand break is introduced (e.g., positioned by one gRNA molecule) at or in close proximity to a CXCR4 target position in the CXCR4 gene. In certain embodiments, a single gRNA molecule (e.g., with a Cas9 nickase) is used to create a single strand break at or in close proximity to the CXCR4 target position, e.g., the gRNA is configured such that the single strand break is positioned either upstream (e.g., within 500 bp upstream, e.g., within 200 bp upstream) or downstream (e.g., within 500 bp downstream, e.g., within 200 bp downstream) of the CXCR4 target position. In certain embodiments, the break is positioned to avoid unwanted target chromosome elements, such as repeat elements, e.g., an Alu repeat.

In certain embodiments, a double strand break is introduced (e.g., positioned by one gRNA molecule) at or in close proximity to a CXCR4 target position in the CXCR4 gene. In certain embodiments, a single gRNA molecule (e.g., with a Cas9 nuclease other than a Cas9 nickase) is used to create a double strand break at or in close proximity to the CXCR4 target position, e.g., the gRNA molecule is configured such that the double strand break is positioned either upstream (e.g., within 500 bp upstream, e.g., within 200 bp upstream) or downstream of (e.g., within 500 bp downstream, e.g., within 200 bp downstream) of a CXCR4 target position. In certain embodiments, the break is positioned to avoid unwanted target chromosome elements, such as repeat elements, e.g., an Alu repeat.

In certain embodiments, two single strand breaks are introduced (e.g., positioned by two gRNA molecules) at or in close proximity to a CXCR4 target position in the CXCR4 gene. In certain embodiments, two gRNA molecules (e.g., with one or two Cas9 nickcases) are used to create two single strand breaks at or in close proximity to the CXCR4 target position, e.g., the gRNAs molecules are configured such that both of the single strand breaks are positioned e.g., within 500 bp upstream, e.g., within 200 bp upstream) or downstream (e.g., within 500 bp downstream, e.g., within 200 bp downstream) of the CXCR4 target position. In certain embodiments, two gRNA molecules (e.g., with two Cas9 nickcases) are used to create two single strand breaks at or in close proximity to the CXCR4 target position, e.g., the gRNAs molecules are configured such that one single strand break is positioned upstream (e.g., within 200 bp upstream) and a second single strand break is positioned downstream (e.g., within 200 bp downstream) of the CXCR4 target position. In certain embodiments, the breaks are positioned to avoid unwanted target chromosome elements, such as repeat elements, e.g., an Alu repeat.

In certain embodiments, two double strand breaks are introduced (e.g., positioned by two gRNA molecules) at or in close proximity to a CXCR4 target position in the CXCR4 gene. In certain embodiments, two gRNA molecules (e.g., with one or two Cas9 nucleases that are not Cas9 nickases) are used to create two double strand breaks to flank a CXCR4 target position, e.g., the gRNA molecules are configured such that one double strand break is positioned upstream (e.g., within 500 bp upstream, e.g., within 200 bp upstream) and a second double strand break is positioned downstream (e.g., within 500 bp downstream, e.g., within 200 bp downstream) of the CXCR4 target position. In certain embodiments, the breaks are positioned to avoid unwanted target chromosome elements, such as repeat elements, e.g., an Alu repeat.

In certain embodiments, one double strand break and two single strand breaks are introduced (e.g., positioned by three gRNA molecules) at or in close proximity to a CXCR4 target position in the CXCR4 gene. In certain embodiments, three gRNA molecules (e.g., with a Cas9 nuclease other than a Cas9 nickase and one or two Cas9 nickases) to create one double strand break and two single strand breaks to flank a CXCR4 target position, e.g., the gRNA molecules are configured such that the double strand break is positioned upstream or downstream of (e.g., within 500 bp, e.g., within 200 bp upstreamor downstream) of the CXCR4 target position, and the two single strand breaks are positioned at the opposite site, e.g., downstream or upstrea m (e.g., within 500 bp, e.g., within 200 bp downstream or upstream), of the CXCR4 target position. In certain embodiments, the breaks are positioned to avoid unwanted target chromosome elements, such as repeat elements, e.g., an Alu repeat.

In certain embodiments, four single strand breaks are introduced (e.g., positioned by four gRNA molecules) at or in close proximity to a CXCR4 target position in the CXCR4 gene. In certain embodiments, four gRNA molecule (e.g., with one or more Cas9 nickases are used to create four single strand breaks to flank a CXCR4 target position in the CXCR4 gene, e.g., the gRNA molecules are configured such that a first and second single strand breaks are positioned upstream (e.g., within 500 bp upstream, e.g., within 200 bp upstream) of the CXCR4 target position, and a third and a fourth single stranded breaks are positioned downstream (e.g., within 500 bp downstream, e.g., within 200 bp downstream) of the CXCR4 target position. In certain embodiments, the breaks are positioned to avoid unwanted target chromosome elements, such as repeat elements, e.g., an Alu repeat.

In certain embodiments, two or more (e.g., three or four) gRNA molecules are used with one Cas9 molecule. In certain embodiments, when two ore more (e.g., three or four) gRNAs are used with two or more Cas9 molecules, at least one Cas9 molecule is from a different species than the other Cas9 molecule(s). For example, when two gRNA molecules are used with two Cas9 molecules, one Cas9 molecule can be from one species and the other Cas9 molecule can be from a different species. Both Cas9 species are used to generate a single or double-strand break, as desired.

### (5.1b) Knocking Out CXCR4 by Deleting a Genomic Sequence Including at Least a Portion of the CXCR4 Gene

In certain embodiments, the method comprises deleting (e.g., NHEJ-mediated deletion) a genomic sequence including at least a portion of the CXCR4 gene. As described herein, in certain embodiments, the method comprises the introduction two sets of breaks (e.g., a pair of double strand breaks, one double strand break or a pair of single strand breaks, or two pairs of single strand breaks) to flank a region of the CXCR4 gene (e.g., a coding region, e.g., an early coding region, or a non-coding region, e.g., a non-coding sequence of the CXCR4 gene, e.g., a promoter, an enhancer, an intron, a 3′UTR, and/or a polyadenylation signal). In certain embodiments, NHEJ-mediated repair of the break(s) allows for alteration of the CXCR4 gene as described herein, which reduces or eliminates expression of the gene, e.g., to knock out one or both alleles of the CXCR4 gene.

In certain embodiments, two double strand breaks are introduced (e.g., positioned by two gRNA molecules) at or in close proximity to a CXCR4 target position in the CXCR4 gene. In certain embodiments, two gRNA molecules (e.g., with one or two Cas9 nucleases that are not Cas9 nickases) are used to create two double strand breaks to flank a CXCR4 target position, e.g., the gRNA molecules are configured such that one double strand break is positioned upstream (e.g., within 500 bp upstream, e.g., within 200 bp upstream) and a second double strand break is positioned downstream (e.g., within 500 bp downstream, e.g., within 200 bp downstream) of the CXCR4 target position. In certain embodiments, the breaks are positioned to avoid unwanted target chromosome elements, such as repeat elements, e.g., an Alu repeat.

In certain embodiments, one double strand break and two single strand breaks are introduced (e.g., positioned by three gRNA molecules) at or in close proximity to a CXCR4 target position in the CXCR4 gene. In certain embodiments, three gRNA molecules (e.g., with a Cas9 nuclease other than a Cas9 nickase and one or two Cas9 nickases) to create one double strand break and two single strand breaks to flank a CXCR4 target position, e.g., the gRNA molecules are configured such that the double strand break is positioned upstream or downstream of (e.g., within 500 bp, e.g., within 200 bp upstreamor downstream) of the CXCR4 target position, and the two single strand breaks are positioned at the opposite site, e.g., downstream or upstrea m (e.g., within 500 bp, e.g., within 200 bp downstream or upstream), of the CXCR4 target position. In certain embodiments, the breaks are positioned to avoid unwanted target chromosome elements, such as repeat elements, e.g., an Alu repeat.

In certain embodiments, four single strand breaks are introduced (e.g., positioned by four gRNA molecules) at or in close proximity to a CXCR4 target position in the CXCR4 gene. In certain embodiments, four gRNA molecule (e.g., with one or more Cas9 nickases are used to create four single strand breaks to flank a CXCR4 target position in the CXCR4 gene, e.g., the gRNA molecules are configured such that a first and second single strand breaks are positioned upstream (e.g., within 500 bp upstream, e.g., within 200 bp upstream) of the CXCR4 target position, and a third and a fourth single stranded breaks are positioned downstream (e.g., within 500 bp downstream, e.g., within 200 bp downstream) of the CXCR4 target position. In certain embodiments, the breaks are positioned to avoid unwanted target chromosome elements, such as repeat elements, e.g., an Alu repeat.

In certain embodiments, two or more (e.g., three or four) gRNA molecules are used with one Cas9 molecule. In certain embodiments, when two ore more (e.g., three or four) gRNAs are used with two or more Cas9 molecules, at least one Cas9 molecule is from a different species than the other Cas9 molecule(s). For example, when two gRNA molecules are used with two Cas9 molecules, one Cas9 molecule can be from one species and the other Cas9 molecule can be from a different species. Both Cas9 species are used to generate a single or double-strand break, as desired.

### (5.1c) NHEJ-Mediated Deletion of Amino Acids in N-Terminus in the CXCR4 Gene

In certain embodiments, the method comprises ex vivo modification of autologous or allogeneic T-cells to introduce a deletion in the N-terminus of the CXCR4 gene. (See Example 9 for editing of T cells.) Alternatively or additionally, the method comprises ex vivo modification of autologous or allogeneic HSCs to introduce a deletion in the N-terminus of the CXCR4 gene, followed by differentiation of the modified HSCs into lymphoid progenitor cells and/or T cells. The method can also be harvest of autologous or allogeneic HSCs, differentiation of the modified HSCs into lymphoid progenitor cells and/or T cells and modification to introduce a deletion in the N-terminus of the CXCR4 gene. The modified allogeneic or autologous lymphoid progenitor cells and/or T-cells are dosed to a subject with HIV to ameliorate disease.

In certain embodiments, the method comprises introduction a deletion, e.g., deletion of amino acid residues 2-9, deletion of amino acid residues 2-20, deletion of amino acid residues 2-24, deletion of amino acid residues 4-20, deletion of amino acid residues 4-36, or deletion of amino acid residues 10-20, by NHEJ-mediated CRISPR/Cas9 deletion. The deletion disrupts HIV gp120 binding to coreceptor CXCR4. Creation of a deletion mutation in the CXCR4 coreceptor N-terminus binding domain can alter binding kinetics between CXCR4 and HIV envelope protein gp120, decreasing strength of binding, decreasing efficiency of binding and/or decreasing frequency of binding between CXCR4 and HIV. Alteration of binding between CXCR4 and HIV gp120 by modification of amino acid residues 2-36 on CXCR4 leads to decreased viral entry into cells (Choi et al., J. Virol. 2005;79:15398-15404. Zhou et al., J. Biol, Chem, 2001;276:42826-42833.). The methods create a deletion in the CXCR4 gene in key binding domains for HIV gp120 binding and lead to decreased HIV infectivity, and decreased symptoms of disease. The methods ameliorate or cure HIV infection. The methods can be particularly relevant in late-stage HIV, in which CXCR4 coreceptor binding tends to represent the majority of HIV coreceptor activity in a subject (Connor et al. J Exp Med. 1997 Feb. 17; 185(4):621-8).

Creation of a deletion mutation in the CXCR4 coreceptor N-terminus binding domain can disrupt binding of SDF1 (CXCR12) to CXCR4, as a critical binding domain for SDF1 is the N-terminus of the CXCR4 receptor. CXCR4-SDF1 binding mediates HSC, lymphoid and myeloid cell migration out of the bone marrow and from the peripheral blood into tissue. The main role of CXCR4-SDF1 binding can be migration of myeloid lineage cells out of the bone marrow, as genetic mutations in CXCR4 lead to WHIM syndrome, which is characterized by peripheral neutropenia and abundant mature myeloid cells in the marrow (O'Regan et al., Am. J. Dis. Child. 131: 655-658, 1977). In certain embodiments, the method is used to replace cells in the peripheral compartment that are lymphoid progenitor cells and/or T cells and in an acute or subacute setting. In certain embodiments, HSCs are not modified by this method, thereby permitting cells of the myeloid lineage to preserve migration capabilities.

In certain embodiments, use of this method (e.g., deletion of N-terminal amino acids 2-9, 2-20, 2-24, 4-20 4-36, or 10-20 of the CXCR4 gene) is used in lymphoid cells and/or T-cells in an acute or subacute setting. Benefit of this method in short-term therapy in a subject with severe disease outweighs the risks of interrupting SDF1 interaction with CXCR4. In addition, HSCs derived from the subject bone marrow can retain unmodified CXCR4 receptors, which can interact with SDF1, thereby preserving lymphocyte homing and functionality. The rationale of the method is to generate modified T-cells that are HIV resistant and that function to provide lymphoid immunity in the short term for a subject with severe manifestations of HIV. The modified T-cells can help a subject overcome severe opportunistic infections. Subjects who can benefit from this method include those suffering from severe HIV, refractory HIV, end-stage HIV (e.g., AIDS), treatment resistant HIV, opportunistic infections, and CXCR4-coreceptor predominant HIV. The modified cells can be infused in a single or multiple doses.

(5.2) Knocking Down CXCR4 Mediated by an Enzymatically Inactive Cas9 (eiCas9) Molecule

A targeted knockdown approach reduces or eliminates expression of functional CXCR4 gene product. As described herein, in certain embodiments, a targeted knockdown is mediated by targeting an enzymatically inactive Cas9 (eiCas9) molecule or an eiCas9 fused to a transcription repressor domain or chromatin modifying protein to alter transcription, e.g., to block, reduce, or decrease transcription, of the CXCR4 gene.

Methods and compositions discussed herein may be used to alter the expression of the CXCR4 gene to treat or prevent HIV infection or AIDS by targeting a promoter region of the CXCR4 gene. In certain embodiments, the promoter region is targeted to knock down expression of the CXCR4 gene. A targeted knockdown approach reduces or eliminates expression of functional CXCR4 gene product. As described herein, in certain embodiments, a targeted knockdown is mediated by targeting an enzymatically inactive Cas9 (eiCas9) or an eiCas9 fused to a transcription repressor domain or chromatin modifying protein to alter transcription, e.g., to block, reduce, or decrease transcription, of the CXCR4 gene.

In certain embodiments, one or more eiCas9s may be used to block binding of one or more endogenous transcription factors. In certain embodiments, an eiCas9 can be fused to a chromatin modifying protein. Altering chromatin status can result in decreased expression of the target gene. One or more eiCas9s fused to one or more chromatin modifying proteins may be used to alter chromatin status.

### (5.3) Introduction of One or More Mutations in the CXCR4 Gene

In certain embodiments, the method comprises introducing one or more mutations in the CXCR4 gene. In certain embodiments, the introduction is mediated by HDR. In certain embodiments, the one or more mutations comprise one or more single base substitutions, one or more two base substitutions, or combinations thereof. In certain embodiments, the one or more mutations disrupt HIV gp120 binding to CXCR4.

In certain embodiments, the method introduces a single base substitution or a two base substitution in the CXCR4 gene that disrupts HIV gp120 binding to CXCR4. In certain embodiments, themethod comprises introducing a single base substitution or a two base substitution using homology directed repair by CRISPR/Cas9. Creation of a point mutation or a two base pair substitution in the CXCR4 binding domain can alter binding kinetics between CXCR4 and HIV envelope protein gp120, decrease strength of binding, decrease efficiency of binding and/or decreasing frequency of binding between CXCR4 and HIV. Alteration of binding between CXCR4 and HIV gp120 leads to decreased viral entry into cells (Choi et al., J. Virol. 2005;79:15398-15404. Brelot et al., J. Biol. Chem. 2000;275:23736-23744. Brelot et al., J. Virol. 73:2576-2586(1999). Zhou et al., J. Biol. Chem. 2001;276:42826-42833.). The methods create a single base substitution or a two base substitution in the CXCR4 gene in key HIV gp120 binding domains and lead to decreased HIV infectivity, and decreased symptoms of disease. The method ameliorates or cures HIV infection. The method is particularly relevant in late-stage HIV, in which CXCR4 coreceptor binding tends to represent the majority of HIV coreceptor activity in a subject (Connor et al. J Exp Med. 1997 Feb. 17; 185(4):621-8).

In certain embodiments, the single base substitution or two base substitution in CXCR4 is introduced in regions known to be critical for HIV gp120 binding and interaction with CXCR4 receptor. There is considerable overlap between regions on CXCR4 that interact with HIV gp120 and regions on CXCR4 that interact with SDF1 (also known as CXCL12). Key regions on CXCR4 that are involved with binding to both HIV gp120 and SDF1 include, but are not limited to: amino acids 2-25 and amino acid Glu288. The regions targeted comprise regions of CXCR4 that uniquely interact with HIV gp120 and are not key binding motifs for SDF1, including amino acids Asp171, Asp193, Gln200, Tyr255, Glu268, Glu277. The goal is to interrupt binding between HIV and CXCR4 while preserving binding between SDF1 and CXCR4, preserving critical immune function in a subject. (Suggested alterations to CXCR4 region 2-25 are described elsewhere in the methods; these methods are to be used in the short term treatment of a subject with severe HIV and are to be used to modify lymphoid cells, myeloid cells, T cells, T memory stem cells (TSCMs) and/or HSPCs).

Specific amino acids in CXCR4 have been demonstrated to be regions involved in HIV gp120 binding, including amino acids 171D, 193D, 200Q, 255Y, 268E, 277E. These amino acids are targeted for substitution. (See Table 18 for CXCR4 amino acid residues, proposed change to residue and refererence.) Specific Aspartic acid and Glutamic acid residues on CXCR4 are involved creating salt bridges between CXCR4 and HIV gp120 (Tamamis et al., Biophys J. 2013 Sep. 17; 105(6): 1502-1514). These residues are targeted for alteration. Methods that alter binding of HIV gp120 to CXCR4 but do not disrupt CXCR4 mediated chemotaxis and binding to SDF-1, or HSC homing to, lodging, and retention in the bone marrow are to be used to modify HSCs or HSPCs, followed by genome editing HSC transplantation.

TABLE 18

| CXCR4 | Amino | Proposed | |
|---|---|---|---|
| Position, | Acid, | change | Reference for decreased binding of HIV |
| Amino | wild | to amino | gp120 to CXCR4 at specified amino |
| Acid | type | acid: | acid position |
| 171 | D | A | Choi et al., J. Virol. 2005; 79: |
| | | | 15398-15404. |
| 171 | D | N | Brelot et al., J. Biol. Chem. |
| | | | 2000; 275: 23736-23744. |
| | | | Brelot et al., J. Virol. 73: |
| | | | 2576-2586(1999). |
| | | | Choi et al., J. Virol. 2005; |
| | | | 79: 15398-15404. |
| 193 | D | S | Brelot et al., J. Virol. 73: |
| | | | 2576-2586(1999). |
| 193 | D | A | Brelot et al., J. Biol. Chem. |
| | | | 2000; 275: 23736-23744. Brelot et |
| | | | al., J. Virol. 73: 2576-2586(1999). |
| 200 | Q | N | Zhou et al., J. Biol. Chem. |
| | | | 2001; 276: 42826-42833. |
| 255 | Y | A | Tamamis et al., Biophys J. 2013 |
| | | | Sep 17; 105(6): 1502-1514. |
| | | | Choi et al., J. Virol. 2005; |
| | | | 79: 15398-15404. |
| 268 | E | N | Zhou et al., J. Biol. Chem. |
| | | | 2001; 276: 42826-42833. |
| 268 | E | A | Brelot et al., J. Biol. Chem. |
| | | | 2000; 275: 23736-23744. |
| | | | Choi et al., J. Virol. 2005; |
| | | | 79: 15398-15404. |
| 277 | E | A | Tamamis et al., Biophys J. 2013 |
| | | | Sep 17; 105(6): 1502-1514. |
| | | | Brelot et al., J. Biol. Chem. |
| | | | 2000; 275: 23736-23744. |

In certain embodiments, amino acid 171D on the CXCR4 protein is targeted for substitution. The amino acid is changed to 171A or 171N, with homology directed repair utilizing CRISPR/Cas9 to modify the amino acid based on the required cDNA sequence. Interaction of CXCR4 with HIV gp120 has been demonstrated to be reduced significantly by this amino acid substitution (Choi et al., J. Virol. 2005;79:15398-15404). The method reduces HIV binding to CXCR4, decreases viral entry and ameliorates disease. Methods that alter binding of HIV gp120 to CXCR4 but do not disrupt CXCR4 mediated chemotaxis and binding to SDF-1, or HSC homing to, lodging, and retention in the bone marrow are to be used to modify HSCs or HSPCs, followed by genome editing HSC transplantation.

In certain embodiments, amino acid 193D on the CXCR4 protein is targeted for substitution. The amino acid is changed to 193A or 193S with homology directed repair utilizing CRISPR/Cas9 to modify the amino acid based on the required cDNA sequence. Interaction of CXCR4 with HIV gp120 has been demonstrated to be reduced significantly by this amino acid substitution. (Brelot et al., J. Biol. Chem. 2000;275:23736-23744; Brelot et al., J. Virol. 73:2576-2586(1999)) The method reduces HIV binding to CXCR4, decreases viral entry and ameliorates disease.

In certain embodiments, amino acid 200Q on the CXCR4 protein is targeted for substitution. The amino acid is changed to 200N with homology directed repair utilizing CRISPR/Cas9 to modify the amino acid based on the required cDNA sequence. Interaction of CXCR4 with HIV gp120 has been demonstrated to be reduced significantly by this amino acid substitution (Zhou et al., J. Biol. Chem. 2001;276:42826-42833). The method reduces HIV binding to CXCR4, decreases viral entry and ameliorates disease. Methods that alter binding of HIV gp120 to CXCR4 but do not disrupt CXCR4 mediated chemotaxis and binding to SDF-1, or HSC homing to, lodging, and retention in the bone marrow are to be used to modify HSCs or HSPCs, followed by genome editing HSC transplantation.

In certain embodiments, amino acid 255Y on the CXCR4 protein is targeted for substitution. The amino acid is changed to 255A with homology directed repair utilizing CRISPR/Cas9 to modify the amino acid based on the required cDNA sequence. Interaction of CXCR4 with HIV gp120 has been demonstrated to be reduced significantly by this amino acid substitution (Tamamis et al., Biophys J. 2013 Sep. 17; 105(6): 1502-1514). The method reduces HIV binding to CXCR4, decreases viral entry and ameliorates disease. Methods that alter binding of HIV gp120 to CXCR4 but do not disrupt CXCR4 mediated chemotaxis and binding to SDF-1, or HSC homing to, lodging, and retention in the bone marrow are to be used to modify HSCs or HSPCs, followed by genome editing HSC transplantation.

In certain embodiments, amino acid 268E on the CXCR4 protein is targeted for substitution. The amino acid is changed to 268A or 268N with homology directed repair utilizing CRISPR/Cas9 to modify the amino acid based on the required cDNA sequence. Interaction of CXCR4 with HIV gp120 has been demonstrated to be reduced significantly by this amino acid substitution (Zhou et al., J. Biol. Chem. 2001;276:42826-42833; Brelot et al., J. Biol. Chem. 2000;275:23736-23744.). The method reduces HIV binding to CXCR4, decreases viral entry and ameliorates disease. Methods that alter binding of HIV gp120 to CXCR4 but do not disrupt CXCR4 mediated chemotaxis and binding to SDF-1, or HSC homing to, lodging, and retention in the bone marrow are to be used to modify HSCs or HSPCs, followed by genome editing HSC transplantation.

In certain embodiments, amino acid 277E on the CXCR4 protein is targeted for substitution. The amino acid is changed to 277A with homology directed repair utilizing CRISPR/Cas9 to modify the amino acid based on the required cDNA sequence. Interaction of CXCR4 with HIV gp120 has been demonstrated to be reduced significantly by this amino acid substitution (Tamamis et al., Biophys J. 2013 Sep. 17; 105(6): 1502-1514). The method reduces HIV binding to CXCR4, decreases viral entry and ameliorates disease. Methods that alter binding of HIV gp120 to CXCR4 but do not disrupt CXCR4 mediated chemotaxis and binding to SDF-1, or HSC homing to, lodging, and retention in the bone marrow are to be used to modify HSCs or HSPCs, followed by genome editing HSC transplantation.

In certain embodiments, the targeting domain of the gRNA molecule is configured to provide a cleavage event, e.g., a double strand break or a single strand break, sufficiently close to (e.g., either 5′ or 3′ to) the target position in the CXCR4 gene for introduction of the mutation in the CXCR4 gene e.g., at 171D, 193D, 200Q, 255Y, 268E, or 277E. In certain embodiments, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position in the CXCR4 gene. The break, e.g., a double strand or single strand break, can be positioned upstream or downstream of the target position in the CXCR4 gene.

In certain embodiments, a second, third and/or fourth gRNA molecule is configured to provide a cleavage event, e.g., a double strand break or a single strand break, sufficiently close to (e.g., either 5′ or 3′ to) the target position in the CXCR4 gene for introduction of the mutation in the CXCR4 gene e.g., at 171D, 193D, 200Q, 255Y, 268E, or 277E. In certain embodiments, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position in the CXCR4 gene. The break, e.g., a double strand or single strand break, can be positioned upstream or downstream of the target position in the CXCR4 gene.

In certain embodiments, a single strand break is accompanied by an additional single strand break, positioned by a second, third and/or fourth gRNA molecule, as discussed below. For example, The targeting domains bind configured such that a cleavage event, e.g., the two single strand breaks, are positioned within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position in the CXCR4 gene for introduction of the mutation in the CXCR4 gene e.g., at 171D, 193D, 200Q, 255Y, 268E, or 277E. In certain embodiments, the first and second gRNA molecules are configured such, that when guiding a Cas9 nickase, a single strand break can be accompanied by an additional single strand break, positioned by a second gRNA, sufficiently close to one another to result in an alteration of the target position in the CXCR4 gene. In certain embodiments, the first and second gRNA molecules are configured such that a single strand break positioned by said second gRNA is within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the break positioned by said first gRNA molecule, e.g., when the Cas9 is a nickase. In certain embodiments, the two gRNA molecules are configured to position cuts at the same position, or within a few nucleotides of one another, on different strands, e.g., essentially mimicking a double strand break.

In certain embodiments, a double strand break can be accompanied by an additional double strand break, positioned by a second, third and/or fourth gRNA molecule, as is discussed below. For example, the targeting domain of a first gRNA molecule is configured such that a double strand break is positioned upstream of the target position in the CXCR4 gene within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position; and the targeting domain of a second gRNA molecule is configured such that a double strand break is positioned downstream the target position in the CXCR4 gene, within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position.

In certain embodiments, a double strand break can be accompanied by two additional single strand breaks, positioned by a second gRNA molecule and a third gRNA molecule. For example, the targeting domain of a first gRNA molecule is configured such that a double strand break is positioned upstream of the target position in the CXCR4 gene, e.g., within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position; and the targeting domains of a second and third gRNA molecule are configured such that two single strand breaks are positioned downstream of the target position in the CXCR4 gene, within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position. In certain embodiments, the targeting domain of the first, second and third gRNA molecules are configured such that a cleavage event, e.g., a double strand or single strand break, is positioned, independently for each of the gRNA molecules.

In certain embodiments, a first and second single strand breaks can be accompanied by two additional single strand breaks positioned by a third gRNA molecule and a fourth gRNA molecule. For example, the targeting domain of a first and second gRNA molecule are configured such that two single strand breaks are positioned upstream of the target position in the CXCR4 gene, e.g., within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position in the CXCR4 gene; and the targeting domains of a third and fourth gRNA molecule are configured such that two single strand breaks are positioned downstream of the target position in the CXCR4 gene, e.g., within 1, 2, 3, 4, 5, 10, 15, 20, 25, 30, 35, 40, 45, 50, 60, 70, 80, 90, 100, 150, 200, 250, 300, 350, 400, 450 or 500 nucleotides of the target position in the CXCR4 gene.

In certain embodiments, a mutation in the CXCR4 gene, e.g., at 171D, 193D, 200Q, 255Y, 268E, or 277E is introduced using an exogenously provided template nucleic acid, e.g., by HDR. In certain embodiments, the template nucleic acid is a single strand deoxyoligonucleotide (ssODN). In certain embodiments, the template nuclei acid comprises the mutation at the target position in the CXCR4 gene for introduction of the mutation in the CXCR4 gene e.g., at 171D, 193D, 200Q, 255Y, 268E, or 277E in the CXCR4 gene.

In certain embodiments, an eaCas9 molecule, e.g., an eaCas9 molecule described herein, is used. In an embodiment, the eaCas9 molecule comprises HNH-like domain cleavage activity but has no, or no significant, N-terminal RuvC-like domain cleavage activity. In certain embodiments, the eaCas9 molecule is an HNH-like domain nickase. In certain embodiments, the eaCas9 molecule comprises a mutation at D10 (e.g., D10A). In certain embodiments, the eaCas9 molecule comprises N-terminal RuvC-like domain cleavage activity but has no, or no significant, HNH-like domain cleavage activity. In certain embodiments, the eaCas9 molecule is an N-terminal RuvC-like domain nickase. In certain embodiments, the eaCas9 molecule comprises a mutation at H840 (e.g., H840A) or N863 (e.g., N863A).

### 6. Methods of Multiplexed Alteration of Both CCR5 and CXCR4

As disclosed herein, both the CCR5 gene and the CXCR4 gene can be altered by gene editing, e.g., using the CRISPR-Cas9 mediated methods, genome editing systems, and compositions described herein. The alteration of two or more genes (e.g., CCR5 and CRCX4 genes) is referred to herein as “multiplexing”. In certain embodiments, multiplexing comprisesalteration of at least two genes (e.g., a CCR5 gene and a CRCX4 gene).

Methods, genome editing systems, and compositions discussed herein provide for altering both a CCR5 target position in the CCR5 gene and a CXCR4 target position in the CXCR4 gene.

Any one of the approaches for altering CCR5 described in Section 4 can be combined with any one of the approaches for altering CXCR4 described in Section 5 for multiplexed alteration of CCR5 and CXCR4. For example, multiplexed alteration of CCR5 and CXCR4 can be achieved by one or more of the following approaches:

(i) knocking out the CCR5 gene and knocking out the CXCR4 gene;

(ii) knocking out the CCR5 gene and knocking down the CXCR4 gene;

(iii) knocking down the CCR5 gene and knocking out the CXCR4 gene;

(iv) knocking down the CCR5 gene and knocking down the CXCR4 gene;

(v) introducing one or more mutations in the CCR5 gene and knocking out the CXCR4 gene;

(vi) introducing one or more mutations in the CCR5 gene and knocking down the CXCR4 gene;

(vii) knocking out the CCR5 gene and introducing one or more mutations in the CXCR4 gene;

(viii) knocking down the CCR5 gene and introducing one or more mutations in the CXCR4 gene; and

(ix) introducing one or more mutations in the CCR5 gene and introducing one or more mutations in the CXCR4 gene.

Knocking out the CCR5 gene can be achieved by one or more of the approaches described in Section 4, e.g., insertion or deletion (e.g., NHEJ-mediated insertion or deletion) of one or more nucleotides in close proximity to or within the early coding region of the CCR5 gene (referred to as “(4.1a)” in Section 4), deletion (e.g., NHEJ-mediated deletion) of a genomic sequence including at least a portion of the CCR5 gene (referred to as “(4.1b)” in Section 4), knockout of CCR5 with concomitant knock-in of anti-HIV gene or genes under expression of endogenous promoter or Pol III promoter (referred to as “(4.1c)” in Section 4); and knockout of CCR5 with concomitant knock-in of drug resistance selectable marker for enabling selection of modified HSCs (referred to as “(4.1d)” in Section 4).

Knocking down the CCR5 gene can be achieved by the approach described in Section 4, e.g., mediated by enzymatically inactive Cas9 (eiCas9) molecule or an eiCas9-fusion protein (referred to as “(4.2)” in Section 4).

Introducing one or more mutations in the CCR5 gene can be achieved by one or more approaches described in Section 4, e.g., NHEJ-mediated creation of naturally occurring delta 32 mutation in CCR5 gene (referred to as “(4.3 a)” in Section 4); and HDR-mediated introduction of delta 32 mutation to CCR5 (referred to as “(4.3b)” in Section 4).

Knocking out the CXCR4 gene can be achieved by one or more of the approaches described in Section 5, e.g., insertion or deletion (e.g., NHEJ-mediated insertion or deletion) of one or more nucleotides in close proximity to or within the early coding region of the CXCR4 gene (referred to as “(5.1a)” in Section 5), deletion (e.g., NHEJ-mediated deletion) of a genomic sequence including at least a portion of the CXCR4 gene (referred to as “(5.1b)” in Section 5), and deletion (e.g., NHEJ-mediated deletion) of amino acids in N-terminus in the CXCR4 gene (referred to as “(5.1c)” in Section 5).

Knocking down the CXCR4 gene can be achieved by the approach described in Section 5, e.g., mediated by enzymatically inactive Cas9 (eiCas9) molecule or an eiCas9-fusion protein (referred to as “(5.2)” in Section 5).

Introducing one or more mutations in the CXCR4 gene can be achieved by ne or more of the approaches described in Section 5, e.g., HDR-mediated introduction of one or more mutations (e.g., single or double base subsitutions) in the CXCR4 gene (referred to as “(5.3)” in Section 5).

In certain embodiments, multiplexed alteration of CCR5 and CXCR4 can be achieved by one or more of the following approaches:

(a) insertion or deletion (e.g., NHEJ-mediated insertion or deletion) of one or more nucleotides in close proximity to or within the early coding region of the CCR5 gene (referred to as “(4.1a)” in Section 4), and insertion or deletion (e.g., NHEJ-mediated insertion or deletion) of one or more nucleotides in close proximity to or within the early coding region of the CXCR4 gene (referred to as “(5.1a)” in Section 5);

(b) deletion (e.g., NHEJ-mediated deletion) of a genomic sequence including at least a portion of the CCR5 gene (referred to as “(4.1b)” in Section 4), and insertion or deletion (e.g., NHEJ-mediated insertion or deletion) of one or more nucleotides in close proximity to or within the early coding region of the CXCR4 gene (referred to as “(5.1a)” in Section 5);

(c) knockout of CCR5 with concomitant knock-in of anti-HIV gene or genes under expression of endogenous promoter or Pol III promoter (referred to as “(4.1c)” in Section 4), and insertion or deletion (e.g., NHEJ-mediated insertion or deletion) of one or more nucleotides in close proximity to or within the early coding region of the CXCR4 gene (referred to as “(5.1a)” in Section 5);

(d) knockout of CCR5 with concomitant knock-in of drug resistance selectable marker for enabling selection of modified HSCs (referred to as “(4.1d)” in Section 4), and insertion or deletion (e.g., NHEJ-mediated insertion or deletion) of one or more nucleotides in close proximity to or within the early coding region of the CXCR4 gene (referred to as “(5.1a)” in Section 5);

(e) knockdown of CCR5 mediated by enzymatically inactive Cas9 (eiCas9) molecule or an eiCas9-fusion protein (referred to as “(4.2)” in Section 4), and insertion or deletion (e.g., NHEJ-mediated insertion or deletion) of one or more nucleotides in close proximity to or within the early coding region of the CXCR4 gene (referred to as “(5.1a)” in Section 5);

(f) NHEJ-mediated creation of naturally occurring delta 32 mutation in CCR5 gene (referred to as “(4.3 a)” in Section 4), and insertion or deletion (e.g., NHEJ-mediated insertion or deletion) of one or more nucleotides in close proximity to or within the early coding region of the CXCR4 gene (referred to as “(5.1a)” in Section 5);

(g) HDR-mediated introduction of delta 32 mutation to CCR5 (referred to as “(4.3b)” in Section 4), and insertion or deletion (e.g., NHEJ-mediated insertion or deletion) of one or more nucleotides in close proximity to or within the early coding region of the CXCR4 gene (referred to as “(5.1a)” in Section 5);

(h) insertion or deletion (e.g., NHEJ-mediated insertion or deletion) of one or more nucleotides in close proximity to or within the early coding region of the CCR5 gene (referred to as “(4.1a)” in Section 4), deletion (e.g., NHEJ-mediated deletion) of a genomic sequence including at least a portion of the CXCR4 gene (referred to as “(5.1b)” in Section 5);

(i) deletion (e.g., NHEJ-mediated deletion) of a genomic sequence including at least a portion of the CCR5 gene (referred to as “(4.1b)” in Section 4), deletion (e.g., NHEJ-mediated deletion) of a genomic sequence including at least a portion of the CXCR4 gene (referred to as “(5.1b)” in Section 5);

(j) knockout of CCR5 with concomitant knock-in of anti-HIV gene or genes under expression of endogenous promoter or Pol III promoter (referred to as “(4.1c)” in Section 4), and deletion (e.g., NHEJ-mediated deletion) of a genomic sequence including at least a portion of the CXCR4 gene (referred to as “(5.1b)” in Section 5);

(k) knockout of CCR5 with concomitant knock-in of drug resistance selectable marker for enabling selection of modified HSCs (referred to as “(4.1d)” in Section 4), and deletion (e.g., NHEJ-mediated deletion) of a genomic sequence including at least a portion of the CXCR4 gene (referred to as “(5.1b)” in Section 5);

(l) knockdown of CCR5 mediated by enzymatically inactive Cas9 (eiCas9) molecule or an eiCas9-fusion protein (referred to as “(4.2)” in Section 4), and deletion (e.g., NHEJ-mediated deletion) of a genomic sequence including at least a portion of the CXCR4 gene (referred to as “(5.1b)” in Section 5);

(m) NHEJ-mediated creation of naturally occurring delta 32 mutation in CCR5 gene (referred to as “(4.3 a)” in Section 4), and deletion (e.g., NHEJ-mediated deletion) of a genomic sequence including at least a portion of the CXCR4 gene (referred to as “(5.1b)” in Section 5);

(n) HDR-mediated introduction of delta 32 mutation to CCR5 (referred to as “(4.3b)” in Section 4), and deletion (e.g., NHEJ-mediated deletion) of a genomic sequence including at least a portion of the CXCR4 gene (referred to as “(5.1b)” in Section 5);

(o) insertion or deletion (e.g., NHEJ-mediated insertion or deletion) of one or more nucleotides in close proximity to or within the early coding region of the CCR5 gene (referred to as “(4.1a)” in Section 4), and deletion (e.g., NHEJ-mediated deletion) of amino acids in N-terminus in the CXCR4 gene (referred to as “(5.1c)” in Section 5);

(p) deletion (e.g., NHEJ-mediated deletion) of a genomic sequence including at least a portion of the CCR5 gene (referred to as “(4.1b)” in Section 4), and deletion (e.g., NHEJ-mediated deletion) of amino acids in N-terminus in the CXCR4 gene (referred to as “(5.1c)” in Section 5);

(q) knockout of CCR5 with concomitant knock-in of anti-HIV gene or genes under expression of endogenous promoter or Pol III promoter (referred to as “(4.1c)” in Section 4), and deletion (e.g., NHEJ-mediated deletion) of amino acids in N-terminus in the CXCR4 gene (referred to as “(5.1c)” in Section 5);

(r) knockout of CCR5 with concomitant knock-in of drug resistance selectable marker for enabling selection of modified HSCs (referred to as “(4.1d)” in Section 4), and deletion (e.g., NHEJ-mediated deletion) of amino acids in N-terminus in the CXCR4 gene (referred to as “(5.1c)” in Section 5);

(s) knockdown of CCR5 mediated by enzymatically inactive Cas9 (eiCas9) molecule or an eiCas9-fusion protein (referred to as “(4.2)” in Section 4), and deletion (e.g., NHEJ-mediated deletion) of amino acids in N-terminus in the CXCR4 gene (referred to as “(5.1c)” in Section 5);

(t) NHEJ-mediated creation of naturally occurring delta 32 mutation in CCR5 gene (referred to as “(4.3 a)” in Section 4), and deletion (e.g., NHEJ-mediated deletion) of amino acids in N-terminus in the CXCR4 gene (referred to as “(5.1c)” in Section 5);

(u) HDR-mediated introduction of delta 32 mutation to CCR5 (referred to as “(4.3b)” in Section 4), and deletion (e.g., NHEJ-mediated deletion) of amino acids in N-terminus in the CXCR4 gene (referred to as “(5.1c)” in Section 5);

(v) insertion or deletion (e.g., NHEJ-mediated insertion or deletion) of one or more nucleotides in close proximity to or within the early coding region of the CCR5 gene (referred to as “(4.1a)” in Section 4), and knockdown of the CXCR4 gene mediated by enzymatically inactive Cas9 (eiCas9) molecule or an eiCas9-fusion protein (referred to as “(5.2)” in Section 5);

(w) deletion (e.g., NHEJ-mediated deletion) of a genomic sequence including at least a portion of the CCR5 gene (referred to as “(4.1b)” in Section 4), and knockdown of the CXCR4 gene mediated by enzymatically inactive Cas9 (eiCas9) molecule or an eiCas9-fusion protein (referred to as “(5.2)” in Section 5);

(x) knockout of CCR5 with concomitant knock-in of anti-HIV gene or genes under expression of endogenous promoter or Pol III promoter (referred to as “(4.1c)” in Section 4), and knockdown of the CXCR4 gene mediated by enzymatically inactive Cas9 (eiCas9) molecule or an eiCas9-fusion protein (referred to as “(5.2)” in Section 5);

(y) knockout of CCR5 with concomitant knock-in of drug resistance selectable marker for enabling selection of modified HSCs (referred to as “(4.1d)” in Section 4), and knockdown of the CXCR4 gene mediated by enzymatically inactive Cas9 (eiCas9) molecule or an eiCas9-fusion protein (referred to as “(5.2)” in Section 5);

(z) knockdown of CCR5 mediated by enzymatically inactive Cas9 (eiCas9) molecule or an eiCas9-fusion protein (referred to as “(4.2)” in Section 4), and knockdown of the CXCR4 gene mediated by enzymatically inactive Cas9 (eiCas9) molecule or an eiCas9-fusion protein (referred to as “(5.2)” in Section 5);

(aa) NHEJ-mediated creation of naturally occurring delta 32 mutation in CCR5 gene (referred to as “(4.3 a)” in Section 4), and knockdown of the CXCR4 gene mediated by enzymatically inactive Cas9 (eiCas9) molecule or an eiCas9-fusion protein (referred to as “(5.2)” in Section 5);

(ab) HDR-mediated introduction of delta 32 mutation to CCR5 (referred to as “(4.3b)” in Section 4), and knockdown of the CXCR4 gene mediated by enzymatically inactive Cas9 (eiCas9) molecule or an eiCas9-fusion protein (referred to as “(5.2)” in Section 5);

(ac) insertion or deletion (e.g., NHEJ-mediated insertion or deletion) of one or more nucleotides in close proximity to or within the early coding region of the CCR5 gene (referred to as “(4.1a)” in Section 4), and HDR-mediated introduction of one or more mutations (e.g., single or double base subsitutions) in the CXCR4 gene (referred to as “(5.3)” in Section 5);

(ad) deletion (e.g., NHEJ-mediated deletion) of a genomic sequence including at least a portion of the CCR5 gene (referred to as “(4.1b)” in Section 4), and HDR-mediated introduction of one or more mutations (e.g., single or double base subsitutions) in the CXCR4 gene (referred to as “(5.3)” in Section 5);

(ae) knockout of CCR5 with concomitant knock-in of anti-HIV gene or genes under expression of endogenous promoter or Pol III promoter (referred to as “(4.1c)” in Section 4), and HDR-mediated introduction of one or more mutations (e.g., single or double base subsitutions) in the CXCR4 gene (referred to as “(5.3)” in Section 5);

(af) knockout of CCR5 with concomitant knock-in of drug resistance selectable marker for enabling selection of modified HSCs (referred to as “(4.1d)” in Section 4), and HDR-mediated introduction of one or more mutations (e.g., single or double base subsitutions) in the CXCR4 gene (referred to as “(5.3)” in Section 5);

(ag) knockdown of CCR5 mediated by enzymatically inactive Cas9 (eiCas9) molecule or an eiCas9-fusion protein (referred to as “(4.2)” in Section 4), and HDR-mediated introduction of one or more mutations (e.g., single or double base subsitutions) in the CXCR4 gene (referred to as “(5.3)” in Section 5);

(ah) NHEJ-mediated creation of naturally occurring delta 32 mutation in CCR5 gene (referred to as “(4.3 a)” in Section 4), and HDR-mediated introduction of one or more mutations (e.g., single or double base subsitutions) in the CXCR4 gene (referred to as “(5.3)” in Section 5); and

(ai) HDR-mediated introduction of delta 32 mutation to CCR5 (referred to as “(4.3b)” in Section 4), and HDR-mediated introduction of one or more mutations (e.g., single or double base subsitutions) in the CXCR4 gene (referred to as “(5.3)” in Section 5).

In certain embodiments, multiplexed alteration of CCR5 and CXCR4 can be achieved by knocking out a CCR gene and knocking out a CXCR4 gene.

In certain embodiments, alteration of the CCR5 gene and the CXCR4 gene, decreases or eliminates the expression of both T tropic and M tropic coreceptors for the HIV virus. In certain embodiments, the HIV virus is unable to infect CD4 cells, CD8 cells, T cells, B cells, neutrophils, eosinophils, GALT, dendritic cells, microglia cells, myeloid progenitor cells, and/or lymphoid progenitor cells. In certain embodiments, HIV is unable to spread within the host and/or the disease is treated.In certain embodiments, a single Cas9 molecule is configured, e.g., for the introduction of one or more breaks in a CCR5 target position and a CXCR4 target position; for introduction of one or more breaks in a CXCR4 target position and for the introduction of two sets of breaks in a CCR5 target position; for introduction of one or more breaks in a CXCR4 target position and for the introduction of two sets of breaks in a CCR5 target position; or an eiCas9 targeting the alteration of transcription, e.g., to block, reduce, or decrease transcription, of the CXCR4 and the CCR5 gene. In certain embodiments, two distinct Cas9 molecules are configured, e.g. a Cas9 nickase targeting a CCR5 target position and a Cas9 nickase targeting a CXCR4 target position; an eiCas9 to alter transcription (e.g., to block, reduce, or decrease transcription) of the CCR5 gene and a Cas9 nickase targeting a CXCR4 target position; an eiCas9 molecule to alter transcription (e.g., to block, reduce, or decrease transcription) of the CXCR4 gene and a Cas9 nickase targeting a CCR5 target position; or an eiCas9 targeting the alteration of transcription (e.g., to block, reduce, or decrease transcription) of the CXCR4 gene and an eiCas9 targeting the alteration of transcription (e.g., to block, reduce, or decrease transcription) of the CCR5 gene.

When two or more genes (e.g., CCR5 and CXCR4) are targeted for alteration, the two or more genes (e.g., CCR5 and CXCR4) can be altered sequentially or simultaneously. In certain embodiments, the the CCR5 gene and the CXCR4 gene are altered simultaneously. In certain embodiments, the the CCR5 gene and the CXCR4 gene are altered sequentially. In certain embodiments, the alteration of the CXCR4 gene is prior to the alteration of the CCR5 gene. In certain embodiments, the alteration of the CXCR4 gene is concurrent with the alteration of the CCR5 gene. In certain embodiments, the alteration of the CXCR4 gene is subsequent to the alteration of the CCR5 gene. In certain embodiments, the effect of the alterations is synergistic. In certain embodiments, the two or more genes (e.g., CCR5 and CXCR4) are altered sequentially in order to reduce the probability of introducing genomic rearrangements (e.g., translocations) involving the two target positions.

7. Guide RNA (gRNA) Molecules

A gRNA molecule, as that term is used herein, refers to a nucleic acid that promotes the specific targeting or homing of a gRNA molecule/Cas9 molecule complex to a target nucleic acid. gRNA molecules can be unimolecular (having a single RNA molecule) (e.g., chimeric), or modular (comprising more than one, and typically two, separate RNA molecules). The gRNA molecules provided herein comprise a targeting domain comprising, consisting of, or consisting essentially of a nucleic acid sequence fully or partially complementary to a target domain (also referred to as “target sequence”). In certain embodiments, the gRNA molecule further comprises one or more additional domains, including for example a first complementarity domain, a linking domain, a second complementarity domain, a proximal domain, a tail domain, and a 5′ extension domain. Each of these domains is discussed in detail below. In certain embodiments, one or more of the domains in the gRNA molecule comprises a nucleotide sequecne identical to or sharing sequence homology with a naturally occurring sequence, e.g., from S. pyogenes, S. aureus, or S. thermophilus. In certain embodiments, one or more of the domains in the gRNA molecule comprises a nucleotide sequecne identical to or sharing sequence homology with a naturally occurring sequence, e.g., from S. pyogenes or S. aureus,

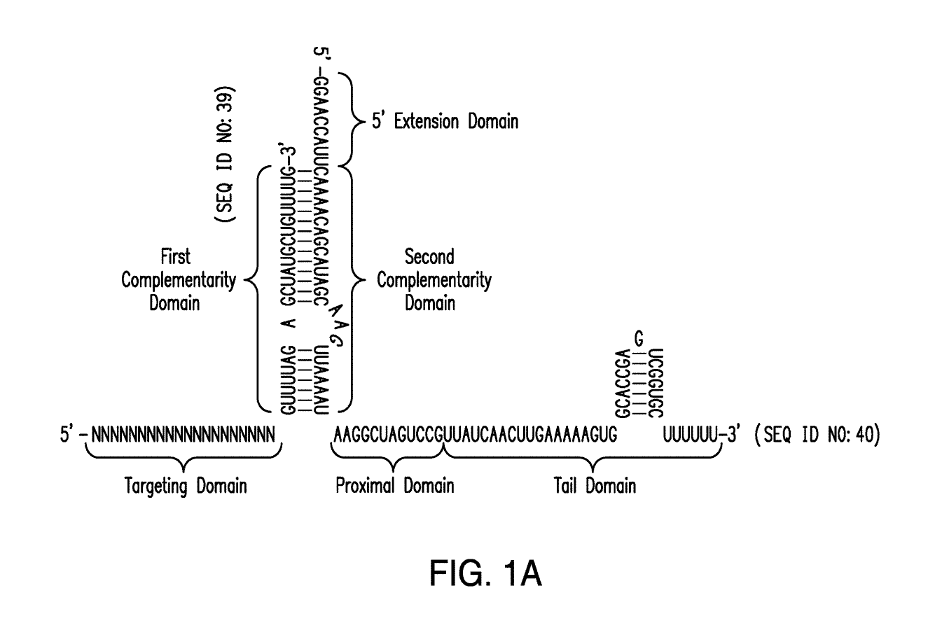

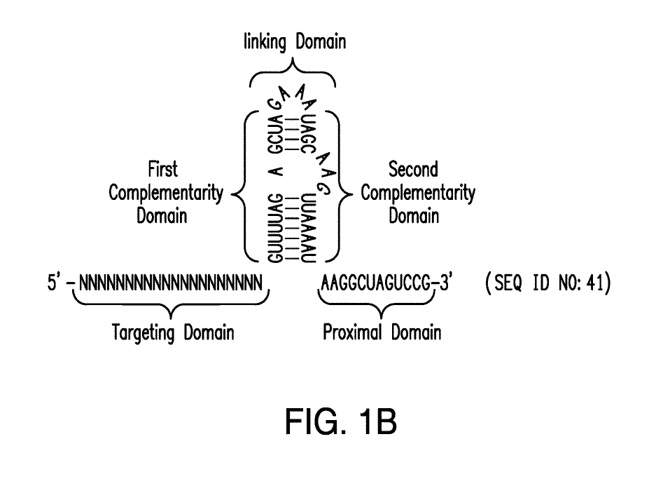

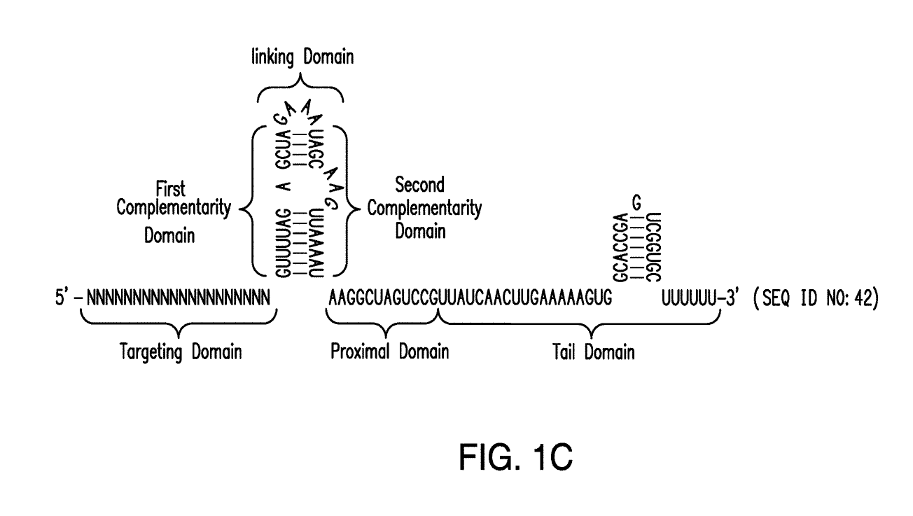

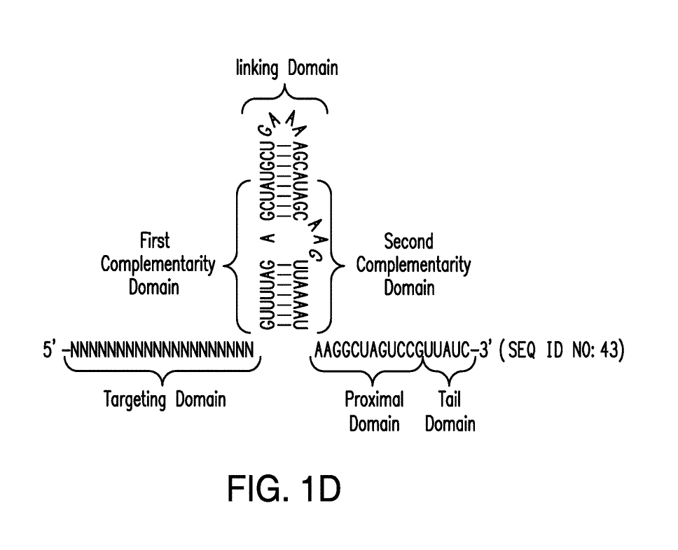

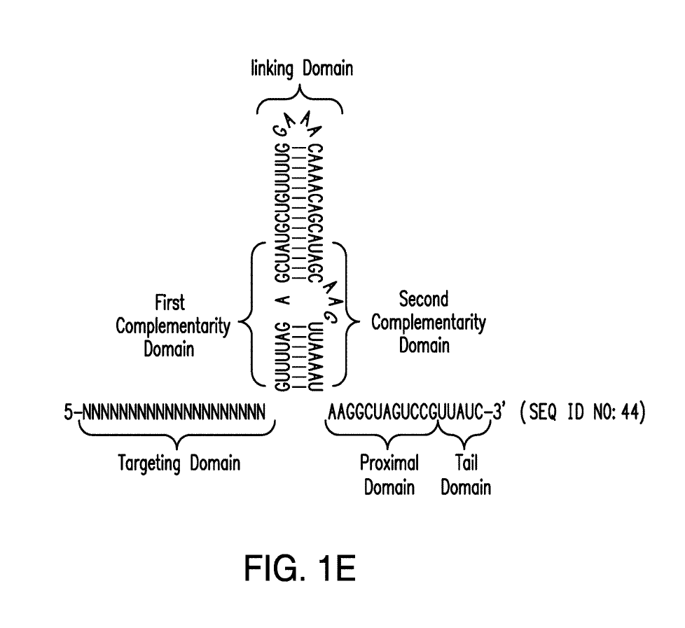

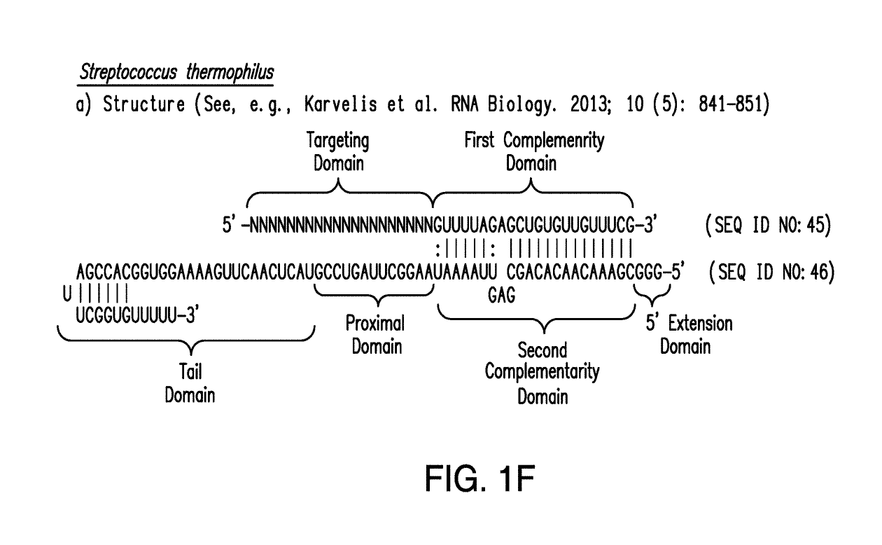

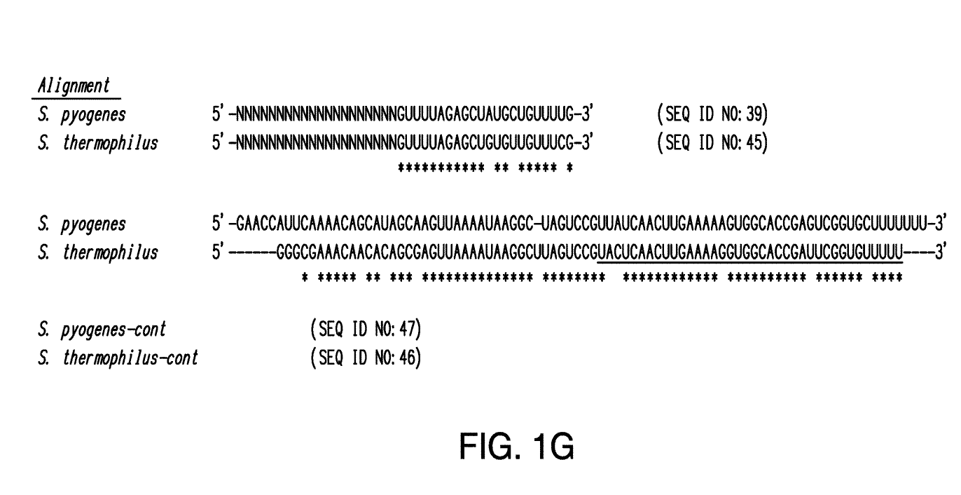

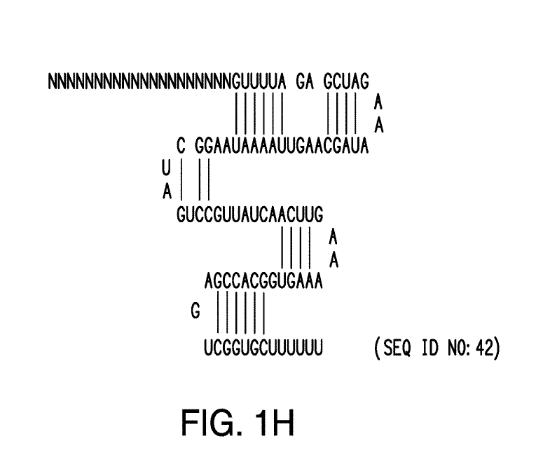

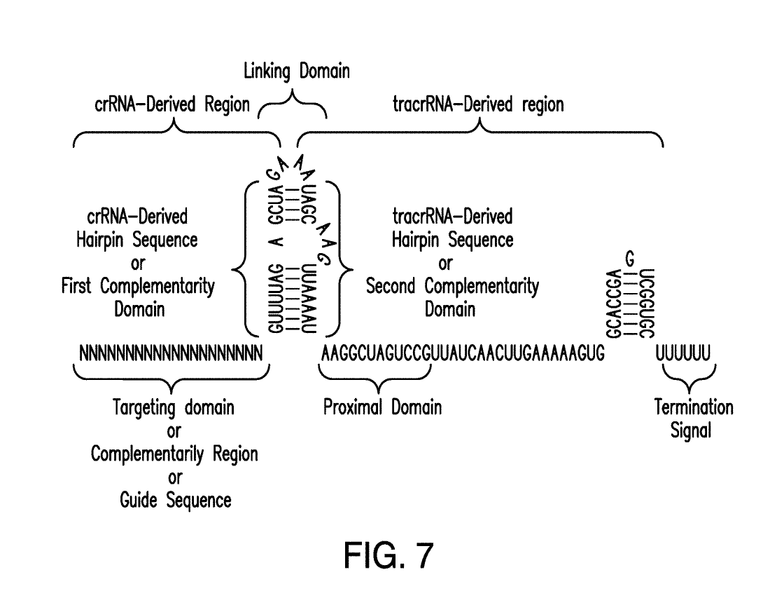

Several exemplary gRNA structures are provided in FIGS. 1A-1I. With regard to the three-dimensional form, or intra- or inter-strand interactions of an active form of a gRNA, regions of high complementarity are sometimes shown as duplexes in FIGS. 1A-1I and other depictions provided herein. FIG. 7 illustrates gRNA domain nomenclature using the gRNA sequence of SEQ ID NO:42, which contains one hairpin loop in the tracrRNA-derived region. In certain embodiments, a gRNA may contain more than one (e.g., two, three, or more) hairpin loops in this region (see, e.g., FIGS. 1H-1I).

In certain embodiments, a unimolecular, or chimeric, gRNA comprises, preferably from 5′ to 3′:

a targeting domain complementary to a target domain in a CCR5 gene or a CXCR4 gene, e.g., a targeting domain comprising a nucleotide sequence selected from SEQ ID NOs: 208 to 3739 (e.g., SEQ ID NOs: 208 to 1569 and 1617 to 3663) or SEQ ID NOs: 3740 to 8407 (e.g., SEQ ID NOs: 3740 to 5208 and 5241 to 8355);

a first complementarity domain;

a linking domain;

a second complementarity domain (which is complementary to the first complementarity domain);

a proximal domain; and

optionally, a tail domain.

In certain embodiments, a modular gRNA comprises:

a first strand comprising, preferably from 5′ to 3′:

a targeting domain complementary to a target domain in a CCR5 gene or a CXCR4 gene, e.g., a targeting domain comprising a nucleotide sequence selected from SEQ ID NOs: 208 to 3739 (e.g., SEQ ID NOs: 208 to 1569 and 1617 to 3663) or SEQ ID NOs: 3740 to 8407 (e.g., SEQ ID NOs: 3740 to 5208 and 5241 to 8355); and

a first complementarity domain; and

a second strand, comprising, preferably from 5′ to 3′:

optionally, a 5′ extension domain;

a second complementarity domain;

a proximal domain; and

optionally, a tail domain.

7.1 Targeting Domain

The targeting domain (sometimes referred to alternatively as the guide sequence) comprises, consists of, or consists essentially of a nucleic acid sequence that is complementary or partially complementary to a target nucleic acid sequence in a CCR5 gene or a CXCR4 gene. The nucleic acid sequence in a CCR5 gene or a CXCR4 gene to which all or a portion of the targeting domain is complementary or partially complementary is referred to herein as the target domain.

Methods for selecting targeting domains are known in the art (see, e.g., Fu 2014; Sternberg 2014). Examples of suitable targeting domains for use in the methods, compositions, and kits described herein comprise nucleotide sequences set forth in SEQ ID NOs: 208 to 8407.

The strand of the target nucleic acid comprising the target domain is referred to herein as the complementary strand because it is complementary to the targeting domain sequence. Since the targeting domain is part of a gRNA molecule, it comprises the base uracil (U) rather than thymine (T); conversely, any DNA molecule encoding the gRNA molecule can comprise thymine rather than uracil. In a targeting domain/target domain pair, the uracil bases in the targeting domain will pair with the adenine bases in the target domain. In certain embodiments, the degree of complementarity between the targeting domain and target domain is sufficient to allow targeting of a Cas9 molecule to the target nucleic acid.

In certain embodiments, the targeting domain comprises a core domain and an optional secondary domain. In certain of these embodiments, the core domain is located 3′ to the secondary domain, and in certain of these embodiments the core domain is located at or near the 3′ end of the targeting domain. In certain of these embodiments, the core domain consists of or consists essentially of about 8 to about 13 nucleotides at the 3′ end of the targeting domain. In certain embodiments, only the core domain is complementary or partially complementary to the corresponding portion of the target domain, and in certain of these embodiments the core domain is fully complementary to the corresponding portion of the target domain. In certain embodiments, the secondary domain is also complementary or partially complementary to a portion of the target domain. In certain embodiments, the core domain is complementary or partially complementary to a core domain target in the target domain, while the secondary domain is complementary or partially complementary to a secondary domain target in the target domain. In certain embodiments, the core domain and secondary domain have the same degree of complementarity with their respective corresponding portions of the target domain. In certain embodiments, the degree of complementarity between the core domain and its target and the degree of complementarity between the secondary domain and its target may differ. In certain of these embodiments, the core domain may have a higher degree of complementarity for its target than the secondary domain, whereas in other embodiments the secondary domain may have a higher degree of complementarity than the core domain.

In certain embodiments, the targeting domain and/or the core domain within the targeting domain is 3 to 100, 5 to 100, 10 to 100, or 20 to 100 nucleotides in length, and in certain of these embodiments the targeting domain or core domain is 3 to 15, 3 to 20, 5 to 20, 10 to 20, 15 to 20, 5 to 50, 10 to 50, or 20 to 50 nucleotides in length. In certain embodiments, the targeting domain and/or the core domain within the targeting domain is 6, 7, 8, 9, 10, 11, 12, 13, 14, 15, 16, 17, 18, 19, 20, 21, 22, 23, 24, 25, or 26 nucleotides in length. In certain embodiments, the targeting domain and/or the core domain within the targeting domain is 6+/−2, 7+/−2, 8+/−2, 9+/−2, 10+/−2, 10+/−4, 10+/−5, 11+/−2, 12+/−2, 13+/−2, 14+/−2, 15+/−2, or 16+−2, 20+/−5, 30+/−5, 40+/−5, 50+/−5, 60+/−5, 70+/−5, 80+/−5, 90+/−5, or 100+/−5 nucleotides in length.

In certain embodiments wherein the targeting domain includes a core domain, the core domain is 3 to 20 nucleotides in length, and in certain of these embodiments the core domain 5 to 15 or 8 to 13 nucleotides in length. In certain embodiments wherein the targeting domain includes a secondary domain, the secondary domain is 0, 1, 2, 3, 4, 5, 6, 7, 8, 9, 10, 11, 12, 13, 14 or 15 nucleotides in length. In certain embodiments wherein the targeting domain comprises a core domain that is 8 to 13 nucleotides in length, the targeting domain is 26, 25, 24, 23, 22, 21, 20, 19, 18, 17, or 16 nucleotides in length, and the secondary domain is 13 to 18, 12 to 17, 11 to 16, 10 to 15, 9 to 14, 8 to 13, 7 to 12, 6 to 11, 5 to 10, 4 to 9, or 3 to 8 nucleotides in length, respectively.

In certain embodiments, the targeting domain is fully complementary to the target domain. Likewise, where the targeting domain comprises a core domain and/or a secondary domain, in certain embodiments one or both of the core domain and the secondary domain are fully complementary to the corresponding portions of the target domain. In certain embodiments, the targeting domain is partially complementary to the target domain, and in certain of these embodiments where the targeting domain comprises a core domain and/or a secondary domain, one or both of the core domain and the secondary domain are partially complementary to the corresponding portions of the target domain. In certain of these embodiments, the nucleic acid sequence of the targeting domain, or the core domain or targeting domain within the targeting domain, is at least about 80%, about 85%, about 90%, or about 95% complementary to the target domain or to the corresponding portion of the target domain. In certain embodiments, the targeting domain and/or the core or secondary domains within the targeting domain include one or more nucleotides that are not complementary with the target domain or a portion thereof, and in certain of these embodiments the targeting domain and/or the core or secondary domains within the targeting domain include 1, 2, 3, 4, 5, 6, 7, or 8 nucleotides that are not complementary with the target domain. In certain embodiments, the core domain includes 1, 2, 3, 4, or 5 nucleotides that are not complementary with the corresponding portion of the target domain. In certain embodiments wherein the targeting domain includes one or more nucleotides that are not complementary with the target domain, one or more of said non-complementary nucleotides are located within five nucleotides of the 5′ or 3′ end of the targeting domain. In certain of these embodiments, the targeting domain includes 1, 2, 3, 4, or 5 nucleotides within five nucleotides of its 5′ end, 3′ end, or both its 5′ and 3′ ends that are not complementary to the target domain. In certain embodiments wherein the targeting domain includes two or more nucleotides that are not complementary to the target domain, two or more of said non-complementary nucleotides are adjacent to one another, and in certain of these embodiments the two or more consecutive non-complementary nucleotides are located within five nucleotides of the 5′ or 3′ end of the targeting domain. In certain embodiments, the two or more consecutive non-complementary nucleotides are both located more than five nucleotides from the 5′ and 3′ ends of the targeting domain.

In certain embodiments, the targeting domain, core domain, and/or secondary domain do not comprise any modifications. In certain embodiments, the targeting domain, core domain, and/or secondary domain, or one or more nucleotides therein, have a modification, including but not limited to the modifications set forth below. In certain embodiments, one or more nucleotides of the targeting domain, core domain, and/or secondary domain may comprise a 2′ modification (e.g., a modification at the 2′ position on ribose), e.g., a 2-acetylation, e.g., a 2′ methylation. In certain embodiments, the backbone of the targeting domain can be modified with a phosphorothioate. In certain embodiments, modifications to one or more nucleotides of the targeting domain, core domain, and/or secondary domain render the targeting domain and/or the gRNA comprising the targeting domain less susceptible to degradation or more bio-compatible, e.g., less immunogenic. In certain embodiments, the targeting domain and/or the core or secondary domains include 1, 2, 3, 4, 5, 6, 7, or 8 or more modifications, and in certain of these embodiments the targeting domain and/or core or secondary domains include 1, 2, 3, or 4 modifications within five nucleotides of their respective 5′ ends and/or 1, 2, 3, or 4 modifications within five nucleotides of their respective 3′ ends. In certain embodiments, the targeting domain and/or the core or secondary domains comprise modifications at two or more consecutive nucleotides.

In certain embodiments wherein the targeting domain includes core and secondary domains, the core and secondary domains contain the same number of modifications. In certain of these embodiments, both domains are free of modifications. In other embodiments, the core domain includes more modifications than the secondary domain, or vice versa.

In certain embodiments, modifications to one or more nucleotides in the targeting domain, including in the core or secondary domains, are selected to not interfere with targeting efficacy, which can be evaluated by testing a candidate modification using a system as set forth below. gRNAs having a candidate targeting domain having a selected length, sequence, degree of complementarity, or degree of modification can be evaluated using a system as set forth below. The candidate targeting domain can be placed, either alone or with one or more other candidate changes in a gRNA molecule/Cas9 molecule system known to be functional with a selected target, and evaluated.

In certain embodiments, all of the modified nucleotides are complementary to and capable of hybridizing to corresponding nucleotides present in the target domain. In certain embodiments, 1, 2, 3, 4, 5, 6, 7 or 8 or more modified nucleotides are not complementary to or capable of hybridizing to corresponding nucleotides present in the target domain.

7.2 First and Second Complementarity Domains

The first and second complementarity (sometimes referred to alternatively as the crRNA-derived hairpin sequence and tracrRNA-derived hairpin sequences, respectively) domains are fully or partially complementary to one another. In certain embodiments, the degree of complementarity is sufficient for the two domains to form a duplexed region under at least some physiological conditions. In certain embodiments, the degree of complementarity between the first and second complementarity domains, together with other properties of the gRNA, is sufficient to allow targeting of a Cas9 molecule to a target nucleic acid. Examples of first and second complementary domains are set forth in FIGS. 1A-1G.

In certain embodiments (see, e.g., FIGS. 1A-1B) the first and/or second complementarity domain includes one or more nucleotides that lack complementarity with the corresponding complementarity domain. In certain embodiments, the first and/or second complementarity domain includes 1, 2, 3, 4, 5, or 6 nucleotides that do not complement with the corresponding complementarity domain. For example, the second complementarity domain may contain 1, 2, 3, 4, 5, or 6 nucleotides that do not pair with corresponding nucleotides in the first complementarity domain. In certain embodiments, the nucleotides on the first or second complementarity domain that do not complement with the corresponding complementarity domain loop out from the duplex formed between the first and second complementarity domains. In certain of these embodiments, the unpaired loop-out is located on the second complementarity domain, and in certain of these embodiments the unpaired region begins 1, 2, 3, 4, 5, or 6 nucleotides from the 5′ end of the second complementarity domain.

In certain embodiments, the first complementarity domain is 5 to 30, 5 to 25, 7 to 25, 5 to 24, 5 to 23, 7 to 22, 5 to 22, 5 to 21, 5 to 20, 7 to 18, 7 to 15, 9 to 16, or 10 to 14 nucleotides in length, and in certain of these embodiments the first complementarity domain is 5, 6, 7, 8, 9, 10, 11, 12, 13, 14, 15, 16, 17, 18, 19, 20, 21, 22, 23, 24, or 25 nucleotides in length. In certain embodiments, the second complementarity domain is 5 to 27, 7 to 27, 7 to 25, 5 to 24, 5 to 23, 5 to 22, 5 to 21, 7 to 20, 5 to 20, 7 to 18, 7 to 17, 9 to 16, or 10 to 14 nucleotides in length, and in certain of these embodiments the second complementarity domain is 5, 6, 7, 8, 9, 10, 11, 12, 13, 14, 15, 16, 17, 18, 19, 20, 21, 22, 23, 24, 25, or 26 nucleotides in length. In certain embodiments, the first and second complementarity domains are each independently 6+/−2, 7+/−2, 8+/−2, 9+/−2, 10+/−2, 11+/−2, 12+/−2, 13+/−2, 14+/−2, 15+/−2, 16+/−2, 17+/−2, 18+/−2, 19+/−2, or 20+/−2, 21+/−2, 22+/−2, 23+/−2, or 24+/−2 nucleotides in length. In certain embodiments, the second complementarity domain is longer than the first complementarity domain, e.g., 2, 3, 4, 5, or 6 nucleotides longer.

In certain embodiments, the first and/or second complementarity domains each independently comprise three subdomains, which, in the 5′ to 3′ direction are: a 5′ subdomain, a central subdomain, and a 3′ subdomain. In certain embodiments, the 5′ subdomain and 3′ subdomain of the first complementarity domain are fully or partially complementary to the 3′ subdomain and 5′ subdomain, respectively, of the second complementarity domain. In certain embodiments, the 5′ subdomain of the first complementarity domain is 4 to 9 nucleotides in length, and in certain of these embodiments the 5′ domain is 4, 5, 6, 7, 8, or 9 nucleotides in length. In certain embodiments, the 5′ subdomain of the second complementarity domain is 3 to 25, 4 to 22, 4 to 18, or 4 to 10 nucleotides in length, and in certain of these embodiments the 5′ domain is 3, 4, 5, 6, 7, 8, 9, 10, 11, 12, 13, 14, 15, 16, 17, 18, 19, 20, 21, 22, 23, 24, or 25 nucleotides in length. In certain embodiments, the central subdomain of the first complementarity domain is 1, 2, or 3 nucleotides in length. In certain embodiments, the central subdomain of the second complementarity domain is 1, 2, 3, 4, or 5 nucleotides in length. In certain embodiments, the 3′ subdomain of the first complementarity domain is 3 to 25, 4 to 22, 4 to 18, or 4 to 10 nucleotides in length, and in certain of these embodiments the 3′ subdomain is 3, 4, 5, 6, 7, 8, 9, 10, 11, 12, 13, 14, 15, 16, 17, 18, 19, 20, 21, 22, 23, 24, or 25 nucleotides in length. In certain embodiments, the 3′ subdomain of the second complementarity domain is 4 to 9, e.g., 4, 5, 6, 7, 8 or 9 nucleotides in length.

The first and/or second complementarity domains can share homology with, or be derived from, naturally occurring or reference first and/or second complementarity domain. In certain of these embodiments, the first and/or second complementarity domains have at least about 50%, about 60%, about 70%, about 80%, about 85%, about 90%, or about 95% homology with, or differ by no more than 1, 2, 3, 4, 5, or 6 nucleotides from, the naturally occurring or reference first and/or second complementarity domain. In certain of these embodiments, the first and/or second complementarity domains may have at least about 50%, about 60%, about 70%, about 80%, about 85%, about 90%, or about 95% homology with homology with a first and/or second complementarity domain from S. pyogenes or S. aureus.

In certain embodiments, the first and/or second complementarity domains do not comprise any modifications. In other embodiments, the first and/or second complementarity domains or one or more nucleotides therein have a modification, including but not limited to a modification set forth below. In certain embodiments, one or more nucleotides of the first and/or second complementarity domain may comprise a 2′ modification (e.g., a modification at the 2′ position on ribose), e.g., a 2-acetylation, e.g., a 2′ methylation. In certain embodiments, the backbone of the targeting domain can be modified with a phosphorothioate. In certain embodiments, modifications to one or more nucleotides of the first and/or second complementarity domain render the first and/or second complementarity domain and/or the gRNA comprising the first and/or second complementarity less susceptible to degradation or more bio-compatible, e.g., less immunogenic. In certain embodiments, the first and/or second complementarity domains each independently include 1, 2, 3, 4, 5, 6, 7, or 8 or more modifications, and in certain of these embodiments the first and/or second complementarity domains each independently include 1, 2, 3, or 4 modifications within five nucleotides of their respective 5′ ends, 3′ ends, or both their 5′ and 3′ ends. In certain embodiments, the first and/or second complementarity domains each independently contain no modifications within five nucleotides of their respective 5′ ends, 3′ ends, or both their 5′ and 3′ ends. In certain embodiments, one or both of the first and second complementarity domains comprise modifications at two or more consecutive nucleotides.

In certain embodiments, modifications to one or more nucleotides in the first and/or second complementarity domains are selected to not interfere with targeting efficacy, which can be evaluated by testing a candidate modification in a system as set forth below. gRNAs having a candidate first or second complementarity domain having a selected length, sequence, degree of complementarity, or degree of modification can be evaluated in a system as set forth below. The candidate complementarity domain can be placed, either alone or with one or more other candidate changes in a gRNA molecule/Cas9 molecule system known to be functional with a selected target, and evaluated.

In certain embodiments, the duplexed region formed by the first and second complementarity domains is, for example, 6, 7, 8, 9, 10, 11, 12, 13, 14, 15, 16, 17, 18, 19, 20, 21, or 22 bp in length, excluding any looped out or unpaired nucleotides.

In certain embodiments, the first and second complementarity domains, when duplexed, comprise 11 paired nucleotides (see, for e.g., gRNA of SEQ ID NO:48). In certain embodiments, the first and second complementarity domains, when duplexed, comprise 15 paired nucleotides (see, e.g., gRNA of SEQ ID NO:50). In certain embodiments, the first and second complementarity domains, when duplexed, comprise 16 paired nucleotides (see, e.g., gRNA of SEQ ID NO:51). In certain embodiments, the first and second complementarity domains, when duplexed, comprise 21 paired nucleotides (see, e.g., gRNA of SEQ ID NO:29).

In certain embodiments, one or more nucleotides are exchanged between the first and second complementarity domains to remove poly-U tracts. For example, nucleotides 23 and 48 or nucleotides 26 and 45 of the gRNA of SEQ ID NO:48 may be exchanged to generate the gRNA of SEQ ID NOs:49 or 31, respectively. Similarly, nucleotides 23 and 39 of the gRNA of SEQ ID NO:29 may be exchanged with nucleotides 50 and 68 to generate the gRNA of SEQ ID NO:30.

7.3 Linking Domain

The linking domain is disposed between and serves to link the first and second complementarity domains in a unimolecular or chimeric gRNA. FIGS. 1B-1E provide examples of linking domains. In certain embodiments, part of the linking domain is from a crRNA-derived region, and another part is from a tracrRNA-derived region.

In certain embodiments, the linking domain links the first and second complementarity domains covalently. In certain of these embodiments, the linking domain consists of or comprises a covalent bond. In other embodiments, the linking domain links the first and second complementarity domains non-covalently. In certain embodiments, the linking domain is ten or fewer nucleotides in length, e.g., 1, 2, 3, 4, 5, 6, 7, 8, 9, or 10 nucleotides. In other embodiments, the linking domain is greater than 10 nucleotides in length, e.g., 11, 12, 13, 14, 15, 16, 17, 18, 19, 20, 21, 22, 23, 24, or 25 or more nucleotides. In certain embodiments, the linking domain is 2 to 50, 2 to 40, 2 to 30, 2 to 20, 2 to 10, 2 to 5, 10 to 100, 10 to 90, 10 to 80, 10 to 70, 10 to 60, 10 to 50, 10 to 40, 10 to 30, 10 to 20, 10 to 15, 20 to 100, 20 to 90, 20 to 80, 20 to 70, 20 to 60, 20 to 50, 20 to 40, 20 to 30, or 20 to 25 nucleotides in length. In certain embodiments, the linking domain is 10+/−5, 20+/−5, 20+/−10, 30+/−5, 30+/−10, 40+/−5, 40+/−10, 50+/−5, 50+/−10, 60+/−5, 60+/−, 70+/−70+/−10, 80+/−5, 80+/−10, 90+/−5, 90+/−10, 100+/−5, or 100+/−10 nucleotides in length.

In certain embodiments, the linking domain shares homology with, or is derived from, a naturally occurring sequence, e.g., the sequence of a tracrRNA that is 5′ to the second complementarity domain. In certain embodiments, the linking domain has at least about 50%, about 60%, about 70%, about 80%, about 90%, or about 95% homology with or differs by no more than 1, 2, 3, 4, 5, or 6 nucleotides from a linking domain disclosed herein, e.g., the linking domains of FIGS. 1B-1E.

In certain embodiments, the linking domain does not comprise any modifications. In other embodiments, the linking domain or one or more nucleotides therein have a modification, including but not limited to the modifications set forth below. In certain embodiments, one or more nucleotides of the linking domain may comprise a 2′ modification (e.g., a modification at the 2′ position on ribose), e.g., a 2-acetylation, e.g., a 2′ methylation. In certain embodiments, the backbone of the linking domain can be modified with a phosphorothioate. In certain embodiments, modifications to one or more nucleotides of the linking domain render the linking domain and/or the gRNA comprising the linking domain less susceptible to degradation or more bio-compatible, e.g., less immunogenic. In certain embodiments, the linking domain includes 1, 2, 3, 4, 5, 6, 7, or 8 or more modifications, and in certain of these embodiments the linking domain includes 1, 2, 3, or 4 modifications within five nucleotides of its 5′ and/or 3′ end. In certain embodiments, the linking domain comprises modifications at two or more consecutive nucleotides.

In certain embodiments, modifications to one or more nucleotides in the linking domain are selected to not interfere with targeting efficacy, which can be evaluated by testing a candidate modification in a system as set forth below. gRNAs having a candidate linking domain having a selected length, sequence, degree of complementarity, or degree of modification can be evaluated in a system as set forth below. The candidate linking domain can be placed, either alone or with one or more other candidate changes in a gRNA molecule/Cas9 molecule system known to be functional with a selected target, and evaluated.

In certain embodiments, the linking domain comprises a duplexed region, typically adjacent to or within 1, 2, or 3 nucleotides of the 3′ end of the first complementarity domain and/or the 5′ end of the second complementarity domain. In certain of these embodiments, the duplexed region of the linking region is 10+/−5, 15+/−5, 20+/−5, 20+/−10, or 30+/−5 bp in length. In certain embodiments, the duplexed region of the linking domain is 1, 2, 3, 4, 5, 6, 7, 8, 9, 10, 11, 12, 13, 14, or 15 bp in length. In certain embodiments, the sequences forming the duplexed region of the linking domain are fully complementarity. In other embodiments, one or both of the sequences forming the duplexed region contain one or more nucleotides (e.g., 1, 2, 3, 4, 5, 6, 7, or 8 nucleotides) that are not complementary with the other duplex sequence.

7.4 5′ Extension Domain

In certain embodiments, a modular gRNA as disclosed herein comprises a 5′ extension domain, i.e., one or more additional nucleotides 5′ to the second complementarity domain (see, e.g., FIG. 1A). In certain embodiments, the 5′ extension domain is 2 to 10 or more, 2 to 9, 2 to 8, 2 to 7, 2 to 6, 2 to 5, or 2 to 4 nucleotides in length, and in certain of these embodiments the 5′ extension domain is 2, 3, 4, 5, 6, 7, 8, 9, or 10 or more nucleotides in length.

In certain embodiments, the 5′ extension domain nucleotides do not comprise modifications, e.g., modifications of the type provided below. However, in certain embodiments, the 5′ extension domain comprises one or more modifications, e.g., modifications that it render it less susceptible to degradation or more bio-compatible, e.g., less immunogenic. By way of example, the backbone of the 5′ extension domain can be modified with a phosphorothioate, or other modification(s) as set forth below. In certain embodiments, a nucleotide of the 5′ extension domain can comprise a 2′ modification (e.g., a modification at the 2′ position on ribose), e.g., a 2-acetylation, e.g., a 2′ methylation, or other modification(s) as set forth below.

In certain embodiments, the 5′ extension domain can comprise as many as 1, 2, 3, 4, 5, 6, 7, or 8 modifications. In certain embodiments, the 5′ extension domain comprises as many as 1, 2, 3, or 4 modifications within 5 nucleotides of its 5′ end, e.g., in a modular gRNA molecule. In certain embodiments, the 5′ extension domain comprises as many as 1, 2, 3, or 4 modifications within 5 nucleotides of its 3′ end, e.g., in a modular gRNA molecule.

In certain embodiments, the 5′ extension domain comprises modifications at two consecutive nucleotides, e.g., two consecutive nucleotides that are within 5 nucleotides of the 5′ end of the 5′ extension domain, within 5 nucleotides of the 3′ end of the 5′ extension domain, or more than 5 nucleotides away from one or both ends of the 5′ extension domain. In certain embodiments, no two consecutive nucleotides are modified within 5 nucleotides of the 5′ end of the 5′ extension domain, within 5 nucleotides of the 3′ end of the 5′ extension domain, or within a region that is more than 5 nucleotides away from one or both ends of the 5′ extension domain. In certain embodiments, no nucleotide is modified within 5 nucleotides of the 5′ end of the 5′ extension domain, within 5 nucleotides of the 3′ end of the 5′ extension domain, or within a region that is more than 5 nucleotides away from one or both ends of the 5′ extension domain.

Modifications in the 5′ extension domain can be selected so as to not interfere with gRNA molecule efficacy, which can be evaluated by testing a candidate modification in a system as set forth below. gRNAs having a candidate 5′ extension domain having a selected length, sequence, degree of complementarity, or degree of modification, can be evaluated in a system as set forth below. The candidate 5′ extension domain can be placed, either alone, or with one or more other candidate changes in a gRNA molecule/Cas9 molecule system known to be functional with a selected target and evaluated.

In certain embodiments, the 5′ extension domain has at least about 60%, about 70%, about 80%, about 85%, about 90%, or about 95% homology with, or differs by no more than 1, 2, 3, 4, 5, or 6 nucleotides from, a reference 5′ extension domain, e.g., a naturally occurring, e.g., an S. pyogenes, S. aureus, or S. thermophilus, 5′ extension domain, or a 5′ extension domain described herein, e.g., from FIGS. 1A-1G.

7.5 Proximal Domain

FIGS. 1A-1G provide examples of proximal domains.

In certain embodiments, the proximal domain is 5 to 20 or more nucleotides in length, e.g., 5, 6, 7, 8, 9, 10, 11, 12, 13, 14, 15, 16, 17, 18, 19, 20, 21, 22, 23, 24, 25, or 26 nucleotides in length. In certain of these embodiments, the proximal domain is 6+/−2, 7+/−2, 8+/−2, 9+/−2, 10+/−2, 11+/−2, 12+/−2, 13+/−2, 14+/−2, 14+/−2, 16+/−2, 17+/−2, 18+/−2, 19+/−2, or 20+/−2 nucleotides in length. In certain embodiments, the proximal domain is 5 to 20, 7, to 18, 9 to 16, or 10 to 14 nucleotides in length.

In certain embodiments, the proximal domain can share homology with or be derived from a naturally occurring proximal domain. In certain of these embodiments, the proximal domain has at least about 50%, about 60%, about 70%, about 80%, about 85%, about 90%, or about 95% homology with or differs by no more than 1, 2, 3, 4, 5, or 6 nucleotides from a proximal domain disclosed herein, e.g., an S. pyogenes, S. aureus, or S. thermophilus proximal domain, including those set forth in FIGS. 1A-1G.

In certain embodiments, the proximal domain does not comprise any modifications. In other embodiments, the proximal domain or one or more nucleotides therein have a modification, including but not limited to the modifications set forth in herein. In certain embodiments, one or more nucleotides of the proximal domain may comprise a 2′ modification (e.g., a modification at the 2′ position on ribose), e.g., a 2-acetylation, e.g., a 2′ methylation. In certain embodiments, the backbone of the proximal domain can be modified with a phosphorothioate. In certain embodiments, modifications to one or more nucleotides of the proximal domain render the proximal domain and/or the gRNA comprising the proximal domain less susceptible to degradation or more bio-compatible, e.g., less immunogenic. In certain embodiments, the proximal domain includes 1, 2, 3, 4, 5, 6, 7, or 8 or more modifications, and in certain of these embodiments the proximal domain includes 1, 2, 3, or 4 modifications within five nucleotides of its 5′ and/or 3′ end. In certain embodiments, the proximal domain comprises modifications at two or more consecutive nucleotides.

In certain embodiments, modifications to one or more nucleotides in the proximal domain are selected to not interfere with targeting efficacy, which can be evaluated by testing a candidate modification in a system as set forth below. gRNAs having a candidate proximal domain having a selected length, sequence, degree of complementarity, or degree of modification can be evaluated in a system as set forth below. The candidate proximal domain can be placed, either alone or with one or more other candidate changes in a gRNA molecule/Cas9 molecule system known to be functional with a selected target, and evaluated.

7.6 Tail Domain

A broad spectrum of tail domains are suitable for use in the gRNA molecules disclosed herein. FIGS. 1A and 1C-1G provide examples of such tail domains.

In certain embodiments, the tail domain is absent. In other embodiments, the tail domain is 1 to 100 or more nucleotides in length, e.g., 1, 2, 3, 4, 5, 6, 7, 8, 9, 10, 20, 30, 40, 50, 60, 70, 80, 90, or 100 nucleotides in length. In certain embodiments, the tail domain is 1 to 5, 1 to 10, 1 to 15, 1 to 20, 1 to 50, 10 to 100, 20 to 100, 10 to 90, 20 to 90, 10 to 80, 20 to 80, 10 to 70, 20 to 70, 10 to 60, 20 to 60, 10 to 50, 20 to 50, 10 to 40, 20 to 40, 10 to 30, 20 to 30, 20 to 25, 10 to 20, or 10 to 15 nucleotides in length. In certain embodiments, the tail domain is 5+/−5, 10+/−5, 20+/−10, 20+/−5, 25+/−10, 30+/−10, 30+/−5, 40+/−10, 40+/−5, 50+/−10, 50+/−5, 60+/−10, 60+/−5, 70+/−10, 70+/−5, 80+/−10, 80+/−5, 90+/−10, 90+/−5, 100+/−10, or 100+/−5 nucleotides in length,

In certain embodiments, the tail domain can share homology with or be derived from a naturally occurring tail domain or the 5′ end of a naturally occurring tail domain. In certain of these embodiments, the proximal domain has at least about 50%, about 60%, about 70%, about 80%, about 85%, about 90%, or about 95% homology with or differs by no more than 1, 2, 3, 4, 5, or 6 nucleotides from a naturally occurring tail domain disclosed herein, e.g., an S. pyogenes, S. aureus, or S. thermophilus tail domain, including those set forth in FIGS. 1A and 1C-1G.

In certain embodiments, the tail domain includes sequences that are complementary to each other and which, under at least some physiological conditions, form a duplexed region. In certain of these embodiments, the tail domain comprises a tail duplex domain which can form a tail duplexed region. In certain embodiments, the tail duplexed region is 3, 4, 5, 6, 7, 8, 9, 10, 11, or 12 bp in length. In certain embodiments, the tail domain comprises a single stranded domain 3′ to the tail duplex domain that does not form a duplex. In certain of these embodiments, the single stranded domain is 3 to 10 nucleotides in length, e.g., 3, 4, 5, 6, 7, 8, 9, 10, or 4 to 6 nucleotides in length.

In certain embodiments, the tail domain does not comprise any modifications. In other embodiments, the tail domain or one or more nucleotides therein have a modification, including but not limited to the modifications set forth herein. In certain embodiments, one or more nucleotides of the tail domain may comprise a 2′ modification (e.g., a modification at the 2′ position on ribose), e.g., a 2-acetylation, e.g., a 2′ methylation. In certain embodiments, the backbone of the tail domain can be modified with a phosphorothioate. In certain embodiments, modifications to one or more nucleotides of the tail domain render the tail domain and/or the gRNA comprising the tail domain less susceptible to degradation or more bio-compatible, e.g., less immunogenic. In certain embodiments, the tail domain includes 1, 2, 3, 4, 5, 6, 7, or 8 or more modifications, and in certain of these embodiments the tail domain includes 1, 2, 3, or 4 modifications within five nucleotides of its 5′ and/or 3′ end. In certain embodiments, the tail domain comprises modifications at two or more consecutive nucleotides.

In certain embodiments, modifications to one or more nucleotides in the tail domain are selected to not interfere with targeting efficacy, which can be evaluated by testing a candidate modification as set forth below. gRNAs having a candidate tail domain having a selected length, sequence, degree of complementarity, or degree of modification can be evaluated using a system as set forth below. The candidate tail domain can be placed, either alone or with one or more other candidate changes in a gRNA molecule/Cas9 molecule system known to be functional with a selected target, and evaluated.

In certain embodiments, the tail domain includes nucleotides at the 3′ end that are related to the method of in vitro or in vivo transcription. When a T7 promoter is used for in vitro transcription of the gRNA, these nucleotides may be any nucleotides present before the 3′ end of the DNA template. In certain embodiments, the gRNA molecule includes a 3′ polyA tail that is prepared by in vitro transcription from a DNA template. In certain embodiments, the 5′ nucleotide of the targeting domain of the gRNA molecule is a guanine nucleotide, the DNA template comprises a T7 promoter sequence located immediately upstream of the sequence that corresponds to the targeting domain, and the 3′ nucleotide of the T7 promoter sequence is not a guanine nucleotide. In certain embodiments, the 5′ nucleotide of the targeting domain of the gRNA molecule is not a guanine nucleotide, the DNA template comprises a T7 promoter sequence located immediately upstream of the sequence that corresponds to the targeting domain, and the 3′ nucleotide of the T7 promoter sequence is a guanine nucleotide which is downstream of a nucleotide other than a guanine nucleotide.

When a U6 promoter is used for in vivo transcription, these nucleotides may be the sequence UUUUUU. When an H1 promoter is used for transcription, these nucleotides may be the sequence UUUU. When alternate pol-III promoters are used, these nucleotides may be various numbers of uracil bases depending on, e.g., the termination signal of the pol-III promoter, or they may include alternate bases.

In certain embodiments, the proximal and tail domain taken together comprise, consist of, or consist essentially of the sequence set forth in SEQ ID NOs:32, 33, 34, 35, 36, or 37.

7.7 Exemplary Unimolecular/Chimeric gRNAs

In certain embodiments, a gRNA as disclosed herein has the structure: 5′ [targeting domain]-[first complementarity domain]-[linking domain]-[second complementarity domain]-[proximal domain]-[tail domain]-3′, wherein:

the targeting domain comprises a core domain and optionally a secondary domain, and is 10 to 50 nucleotides in length;

the first complementarity domain is 5 to 25 nucleotides in length and, in certain embodiments has at least about 50%, about 60%, about 70%, about 80%, about 85%, about 90%, or about 95% homology with a reference first complementarity domain disclosed herein; the linking domain is 1 to 5 nucleotides in length;

the second complementarity domain is 5 to 27 nucleotides in length and, in certain embodiments has at least about 50%, about 60%, about 70%, about 80%, about 85%, about 90%, or about 95% homology with a reference second complementarity domain disclosed herein; the proximal domain is 5 to 20 nucleotides in length and, in certain embodiments has at least about 50%, about 60%, about 70%, about 80%, about 85%, about 90%, or about 95% homology with a reference proximal domain disclosed herein; and

the tail domain is absent or a nucleotide sequence is 1 to 50 nucleotides in length and, in certain embodiments has at least about 50%, about 60%, about 70%, about 80%, about 85%, about 90%, or about 95% homology with a reference tail domain disclosed herein.

In certain embodiments, a unimolecular gRNA as disclosed herein comprises, preferably from 5′ to 3′:

a targeting domain, e.g., comprising 10-50 nucleotides;

a first complementarity domain, e.g., comprising 15, 16, 17, 18, 19, 20, 21, 22, 23, 24, 25, or 26 nucleotides;

a linking domain;

a second complementarity domain;

a proximal domain; and

a tail domain,

wherein,

(a) the proximal and tail domain, when taken together, comprise at least 15, 18, 20, 25, 30, 31, 35, 40, 45, 49, 50, or 53 nucleotides;

(b) there are at least 15, 18, 20, 25, 30, 31, 35, 40, 45, 49, 50, or 53 nucleotides 3′ to the last nucleotide of the second complementarity domain; or

(c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the sequence from (a), (b), and/or (c) has at least about 50%, about 60%, about 70%, about 75%, about 60%, about 70%, about 80%, about 85%, about 90%, about 95%, or about 99% homology with the corresponding sequence of a naturally occurring gRNA, or with a gRNA described herein.

In certain embodiments, the proximal and tail domain, when taken together, comprise at least 15, 18, 20, 25, 30, 31, 35, 40, 45, 49, 50, or 53 nucleotides.

In certain embodiments, there are at least 15, 18, 20, 25, 30, 31, 35, 40, 45, 49, 50, or 53 nucleotides 3′ to the last nucleotide of the second complementarity domain.

In certain embodiments, there are at least 16, 19, 21, 26, 31, 32, 36, 41, 46, 50, 51, or 54 nucleotides 3′ to the last nucleotide of the second complementarity domain that are complementary to the corresponding nucleotides of the first complementarity domain.

In certain embodiments, the targeting domain consists of, consists essentially of, or comprises 16, 17, 18, 19, 20, 21, 22, 23, 24, 25, or 26 nucleotides (e.g., 16, 17, 18, 19, 20, 21, 22, 23, 24, 25, or 26 consecutive nucleotides) complementary or partially complementary to the target domain or a portion thereof, e.g., the targeting domain is 16, 17, 18, 19, 20, 21, 22, 23, 24, 25, or 26 nucleotides in length. In certain of these embodiments, the targeting domain is complementary to the target domain over the entire length of the targeting domain, the entire length of the target domain, or both.

In certain embodiments, a unimolecular or chimeric gRNA molecule disclosed herein (comprising a targeting domain, a first complementary domain, a linking domain, a second complementary domain, a proximal domain and, optionally, a tail domain) comprises the amino acid sequence set forth in SEQ ID NO:42, wherein the targeting domain is listed as 20 N's (residues 1-20) but may range in length from 16 to 26 nucleotides, and wherein the final six residues (residues 97-102) represent a termination signal for the U6 promoter buy may be absent or fewer in number. In certain embodiments, the unimolecular, or chimeric, gRNA molecule is a S. pyogenes gRNA molecule.

In certain embodiments, a unimolecular or chimeric gRNA molecule disclosed herein (comprising a targeting domain, a first complementary domain, a linking domain, a second complementary domain, a proximal domain and, optionally, a tail domain) comprises the amino acid sequence set forth in SEQ ID NO:38, wherein the targeting domain is listed as 20 Ns (residues 1-20) but may range in length from 16 to 26 nucleotides, and wherein the final six residues (residues 97-102) represent a termination signal for the U6 promoter but may be absent or fewer in number. In certain embodiments, the unimolecular or chimeric gRNA molecule is an S. aureus gRNA molecule.

The sequences and structures of exemplary chimeric gRNAs are also shown in FIGS. 1H-1I.

7.8 Exemplary Modular gRNAs

In certain embodiments, a modular gRNA disclosed herein comprises:

a first strand comprising, preferably from 5′ to 3′;

a targeting domain, e.g., comprising 15, 16, 17, 18, 19, 20, 21, 22, 23, 24, 25, or 26 nucleotides;

a first complementarity domain; and

a second strand, comprising, preferably from 5′ to 3′:

optionally a 5′ extension domain;

a second complementarity domain;

a proximal domain; and

a tail domain,

wherein:

(a) the proximal and tail domain, when taken together, comprise at least 15, 18, 20, 25, 30, 31, 35, 40, 45, 49, 50, or 53 nucleotides;

(b) there are at least 15, 18, 20, 25, 30, 31, 35, 40, 45, 49, 50, or 53 nucleotides 3′ to the last nucleotide of the second complementarity domain; or

(c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the sequence from (a), (b), or (c), has at least about 50%, about 60%, about 70%, about 80%, about 85%, about 90%, about 95%, or about 99% homology with the corresponding sequence of a naturally occurring gRNA, or with a gRNA described herein. In certain embodiments, the proximal and tail domain, when taken together, comprise at least 15, 18, 20, 25, 30, 31, 35, 40, 45, 49, 50, or 53 nucleotides.

In certain embodiments, there are at least 15, 18, 20, 25, 30, 31, 35, 40, 45, 49, 50, or 53 nucleotides 3′ to the last nucleotide of the second complementarity domain. In certain embodiments, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 16, 17, 18, 19, 20, 21, 22, 23, 24, 25, or 26 nucleotides (e.g., 16, 17, 18, 19, 20, 21, 22, 23, 24, 25, or 26 consecutive nucleotides) having complementarity with the target domain, e.g., the targeting domain is 16, 17, 18, 19, 20, 21, 22, 23, 24, 25, or 26 nucleotides in length.

In certain embodiments, the targeting domain consists of, consists essentially of, or comprises 16, 17, 18, 19, 20, 21, 22, 23, 24, 25, or 26 nucleotides (e.g., 16, 17, 18, 19, 20, 21, 22, 23, 24, 25, or 26 consecutive nucleotides) complementary to the target domain or a portion thereof. In certain of these embodiments, the targeting domain is complementary to the target domain over the entire length of the targeting domain, the entire length of the target domain, or both.

In certain embodiments, the targeting domain comprises, has, or consists of, 16 nucleotides (e.g., 16 consecutive nucleotides) having complementarity with the target domain, e.g., the targeting domain is 16 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 16 nucleotides (e.g., 16 consecutive nucleotides) having complementarity with the target domain, e.g., the targeting domain is 16 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 16 nucleotides (e.g., 16 consecutive nucleotides) having complementarity with the target domain, e.g., the targeting domain is 16 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain has, or consists of, 17 nucleotides (e.g., 17 consecutive nucleotides) having complementarity with the target domain, e.g., the targeting domain is 17 nucleotides in length; and the proximal and tail domain, when taken together, comprise at least 15, 18, 20, 25, 30, 31, 35, 40, 45, 49, 50, or 53 nucleotides.

In certain embodiments, the targeting domain has, or consists of, 17 nucleotides (e.g., 17 consecutive nucleotides) having complementarity with the target domain, e.g., the targeting domain is 17 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain has, or consists of, 17 nucleotides (e.g., 17 consecutive nucleotides) having complementarity with the target domain, e.g., the targeting domain is 17 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain has, or consists of, 18 nucleotides (e.g., 18 consecutive nucleotides) having complementarity with the target domain, e.g., the targeting domain is 18 nucleotides in length; and the proximal and tail domain, when taken together, comprise at least 15, 18, 20, 25, 30, 31, 35, 40, 45, 49, 50, or 53 nucleotides.

In certain embodiments, the targeting domain has, or consists of, 18 nucleotides (e.g., 18 consecutive nucleotides) having complementarity with the target domain, e.g., the targeting domain is 18 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain has, or consists of, 18 nucleotides (e.g., 18 consecutive nucleotides) having complementarity with the target domain, e.g., the targeting domain is 18 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 19 nucleotides (e.g., 19 consecutive nucleotides) having complementarity with the target domain, e.g., the targeting domain is 19 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 19 nucleotides (e.g., 19 consecutive nucleotides) having complementarity with the target domain, e.g., the targeting domain is 19 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 19 nucleotides (e.g., 19 consecutive nucleotides) having complementarity with the target domain, e.g., the targeting domain is 19 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 20 nucleotides (e.g., 20 consecutive nucleotides) having complementarity with the target domain, e.g., the targeting domain is 20 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 20 nucleotides (e.g., 20 consecutive nucleotides) having complementarity with the target domain, e.g., the targeting domain is 20 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 20 nucleotides (e.g., 20 consecutive nucleotides) having complementarity with the target domain, e.g., the targeting domain is 20 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 21 nucleotides (e.g., 21 consecutive nucleotides) having complementarity with the target domain, e.g., the targeting domain is 21 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 21 nucleotides (e.g., 21 consecutive nucleotides) having complementarity with the target domain, e.g., the targeting domain is 21 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 21 nucleotides (e.g., 21 consecutive nucleotides) having complementarity with the target domain, e.g., the targeting domain is 21 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 22 nucleotides (e.g., 22 consecutive nucleotides) having complementarity with the target domain, e.g., the targeting domain is 22 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 22 nucleotides (e.g., 22 consecutive nucleotides) having complementarity with the target domain, e.g., the targeting domain is 22 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 22 nucleotides (e.g., 22 consecutive nucleotides) having complementarity with the target domain, e.g., the targeting domain is 22 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 23 nucleotides (e.g., 23 consecutive nucleotides) having complementarity with the target domain, e.g., the targeting domain is 23 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 23 nucleotides (e.g., 23 consecutive nucleotides) having complementarity with the target domain, e.g., the targeting domain is 23 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 23 nucleotides (e.g., 23 consecutive nucleotides) having complementarity with the target domain, e.g., the targeting domain is 23 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 24 nucleotides (e.g., 24 consecutive nucleotides) having complementarity with the target domain, e.g., the targeting domain is 24 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 24 nucleotides (e.g., 24 consecutive nucleotides) having complementarity with the target domain, e.g., the targeting domain is 24 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 24 nucleotides (e.g., 24 consecutive nucleotides) having complementarity with the target domain, e.g., the targeting domain is 24 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 25 nucleotides (e.g., 25 consecutive nucleotides) having complementarity with the target domain, e.g., the targeting domain is 25 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 25 nucleotides (e.g., 25 consecutive nucleotides) having complementarity with the target domain, e.g., the targeting domain is 25 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 25 nucleotides (e.g., 25 consecutive nucleotides) having complementarity with the target domain, e.g., the targeting domain is 25 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In certain embodiments, the targeting domain comprises, has, or consists of, 26 nucleotides (e.g., 26 consecutive nucleotides) having complementarity with the target domain, e.g., the targeting domain is 26 nucleotides in length; and the proximal and tail domain, when taken together, comprise at least 15, 18, 20, 25, 30, 31, 35, 40, 45, 49, 50, or 53 nucleotides.

In certain embodiments, the targeting domain comprises, has, or consists of, 26 nucleotides (e.g., 26 consecutive nucleotides) having complementarity with the target domain, e.g., the targeting domain is 26 nucleotides in length; and there are at least 15, 18, 20, 25, 30, 31, 35, 40, 45, 49, 50, or 53 nucleotides 3′ to the last nucleotide of the second complementarity domain.

In certain embodiments, the targeting domain comprises, has, or consists of, 26 nucleotides (e.g., 26 consecutive nucleotides) having complementarity with the target domain, e.g., the targeting domain is 26 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

7.9 gRNA Delivery

In certain embodiments of the methods provided herein, the methods comprise delivery of one or more (e.g., two, three, or four) gRNA molecules as described herein. In certain of these embodiments, the gRNA molecules are delivered by intravenous injection, intramuscular injection, subcutaneous injection, or inhalation. In certain embodiments, the gRNA molecules are delivered with a Cas9 molecule in a genome editing system.

8. Methods for Designing gRNAs

Methods for selecting, designing, and validating targeting domains for use in the gRNAs described herein are provided. Exemplary targeting domains for incorporation into gRNAs are also provided herein.

Methods for selection and validation of target sequences as well as off-target analyses have been described previously (see, e.g., Mali 2013; Hsu 2013; Fu 2014; Heigwer 2014; Bae 2014; Xiao 2014). For example, a software tool can be used to optimize the choice of potential targeting domains corresponding to a user's target sequence, e.g., to minimize total off-target activity across the genome. Off-target activity may be other than cleavage. For each possible targeting domain choice using S. pyogenes Cas9, the tool can identify all off-target sequences (preceding either NAG or NGG PAMs) across the genome that contain up to certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible targeting domain is then ranked according to its total predicted off-target cleavage; the top-ranked targeting domains represent those that are likely to have the greatest on-target cleavage and the least off-target cleavage. Other functions, e.g., automated reagent design for CRISPR construction, primer design for the on-target Surveyor assay, and primer design for high-throughput detection and quantification of off-target cleavage via next-gen sequencing, can also be included in the tool. Candidate targeting domains and gRNAs comprising those targeting domains can be functionally evaluated using methods known in the art and/or as set forth herein.

As a non-limiting example, targeting domains for use in gRNAs for use with S. pyogenes, S. aureus, and N. meningitidis Cas9s were identified using a DNA sequence searching algorithm. 17-mer and 20-mer targeting domains were designed for S. pyogenes and N. meningitidis targets, while 18-mer, 19-mer, 20-mer, 21-mer, 22-mer, 23-mer, and 24-mer targeting domains were designed for S. aureus targets. gRNA design was carried out using custom gRNA design software based on the public tool cas-offinder (Bae 2014). This software scores guides after calculating their genome-wide off-target propensity. Typically matches ranging from perfect matches to 7 mismatches are considered for guides ranging in length from 17 to 24. Once the off-target sites are computationally determined, an aggregate score is calculated for each guide and summarized in a tabular output using a web-interface. In addition to identifying potential target sites adjacent to PAM sequences, the software also identifies all PAM adjacent sequences that differ by 1, 2, 3, or more than 3 nucleotides from the selected target sites. Genomic DNA sequences for each gene (e.g., DMD gene) were obtained from the UCSC Genome browser and sequences were screened for repeat elements using the publically available RepeatMasker program. RepeatMasker searches input DNA sequences for repeated elements and regions of low complexity. The output is a detailed annotation of the repeats present in a given query sequence.

Following identification, targeting domain were ranked into tiers based on their distance to the target site, their orthogonality, and presence of a 5′ G (based on identification of close matches in the human genome containing a relevant PAM, e.g., an NGG PAM for S. pyogenes, an NNGRRT (SEQ ID NO:204) or NNGRRV (SEQ ID NO:205) PAM for S. aureus, or a NNNNGATT (SEQ ID NO: 8408) or NNNNGCTT (SEQ ID NO: 8409) PAM for N. meningitidis). Orthogonality refers to the number of sequences in the human genome that contain a minimum number of mismatches to the target sequence. A “high level of orthogonality” or “good orthogonality” may, for example, refer to 20-mer targeting domain that have no identical sequences in the human genome besides the intended target, nor any sequences that contain one or two mismatches in the target sequence. Targeting domains with good orthogonality are selected to minimize off-target DNA cleavage.

Targeting domains were identified for both single-gRNA nuclease cleavage and for a dual-gRNA paired “nickase” strategy. Criteria for selecting targeting domains and the determination of which targeting domains can be used for the dual-gRNA paired “nickase” strategy is based on two considerations:

(1) Targeting domain pairs should be oriented on the DNA such that PAMs are facing out and cutting with the D10A Cas9 nickase can result in 5′ overhangs; and

(2) An assumption that cleaving with dual nickase pairs will result in deletion of the entire intervening sequence at a reasonable frequency. However, cleaving with dual nickase pairs can also result in indel mutations at the site of only one of the gRNAs. Candidate pair members can be tested for how efficiently they remove the entire sequence versus causing indel mutations at the target site of one targeting domain.

8.1 Targeting Domains For Use In Knocking Out the CCR5 Gene

Targeting domains for use in gRNAs for knocking out the CCR5 gene in conjunction with the methods disclosed herein were identified and ranked into 3 tiers for S. pyogenes, 5 tiers for S. aureus, and 3 tiers for N. meningitidis.

For S. pyogenes, tier 1 targeting domains were selected based on (1) distance to a target site (e.g., start codon), e.g., within 500 bp (e.g., downstream) of the target site (e.g., start codon) and (2) a high level of orthogonality. Tier 2 targeting domains were selected based on (1) distance to the target site (e.g., start codon), e.g., within 500 bp (e.g., downstream) of the target site (e.g., start codon). Tier 3 targeting domains were selected based on distance to the target site (e.g., start codon), e.g., within reminder of the coding sequence, e.g., downstream of the first 500 bp of coding sequence (e.g., anywhere from +500 (relative to the start codon) to the stop codon).

For S. aureus, tier 1 targeting domains were selected based on (1) distance to the target site (e.g., start codon), e.g., within 500 bp (e.g., downstream) of the target site (e.g., start codon), (2) a high level of orthogonality, and (3) PAM is NNGRRT. Tier 2 targeting domains were selected based on (1) distance to the target site (e.g., start codon), e.g., within 500 bp (e.g., downstream) of the target site (e.g., start codon), and (2) PAM is NNGRRT. Tier 3 targeting domains were selected based on (1) distance to a the target site (e.g., start codon), e.g., within 500 bp (e.g., downstream) of the target site (e.g., start codon), and (2) PAM is NNGRRV. Tier 4 targeting domains were selected based on (1) distance to the target site (e.g., start codon), e.g., within reminder of the coding sequence, e.g., downstream of the first 500 bp of coding sequence (e.g., anywhere from +500 (relative to the start codon) to the stop codon), and (2) PAM is NNGRRT. Tier 5 targeting domains were selected based on (1) distance to the target site (e.g., start codon), e.g., within reminder of the coding sequence, e.g., downstream of the first 500 bp of coding sequence (e.g., anywhere from +500 (relative to the start codon) to the stop codon), and (2) PAM is NNGRRV.

For N. meningitidis, tier 1 targeting domains were selected based on (1) distance to the target site, e.g., within 500 bp (e.g., downstream) of the target site (e.g., start codon) and (2) a high level of orthogonality. Tier 2 targeting domains were selected based on (1) distance to the target site (e.g., start codon), e.g., within 500 bp (e.g., downstream) of the target site (e.g., start codon). Tier 3 targeting domains were selected based on distance to the target site (e.g., start codon), e.g., within reminder of the coding sequence, e.g., downstream of the first 500 bp of coding sequence (e.g., anywhere from +500 (relative to the start codon) to the stop codon).

Note that tiers are non-inclusive (each targeting domain is listed only once for the strategy). In certain instances, no targeting domain was identified based on the criteria of the particular tier. The identified targeting domains are summarized below in Table 1.

TABLE 1
Nucleotide sequences of S. pyogenes, S. aureus, and
N. meningitidis targeting domains for knocking out the CCR5 gene

| | S. pyogenes | S. aureus | N. meningitidis |
|---|---|---|---|
| Tier 1 | SEQ ID NOS: 208 | SEQ ID NOS: | SEQ ID NOS: |
| | to 213 | 476 to 496 | 1570 to 1582 |
| Tier 2 | SEQ ID NOS: 214 | SEQ ID NOS: | SEQ ID NOS: |
| | to 339 | 497 to 545 | 1583 to 1591 |
| Tier 3 | SEQ ID NOS: 340 | SEQ ID NOS: | SEQ ID NOS: |
| | to 475 | 546 to 911 | 1592 to 1613 |
| Tier 4 | Not applicable | SEQ ID NOS: | Not applicable |
| | | 912 to 1009 | |
| Tier 5 | Not applicable | SEQ ID NOS: | Not applicable |
| | | 1010 to 1569 | |

In certain embodiments, when a single gRNA molecule is used to target a Cas9 nickase to create a single strand break in close proximity to the CCR5 target position, e.g., the gRNA is used to target either upstream of (e.g., within 500 bp upstream of the CCR5 target position), or downstream of (e.g., within 500 bp downstream of the CCR5 target position) in the CCR5 gene.

In certain embodiments, when a single gRNA molecule is used to target a Cas9 nuclease to create a double strand break to in close proximity to the CCR5 target position, e.g., the gRNA is used to target either upstream of (e.g., within 500 bp upstream of the CCR5 target position), or downstream of (e.g., within 500 bp downstream of the CCR5 target position) in the CCR5 gene.

In certain embodiments, dual targeting is used to create two double strand breaks to in close proximity to the mutation, e.g., the gRNA is used to target either upstream of (e.g., within 500 bp upstream of the CCR5 target position), or downstream of (e.g., within 500 bp downstream of the CCR5 target position) in the CCR5 gene. In certain embodiments, the first and second gRNAs are used to target two Cas9 nucleases to flank, e.g., the first of gRNA is used to target upstream of (e.g., within 500 bp upstream of the CCR5 target position), and the second gRNA is used to target downstream of (e.g., within 500 bp downstream of the CCR5 target position) in the CCR5 gene.

In certain embodiments, dual targeting is used to create a double strand break and a pair of single strand breaks to delete a genomic sequence including the CCR5 target position. In certain embodiments, the first, second and third gRNAs are used to target one Cas9 nuclease and two Cas9 nickases to flank, e.g., the first gRNA that can be used with the Cas9 nuclease is used to target upstream of (e.g., within 500 bp upstream of the CCR5 target position) or downstream of (e.g., within 500 bp downstream of the CCR5 target position), and the second and third gRNAs that can be used with the Cas9 nickase pair are used to target the opposite side of the mutation (e.g., within 500 bp upstream or downstream of the CCR5 target position) in the CCR5 gene.

In certain embodiments, when four gRNAs (e.g., two pairs) are used to target four Cas9 nickases to create four single strand breaks to delete genomic sequence including the mutation, the first pair and second pair of gRNAs are used to target four Cas9 nickases to flank, e.g., the first pair of gRNAs are used to target upstream of (e.g., within 500 bp upstream of the CCR5 target position), and the second pair of gRNAs are used to target downstream of (e.g., within 500 bp downstream of the CCR5 target position) in the CCR5 gene.

Any of the targeting domains in the tables described herein can be used with a Cas9 nickase molecule to generate a single strand break.

Any of the targeting domains in the tables described herein can be used with a Cas9 nuclease molecule to generate a double strand break.

In certain embodiments, dual targeting (e.g., dual nicking) is used to create two nicks on opposite DNA strands by using S. pyogenes, S. aureus and N. meningitidis Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

When two gRNAs designed for use to target two Cas9 molecules, one Cas9 can be one species, the second Cas9 can be from a different species. Both Cas9 species are used to generate a single or double-strand break, as desired.

8.2 Targeting Domains For Use In Knocking Down the CCR5 Gene

Targeting domains for use in gRNAs for knocking down the CCR5 gene in conjunction with the methods disclosed herein were identified and ranked into 3 tiers for S. pyogenes, 5 tiers for S. aureus, and 3 tiers for N. meningitidis.

For S. pyogenes, tier 1 targeting domains were selected based on (1) distance to a target site (e.g., the transcription start site), e.g., within 500 bp (e.g., upstream or downstream) of the target site (e.g., the transcription start site) and (2) a high level of orthogonality. Tier 2 targeting domains were selected based on (1) distance to the target site (e.g., the transcription start site), e.g., within 500 bp (e.g., upstream or downstream) of the target site (e.g., the transcription start site). Tier 3 targeting domains were selected based on distance to the target site (e.g., the transcription start site), e.g., within the additional 500 bp upstream and downstream of the transcription start site (i.e., extending to 1 kb upstream and downstream of the transcription start site.

For S. aureus, tier 1 targeting domains were selected based on (1) distance to the target site (e.g., the transcription start site), e.g., within 500 bp (e.g., upstream or downstream) of the target site (e.g., the transcription start site), (2) a high level of orthogonality, and (3) PAM is NNGRRT. Tier 2 targeting domains were selected based on (1) distance to the target site (e.g., the transcription start site), e.g., within 500 bp (e.g., upstream or downstream) of the target site (e.g., the transcription start site), and (2) PAM is NNGRRT. Tier 3 targeting domains were selected based on (1) distance to a target site (e.g., the transcription start site), e.g., within 500 bp (e.g., upstream or downstream) of the target site (e.g., the transcription start site), and (2) PAM is NNGRRV. Tier 4 targeting domains were selected based on (1) distance to the target site (e.g., the transcription start site), e.g., within the additional 500 bp upstream and downstream of the transcription start site (i.e., extending to 1 kb upstream and downstream of the transcription start site, and (2) PAM is NNGRRT. Tier 5 targeting domains were selected based on (1) distance to the target site (e.g., the transcription start site), e.g., within the additional 500 bp upstream and downstream of the transcription start site (i.e., extending to 1 kb upstream and downstream of the transcription start site, and (2) PAM is NNGRRV.

For N. meningitidis, tier 1 targeting domains were selected based on (1) distance to a target site (e.g., the transcription start site), e.g., within 500 bp (e.g., upstream or downstream) of the target site (e.g., the transcription start site) and (2) a high level of orthogonality. Tier 2 targeting domains were selected based on (1) distance to the target site (e.g., the transcription start site), e.g., within 500 bp (e.g., upstream or downstream) of the target site (e.g., the transcription start site). Tier 3 targeting domains were selected based on distance to the target site (e.g., the transcription start site), e.g., within the additional 500 bp upstream and downstream of the transcription start site (i.e., extending to 1 kb upstream and downstream of the transcription start site.

Note that tiers are non-inclusive (each targeting domain is listed only once for the strategy). In certain instances, no targeting domain was identified based on the criteria of the particular tier. The identified targeting domains are summarized below in Table 2.

TABLE 2
Nucleotide sequences of S. pyogenes, S. aureus, and
N. meningitidis targeting domains for knocking down the CCR5 gene

| | S. pyogenes | S. aureus | N. meningitidis |
|---|---|---|---|
| Tier 1 | SEQ ID NOS: | SEQ ID NOS: | SEQ ID NOS: |
| | 1614 to 1626 | 1947 to 2045 | 3664 to 3698 |
| Tier 2 | SEQ ID NOS: | SEQ ID NOS: | SEQ ID NOS: |
| | 1627 to 1781 | 2046 to 2180 | 3699 to 3709 |
| Tier 3 | SEQ ID NOS: | SEQ ID NOS: | SEQ ID NOS: |
| | 1782 to 1946 | 2181 to 2879 | 3710 to 3739 |
| Tier 4 | Not applicable | SEQ ID NOS: | Not applicable |
| | | 2880 to 3047 | |
| Tier 5 | Not applicable | SEQ ID NOS: | Not applicable |
| | | 3048 to 3663 | |

8.3 Targeting Domains For Use In Knocking Out the CXCR4 Gene

Targeting domains for use in gRNAs for knocking out the CXCR4 gene in conjunction with the methods disclosed herein were identified and ranked into 3 tiers for S. pyogenes, 5 tiers for S. aureus, and 3 tiers for N. meningitidis.

For S. pyogenes, tier 1 targeting domains were selected based on (1) distance to a target site (e.g., start codon), e.g., within 500 bp (e.g., downstream) of the target site (e.g., start codon) and (2) a high level of orthogonality. Tier 2 targeting domains were selected based on (1) distance to the target site (e.g., start codon), e.g., within 500 bp (e.g., downstream) of the target site (e.g., start codon). Tier 3 targeting domains were selected based on distance to the target site (e.g., start codon), e.g., within reminder of the coding sequence, e.g., downstream of the first 500 bp of coding sequence (e.g., anywhere from +500 (relative to the start codon) to the stop codon).

For S. aureus, tier 1 targeting domains were selected based on (1) distance to the target site (e.g., start codon), e.g., within 500 bp (e.g., downstream) of the target site (e.g., start codon), (2) a high level of orthogonality, and (3) PAM is NNGRRT. Tier 2 targeting domains were selected based on (1) distance to the target site (e.g., start codon), e.g., within 500 bp (e.g., downstream) of the target site (e.g., start codon), and (2) PAM is NNGRRT. Tier 3 targeting domains were selected based on (1) distance to a the target site (e.g., start codon), e.g., within 500 bp (e.g., downstream) of the target site (e.g., start codon), and (2) PAM is NNGRRV. Tier 4 targeting domains were selected based on (1) distance to the target site (e.g., start codon), e.g., within reminder of the coding sequence, e.g., downstream of the first 500 bp of coding sequence (e.g., anywhere from +500 (relative to the start codon) to the stop codon), and (2) PAM is NNGRRT. Tier 5 targeting domains were selected based on (1) distance to the target site (e.g., start codon), e.g., within reminder of the coding sequence, e.g., downstream of the first 500 bp of coding sequence (e.g., anywhere from +500 (relative to the start codon) to the stop codon), and (2) PAM is NNGRRV.

For N. meningitidis, tier 1 targeting domains were selected based on (1) distance to the target site, e.g., within 500 bp (e.g., downstream) of the target site (e.g., start codon) and (2) a high level of orthogonality. Tier 2 targeting domains were selected based on (1) distance to the target site (e.g., start codon), e.g., within 500 bp (e.g., downstream) of the target site (e.g., start codon). Tier 3 targeting domains were selected based on distance to the target site (e.g., start codon), e.g., within reminder of the coding sequence, e.g., downstream of the first 500 bp of coding sequence (e.g., anywhere from +500 (relative to the start codon) to the stop codon).

Note that tiers are non-inclusive (each targeting domain is listed only once for the strategy). In certain instances, no targeting domain was identified based on the criteria of the particular tier. The identified targeting domains are summarized below in Table 3.

TABLE 3
Nucleotide sequences of S. pyogenes, S. aureus, and
N. meningitidis targeting domains for knocking out the CXCR4 gene

| | S. pyogenes | S. aureus | N. meningitidis |
|---|---|---|---|
| Tier 1 | SEQ ID NOS: | SEQ ID NOS: | SEQ ID NOS: |
| | 3740 to 3772 | 4064 to 4125 | 5209 to 5219 |
| Tier 2 | SEQ ID NOS: | SEQ ID NOS: | SEQ ID NO: 5220 |
| | 3773 to 3911 | 4126 to 4147 | |
| Tier 3 | SEQ ID NOS: | SEQ ID NOS: | SEQ ID NOS: |
| | 3912 to 4063 | 4148 to 4592 | 5221 to 5240 |
| Tier 4 | Not applicable | SEQ ID NOS: | Not applicable |
| | | 4593 to 4753 | |
| Tier 5 | Not applicable | SEQ ID NOS: | Not applicable |
| | | 4754 to 5208 | |

In certain embodiments, when a single gRNA molecule is used to target a Cas9 nickase to create a single strand break in close proximity to the CXCR4 target position, e.g., the gRNA is used to target either upstream of (e.g., within 500 bp upstream of the CXCR4 target position), or downstream of (e.g., within 500 bp downstream of the CXCR4 target position) in the CXCR4 gene.

In certain embodiments, when a single gRNA molecule is used to target a Cas9 nuclease to create a double strand break to in close proximity to the CXCR4 target position, e.g., the gRNA is used to target either upstream of (e.g., within 500 bp upstream of the CXCR4 target position), or downstream of (e.g., within 500 bp downstream of the CXCR4 target position) in the CXCR4 gene.

In certain embodiments, dual targeting is used to create two double strand breaks to in close proximity to the mutation, e.g., the gRNA is used to target either upstream of (e.g., within 500 bp upstream of the CXCR4 target position), or downstream of (e.g., within 500 bp downstream of the CXCR4 target position) in the CXCR4 gene. In certain embodiments, the first and second gRNAs are used to target two Cas9 nucleases to flank, e.g., the first of gRNA is used to target upstream of (e.g., within 500 bp upstream of the CXCR4 target position), and the second gRNA is used to target downstream of (e.g., within 500 bp downstream of the CXCR4 target position) in the CXCR4 gene.

In certain embodiments, dual targeting is used to create a double strand break and a pair of single strand breaks to delete a genomic sequence including the CXCR4 target position. In certain embodiments, the first, second and third gRNAs are used to target one Cas9 nuclease and two Cas9 nickases to flank, e.g., the first gRNA that can be used with the Cas9 nuclease is used to target upstream of (e.g., within 500 bp upstream of the CXCR4 target position) or downstream of (e.g., within 500 bp downstream of the CXCR4 target position), and the second and third gRNAs that can be used with the Cas9 nickase pair are used to target the opposite side of the mutation (e.g., within 500 bp upstream or downstream of the CXCR4 target position) in the CXCR4 gene.

In certain embodiments, when four gRNAs (e.g., two pairs) are used to target four Cas9 nickases to create four single strand breaks to delete genomic sequence including the mutation, the first pair and second pair of gRNAs are used to target four Cas9 nickases to flank, e.g., the first pair of gRNAs are used to target upstream of (e.g., within 500 bp upstream of the CXCR4 target position), and the second pair of gRNAs are used to target downstream of (e.g., within 500 bp downstream of the CXCR4 target position) in the CXCR4 gene.

Any of the targeting domains in the tables described herein can be used with a Cas9 nickase molecule to generate a single strand break.

Any of the targeting domains in the tables described herein can be used with a Cas9 nuclease molecule to generate a double strand break.

In certain embodiments, dual targeting (e.g., dual nicking) is used to create two nicks on opposite DNA strands by using S. pyogenes, S. aureus and N. meningitidis Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

When two gRNAs designed for use to target two Cas9 molecules, one Cas9 can be one species, the second Cas9 can be from a different species. Both Cas9 species are used to generate a single or double-strand break, as desired.

8.4 Targeting Domains For Use In Knocking Down the CXCR4 Gene

Targeting domains for use in gRNAs for knocking down the CXCR4 gene in conjunction with the methods disclosed herein were identified and ranked into 3 tiers for S. pyogenes, 5 tiers for S. aureus, and 3 tiers for N. meningitidis.

For S. pyogenes, tier 1 targeting domains were selected based on (1) distance to a target site (e.g., the transcription start site), e.g., within 500 bp (e.g., upstream or downstream) of the target site (e.g., the transcription start site) and (2) a high level of orthogonality. Tier 2 targeting domains were selected based on (1) distance to the target site (e.g., the transcription start site), e.g., within 500 bp (e.g., upstream or downstream) of the target site (e.g., the transcription start site). Tier 3 targeting domains were selected based on distance to the target site (e.g., the transcription start site), e.g., within the additional 500 bp upstream and downstream of the transcription start site (i.e., extending to 1 kb upstream and downstream of the transcription start site.

For S. aureus, tier 1 targeting domains were selected based on (1) distance to the target site (e.g., the transcription start site), e.g., within 500 bp (e.g., upstream or downstream) of the target site (e.g., the transcription start site), (2) a high level of orthogonality, and (3) PAM is NNGRRT. Tier 2 targeting domains were selected based on (1) distance to the target site (e.g., the transcription start site), e.g., within 500 bp (e.g., upstream or downstream) of the target site (e.g., the transcription start site), and (2) PAM is NNGRRT. Tier 3 targeting domains were selected based on (1) distance to a target site (e.g., the transcription start site), e.g., within 500 bp (e.g., upstream or downstream) of the target site (e.g., the transcription start site), and (2) PAM is NNGRRV. Tier 4 targeting domains were selected based on (1) distance to the target site (e.g., the transcription start site), e.g., within the additional 500 bp upstream and downstream of the transcription start site (i.e., extending to 1 kb upstream and downstream of the transcription start site, and (2) PAM is NNGRRT. Tier 5 targeting domains were selected based on (1) distance to the target site (e.g., the transcription start site), e.g., within the additional 500 bp upstream and downstream of the transcription start site (i.e., extending to 1 kb upstream and downstream of the transcription start site, and (2) PAM is NNGRRV.

For N. meningitidis, tier 1 targeting domains were selected based on (1) distance to a target site (e.g., the transcription start site), e.g., within 500 bp (e.g., upstream or downstream) of the target site (e.g., the transcription start site) and (2) a high level of orthogonality. Tier 2 targeting domains were selected based on (1) distance to the target site (e.g., the transcription start site), e.g., within 500 bp (e.g., upstream or downstream) of the target site (e.g., the transcription start site). Tier 3 targeting domains were selected based on distance to the target site (e.g., the transcription start site), e.g., within the additional 500 bp upstream and downstream of the transcription start site (i.e., extending to 1 kb upstream and downstream of the transcription start site.

Note that tiers are non-inclusive (each targeting domain is listed only once for the strategy). In certain instances, no targeting domain was identified based on the criteria of the particular tier. The identified targeting domains are summarized below in Table 4.

TABLE 4
Nucleotide sequences of S. pyogenes, S. aureus, and
N. meningitidis targeting domains for knocking down the CXCR4 gene

| | S. pyogenes | S. aureus | N. meningitidis |
|---|---|---|---|
| Tier 1 | SEQ ID NOS: | SEQ ID NOS: | SEQ ID NOS: |
| | 5241 to 5349 | 5921 to 6046 | 8356 to 8377 |
| Tier 2 | SEQ ID NOS: | SEQ ID NOS: | SEQ ID NOS: |
| | 5350 to 5615 | 6047 to 6126 | 8378 to 8379 |
| Tier 3 | SEQ ID NOS: | SEQ ID NOS: | SEQ ID NOS: |
| | 5616 to 5920 | 6127 to 7288 | 8380 to 8407 |
| Tier 4 | Not applicable | SEQ ID NOS: | Not applicable |
| | | 7289 to 7575 | |
| Tier 5 | Not applicable | SEQ ID NOS: | Not applicable |
| | | 7576 to 8355 | |

One or more of the gRNA molecules described herein, e.g., those comprising the targeting domains described in Tables 1-4 can be used with at least one Cas9 molecule (e.g., a S. pyogenes Cas9 molecule and/or a S. aureus Cas9 molecule) to form a single or a double stranded cleavage. In certain embodiments, dual targeting is used to create two double strand breaks (e.g., by using at least one Cas9 nuclease, e.g., a S. pyogenes Cas9 nuclease and/or a S. aureus Cas9 nuclease) or two nicks (e.g., by using at least one Cas9 nickase, e.g., a S. pyogenes Cas9 nickase and/or a S. aureus Cas9 nickase) on opposite DNA strands with two gRNA molecules. In certain embodiments, a presently disclosed compositio or genome editing system comprises two gRNA molecules comprising targeting domains that are complementary to opposite DNA strands, e.g., a gRNA molecule comprising any minus strand targeting domain that can be paired with a gRNA molecule comprising a plus strand targeting domain provided that the two gRNA molecules are oriented on the DNA such that PAMs face outward. In certain embodiments, two gRNA molecules are used to target two Cas9 nucleases (e.g., two S. pyogenes Cas9 nucleases, two S. aureus Cas9 nucleases, or one S. aureus Cas9 nuclease and one S. pyogenes Cas9 nuclease) or two Cas9 nickases (e.g., two S. pyogenes Cas9 nickases, two S. aureus Cas9 nickases, or one S. aureus Cas9 nickase and one Cas9 nickase). One or more of the gRNA molecules described herein, e.g., those comprising the targeting domains described in Tables 1-4 can be used with at least one Cas9 molecule to mediate the alteration of a CCR5 gene, alteration of a CXCR4 gene, or alteration of a CCR5 gene and a CXCR4 gene, described in Sections 4, 5 and 6.

### 9. Cas9 Molecules

Cas9 molecules of a variety of species can be used in the methods and compositions described herein. While the S. pyogenes, S. aureus, and N. meningitidis Cas9 molecules are the subject of much of the disclosure herein, Cas9 molecules, derived from, or based on the Cas9 proteins of other species listed herein can be used as well. These include, for example, Cas9 molecules from Acidovorax avenae, Actinobacillus pleuropneumonias, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum p., Tistrella mobilis, Treponema sp., or Verminephrobacter eiseniae.

9.1 Cas9 Domains

Crystal structures have been determined for two different naturally occurring bacterial Cas9 molecules (Jinek 2014) and for S. pyogenes Cas9 with a guide RNA (e.g., a synthetic fusion of crRNA and tracrRNA) (Nishimasu 2014; Anders 2014).

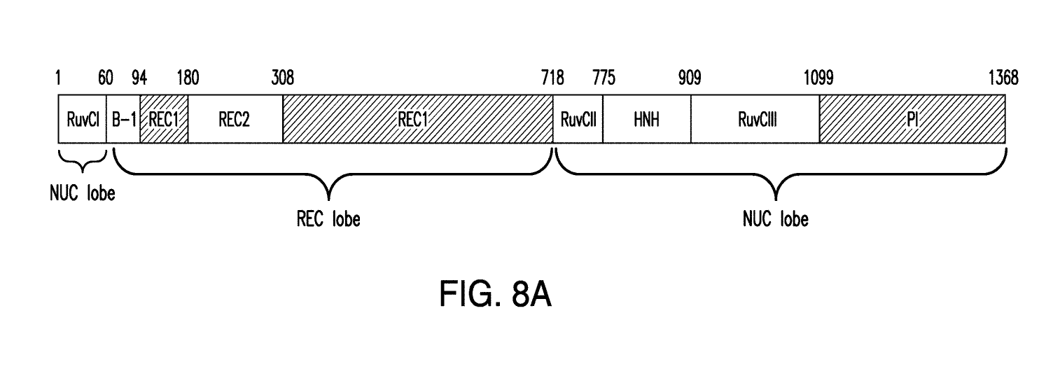

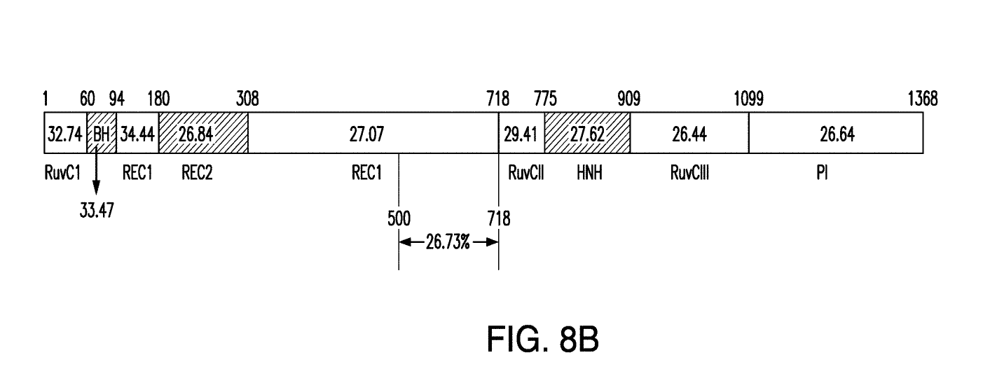

A naturally occurring Cas9 molecule comprises two lobes: a recognition (REC) lobe and a nuclease (NUC) lobe; each of which further comprise domains described herein. FIGS. 8A-8B provide a schematic of the organization of important Cas9 domains in the primary structure. The domain nomenclature and the numbering of the amino acid residues encompassed by each domain used throughout this disclosure is as described previously (Nishimasu 2014). The numbering of the amino acid residues is with reference to Cas9 from S. pyogenes.

The REC lobe comprises the arginine-rich bridge helix (BH), the REC1 domain, and the REC2 domain. The REC lobe does not share structural similarity with other known proteins, indicating that it is a Cas9-specific functional domain. The BH domain is a long α helix and arginine rich region and comprises amino acids 60-93 of the sequence of S. pyogenes Cas9. The REC1 domain is important for recognition of the repeat:anti-repeat duplex, e.g., of a gRNA or a tracrRNA, and is therefore critical for Cas9 activity by recognizing the target sequence. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat:anti-repeat duplex. The REC2 domain comprises amino acids 180-307 of the sequence of S. pyogenes Cas9.

The NUC lobe comprises the RuvC domain, the HNH domain, and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule, and comprises amino acids 1099-1368 of the sequence of S. pyogenes Cas9.

9.1.1 RuvC-Like Domain and HNH-Like Domain

In certain embodiments, a Cas9 molecule or Cas9 polypeptide comprises an HNH-like domain and a RuvC-like domain, and in certain of these embodiments cleavage activity is dependent on the RuvC-like domain and the HNH-like domain. A Cas9 molecule or Cas9 polypeptide can comprise one or more of a RuvC-like domain and an HNH-like domain. In certain embodiments, a Cas9 molecule or Cas9 polypeptide comprises a RuvC-like domain, e.g., a RuvC-like domain described below, and/or an HNH-like domain, e.g., an HNH-like domain described below.

### RuvC-Like Domains

In certain embodiments, a RuvC-like domain cleaves a single strand, e.g., the non-complementary strand of the target nucleic acid molecule. The Cas9 molecule or Cas9 polypeptide can include more than one RuvC-like domain (e.g., one, two, three or more RuvC-like domains). In certain embodiments, a RuvC-like domain is at least 5, 6, 7, 8 amino acids in length but not more than 20, 19, 18, 17, 16 or 15 amino acids in length. In certain embodiments, the Cas9 molecule or Cas9 polypeptide comprises an N-terminal RuvC-like domain of about 10 to 20 amino acids, e.g., about 15 amino acids in length.

9.1.2 N-Terminal RuvC-Like Domains

Some naturally occurring Cas9 molecules comprise more than one RuvC-like domain with cleavage being dependent on the N-terminal RuvC-like domain. Accordingly, a Cas9 molecule or Cas9 polypeptide can comprise an N-terminal RuvC-like domain. Exemplary N-terminal RuvC-like domains are described below.

In certain embodiments, a Cas9 molecule or Cas9 polypeptide comprises an N-terminal RuvC-like domain comprising an amino acid sequence of Formula I:

(SEQ ID NO: 20)

D-X₁-G-X₂-X₃-X₄-X₅-G-X₆-X₇-X₈-X₉,

wherein,

X₁ is selected from I, V, M, L, and T (e.g., selected from I, V, and L);

X₂ is selected from T, I, V, S, N, Y, E, and L (e.g., selected from T, V, and I);

X₃ is selected from N, S, G, A, D, T, R, M, and F (e.g., A or N);

X₄ is selected from S, Y, N, and F (e.g., S);

X₅ is selected from V, I, L, C, T, and F (e.g., selected from V, I and L);

X₆ is selected from W, F, V, Y, S, and L (e.g., W);

X₇ is selected from A, S, C, V, and G (e.g., selected from A and S);

X₈ is selected from V, I, L, A, M, and H (e.g., selected from V, I, M and L); and

X₉ is selected from any amino acid or is absent (e.g., selected from T, V, I, L, Δ, F, S, A, Y, M, and R, or, e.g., selected from T, V, I, L, and Δ).

In certain embodiments, the N-terminal RuvC-like domain differs from a sequence of SEQ ID NO:20 by as many as 1 but no more than 2, 3, 4, or 5 residues.

In certain embodiments, the N-terminal RuvC-like domain is cleavage competent. In other embodiments, the N-terminal RuvC-like domain is cleavage incompetent.

In certain embodiments, a Cas9 molecule or Cas9 polypeptide comprises an N-terminal RuvC-like domain comprising an amino acid sequence of Formula II:

(SEQ ID NO: 21)

D-X₁-G-X₂-X₃-S-X₅-G-X₆-X₇-X₈-X₉,,

wherein

X₁ is selected from I, V, M, L, and T (e.g., selected from I, V, and L);

X₂ is selected from T, I, V, S, N, Y, E, and L (e.g., selected from T, V, and I);

X₃ is selected from N, S, G, A, D, T, R, M and F (e.g., A or N);

X₅ is selected from V, I, L, C, T, and F (e.g., selected from V, I and L);

X₆ is selected from W, F, V, Y, S, and L (e.g., W);

X₇ is selected from A, S, C, V, and G (e.g., selected from A and S);

X₈ is selected from V, I, L, A, M, and H (e.g., selected from V, I, M and L); and

X₉ is selected from any amino acid or is absent (e.g., selected from T, V, I, L, Δ, F, S, A, Y, M, and R or selected from e.g., T, V, I, L, and Δ).

In certain embodiments, the N-terminal RuvC-like domain differs from a sequence of SEQ ID NO:21 by as many as 1 but not more than 2, 3, 4, or 5 residues.

In certain embodiments, the N-terminal RuvC-like domain comprises an amino acid sequence of Formula III:

(SEQ ID NO: 22)

D-I-G-X₂-X₃-S-V-G-W-A-X₈-X₉,

wherein

X₂ is selected from T, I, V, S, N, Y, E, and L (e.g., selected from T, V, and I);

X₃ is selected from N, S, G, A, D, T, R, M, and F (e.g., A or N);

X₈ is selected from V, I, L, A, M, and H (e.g., selected from V, I, M and L); and

X₉ is selected from any amino acid or is absent (e.g., selected from T, V, I, L, Δ, F, S, A, Y, M, and R or selected from e.g., T, V, I, L, and Δ).

In certain embodiments, the N-terminal RuvC-like domain differs from a sequence of SEQ ID NO:22 by as many as 1 but not more than, 2, 3, 4, or 5 residues.

In certain embodiments, the N-terminal RuvC-like domain comprises an amino acid sequence of Formula IV:

(SEQ ID NO: 23)

D-I-G-T-N-S-V-G-W-A-V-X,

wherein

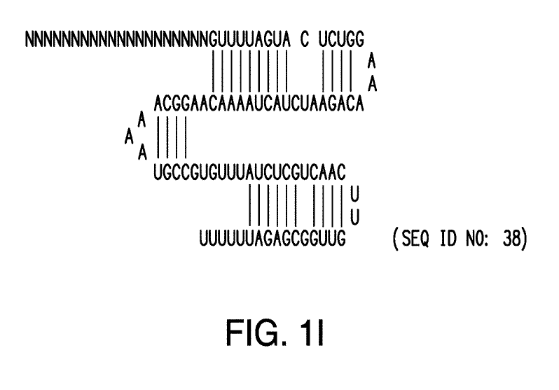

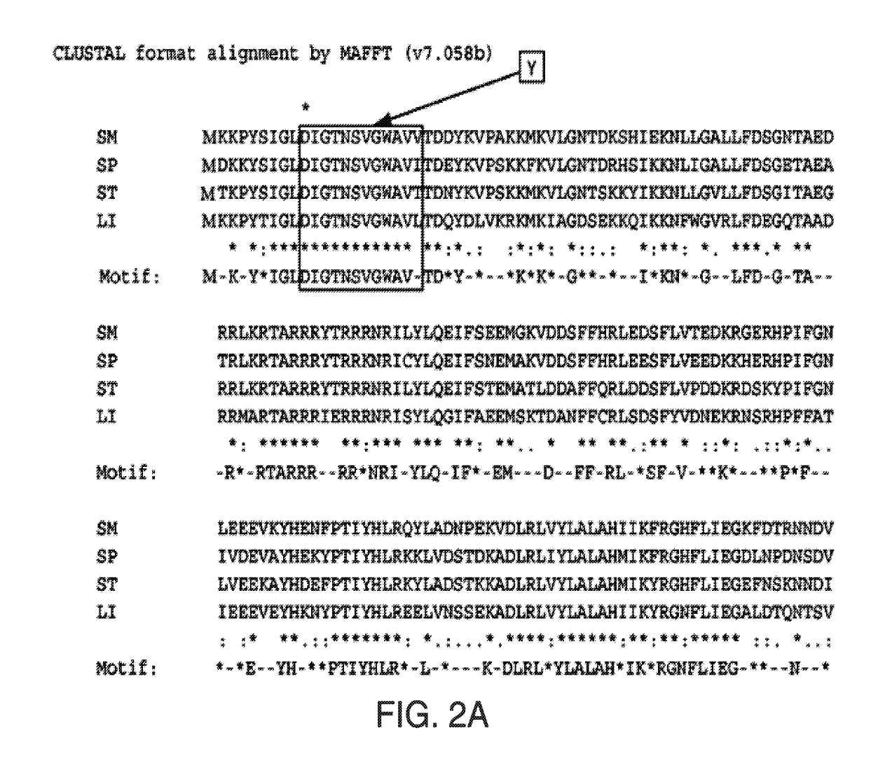

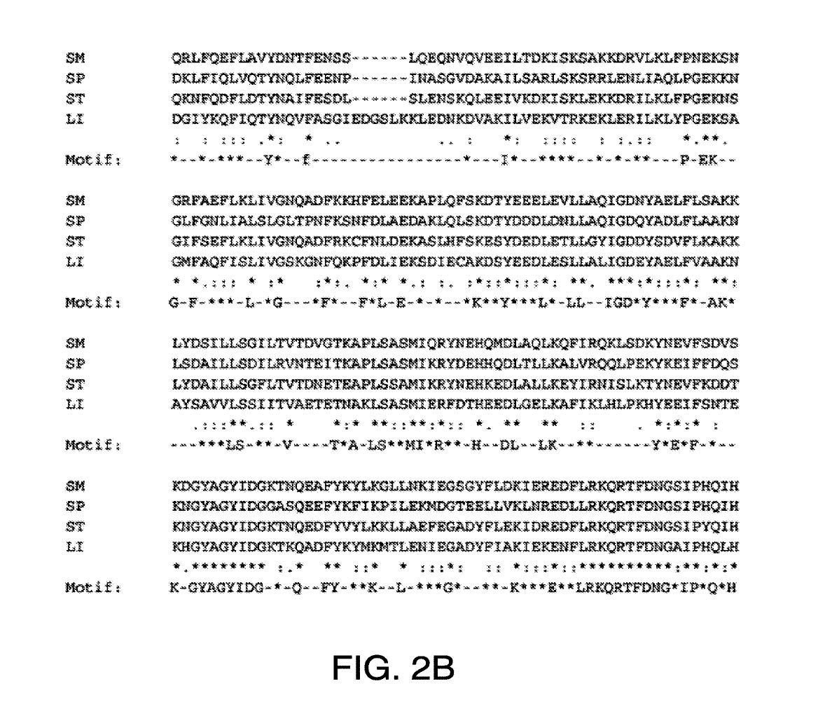

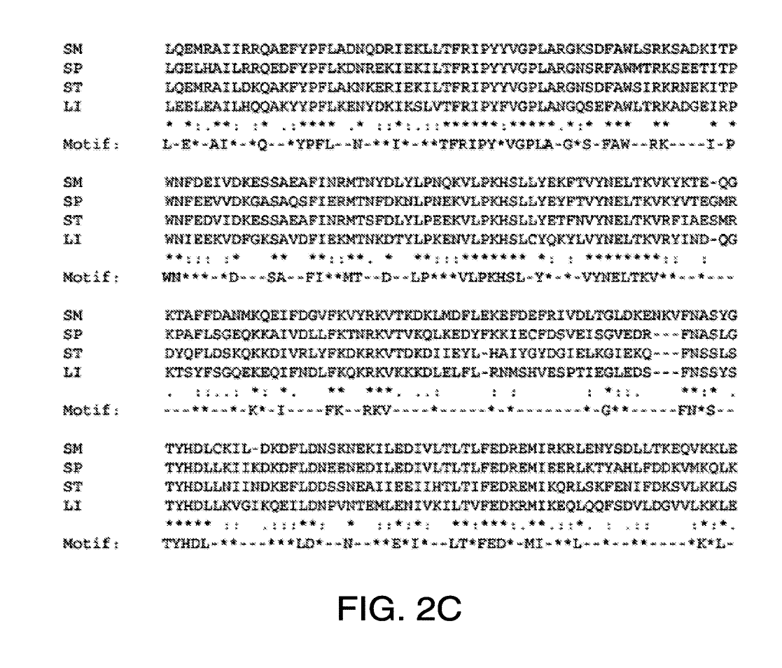

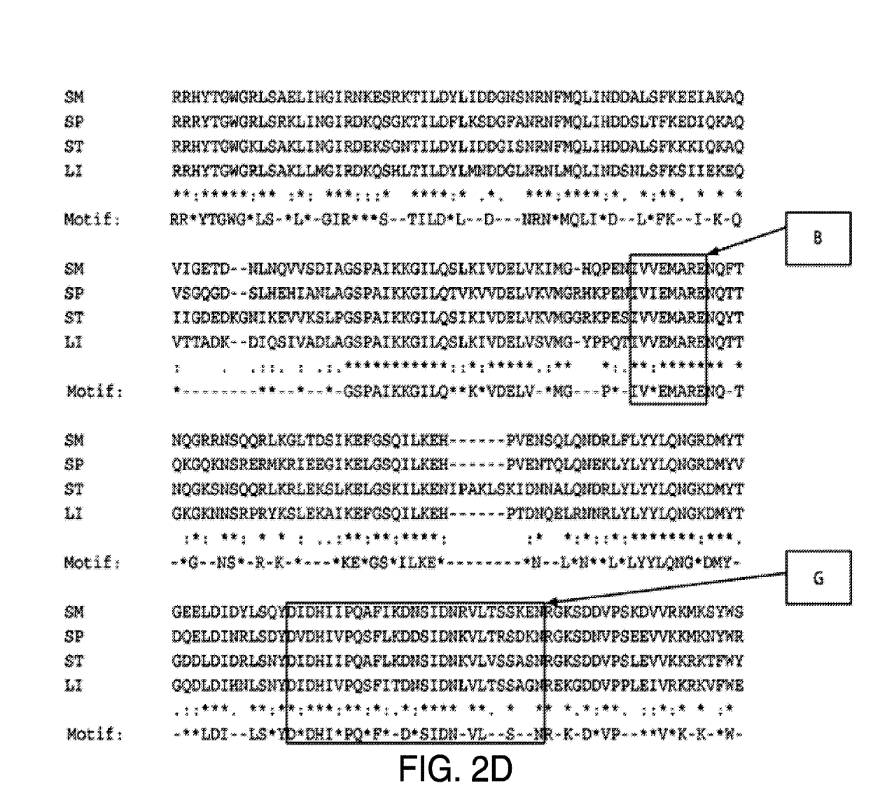

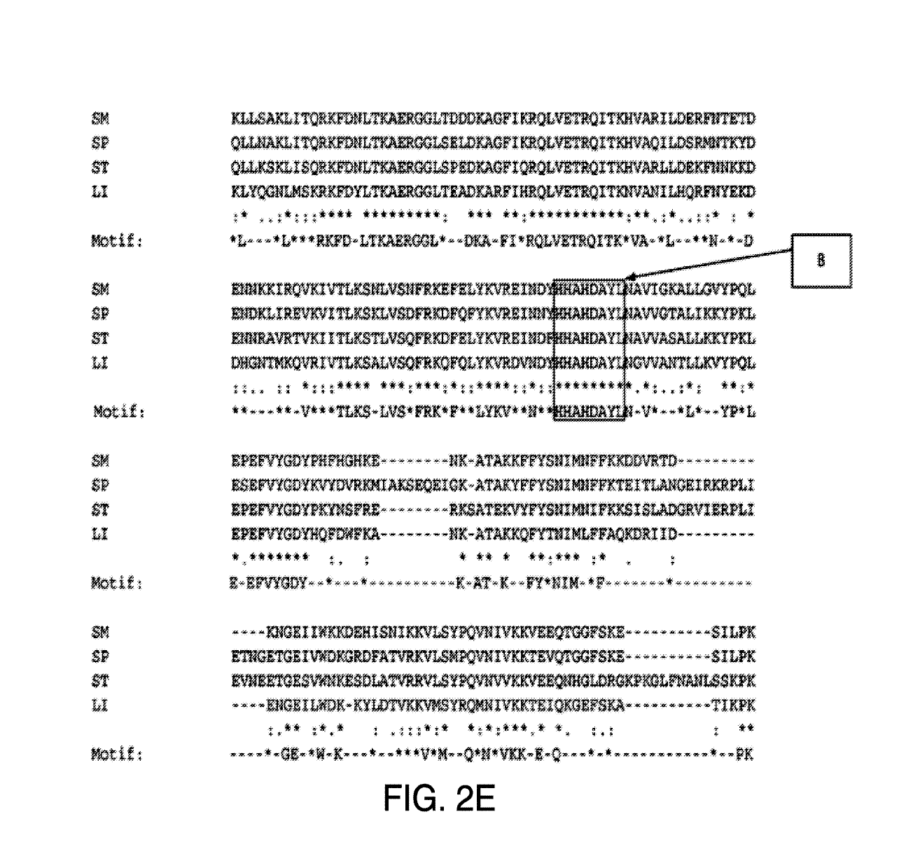

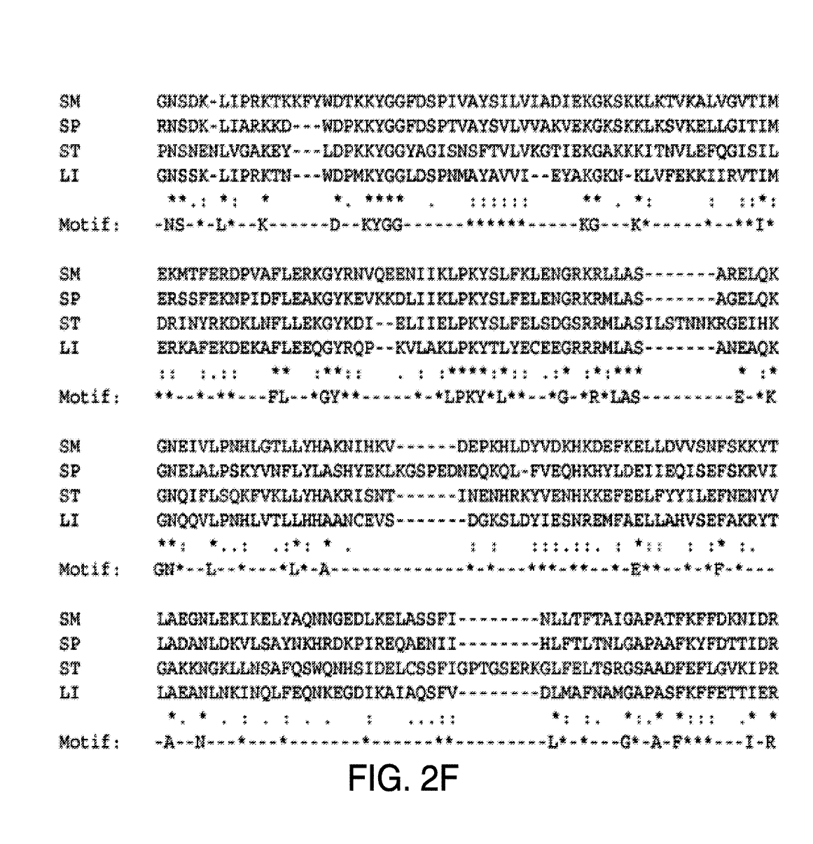

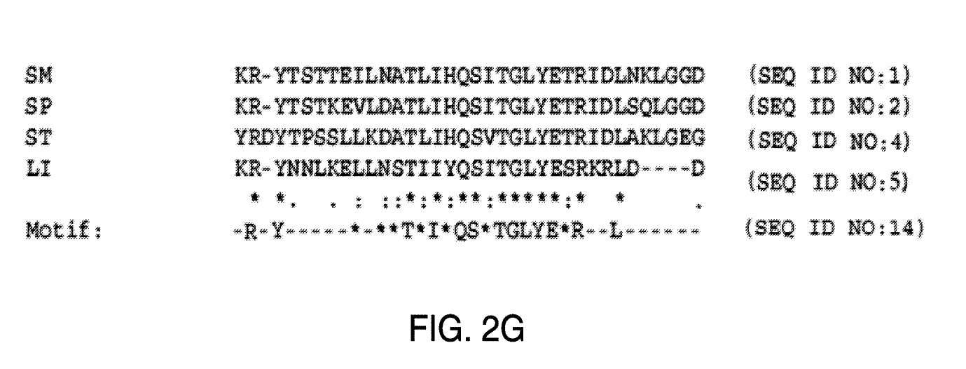

X is a non-polar alkyl amino acid or a hydroxyl amino acid, e.g., X is selected from V, I, L, and T (e.g., the Cas9 molecule can comprise an N-terminal RuvC-like domain shown in FIGS. 2A-2G (depicted as Y)).

In certain embodiments, the N-terminal RuvC-like domain differs from a sequence of SEQ ID NO:23 by as many as 1 but not more than, 2, 3, 4, or 5 residues.

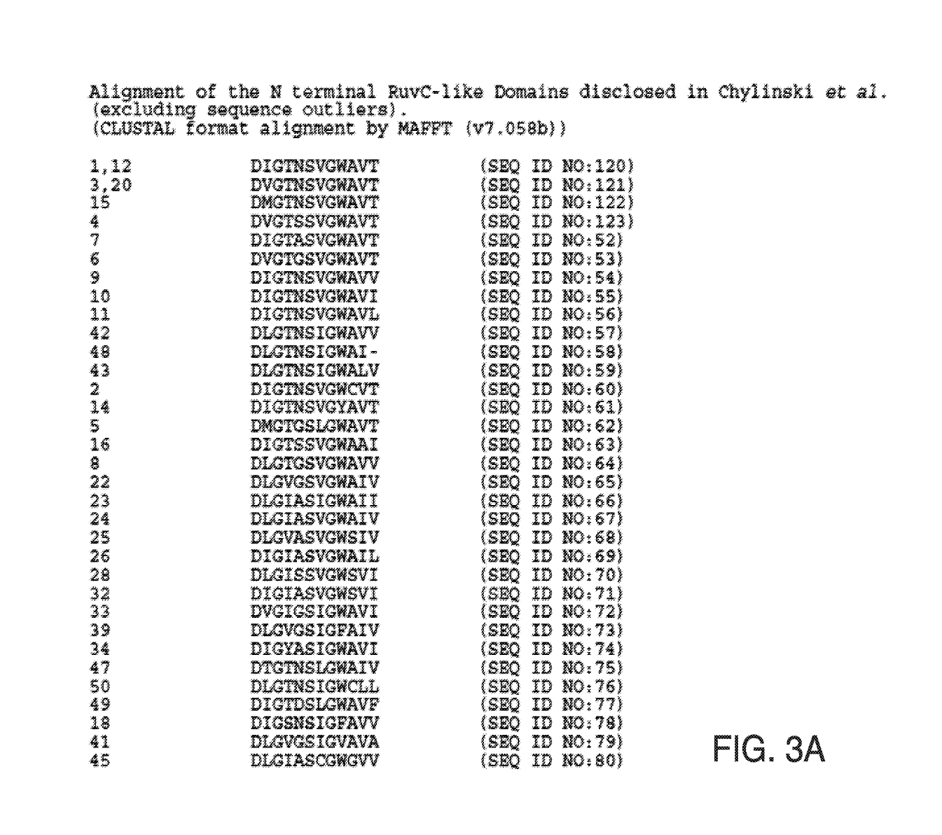

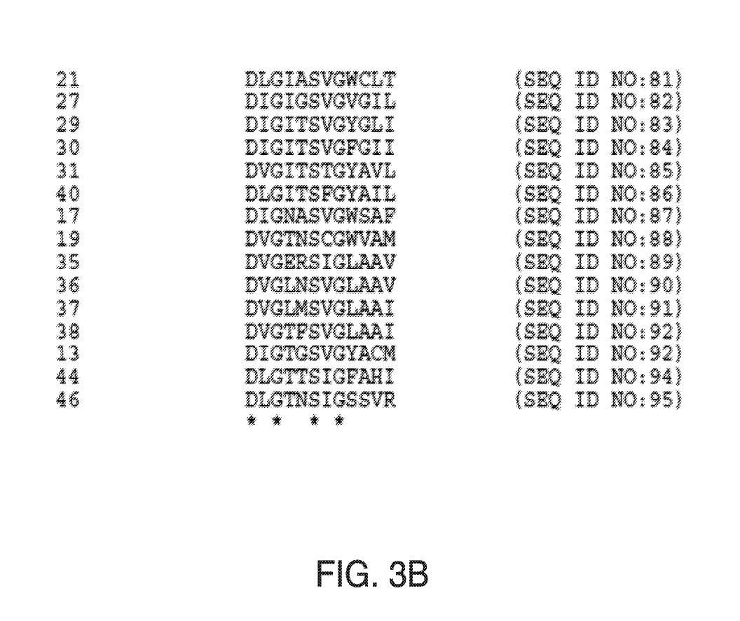

In certain embodiments, the N-terminal RuvC-like domain differs from a sequence of an N-terminal RuvC like domain disclosed herein, e.g., in FIGS. 3A-3B, as many as 1 but no more than 2, 3, 4, or 5 residues. In certain embodiments, 1, 2, 3 or all of the highly conserved residues identified in FIGS. 3A-3B are present.

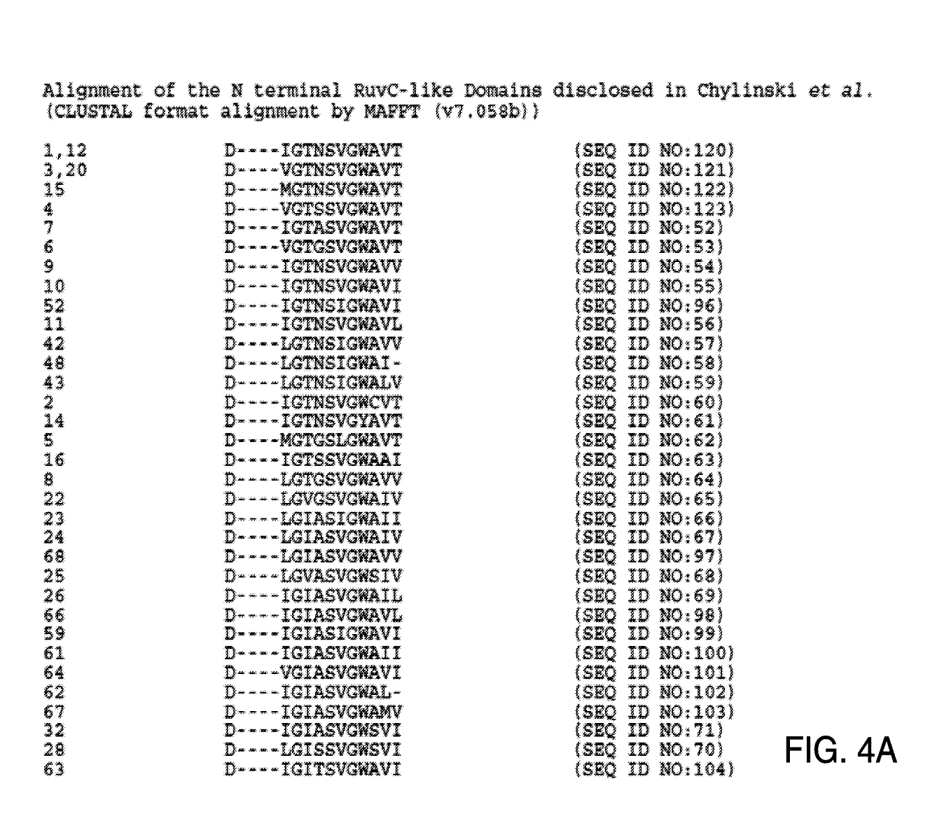

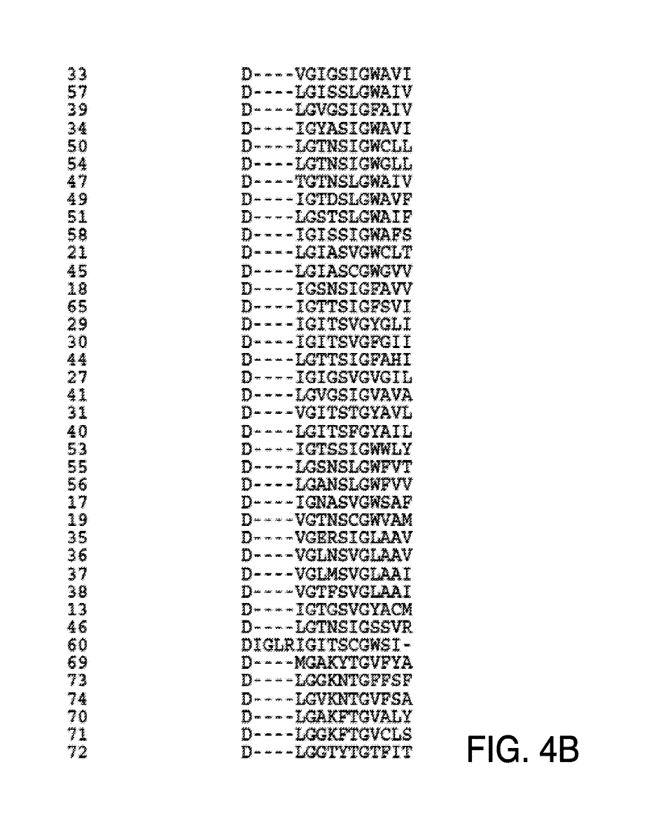

In certain embodiments, the N-terminal RuvC-like domain differs from a sequence of an N-terminal RuvC-like domain disclosed herein, e.g., in FIGS. 4A-4B, as many as 1 but no more than 2, 3, 4, or 5 residues. In certain embodiments, 1, 2, or all of the highly conserved residues identified in FIGS. 4A-4B are present.

9.1.3 Additional RuvC-Like Domains

In addition to the N-terminal RuvC-like domain, the Cas9 molecule or Cas9 polypeptide can comprise one or more additional RuvC-like domains. In certain embodiments, the Cas9 molecule or Cas9 polypeptide comprises two additional RuvC-like domains. In certain embodiments, the additional RuvC-like domain is at least 5 amino acids in length and, e.g., less than 15 amino acids in length, e.g., 5 to 10 amino acids in length, e.g., 8 amino acids in length.

An additional RuvC-like domain can comprise an amino acid sequence of Formula V:

(SEQ ID NO: 15)

I-X₁-X₂-E-X₃-A-R-E

wherein,

X₁ is V or H;

X₂ is I, L or V (e.g., I or V); and

X₃ is M or T.

In certain embodiments, the additional RuvC-like domain comprises an amino acid sequence of Formula VI:

(SEQ ID NO: 16)

I-V-X₂-E-M-A-R-E,

wherein

X₂ is I, L or V (e.g., I or V) (e.g., the Cas9 molecule or Cas9 polypeptide can comprise an additional RuvC-like domain shown in FIG. 2A-2G (depicted as B)).

An additional RuvC-like domain can comprise an amino acid sequence of Formula VII:

(SEQ ID NO: 17)

H-H-A-X₁-D-A-X₂-X₃,

wherein

X₁ is H or L;

X₂ is R or V; and

In certain embodiments, the additional RuvC-like domain comprises the amino acid sequence: H-H-A-H-D-A-Y-L (SEQ ID NO:18).

In certain embodiments, the additional RuvC-like domain differs from a sequence of SEQ ID NOs:15-18 by as many as 1 but not more than 2, 3, 4, or 5 residues.

In certain embodiments, the sequence flanking the N-terminal RuvC-like domain has the amino acid sequence of Formula VIII:

(SEQ ID NO: 19)

K-X₁′-Y-X₂′-X₃′-X₄′-Z-T-D-X₉′-Y,

wherein

X₁′ is selected from K and P;

X₂′ is selected from V, L, I, and F (e.g., V, I and L);

X₃′ is selected from G, A and S (e.g., G);

X₄′ is selected from L, I, V, and F (e.g., L);

X₉′ is selected from D, E, N, and Q; and

Z is an N-terminal RuvC-like domain, e.g., as described above, e.g., having 5 to 20 amino acids.

9.1.4 HNH-Like Domains

In certain embodiments, an HNH-like domain cleaves a single stranded complementary domain, e.g., a complementary strand of a double stranded nucleic acid molecule. In certain embodiments, an HNH-like domain is at least 15, 20, or 25 amino acids in length but not more than 40, 35, or 30 amino acids in length, e.g., 20 to 35 amino acids in length, e.g., 25 to 30 amino acids in length. Exemplary HNH-like domains are described below.

In certain embodiments, a Cas9 molecule or Cas9 polypeptide comprises an HNH-like domain having an amino acid sequence of Formula IX:

(SEQ ID NO: 25)

X₁-X₂-X₃-H-X₄-X₅-P-X₆-X₇-X₈-X⁹-X¹⁰-X¹¹-X¹²-X¹³-
X¹⁴-X¹⁵-N-X¹⁶-X¹⁷-X¹⁸-X¹⁹-X₂₀-X₂₁-X₂₂-X₂₃-N,

wherein

X₁ is selected from D, E, Q and N (e.g., D and E);

X² is selected from L, I, R, Q, V, M, and K;

X₃ is selected from D and E;

X₄ is selected from I, V, T, A, and L (e.g., A, I and V);

X₅ is selected from V, Y, I, L, F, and W (e.g., V, I and L);

X₆ is selected from Q, H, R, K, Y, I, L, F, and W;

X₇ is selected from S, A, D, T, and K (e.g., S and A);

X₈ is selected from F, L, V, K, Y, M, I, R, A, E, D, and Q (e.g., F);

X₉ is selected from L, R, T, I, V, S, C, Y, K, F, and G;

X₁₀ is selected from K, Q, Y, T, F, L, W, M, A, E, G, and S;

X₁₁ is selected from D, S, N, R, L, and T (e.g., D);

X₁₂ is selected from D, N and S;

X₁₃ is selected from S, A, T, G, and R (e.g., S);

X₁₄ is selected from I, L, F, S, R, Y, Q, W, D, K, and H (e.g., I, L and F);

X₁₅ is selected from D, S, I, N, E, A, H, F, L, Q, M, G, Y, and V;

X₁₆ is selected from K, L, R, M, T, and F (e.g., L, R and K);

X₁₇ is selected from V, L, I, A and T;

X₁₈ is selected from L, I, V, and A (e.g., L and I);

X₁₉ is selected from T, V, C, E, S, and A (e.g., T and V);

X₂₀ is selected from R, F, T, W, E, L, N, C, K, V, S, Q, I, Y, H, and A;

X₂₁ is selected from S, P, R, K, N, A, H, Q, G, and L;

X₂₂ is selected from D, G, T, N, S, K, A, I, E, L, Q, R, and Y; and

X₂₃ is selected from K, V, A, E, Y, I, C, L, S, T, G, K, M, D, and F.

In certain embodiments, a HNH-like domain differs from a sequence of SEQ ID NO:25 by at least one but not more than, 2, 3, 4, or 5 residues.

In certain embodiments, the HNH-like domain is cleavage competent. In certain embodiments, the HNH-like domain is cleavage incompetent.

In certain embodiments, a Cas9 molecule or Cas9 polypeptide comprises an HNH-like domain comprising an amino acid sequence of Formula X:

(SEQ ID NO: 26)

X₁-X₂-X₃-H-X₄-X₅-P-X₆-S-X₈-X₉-X₁₀-D-D-S-X₁₄-X₁₅-
N-K-V-L-X₁₉-X₂₀-X₂₁-X₂₂-X₂₃-N,

wherein

X₁ is selected from D and E;

X₂ is selected from L, I, R, Q, V, M, and K;

X₃ is selected from D and E;

X₄ is selected from I, V, T, A, and L (e.g., A, I and V);

X₅ is selected from V, Y, I, L, F, and W (e.g., V, I and L);

X₆ is selected from Q, H, R, K, Y, I, L, F, and W;

X₈ is selected from F, L, V, K, Y, M, I, R, A, E, D, and Q (e.g., F);

X₉ is selected from L, R, T, I, V, S, C, Y, K, F, and G;

X₁₀ is selected from K, Q, Y, T, F, L, W, M, A, E, G, and S;

X₁₄ is selected from I, L, F, S, R, Y, Q, W, D, K and H (e.g., I, L and F);

X₁₅ is selected from D, S, I, N, E, A, H, F, L, Q, M, G, Y, and V;

X₁₉ is selected from T, V, C, E, S, and A (e.g., T and V);

X₂₀ is selected from R, F, T, W, E, L, N, C, K, V, S, Q, I, Y, H, and A;

X₂₁ is selected from S, P, R, K, N, A, H, Q, G, and L;

X₂₂ is selected from D, G, T, N, S, K, A, I, E, L, Q, R, and Y; and

X₂₃ is selected from K, V, A, E, Y, I, C, L, S, T, G, K, M, D, and F.

In certain embodiment, the HNH-like domain differs from a sequence of SEQ ID NO:26 by 1, 2, 3, 4, or 5 residues.

In certain embodiments, a Cas9 molecule or Cas9 polypeptide comprises an HNH-like domain comprising an amino acid sequence of Formula XI:

(SEQ ID NO: 27)

X₁-V-X₃-H-I-V-P-X₆-S-X₈-X₉-X₁₀-D-D-S-X₁₄-X₁₅-N-K-
V-L-T-X₂₀-X₂₁-X₂₂-X₂₃-N,

wherein

X₁ is selected from D and E;

X₃ is selected from D and E;

X₆ is selected from Q, H, R, K, Y, I, L, and W;

X₈ is selected from F, L, V, K, Y, M, I, R, A, E, D, and Q (e.g., F);

X₉ is selected from L, R, T, I, V, S, C, Y, K, F, and G;

X₁₀ is selected from K, Q, Y, T, F, L, W, M, A, E, G, and S;

X₁₄ is selected from I, L, F, S, R, Y, Q, W, D, K, and H (e.g., I, L and F);

X₁₅ is selected from D, S, I, N, E, A, H, F, L, Q, M, G, Y, and V;

X₂₀ is selected from R, F, T, W, E, L, N, C, K, V, S, Q, I, Y, H, and A;

X₂₁ is selected from S, P, R, K, N, A, H, Q, G, and L;

X₂₂ is selected from D, G, T, N, S, K, A, I, E, L, Q, R, and Y; and

X₂₃ is selected from K, V, A, E, Y, I, C, L, S, T, G, K, M, D, and F.

In certain embodiments, the HNH-like domain differs from a sequence of SEQ ID NO:27 by 1, 2, 3, 4, or 5 residues.

In certain embodiments, a Cas9 molecule or Cas9 polypeptide comprises an HNH-like domain having an amino acid sequence of Formula XII:

(SEQ ID NO: 28)

D-X₂-D-H-I-X₅-P-Q-X₇-F-X₉-X₁₀-D-X₁₂-S-I-D-N-X₁₆-
V-L-X₁₉-X₂₀-S-X₂₂-X₂₃-N,

wherein

X₂ is selected from I and V;

X₅ is selected from I and V;

X₇ is selected from A and S;

X₉ is selected from I and L;

X₁₀ is selected from K and T;

X₁₂ is selected from D and N;

X₁₆ is selected from R, K, and L;

X₁₉ is selected from T and V;

X₂₀ is selected from S, and R;

X₂₂ is selected from K, D, and A; and

X₂₃ is selected from E, K, G, and N (e.g., the Cas9 molecule or Cas9 polypeptide can comprise an HNH-like domain as described herein).

In certain embodiments, the HNH-like domain differs from a sequence of SEQ ID NO:28 by as many as 1 but no more than 2, 3, 4, or 5 residues.

In certain embodiments, a Cas9 molecule or Cas9 polypeptide comprises the amino acid sequence of Formula XIII:

(SEQ ID NO: 24)

L-Y-Y-L-Q-N-G-X₁′-D-M-Y-X₂′-X₃′-X₄′-X₅′-L-D-I-X₆′-
X₇′-L-S-X₈′-Y-Z-N-R-X₉′-K-X₁₀′-D-X₁₁′-V-P,

wherein

X₁′ is selected from K and R;

X₂′ is selected from V and T;

X₃′ is selected from G and D;

X₄′ is selected from E, Q and D;

X₅′ is selected from E and D;

X₆′ is selected from D, N, and H;

X₇′ is selected from Y, R, and N;

X₈′ is selected from Q, D, and N;

X₉′ is selected from G and E;

X₁₀′ is selected from S and G;

X₁₁′ is selected from D and N; and

Z is an HNH-like domain, e.g., as described above.

In certain embodiments, the Cas9 molecule or Cas9 polypeptide comprises an amino acid sequence that differs from a sequence of SEQ ID NO:24 by as many as 1 but not more than 2, 3, 4, or 5 residues.

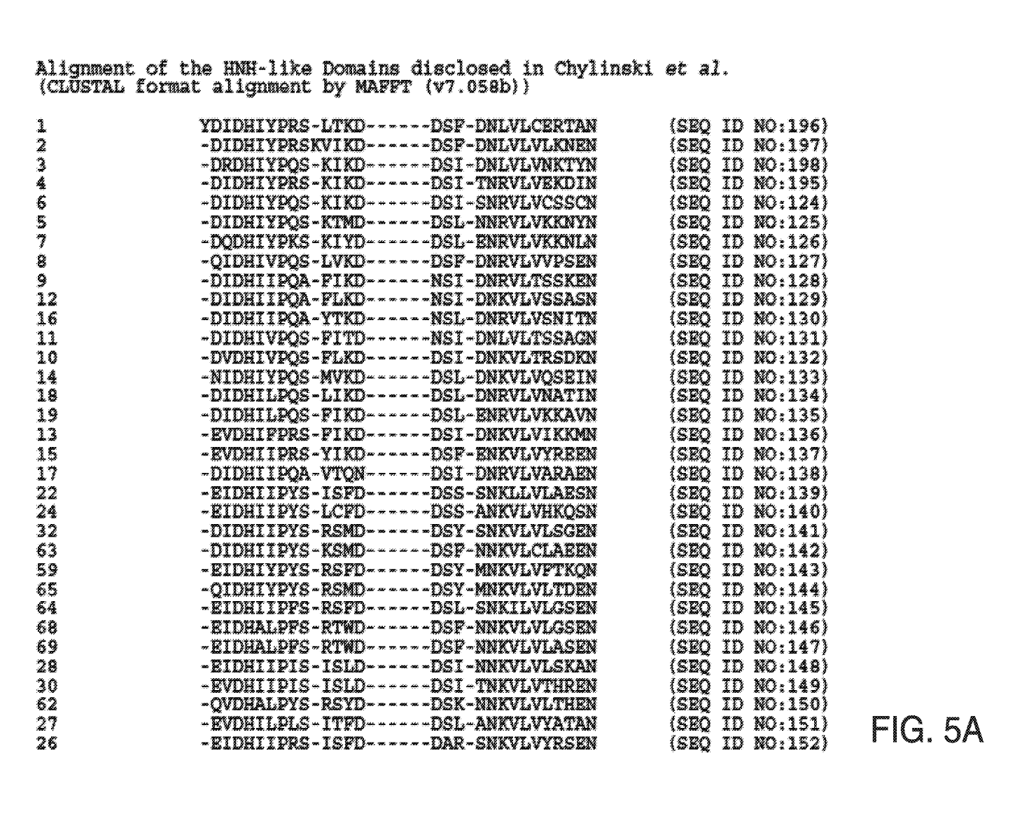

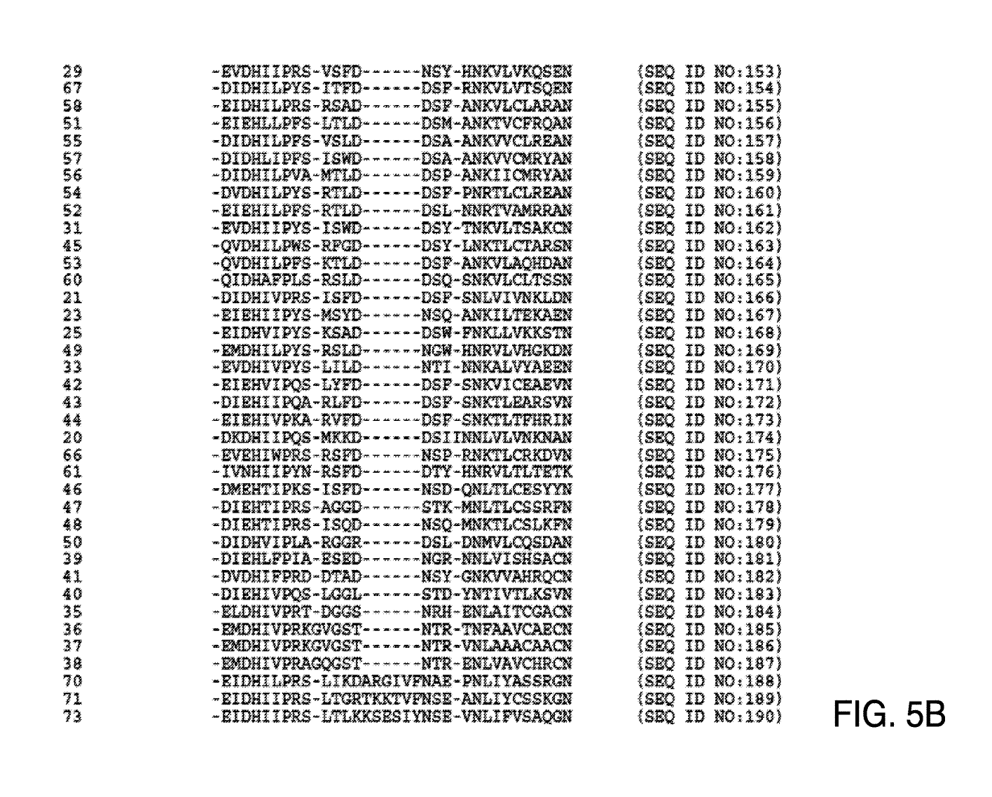

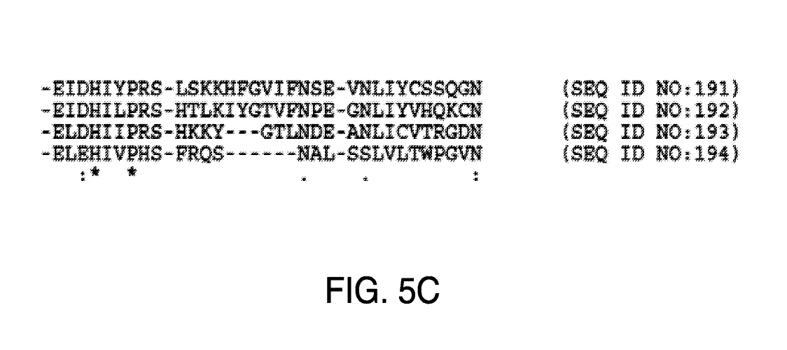

In certain embodiments, the HNH-like domain differs from a sequence of an HNH-like domain disclosed herein, e.g., in FIGS. 5A-5C, by as many as 1 but not more than 2, 3, 4, or 5 residues. In certain embodiments, 1 or both of the highly conserved residues identified in FIGS. 5A-5C are present.

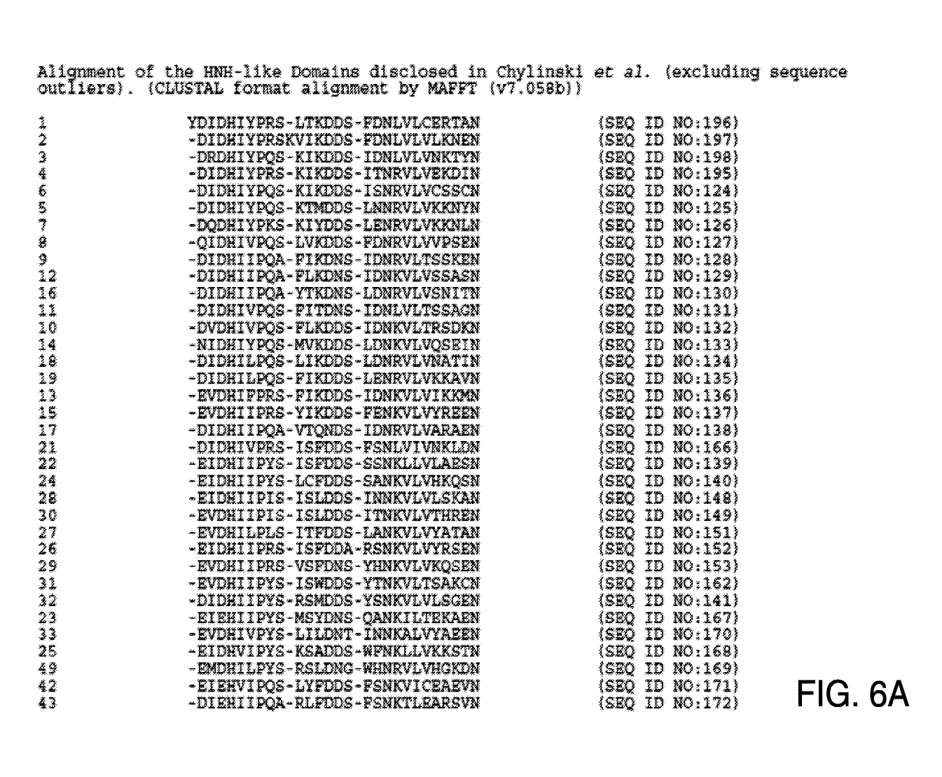

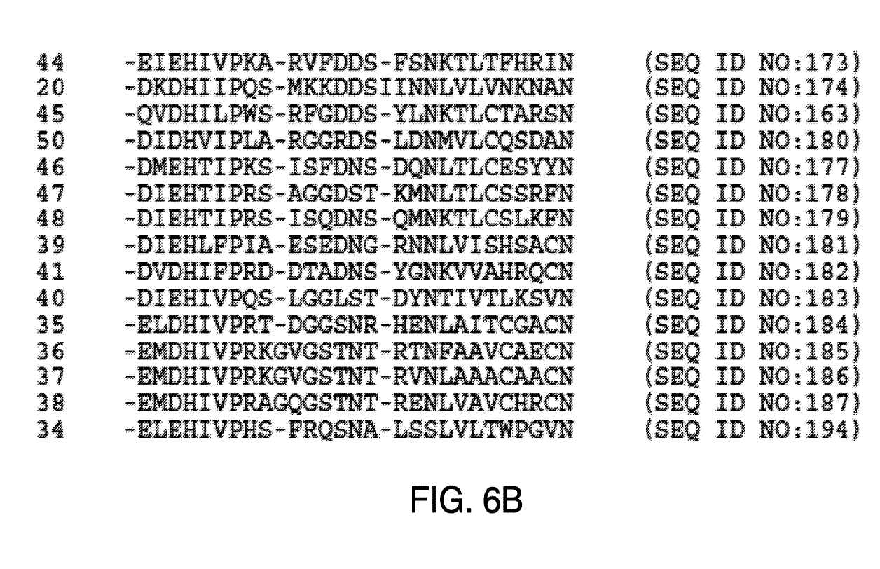

In certain embodiments, the HNH -like domain differs from a sequence of an HNH-like domain disclosed herein, e.g., in FIGS. 6A-6B, by as many as 1 but not more than 2, 3, 4, or 5 residues. In certain embodiments, 1, 2, or all 3 of the highly conserved residues identified in FIGS. 6A-6B are present.

9.2 Cas9 Activities

In certain embodiments, the Cas9 molecule or Cas9 polypeptide is capable of cleaving a target nucleic acid molecule. Typically wild-type Cas9 molecules cleave both strands of a target nucleic acid molecule. Cas9 molecules and Cas9 polypeptides can be engineered to alter nuclease cleavage (or other properties), e.g., to provide a Cas9 molecule or Cas9 polypeptide which is a nickase, or which lacks the ability to cleave target nucleic acid. A Cas9 molecule or Cas9 polypeptide that is capable of cleaving a target nucleic acid molecule is referred to herein as an eaCas9 (an enzymatically active Cas9) molecule or eaCas9 polypeptide.

In certain embodiments, an eaCas9 molecule or eaCas9 polypeptide comprises one or more of the following enzymatic activities:

a nickase activity, i.e., the ability to cleave a single strand, e.g., the non-complementary strand or the complementary strand, of a nucleic acid molecule;

a double stranded nuclease activity, i.e., the ability to cleave both strands of a double stranded nucleic acid and create a double stranded break, which in certain embodiments is the presence of two nickase activities;

an endonuclease activity;

an exonuclease activity; and

a helicase activity, i.e., the ability to unwind the helical structure of a double stranded nucleic acid.

In certain embodiments, an enzymatically active Cas9 (“eaCas9”) molecule or eaCas9 polypeptide cleaves both DNA strands and results in a double stranded break. In certain embodiments, an eaCas9 molecule or eaCas9 polypeptide cleaves only one strand, e.g., the strand to which the gRNA hybridizes to, or the strand complementary to the strand the gRNA hybridizes with. In certain embodiments, an eaCas9 molecule or eaCas9 polypeptide comprises cleavage activity associated with an HNH domain. In certain embodiments, an eaCas9 molecule or eaCas9 polypeptide comprises cleavage activity associated with a RuvC domain. In certain embodiments, an eaCas9 molecule or eaCas9 polypeptide comprises cleavage activity associated with an HNH domain and cleavage activity associated with a RuvC domain. In certain embodiments, an eaCas9 molecule or eaCas9 polypeptide comprises an active, or cleavage competent, HNH domain and an inactive, or cleavage incompetent, RuvC domain. In certain embodiments, an eaCas9 molecule or eaCas9 polypeptide comprises an inactive, or cleavage incompetent, HNH domain and an active, or cleavage competent, RuvC domain.

In certain embodiments, the Cas9 molecules or Cas9 polypeptides have the ability to interact with a gRNA molecule, and in conjunction with the gRNA molecule localize to a core target domain, but are incapable of cleaving the target nucleic acid, or incapable of cleaving at efficient rates. Cas9 molecules having no, or no substantial, cleavage activity are referred to herein as an enzymatically inactive Cas9 (“eiCas9”) molecule or eiCas9 polypeptide. For example, an eiCas9 molecule or eiCas9 polypeptide can lack cleavage activity or have substantially less, e.g., less than 20, 10, 5, 1 or 0.1% of the cleavage activity of a reference Cas9 molecule or eiCas9 polypeptide, as measured by an assay described herein.

9.3 Targeting and PAMs

A Cas9 molecule or Cas9 polypeptide can interact with a gRNA molecule and, in concert with the gRNA molecule, localizes to a site which comprises a target domain, and in certain embodiments, a PAM sequence.

In certain embodiments, the ability of an eaCas9 molecule or eaCas9 polypeptide to interact with and cleave a target nucleic acid is PAM sequence dependent. A PAM sequence is a sequence in the target nucleic acid. In certain embodiments, cleavage of the target nucleic acid occurs upstream from the PAM sequence. eaCas9 molecules from different bacterial species can recognize different sequence motifs (e.g., PAM sequences). In certain embodiments, an eaCas9 molecule of S. pyogenes recognizes the sequence motif NGG and directs cleavage of a target nucleic acid sequence 1 to 10, e.g., 3 to 5, bp upstream from that sequence (see, e.g., Mali 2013). In certain embodiments, an eaCas9 molecule of S. thermophilus recognizes the sequence motif NGGNG (SEQ ID NO:199) and/or NNAGAAW (W=A or T) (SEQ ID NO:200) and directs cleavage of a target nucleic acid sequence 1 to 10, e.g., 3 to 5, bp upstream from these sequences (see, e.g., Horvath 2010; Deveau 2008). In certain embodiments, an eaCas9 molecule of S. mutans recognizes the sequence motif NGG and/or NAAR (R =A or G) (SEQ ID NO:201) and directs cleavage of a target nucleic acid sequence 1 to 10, e.g., 3 to 5 bp, upstream from this sequence (see, e.g., Deveau 2008). In certain embodiments, an eaCas9 molecule of S. aureus recognizes the sequence motif NNGRR (R=A or G) (SEQ ID NO:202) and directs cleavage of a target nucleic acid sequence 1 to 10, e.g., 3 to 5, bp upstream from that sequence. In certain embodiments, an eaCas9 molecule of S. aureus recognizes the sequence motif NNGRRN (R=A or G) (SEQ ID NO:203) and directs cleavage of a target nucleic acid sequence 1 to 10, e.g., 3 to 5, bp upstream from that sequence. In certain embodiments, an eaCas9 molecule of S. aureus recognizes the sequence motif NNGRRT (R=A or G) (SEQ ID NO:204) and directs cleavage of a target nucleic acid sequence 1 to 10, e.g., 3 to 5, bp upstream from that sequence. In certain embodiments, an eaCas9 molecule of S. aureus recognizes the sequence motif NNGRRV (R=A or G) (SEQ ID NO:205) and directs cleavage of a target nucleic acid sequence 1 to 10, e.g., 3 to 5, bp upstream from that sequence. In certain embodiments, an eaCas9 molecule of Neisseria meningitidis recognizes the sequence motif NNNNGATT (SEQ ID NO: 8408) or NNNGCTT (SEQ ID NO: 8409) and directs cleavage of a target nucleic acid sequence 1 to 10, e.g., 3 to 5, base pairs upstream from that sequence. See, e.g., Hou et al., PNAS Early Edition 2013, 1-6. The ability of a Cas9 molecule to recognize a PAM sequence can be determined, e.g., using a transformation assay as described previously (Jinek 2012). In the aforementioned embodiments, N can be any nucleotide residue, e.g., any of A, G, C, or T.

As is discussed herein, Cas9 molecules can be engineered to alter the PAM specificity of the Cas9 molecule.

Exemplary naturally occurring Cas9 molecules have been described previously (see, e.g., Chylinski 2013). Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 15 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

Exemplary naturally occurring Cas9 molecules include a Cas9 molecule of a cluster 1 bacterial family. Examples include a Cas9 molecule of: S. aureus, S. pyogenes (e.g., strain SF370, MGAS10270, MGAS10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA159, NN2025), S. macacae (e.g., strain NCTC11558), S. gallolyticus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g., strain F0211), S. agalactiae (e.g., strain NEM316, A909), Listeria monocytogenes (e.g., strain F6854), Listeria innocua (L. innocua, e.g., strain Clip11262), Enterococcus italicus (e.g., strain DSM 15952), or Enterococcus faecium (e.g., strain 1,231,408).

Additional exemplary Cas9 molecules are a Cas9 molecule of Neisseria meningitides (Hou et al., PNAS Early Edition 2013, 1-6 and a S. aureus cas9 molecule.

In certain embodiments, a Cas9 molecule or Cas9 polypeptide comprises an amino acid sequence:

having about 60%, about 65%, about 70%, about 75%, about 80%, about 85%, about 90%, about 95%, about 96%, about 97%, about 98% or about 99% homology with;

differs at no more than, about 2%, about 5%, about 10%, about 15%, about 20%, about 30%, or about 40% of the amino acid residues when compared with;

differs by at least 1, 2, 5, 10 or 20 amino acids, but by no more than 100, 80, 70, 60, 50, 40 or 30 amino acids from; or

identical to any Cas9 molecule sequence described herein, or to a naturally occurring Cas9 molecule sequence, e.g., a Cas9 molecule from a species listed herein (e.g., SEQ ID NOs:1, 2, 4-6, or 12) or described in Chylinski 2013. In certain embodiments, the Cas9 molecule or Cas9 polypeptide comprises one or more of the following activities: a nickase activity; a double stranded cleavage activity (e.g., an endonuclease and/or exonuclease activity); a helicase activity; or the ability, together with a gRNA molecule, to localize to a target nucleic acid.

In certain embodiments, a Cas9 molecule or Cas9 polypeptide comprises any of the amino acid sequence of the consensus sequence of FIGS. 2A-2G, wherein “*” indicates any amino acid found in the corresponding position in the amino acid sequence of a Cas9 molecule of S. pyogenes, S. thermophilus, S. mutans, or L. innocua, and “-” indicates absent. In certain embodiments, a Cas9 molecule or Cas9 polypeptide differs from the sequence of the consensus sequence disclosed in FIGS. 2A-2G by at least 1, but no more than 2, 3, 4, 5, 6, 7, 8, 9, or 10 amino acid residues. In certain embodiments, a Cas9 molecule or Cas9 polypeptide comprises the amino acid sequence of SEQ ID NO:2. In other embodiments, a Cas9 molecule or Cas9 polypeptide differs from the sequence of SEQ ID NO:2 by at least 1, but no more than 2, 3, 4, 5, 6, 7, 8, 9, or 10 amino acid residues.

A comparison of the sequence of a number of Cas9 molecules indicate that certain regions are conserved. These are identified below as:

region 1 (residues 1 to 180, or in the case of region 1′residues 120 to 180)

region 2 (residues 360 to 480);

region 3 (residues 660 to 720);

region 4 (residues 817 to 900); and

region 5 (residues 900 to 960).

In certain embodiments, a Cas9 molecule or Cas9 polypeptide comprises regions 1-5, together with sufficient additional Cas9 molecule sequence to provide a biologically active molecule, e.g., a Cas9 molecule having at least one activity described herein. In certain embodiments, each of regions 1-5, independently, have about 50%, about 60%, about 70%, about 80%, about 85%, about 90%, about 95%, about 96%, about 97%, about 98% or about 99% homology with the corresponding residues of a Cas9 molecule or Cas9 polypeptide described herein, e.g., a sequence from FIGS. 2A-2G.

In certain embodiments, a Cas9 molecule or Cas9 polypeptide comprises an amino acid sequence referred to as region 1:

having about 50%, about 60%, about 70%, about 80%, about 85%, about 90%, about 95%, about 96%, about 97%, about 98% or about 99% homology with amino acids 1-180 (the numbering is according to the motif sequence in FIG. 2; 52% of residues in the four Cas9 sequences in FIGS. 2A-2G are conserved) of the amino acid sequence of Cas9 of S. pyogenes;

differs by at least 1, 2, 5, 10 or 20 amino acids but by no more than 90, 80, 70, 60, 50, 40 or 30 amino acids from amino acids 1-180 of the amino acid sequence of Cas9 of S. pyogenes, S. thermophilus, S. mutans, or Listeria innocua; or

is identical to amino acids 1-180 of the amino acid sequence of Cas9 of S. pyogenes, S. thermophilus, S. mutans, or L. innocua.

In certain embodiments, a Cas9 molecule or Cas9 polypeptide comprises an amino acid sequence referred to as region 1′:

having about 55%, about 60%, about 65%, about 70%, about 75%, about 80%, about 85%, about 90%, about 95%, about 96%, about 97%, about 98% or about 99% homology with amino acids 120-180 (55% of residues in the four Cas9 sequences in FIG. 2 are conserved) of the amino acid sequence of Cas9 of S. pyogenes, S. thermophilus, S. mutans or L. innocua;

differs by at least 1, 2, or 5 amino acids but by no more than 35, 30, 25, 20 or 10 amino acids from amino acids 120-180 of the amino acid sequence of Cas9 of S. pyogenes, S. thermophilus, S. mutans, or L. innocua; or

is identical to amino acids 120-180 of the amino acid sequence of Cas9 of S. pyogenes, S. thermophilus, S. mutans, or L. innocua.

In certain embodiments, a Cas9 molecule or Cas9 polypeptide comprises an amino acid sequence referred to as region 2:

having about 50%, about 55%, about 60%, about 65%, about 70%, about 75%, about 80%, about 85%, about 90%, about 95%, about 96%, about 97%, about 98% or about 99% homology with amino acids 360-480 (52% of residues in the four Cas9 sequences in FIG. 2 are conserved) of the amino acid sequence of Cas9 of S. pyogenes, S. thermophilus, S. mutans, or L. innocua;

differs by at least 1, 2, or 5 amino acids but by no more than 35, 30, 25, 20 or 10 amino acids from amino acids 360-480 of the amino acid sequence of Cas9 of S. pyogenes, S. thermophilus, S. mutans, or L. innocua; or

is identical to amino acids 360-480 of the amino acid sequence of Cas9 of S. pyogenes, S. thermophilus, S. mutans, or L. innocua.

In certain embodiments, a Cas9 molecule or Cas9 polypeptide comprises an amino acid sequence referred to as region 3:

having about 55%, about 60%, about 65%, about 70%, about 75%, about 80%, about 85%, about 90%, about 95%, about 96%, about 97%, about 98%, or about 99% homology with amino acids 660-720 (56% of residues in the four Cas9 sequences in FIG. 2 are conserved) of the amino acid sequence of Cas9 of S. pyogenes, S. thermophilus, S. mutans or L. innocua;

differs by at least 1, 2, or 5 amino acids but by no more than 35, 30, 25, 20 or 10 amino acids from amino acids 660-720 of the amino acid sequence of Cas9 of S. pyogenes, S. thermophilus, S. mutans or L. innocua; or

is identical to amino acids 660-720 of the amino acid sequence of Cas9 of S. pyogenes, S. thermophilus, S. mutans or L. innocua.

In certain embodiments, a Cas9 molecule or Cas9 polypeptide comprises an amino acid sequence referred to as region 4:

having about 50%, about 55%, about 60%, about 65%, about 70%, about 75%, about 80%, about 85%, about 90%, about 95%, about 96%, about 97%, about 98%, or about 99% homology with amino acids 817-900 (55% of residues in the four Cas9 sequences in FIGS. 2A-2G are conserved) of the amino acid sequence of Cas9 of S. pyogenes, S. thermophilus, S. mutans, or L. innocua;

differs by at least 1, 2, or 5 amino acids but by no more than 35, 30, 25, 20 or 10 amino acids from amino acids 817-900 of the amino acid sequence of Cas9 of S. pyogenes, S. thermophilus, S. mutans, or L. innocua; or

is identical to amino acids 817-900 of the amino acid sequence of Cas9 of S. pyogenes, S. thermophilus, S. mutans, or L. innocua.

In certain embodiments, a Cas9 molecule or Cas9 polypeptide comprises an amino acid sequence referred to as region 5:

having about 50%, about 55%, about 60%, about 65%, about 70%, about 75%, about 80%, about 85%, about 90%, about 95%, about 96%, about 97%, about 98%, or about 99% homology with amino acids 900-960 (60% of residues in the four Cas9 sequences in FIGS. 2A-2G are conserved) of the amino acid sequence of Cas9 of S. pyogenes, S. thermophilus, S. mutans, or L. innocua;

differs by at least 1, 2, or 5 amino acids but by no more than 35, 30, 25, 20 or 10 amino acids from amino acids 900-960 of the amino acid sequence of Cas9 of S. pyogenes, S. thermophilus, S. mutans, or L. innocua; or

is identical to amino acids 900-960 of the amino acid sequence of Cas9 of S. pyogenes, S. thermophilus, S. mutans, or L. innocua.

9.4 Engineered or Altered Cas9

Cas9 molecules and Cas9 polypeptides described herein can possess any of a number of properties, including nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In certain embodiments, a Cas9 molecule or Cas9 polypeptide can include all or a subset of these properties. In certain embodiments, a Cas9 molecule or Cas9 polypeptide has the ability to interact with a gRNA molecule and, in concert with the gRNA molecule, localize to a site in a nucleic acid. Other activities, e.g., PAM specificity, cleavage activity, or helicase activity can vary more widely in Cas9 molecules and Cas9 polypeptides.

Cas9 molecules include engineered Cas9 molecules and engineered Cas9 polypeptides (engineered, as used in this context, means merely that the Cas9 molecule or Cas9 polypeptide differs from a reference sequences, and implies no process or origin limitation). An engineered Cas9 molecule or Cas9 polypeptide can comprise altered enzymatic properties, e.g., altered nuclease activity, (as compared with a naturally occurring or other reference Cas9 molecule) or altered helicase activity. As discussed herein, an engineered Cas9 molecule or Cas9 polypeptide can have nickase activity (as opposed to double strand nuclease activity). In certain embodiments, an engineered Cas9 molecule or Cas9 polypeptide can have an alteration that alters its size, e.g., a deletion of amino acid sequence that reduces its size, e.g., without significant effect on one or more, or any Cas9 activity. In certain embodiments, an engineered Cas9 molecule or Cas9 polypeptide can comprise an alteration that affects PAM recognition. In certain embodiments, an engineered Cas9 molecule is altered to recognize a PAM sequence other than that recognized by the endogenous wild-type PI domain. In certain embodiments, a Cas9 molecule or Cas9 polypeptide can differ in sequence from a naturally occurring Cas9 molecule but not have significant alteration in one or more Cas9 activities.

Cas9 molecules or Cas9 polypeptides with desired properties can be made in a number of ways, e.g., by alteration of a parental, e.g., naturally occurring, Cas9 molecules or Cas9 polypeptides, to provide an altered Cas9 molecule or Cas9 polypeptide having a desired property. For example, one or more mutations or differences relative to a parental Cas9 molecule, e.g., a naturally occurring or engineered Cas9 molecule, can be introduced. Such mutations and differences comprise: substitutions (e.g., conservative substitutions or substitutions of non-essential amino acids); insertions; or deletions. In certain embodiments, a Cas9 molecule or Cas9 polypeptide can comprises one or more mutations or differences, e.g., at least 1, 2, 3, 4, 5, 10, 15, 20, 30, 40 or 50 mutations but less than 200, 100, or 80 mutations relative to a reference, e.g., a parental, Cas9 molecule.

In certain embodiments, a mutation or mutations do not have a substantial effect on a Cas9 activity, e.g. a Cas9 activity described herein. In certain embodiments, a mutation or mutations have a substantial effect on a Cas9 activity, e.g. a Cas9 activity described herein.

9.5 Modified-Cleavage Cas9

In certain embodiments, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S. pyogenes, as follows: its ability to modulate, e.g., decreased or increased, cleavage of a double stranded nucleic acid (endonuclease and/or exonuclease activity), e.g., as compared to a naturally occurring Cas9 molecule (e.g., a Cas9 molecule of S. pyogenes); its ability to modulate, e.g., decreased or increased, cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S. pyogenes); or the ability to cleave a nucleic acid molecule, e.g., a double stranded or single stranded nucleic acid molecule, can be eliminated.

In certain embodiments, an eaCas9 molecule or eaCas9 polypeptide comprises one or more of the following activities: cleavage activity associated with an N-terminal RuvC-like domain; cleavage activity associated with an HNH-like domain; cleavage activity associated with an HNH-like domain and cleavage activity associated with an N-terminal RuvC-like domain.

In certain embodiments, an eaCas9 molecule or eaCas9 polypeptide comprises an active, or cleavage competent, HNH-like domain (e.g., an HNH-like domain described herein, e.g., SEQ ID NOs:24-28) and an inactive, or cleavage incompetent, N-terminal RuvC-like domain. An exemplary inactive, or cleavage incompetent N-terminal RuvC-like domain can have a mutation of an aspartic acid in an N-terminal RuvC-like domain, e.g., an aspartic acid at position 9 of the consensus sequence disclosed in FIGS. 2A-2G or an aspartic acid at position 10 of SEQ ID NO:2, e.g., can be substituted with an alanine. In certain embodiments, the eaCas9 molecule or eaCas9 polypeptide differs from wild-type in the N-terminal RuvC-like domain and does not cleave the target nucleic acid, or cleaves with significantly less efficiency, e.g., less than about 20%, about 10%, about 5%, about 1% or about 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, S. aureus, or S. thermophilus. In certain embodiments, the reference Cas9 molecule is the naturally occurring Cas9 molecule having the closest sequence identity or homology.

In certain embodiments, an eaCas9 molecule or eaCas9 polypeptide comprises an inactive, or cleavage incompetent, HNH domain and an active, or cleavage competent, N-terminal RuvC-like domain (e.g., a RuvC-like domain described herein, e.g., SEQ ID NOs:15-23). Exemplary inactive, or cleavage incompetent HNH-like domains can have a mutation at one or more of: a histidine in an HNH-like domain, e.g., a histidine shown at position 856 of the consensus sequence disclosed in FIGS. 2A-2G, e.g., can be substituted with an alanine; and one or more asparagines in an HNH-like domain, e.g., an asparagine shown at position 870 of the consensus sequence disclosed in FIGS. 2A-2G and/or at position 879 of the consensus sequence disclosed in FIGS. 2A-2G, e.g., can be substituted with an alanine. In certain embodiments, the eaCas9 differs from wild-type in the HNH-like domain and does not cleave the target nucleic acid, or cleaves with significantly less efficiency, e.g., less than about 20%, about 10%, about 5%, about 1% or about 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, S. aureus, or S. thermophilus. In certain embodiments, the reference Cas9 molecule is the naturally occurring Cas9 molecule having the closest sequence identity or homology.

In certain embodiments, exemplary Cas9 activities comprise one or more of PAM specificity, cleavage activity, and helicase activity. A mutation(s) can be present, e.g., in: one or more RuvC domains, e.g., an N-terminal RuvC domain; an HNH domain; a region outside the RuvC domains and the HNH domain. In certain embodiments, a mutation(s) is present in a RuvC domain. In certain embodiments, a mutation(s) is present in an HNH domain. In certain embodiments, mutations are present in both a RuvC domain and an HNH domain.

Exemplary mutations that may be made in the RuvC domain or HNH domain with reference to the S. pyogenes Cas9 sequence include: D10A, E762A, H840A, N854A, N863A and/or D986A. Exemplary mutations that may be made in the RuvC domain with reference to the S. aureus Cas9 sequence include N580A (see, e.g., SEQ ID NO:11).

Whether or not a particular sequence, e.g., a substitution, may affect one or more activity, such as targeting activity, cleavage activity, etc., can be evaluated or predicted, e.g., by evaluating whether the mutation is conservative. In certain embodiments, a “non-essential” amino acid residue, as used in the context of a Cas9 molecule, is a residue that can be altered from the wild-type sequence of a Cas9 molecule, e.g., a naturally occurring Cas9 molecule, e.g., an eaCas9 molecule, without abolishing or more preferably, without substantially altering a Cas9 activity (e.g., cleavage activity), whereas changing an “essential” amino acid residue results in a substantial loss of activity (e.g., cleavage activity).

In certain embodiments, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule can differ from naturally occurring Cas9 molecules, e.g., a Cas9 molecule of S aureus or S. pyogenes, as follows: its ability to modulate, e.g., decreased or increased, cleavage of a double stranded break (endonuclease and/or exonuclease activity), e.g., as compared to a naturally occurring Cas9 molecule (e.g., a Cas9 molecule of S aureus or S. pyogenes); its ability to modulate, e.g., decreased or increased, cleavage of a single strand of a nucleic acid, e.g., a non-complimentary strand of a nucleic acid molecule or a complementary strand of a nucleic acid molecule (nickase activity), e.g., as compared to a naturally occurring Cas9 molecule (e.g., a Cas9 molecule of S aureus or S. pyogenes); or the ability to cleave a nucleic acid molecule, e.g., a double stranded or single stranded nucleic acid molecule, can be eliminated. In certain embodiments, the nickase is S. aureus Cas9-derived nickase comprising the sequence of SEQ ID NO:10 (D10A) or SEQ ID NO:11 (N580A) (Friedland 2015).

In certain embodiments, the altered Cas9 molecule is an eaCas9 molecule comprising one or more of the following activities: cleavage activity associated with a RuvC domain; cleavage activity associated with an HNH domain; cleavage activity associated with an HNH domain and cleavage activity associated with a RuvC domain.

In certain embodiments, the altered Cas9 molecule or Cas9 polypeptide comprises a sequence in which:

the sequence corresponding to the fixed sequence of the consensus sequence disclosed in FIGS. 2A-2G differs at no more than about 1%, about 2%, about 3%, about 4%, about 5%, about 10%, about 15%, or about 20% of the fixed residues in the consensus sequence disclosed in FIGS. 2A-2G; and

the sequence corresponding to the residues identified by “*” in the consensus sequence disclosed in FIGS. 2A-2G differs at no more than about 1%, about 2%, about 3%, about 4%, about 5%, about 10%, about 15%, about 20%, about 25%, about 30%, about 35%, or about 40% of the “*” residues from the corresponding sequence of naturally occurring Cas9 molecule, e.g., an S. pyogenes, S. thermophilus, S. mutans, or L. innocua Cas9 molecule.

In certain embodiments, the altered Cas9 molecule or Cas9 polypeptide is an eaCas9 molecule or eaCas9 polypeptide comprising the amino acid sequence of S. pyogenes Cas9 disclosed in FIGS. 2A-2G with one or more amino acids that differ from the sequence of S. pyogenes (e.g., substitutions) at one or more residues (e.g., 2, 3, 5, 10, 15, 20, 30, 50, 70, 80, 90, 100, or 200 amino acid residues) represented by an “*” in the consensus sequence disclosed in FIGS. 2A-2G.

In certain embodiments, the altered Cas9 molecule or Cas9 polypeptide is an eaCas9 molecule or eaCas9 polypeptide comprising the amino acid sequence of S. thermophilus Cas9 disclosed in FIGS. 2A-2G with one or more amino acids that differ from the sequence of S. thermophilus (e.g., substitutions) at one or more residues (e.g., 2, 3, 5, 10, 15, 20, 30, 50, 70, 80, 90, 100, or 200 amino acid residues) represented by an “*” in the consensus sequence disclosed in FIGS. 2A-2G.

In certain embodiments, the altered Cas9 molecule or Cas9 polypeptide is an eaCas9 molecule or eaCas9 polypeptide comprising the amino acid sequence of S. mutans Cas9 disclosed in FIGS. 2A-2G with one or more amino acids that differ from the sequence of S. mutans (e.g., substitutions) at one or more residues (e.g., 2, 3, 5, 10, 15, 20, 30, 50, 70, 80, 90, 100, or 200 amino acid residues) represented by an “*” in the consensus sequence disclosed in FIGS. 2A-2G.

In certain embodiments, the altered Cas9 molecule or Cas9 polypeptide is an eaCas9 molecule or eaCas9 polypeptide comprising the amino acid sequence of L. innocua Cas9 disclosed in FIGS. 2A-2G with one or more amino acids that differ from the sequence of L. innocua (e.g., substitutions) at one or more residues (e.g., 2, 3, 5, 10, 15, 20, 30, 50, 70, 80, 90, 100, or 200 amino acid residues) represented by an “*” in the consensus sequence disclosed in FIGS. 2A-2G.

In certain embodiments, the altered Cas9 molecule or Cas9 polypeptide, e.g., an eaCas9 molecule or eaCas9 polypeptide, can be a fusion, e.g., of two of more different Cas9 molecules, e.g., of two or more naturally occurring Cas9 molecules of different species. For example, a fragment of a naturally occurring Cas9 molecule of one species can be fused to a fragment of a Cas9 molecule of a second species. As an example, a fragment of a Cas9 molecule of S. pyogenes comprising an N-terminal RuvC-like domain can be fused to a fragment of Cas9 molecule of a species other than S. pyogenes (e.g., S. thermophilus) comprising an HNH-like domain.

9.6 Cas9 with Altered or no PAM Recognition

Naturally occurring Cas9 molecules can recognize specific PAM sequences, for example the PAM recognition sequences described above for, e.g., S. pyogenes, S. thermophilus, S. mutans, and S. aureus.

In certain embodiments, a Cas9 molecule or Cas9 polypeptide has the same PAM specificities as a naturally occurring Cas9 molecule. In certain embodiments, a Cas9 molecule or Cas9 polypeptide has a PAM specificity not associated with a naturally occurring Cas9 molecule, or a PAM specificity not associated with the naturally occurring Cas9 molecule to which it has the closest sequence homology. For example, a naturally occurring Cas9 molecule can be altered, e.g., to alter PAM recognition, e.g., to alter the PAM sequence that the Cas9 molecule or Cas9 polypeptide recognizes in order to decrease off-target sites and/or improve specificity; or eliminate a PAM recognition requirement. In certain embodiments, a Cas9 molecule or Cas9 polypeptide can be altered, e.g., to increase length of PAM recognition sequence and/or improve Cas9 specificity to high level of identity (e.g., about 98%, about 99% or about 100% match between gRNA and a PAM sequence), e.g., to decrease off-target sites and/or increase specificity. In certain embodiments, the length of the PAM recognition sequence is at least 4, 5, 6, 7, 8, 9, 10 or 15 amino acids in length. In certain embodiments, the Cas9 specificity requires at least about 90%, about 95%, about 96%, about 97%, about 98%, or about 99% homology between the gRNA and the PAM sequence. Cas9 molecules or Cas9 polypeptides that recognize different PAM sequences and/or have reduced off-target activity can be generated using directed evolution. Exemplary methods and systems that can be used for directed evolution of Cas9 molecules are described (see, e.g., Esvelt 2011). Candidate Cas9 molecules can be evaluated, e.g., by methods described below.

9.7 Size-Optimized Cas9

Engineered Cas9 molecules and engineered Cas9 polypeptides described herein include a Cas9 molecule or Cas9 polypeptide comprising a deletion that reduces the size of the molecule while still retaining desired Cas9 properties, e.g., essentially native conformation, Cas9 nuclease activity, and/or target nucleic acid molecule recognition. Provided herein are Cas9 molecules or Cas9 polypeptides comprising one or more deletions and optionally one or more linkers, wherein a linker is disposed between the amino acid residues that flank the deletion. Methods for identifying suitable deletions in a reference Cas9 molecule, methods for generating Cas9 molecules with a deletion and a linker, and methods for using such Cas9 molecules will be apparent to one of ordinary skill in the art upon review of this document.

A Cas9 molecule, e.g., a S. aureus or S. pyogenes Cas9 molecule, having a deletion is smaller, e.g., has reduced number of amino acids, than the corresponding naturally-occurring Cas9 molecule. The smaller size of the Cas9 molecules allows increased flexibility for delivery methods, and thereby increases utility for genome editing. A Cas9 molecule can comprise one or more deletions that do not substantially affect or decrease the activity of the resultant Cas9 molecules described herein. Activities that are retained in the Cas9 molecules comprising a deletion as described herein include one or more of the following:

a nickase activity, i.e., the ability to cleave a single strand, e.g., the non-complementary strand or the complementary strand, of a nucleic acid molecule; a double stranded nuclease activity, i.e., the ability to cleave both strands of a double stranded nucleic acid and create a double stranded break, which in certain embodiments is the presence of two nickase activities;

an endonuclease activity;

an exonuclease activity;

a helicase activity, i.e., the ability to unwind the helical structure of a double stranded nucleic acid;

and recognition activity of a nucleic acid molecule, e.g., a target nucleic acid or a gRNA.

Activity of the Cas9 molecules described herein can be assessed using the activity assays described herein or in the art.

9.8 Identifying Regions Suitable for Deletion

Suitable regions of Cas9 molecules for deletion can be identified by a variety of methods. Naturally-occurring orthologous Cas9 molecules from various bacterial species can be modeled onto the crystal structure of S. pyogenes Cas9 (Nishimasu 2014) to examine the level of conservation across the selected Cas9 orthologs with respect to the three-dimensional conformation of the protein. Less conserved or unconserved regions that are spatially located distant from regions involved in Cas9 activity, e.g., interface with the target nucleic acid molecule and/or gRNA, represent regions or domains are candidates for deletion without substantially affecting or decreasing Cas9 activity.

9.9 Nucleic Acids Encoding Cas9 Molecules

Nucleic acids encoding the Cas9 molecules or Cas9 polypeptides, e.g., an eaCas9 molecule or eaCas9 polypeptides are provided herein. Exemplary nucleic acids encoding Cas9 molecules or Cas9 polypeptides have been described previously (see, e.g., Cong 2013; Wang 2013; Mali 2013; Jinek 2012).

In certain embodiments, a nucleic acid encoding a Cas9 molecule or Cas9 polypeptide can be a synthetic nucleic acid sequence. For example, the synthetic nucleic acid molecule can be chemically modified, e.g., as described herein. In certain embodiments, the Cas9 mRNA has one or more (e.g., all of the following properties: it is capped, polyadenylated, substituted with 5-methylcytidine and/or pseudouridine.

Additionally or alternatively, the synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein.

Additionally or alternatively, a nucleic acid encoding a Cas9 molecule or Cas9 polypeptide may comprise a nuclear localization sequence (NLS). Nuclear localization sequences are known in the art.

An exemplary codon optimized nucleic acid sequence encoding a Cas9 molecule of S. pyogenes is set forth in SEQ ID NO:3. The corresponding amino acid sequence of an S. pyogenes Cas9 molecule is set forth in SEQ ID NO:2.

Exemplary codon optimized nucleic acid sequences encoding a Cas9 molecule of S. aureus are set forth in SEQ ID NOs:7-9, 206 and 207. In certain embodiments, the Cas9 molecule is a mutant S. aureus CasO molecule comprising a D10A mutation. In certain embodiments, a codon optimized nucleic acid sequences encoding an S. aureus Cas9 molecule is set forth in SEQ ID NO: 8. An amino acid sequence of an S. aureus Cas9 molecule is set forth in SEQ ID NO:6.

If any of the above Cas9 sequences are fused with a peptide or polypeptide at the C-terminus, it is understood that the stop codon can be removed.

9.10 Other Cas Molecules and Cas Polypeptides

Various types of Cas molecules or Cas polypeptides can be used to practice the inventions disclosed herein. In certain embodiments, Cas molecules of Type II Cas systems are used. In certain embodiments, Cas molecules of other Cas systems are used. For example, Type I or Type III Cas molecules may be used. Exemplary Cas molecules (and Cas systems) have been described previously (see, e.g., Haft 2005 and Makarova 2011). Exemplary Cas molecules (and Cas systems) are also shown in Table 5.

TABLE 5
Cas Systems

| | | | Structure of | Families (and | |
|---|---|---|---|---|---|
| | System | | encoded | superfamily) of | |
| Gene | type or | Name from | protein (PDB | encoded | |
| name^{‡} | subtype | Haft 2005^{§} | accessions)^{¶} | protein^{#}** | Representatives |
| cas1 | Type I | cas1 | 3GOD, 3LFX | COG1518 | SERP2463, |
| | Type II | | and 2YZS | | SPy1047 and ygbT |
| | Type III | | | | |
| cas2 | Type I | cas2 | 2IVY, 2I8E | COG1343 and | SERP2462, |
| | Type II | | and 3EXC | COG3512 | SPy1048, SPy1723 |
| | Type III | | | | (N-terminal domain) |
| | | | | | and ygbF |
| cas3′ | Type I^{‡‡} | cas3 | NA | COG1203 | APE1232 and ygcB |
| cas3″ | Subtype | NA | NA | COG2254 | APE1231 and |
| | I-A | | | | BH0336 |
| | Subtype | | | | |
| | I-B | | | | |
| cas4 | Subtype | cas4 and | NA | COG1468 | APE1239 and |
| | I-A | csa1 | | | BH0340 |
| | Subtype | | | | |
| | I-B | | | | |
| | Subtype | | | | |
| | I-C | | | | |
| | Subtype | | | | |
| | I-D | | | | |
| | Subtype | | | | |
| | II-B | | | | |
| cas5 | Subtype | cas5a, | 3KG4 | COG1688 | APE1234, BH0337, |
| | I-A | cas5d, | | (RAMP) | devS and ygcI |
| | Subtype | cas5e, | | | |
| | I-B | cas5h, | | | |
| | Subtype | cas5p, cas5t | | | |
| | I-C | and cmx5 | | | |
| | Subtype | | | | |
| | I-E | | | | |
| cas6 | Subtype | cas6 and | 3I4H | COG1583 and | PF1131 and slr7014 |
| | I-A | cmx6 | | COG5551 | |
| | Subtype | | | (RAMP) | |
| | I-B | | | | |
| | Subtype | | | | |
| | I-D | | | | |
| | Subtype | | | | |
| | III-A | | | | |
| | Subtype | | | | |
| | III-B | | | | |
| cas6e | Subtype | cse3 | 1WJ9 | (RAMP) | ygcH |
| | I-E | | | | |
| cas6f | Subtype | csy4 | 2XLJ | (RAMP) | y1727 |
| | I-F | | | | |
| cas7 | Subtype | csa2, csd2, | NA | COG1857 and | devR and ygcJ |
| | I-A | cse4, csh2, | | COG3649 | |
| | Subtype | csp1 and | | (RAMP) | |
| | I-B | cst2 | | | |
| | Subtype | | | | |
| | I-C | | | | |
| | Subtype | | | | |
| | I-E | | | | |
| cas8a1 | Subtype | cmx1, cst1, | NA | BH0338-like | LA3191^{§§} and |
| | I-A^{‡‡} | csx8, csx13 | | | PG2018^{§§} |
| | | and CXXC- | | | |
| | | CXXC | | | |
| cas8a2 | Subtype | csa4 and | NA | PH0918 | AF0070, AF1873, |
| | I-A^{‡‡} | csx9 | | | MJ0385, PF0637, |
| | | | | | PH0918 and |
| | | | | | SSO1401 |
| cas8b | Subtype | csh1 and | NA | BH0338-like | MTH1090 and |
| | I-B^{‡‡} | TM1802 | | | TM1802 |
| cas8c | Subtype | csd1 and | NA | BH0338-like | BH0338 |
| | I-C^{‡‡} | csp2 | | | |
| cas9 | Type II^{‡‡} | csn1 and | NA | COG3513 | FTN_0757 and |
| | | csx12 | | | SPy1046 |
| cas10 | Type III^{‡‡} | cmr2, csm1 | NA | COG1353 | MTH326, |
| | | and csx11 | | | Rv2823c^{§§} and |
| | | | | | TM1794^{§§} |
| cas10d | Subtype | csc3 | NA | COG1353 | slr7011 |
| | I-D^{‡‡} | | | | |
| csy1 | Subtype | csy1 | NA | y1724-like | y1724 |
| | I-F^{‡‡} | | | | |
| csy2 | Subtype | csy2 | NA | (RAMP) | y1725 |
| | I-F | | | | |
| csy3 | Subtype | csy3 | NA | (RAMP) | y1726 |
| | I-F | | | | |
| cse1 | Subtype | cse1 | NA | YgcL-like | ygcL |
| | I-E^{‡‡} | | | | |
| cse2 | Subtype | cse2 | 2ZCA | YgcK-like | ygcK |
| | I-E | | | | |
| csc1 | Subtype | csc1 | NA | alr1563-like | alr1563 |
| | I-D | | | (RAMP) | |
| csc2 | Subtype | csc1 and | NA | COG1337 | slr7012 |
| | I-D | csc2 | | (RAMP) | |
| csa5 | Subtype | csa5 | NA | AF1870 | AF1870, MJ0380, |
| | I-A | | | | PF0643 and |
| | | | | | SSO1398 |
| csn2 | Subtype | csn2 | NA | SPy1049-like | SPy1049 |
| | II-A | | | | |
| csm2 | Subtype | csm2 | NA | COG1421 | MTH1081 and |
| | III-A^{‡‡} | | | | SERP2460 |
| csm3 | Subtype | csc2 and | NA | COG1337 | MTH1080 and |
| | III-A | csm3 | | (RAMP) | SERP2459 |
| csm4 | Subtype | csm4 | NA | COG1567 | MTH1079 and |
| | III-A | | | (RAMP) | SERP2458 |
| csm5 | Subtype | csm5 | NA | COG1332 | MTH1078 and |
| | III-A | | | (RAMP) | SERP2457 |
| csm6 | Subtype | APE2256 | 2WTE | COG1517 | APE2256 and |
| | III-A | and csm6 | | | SSO1445 |
| cmr1 | Subtype | cmr1 | NA | COG1367 | PF1130 |
| | III-B | | | (RAMP) | |
| cmr3 | Subtype | cmr3 | NA | COG1769 | PF1128 |
| | III-B | | | (RAMP) | |
| cmr4 | Subtype | cmr4 | NA | COG1336 | PF1126 |
| | III-B | | | (RAMP) | |
| cmr5 | Subtype | cmr5 | 2ZOP and | COG3337 | MTH324 and |
| | III-B^{‡‡} | | 2OEB | | PF1125 |
| cmr6 | Subtype | cmr6 | NA | COG1604 | PF1124 |
| | III-B | | | (RAMP) | |
| csb1 | Subtype | GSU0053 | NA | (RAMP) | Balac_1306 and |
| | I-U | | | | GSU0053 |
| csb2 | Subtype | NA | NA | (RAMP) | Balac_1305 and |
| | I-U^{§§} | | | | GSU0054 |
| csb3 | Subtype | NA | NA | (RAMP) | Balac_1303^{§§} |
| | I-U | | | | |
| csx17 | Subtype | NA | NA | NA | Btus_2683 |
| | I-U | | | | |
| csx14 | Subtype | NA | NA | NA | GSU0052 |
| | I-U | | | | |
| csx10 | Subtype | csx10 | NA | (RAMP) | Caur_2274 |
| | I-U | | | | |
| csx16 | Subtype | VVA1548 | NA | NA | VVA1548 |
| | III-U | | | | |
| csaX | Subtype | csaX | NA | NA | SSO1438 |
| | III-U | | | | |
| csx3 | Subtype | csx3 | NA | NA | AF1864 |
| | III-U | | | | |
| csx1 | Subtype | csa3, csx1, | 1XMX and | COG1517 and | MJ1666, NE0113, |
| | III-U | csx2, | 2I71 | COG4006 | PF1127 and |
| | | DXTHG, | | | TM1812 |
| | | NE0113 and | | | |
| | | TIGR02710 | | | |
| csx15 | Unknown | NA | NA | TTE2665 | TTE2665 |
| csf1 | Type U | csf1 | NA | NA | AFE_1038 |
| csf2 | Type U | csf2 | NA | (RAMP) | AFE_1039 |
| csf3 | Type U | csf3 | NA | (RAMP) | AFE_1040 |
| csf4 | Type U | csf4 | NA | NA | AFE_1037 |

### 10. Functional Analysis of Candidate Molecules

Candidate Cas9 molecules, candidate gRNA molecules, candidate Cas9 molecule/gRNA molecule complexes, can be evaluated by art-known methods or as described herein. For example, exemplary methods for evaluating the endonuclease activity of Cas9 molecule have been described previously (Jinek 2012).

10.1 Binding and Cleavage Assay: Testing Cas9 Endonuclease Activity

The ability of a Cas9 molecule/gRNA molecule complex to bind to and cleave a target nucleic acid can be evaluated in a plasmid cleavage assay. In this assay, synthetic or in vitro-transcribed gRNA molecule is pre-annealed prior to the reaction by heating to 95° C. and slowly cooling down to room temperature. Native or restriction digest-linearized plasmid DNA (300 ng (˜8 nM)) is incubated for 60 min at 37° C. with purified Cas9 protein molecule (50-500 nM) and gRNA (50-500 nM, 1:1) in a Cas9 plasmid cleavage buffer (20 mM HEPES pH 7.5, 150 mM KC1, 0.5 mM DTT, 0.1 mM EDTA) with or without 10 mM MgCl₂. The reactions are stopped with 5× DNA loading buffer (30% glycerol, 1.2% SDS, 250 mM EDTA), resolved by a 0.8 or 1% agarose gel electrophoresis and visualized by ethidium bromide staining. The resulting cleavage products indicate whether the Cas9 molecule cleaves both DNA strands, or only one of the two strands. For example, linear DNA products indicate the cleavage of both DNA strands. Nicked open circular products indicate that only one of the two strands is cleaved.

Alternatively, the ability of a Cas9 molecule/gRNA molecule complex to bind to and cleave a target nucleic acid can be evaluated in an oligonucleotide DNA cleavage assay. In this assay, DNA oligonucleotides (10 pmol) are radiolabeled by incubating with 5 units T4 polynucleotide kinase and ˜3-6 pmol (˜20-40 mCi) [γ-32P]-ATP in 1× T4 polynucleotide kinase reaction buffer at 37° C. for 30 min, in a 50 μL reaction. After heat inactivation (65° C. for 20 min), reactions are purified through a column to remove unincorporated label. Duplex substrates (100 nM) are generated by annealing labeled oligonucleotides with equimolar amounts of unlabeled complementary oligonucleotide at 95° C. for 3 min, followed by slow cooling to room temperature. For cleavage assays, gRNA molecules are annealed by heating to 95° C. for 30 s, followed by slow cooling to room temperature. Cas9 (500 nM final concentration) is pre-incubated with the annealed gRNA molecules (500 nM) in cleavage assay buffer (20 mM HEPES pH 7.5, 100 mM KCl, 5 mM MgCl2, 1 mM DTT, 5% glycerol) in a total volume of 9 μL. Reactions are initiated by the addition of 1 μL target DNA (10 nM) and incubated for 1 h at 37° C. Reactions are quenched by the addition of 20 μL of loading dye (5 mM EDTA, 0.025% SDS, 5% glycerol in formamide) and heated to 95° C. for 5 min. Cleavage products are resolved on 12% denaturing polyacrylamide gels containing 7 M urea and visualized by phosphorimaging. The resulting cleavage products indicate that whether the complementary strand, the non-complementary strand, or both, are cleaved.

One or both of these assays can be used to evaluate the suitability of a candidate gRNA molecule or candidate Cas9 molecule.

10.2 Binding Assay: Testing the Binding of Cas9 Molecule to Target DNA

Exemplary methods for evaluating the binding of Cas9 molecule to target DNA have been described previously , e.g., in Jinek et al., SCIENCE 2012; 337(6096):816-821.

For example, in an electrophoretic mobility shift assay, target DNA duplexes are formed by mixing of each strand (10 nmol) in deionized water, heating to 95° C. for 3 min and slow cooling to room temperature. All DNAs are purified on 8% native gels containing 1× TBE. DNA bands are visualized by UV shadowing, excised, and eluted by soaking gel pieces in DEPC-treated H₂O. Eluted DNA is ethanol precipitated and dissolved in DEPC-treated H₂O. DNA samples are 5′ end labeled with [γ-32P]-ATP using T4 polynucleotide kinase for 30 min at 37° C. Polynucleotide kinase is heat denatured at 65° C. for 20 min, and unincorporated radiolabel is removed using a column. Binding assays are performed in buffer containing 20 mM HEPES pH 7.5, 100 mM KCl, 5 mM MgCl₂, 1 mM DTT and 10% glycerol in a total volume of 10 μL. Cas9 protein molecule is programmed with equimolar amounts of pre-annealed gRNA molecule and titrated from 100 pM to 1 μM. Radiolabeled DNA is added to a final concentration of 20 pM. Samples are incubated for 1 h at 37° C. and resolved at 4° C. on an 8% native polyacrylamide gel containing 1× TBE and 5 mM MgCl₂. Gels are dried and DNA visualized by phosphorimaging.

10.3 Differential Scanning Flourimetry (DSF)

The thermostability of Cas9-gRNA ribonucleoprotein (RNP) complexes can be measured via DSF. This technique measures the thermostability of a protein, which can increase under favorable conditions such as the addition of a binding RNA molecule, e.g., a gRNA.

The assay is performed using two different protocols, one to test the best stoichiometric ratio of gRNA:Cas9 protein and another to determine the best solution conditions for RNP formation.

To determine the best solution to form RNP complexes, a 2 uM solution of Cas9 in water+10× SYPRO Orange® (Life Technologies cat#S-6650) and dispensed into a 384 well plate. An equimolar amount of gRNA diluted in solutions with varied pH and salt is then added. After incubating at room temperature for 10′ and brief centrifugation to remove any bubbles,a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with 2 uM Cas9 in optimal buffer from assay 1 above and incubating at RT for 10′ in a 384 well plate. An equal volume of optimal buffer +10× SYPRO Orange® (Life Technologies cat#S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° increase in temperature every 10 seconds.

### 11. Genome Editing Approaches

Described herein are compositions, genome editing systems and methods for targeted alteration (e.g., knockout) of the CCR5 gene or CXCR4 gene, e.g., one or both alleles of the CCR5 gene or CXCR4 gene, e.g., using one or more of the approaches or pathways described herein, e.g., using NHEJ. Described herein are also methods for targeted knockdown of the CCR5 gene or CXCR4 gene.

11.1 NHEJ Approaches for Gene Targeting

In certain embodiments of the methods provided herein, NHEJ-mediated alteration is used to alter a CCR5 or a CXCR4 target position. As described herein, nuclease-induced non-homologous end-joining (NHEJ) can be used to target gene-specific knockouts. Nuclease-induced NHEJ can also be used to remove (e.g., delete) sequence insertions in a gene of interest.

In certain embodiments, the genomic alterations associated with the methods described herein rely on nuclease-induced NHEJ and the error-prone nature of the NHEJ repair pathway. NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein. The indel mutations generated by NHEJ are unpredictable in nature; however, at a given break site certain indel sequences are favored and are over represented in the population, likely due to small regions of microhomology. The lengths of deletions can vary widely; they are most commonly in the 1-50 bp range, but can reach greater than 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it can also be used to delete small sequence motifs (e.g., motifs less than or equal to 50 nucleotides in length) as long as the generation of a specific final sequence is not required. If a double-strand break is targeted near to a target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. In this way, DNA segments as large as several hundred kilobases can be deleted. Both of these approaches can be used to delete specific DNA sequences; however, the error-prone nature of NHEJ may still produce indel mutations at the site of repair.

Both double strand cleaving eaCas9 molecules and single strand, or nickase, eaCas9 molecules can be used in the methods and compositions described herein to generate NHEJ-mediated indels. NHEJ-mediated indels targeted to the early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

11.2 Placement of Double Strand or Single Strand Breaks Relative to the Target Position

In certain embodiments, in which a gRNA and Cas9 nuclease generate a double strand break for the purpose of inducing NHEJ-mediated indels, a gRNA, e.g., a unimolecular (or chimeric) or modular gRNA molecule, is configured to position one double-strand break in close proximity to a nucleotide of the target position. In certain embodiments, the cleavage site is between 0-30 bp away from the target position (e.g., less than 30, 25, 20, 15, 10, 9, 8, 7, 6, 5, 4, 3, 2 or 1 bp from the target position).

In certain embodiments, in which two gRNAs complexing with Cas9 nickases induce two single strand breaks for the purpose of inducing NHEJ-mediated indels, two gRNAs, e.g., independently, unimolecular (or chimeric) or modular gRNA, are configured to position two single-strand breaks to provide for NHEJ repair a nucleotide of the target position. In certain embodiments, the gRNAs are configured to position cuts at the same position, or within a few nucleotides of one another, on different strands, essentially mimicking a double strand break. In certain embodiments, the closer nick is between 0-30 bp away from the target position (e.g., less than 30, 25, 20, 15, 10, 9, 8, 7, 6, 5, 4, 3, 2 or 1 bp from the target position), and the two nicks are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50 , 45 to 50, 35 to 45, or 40 to 45 bp) and no more than 100 bp away from each other (e.g., no more than 90, 80, 70, 60, 50, 40, 30, 20 or 10 bp). In certain embodiments, the gRNAs are configured to place a single strand break on either side of a nucleotide of the target position.

Both double strand cleaving eaCas9 molecules and single strand, or nickase, eaCas9 molecules can be used in the methods and compositions described herein to generate breaks both sides of a target position. Double strand or paired single strand breaks may be generated on both sides of a target position to remove the nucleic acid sequence between the two cuts (e.g., the region between the two breaks in deleted). In certain embodiments, two gRNAs, e.g., independently, unimolecular (or chimeric) or modular gRNA, are configured to position a double-strand break on both sides of a target position. In an alternate embodiment, three gRNAs, e.g., independently, unimolecular (or chimeric) or modular gRNA, are configured to position a double strand break (i.e., one gRNA complexes with a cas9 nuclease) and two single strand breaks or paired single stranded breaks (i.e., two gRNAs complex with Cas9 nickases) on either side of the target position. In certain embodiments, four gRNAs, e.g., independently, unimolecular (or chimeric) or modular gRNA, are configured to generate two pairs of single stranded breaks (i.e., two pairs of two gRNAs complex with Cas9 nickases) on either side of the target position. The double strand break(s) or the closer of the two single strand nicks in a pair can ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50 , 45 to 50, 35 to 45, or 40 to 45 bp) and no more than 100 bp away from each other (e.g., no more than 90, 80, 70, 60, 50, 40, 30, 20, or 10 bp).

11.3 HDR Repair, HDR-Mediated Knock-In, and Template Nucleic Acids

In certain embodiments of the methods provided herein, HDR-mediated sequence alteration is used to alter the sequence of one or more nucleotides in a DMD gene using an exogenously provided template nucleic acid (also referred to herein as a donor construct). In certain embodiments, HDR-mediated alteration of a DMD target position occurs by HDR with an exogenously provided donor template or template nucleic acid. For example, the donor construct or template nucleic acid provides for alteration of a CCR5 or a CXCR4 target position. In certain embodiments, a plasmid donor is used as a template for homologous recombination. In certain embodiments, a single stranded donor template is used as a template for alteration of the CCR5 or CXCR4 target position by alternate methods of HDR (e.g., single strand annealing) between the target sequence and the donor template. Donor template-effected alteration of a CCR5 or a CXCR4 target position depends on cleavage by a Cas9 molecule. Cleavage by Cas9 can comprise a double strand break or two single strand breaks.

In certain embodiments, HDR-mediated sequence alteration is used to alter the sequence of one or more nucleotides in a CCR5 or a CXCR4 gene without using an exogenously provided template nucleic acid. In certain embodiments, alteration of a CCR5 or a CXCR4 target position occurs by HDR with endogenous genomic donor sequence. For example, the endogenous genomic donor sequence provides for alteration of the CCR5 or CXCR4 target position. In certain embodiments, the endogenous genomic donor sequence is located on the same chromosome as the target sequence. In certain embodiments, the endogenous genomic donor sequence is located on a different chromosome from the target sequence. Alteration of a CCR5 or a CXCR4 target position by endogenous genomic donor sequence depends on cleavage by a Cas9 molecule. Cleavage by Cas9 can comprise a double strand break or two single strand breaks.

In certain embodiments of the methods provided herein, HDR-mediated alteration is used to alter a single nucleotide in a CCR5 or a CXCR4 gene. These embodiments may utilize either one double-strand break or two single-strand breaks. In certain embodiments, a single nucleotide alteration is incorporated using (1) one double-strand break, (2) two single-strand breaks, (3) two double-strand breaks with a break occurring on each side of the target position, (4) one double-strand break and two single strand breaks with the double strand break and two single strand breaks occurring on each side of the target position, (5) four single-strand breaks with a pair of single-strand breaks occurring on each side of the target position, or (6) one single-strand break.

In certain embodiments, wherein a single-stranded template nucleic acid (e.g., a donor template) is used, the target position can be altered by alternative HDR. In certain embodiments, the donor template encodes an HIV fusion inhibitor. Examples of HIV fusion inhibitors include, but are not limited to, N36, T21, CP621-652, CP628-654, C34, DP107, IZN36, N36ccg, SFT, SC22EK, MTSC22, MTSC21, MTSC19, HP23, HP22, HP23E, T-1249, IQN17, IQN23, IQN36, IIN17, IQ22N17, II22N17, II15N17, IZN17, IZN23, IZN36, C46, C46-EHO, C37H6, and CP32M.

Donor template-effected alteration of a CCR5 or a CXCR4 target position depends on cleavage by a Cas9 molecule. Cleavage by Cas9 can comprise a nick, a double-strand break, or two single-strand breaks, e.g., one on each strand of the target nucleic acid. After introduction of the breaks on the target nucleic acid, resection occurs at the break ends resulting in single stranded overhanging DNA regions.

In canonical HDR, a double-stranded donor template is introduced, comprising homologous sequence to the target nucleic acid that can either be directly incorporated into the target nucleic acid or used as a template to change the sequence of the target nucleic acid. After resection at the break, repair can progress by different pathways, e.g., by the double Holliday junction model (or double-strand break repair, DSBR, pathway) or the synthesis-dependent strand annealing (SDSA) pathway. In the double Holliday junction model, strand invasion by the two single stranded overhangs of the target nucleic acid to the homologous sequences in the donor template occurs, resulting in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in alteration of the target nucleic acid. Crossover with the donor template may occur upon resolution of the junctions. In the SDSA pathway, only one single stranded overhang invades the donor template and new DNA is synthesized from the end of the invading strand to fill the gap resulting from resection. The newly synthesized DNA then anneals to the remaining single stranded overhang, new DNA is synthesized to fill in the gap, and the strands are ligated to produce the altered DNA duplex.

In alternative HDR, a single strand donor template, e.g., template nucleic acid, is introduced. A nick, single strand break, or double strand break at the target nucleic acid, for altering a desired target position, is mediated by a Cas9 molecule, e.g., described herein, and resection at the break occurs to reveal single stranded overhangs. Incorporation of the sequence of the template nucleic acid to alter a CCR5 or a CXCR4 target position typically occurs by the SDSA pathway, as described above.

Additional details on template nucleic acids are provided in Section IV entitled “Template nucleic acids” in International Application PCT/US2014/057905.

In certain embodiments, double strand cleavage is effected by a Cas9 molecule having cleavage activity associated with an HNH-like domain and cleavage activity associated with a RuvC-like domain, e.g., an N-terminal RuvC-like domain, e.g., a wild-type Cas9. Such embodiments require only a single gRNA.

In certain embodiments, one single-strand break, or nick, is effected by a Cas9 molecule having nickase activity, e.g., a Cas9 nickase as described herein (such as a D10A Cas9 nickase). A nicked target nucleic acid can be a substrate for alt-HDR.

In certain embodiments, two single-strand breaks, or nicks, are effected by a Cas9 molecule having nickase activity, e.g., cleavage activity associated with an HNH-like domain or cleavage activity associated with an N-terminal RuvC-like domain. Such embodiments usually require two gRNAs, one for placement of each single-strand break. In certain embodiments, the Cas9 molecule having nickase activity cleaves the strand to which the gRNA hybridizes, but not the strand that is complementary to the strand to which the gRNA hybridizes. In certain embodiments, the Cas9 molecule having nickase activity does not cleave the strand to which the gRNA hybridizes, but rather cleaves the strand that is complementary to the strand to which the gRNA hybridizes.

In certain embodiments, the nickase has HNH activity, e.g., a Cas9 molecule having the RuvC activity inactivated, e.g., a Cas9 molecule having a mutation at D10, e.g., the D10A mutation (see, e.g., SEQ ID NO:10). D10A inactivates RuvC; therefore, the Cas9 nickase has (only) HNH activity and can cut on the strand to which the gRNA hybridizes (e.g., the complementary strand, which does not have the NGG PAM on it). In certain embodiments, a Cas9 molecule having an H840, e.g., an H840A, mutation can be used as a nickase. H840A inactivates HNH; therefore, the Cas9 nickase has (only) RuvC activity and cuts on the non-complementary strand (e.g., the strand that has the NGG PAM and whose sequence is identical to the gRNA). In certain embodiments, a Cas9 molecule having an N863 mutation, e.g., the N863A mutation, mutation can be used as a nickase. N863A inactivates HNH therefore the Cas9 nickase has (only) RuvC activity and cuts on the non-complementary strand (the strand that has the NGG PAM and whose sequence is identical to the gRNA). In certain embodiments, a Cas9 molecule having an N580 mutation, e.g., the N580A mutation, mutation can be used as a nickase. N580A inactivates HNH therefore the Cas9 nickase has (only) RuvC activity and cuts on the non-complementary strand (the strand that has the NGG PAM and whose sequence is identical to the gRNA).

In certain embodiments, in which a nickase and two gRNAs are used to position two single strand nicks, one nick is on the+strand and one nick is on the−strand of the target nucleic acid. The PAMs can be outwardly facing. The gRNAs can be selected such that the gRNAs are separated by, from about 0-50, 0-100, or 0-200 nucleotides. In certain embodiments, there is no overlap between the target sequences that are complementary to the targeting domains of the two gRNAs. In certain embodiments, the gRNAs do not overlap and are separated by as much as 50, 100, or 200 nucleotides. In certain embodiments, the use of two gRNAs can increase specificity, e.g., by decreasing off-target binding (Ran 2013).

In certain embodiments, a single nick can be used to induce HDR, e.g., alt-HDR. It is contemplated herein that a single nick can be used to increase the ratio of HR to NHEJ at a given cleavage site. In certain embodiments, a single strand break is formed in the strand of the target nucleic acid to which the targeting domain of said gRNA is complementary. In certain embodiments, a single strand break is formed in the strand of the target nucleic acid other than the strand to which the targeting domain of said gRNA is complementary.

11.4 Placement of Double Strand or Single Strand Breaks Relative to the Target Position

A double strand break or single strand break in one of the strands should be sufficiently close to a CCR5 or a CXCR4 target position that an alteration is produced in the desired region. In certain embodiments, the distance is not more than 50, 100, 200, 300, 350 or 400 nucleotides. In certain embodiments, the break should be sufficiently close to target position such that the target position is within the region that is subject to exonuclease-mediated removal during end resection. If the distance between the CCR5 or a CXCR4 target position and a break is too great, the sequence desired to be altered may not be included in the end resection and, therefore, may not be altered, as donor sequence, either exogenously provided donor sequence or endogenous genomic donor sequence, in certain embodiments is only used to alter sequence within the end resection region.

In certain embodiments, the methods described herein introduce one or more breaks near a CCR5 or a CXCR4 target position. In certain of these embodiments, two or more breaks are introduced that flank a CCR5 or a CXCR4 target position. The two or more breaks remove (e.g., delete) a genomic sequence including a CCR5 or a CXCR4 target position. All methods described herein result in altering a CCR5 or a CXCR4 target position within a CCR5 or a CXCR4 gene.

In certain embodiments, the gRNA targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, or 200 nucleotides of the region desired to be altered, e.g., a mutation. The break, e.g., a double strand or single strand break, can be positioned upstream or downstream of the region desired to be altered, e.g., a mutation. In certain embodiments, a break is positioned within the region desired to be altered, e.g., within a region defined by at least two mutant nucleotides. In certain embodiments, a break is positioned immediately adjacent to the region desired to be altered, e.g., immediately upstream or downstream of a mutation.

In certain embodiments, a single strand break is accompanied by an additional single strand break, positioned by a second gRNA molecule, as discussed below. For example, the targeting domains bind configured such that a cleavage event, e.g., the two single strand breaks, are positioned within 1, 2, 3, 4, 5, 10, 15, 20, 25, 30, 35, 40, 45, 50, 60, 70, 80, 90, 100, 150, or 200 nucleotides of a target position. In certain embodiments, the first and second gRNA molecules are configured such that, when guiding a Cas9 nickase, a single strand break can be accompanied by an additional single strand break, positioned by a second gRNA, sufficiently close to one another to result in alteration of the desired region. In certain embodiments, the first and second gRNA molecules are configured such that a single strand break positioned by said second gRNA is within 10, 20, 30, 40, or 50 nucleotides of the break positioned by said first gRNA molecule, e.g., when the Cas9 is a nickase. In certain embodiments, the two gRNA molecules are configured to position cuts at the same position, or within a few nucleotides of one another, on different strands, e.g., essentially mimicking a double strand break.

In certain embodiments in which a gRNA (unimolecular (or chimeric) or modular gRNA) and Cas9 nuclease induce a double strand break for the purpose of inducing HDR-mediated sequence alteration, the cleavage site is between 0-200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target position. In certain embodiments, the cleavage site is between 0-100 bp (e.g., 0 to 75, 0 to 50, 0 to 25, 25 to 100, 25 to 75, 25 to 50, 50 to 100, 50 to 75 or 75 to 100 bp) away from the target position.

In certain embodiments, one can promote HDR by using nickases to generate a break with overhangs. While not wishing to be bound by theory, the single stranded nature of the overhangs can enhance the cell's likelihood of repairing the break by HDR as opposed to, e.g., NHEJ. Specifically, in certain embodiments, HDR is promoted by selecting a first gRNA that targets a first nickase to a first target sequence, and a second gRNA that targets a second nickase to a second target sequence which is on the opposite DNA strand from the first target sequence and offset from the first nick.

In certain embodiments, the targeting domain of a gRNA molecule is configured to position a cleavage event sufficiently far from a preselected nucleotide that the nucleotide is not altered. In certain embodiments, the targeting domain of a gRNA molecule is configured to position an intronic cleavage event sufficiently far from an intron/exon border, or naturally occurring splice signal, to avoid alteration of the exonic sequence or unwanted splicing events. The gRNA molecule may be a first, second, third and/or fourth gRNA molecule, as described herein.

11.5 Placement of a First Break and a Second Break Relative to Each Other

In certain embodiments, a double strand break can be accompanied by an additional double strand break, positioned by a second gRNA molecule, as is discussed below.

In certain embodiments, a double strand break can be accompanied by two additional single strand breaks, positioned by a second gRNA molecule and a third gRNA molecule.

In certain embodiments, a first and second single strand breaks can be accompanied by two additional single strand breaks positioned by a third gRNA molecule and a fourth gRNA molecule.

When two or more gRNAs are used to position two or more cleavage events, e.g., double strand or single strand breaks, in a target nucleic acid, it is contemplated that the two or more cleavage events may be made by the same or different Cas9 proteins. For example, when two gRNAs are used to position two double stranded breaks, a single Cas9 nuclease may be used to create both double stranded breaks. When two or more gRNAs are used to position two or more single stranded breaks (nicks), a single Cas9 nickase may be used to create the two or more nicks. When two or more gRNAs are used to position at least one double stranded break and at least one single stranded break, two Cas9 proteins may be used, e.g., one Cas9 nuclease and one Cas9 nickase. In certain embodiments, two or more Cas9 proteins are used, and the two or more Cas9 proteins may be delivered sequentially to control specificity of a double stranded versus a single stranded break at the desired position in the target nucleic acid.

In certain embodiments, the targeting domain of the first gRNA molecule and the targeting domain of the second gRNA molecules are complementary to opposite strands of the target nucleic acid molecule. In certain embodiments, the gRNA molecule and the second gRNA molecule are configured such that the PAMs are oriented outward.

In certain embodiments, two gRNA are selected to direct Cas9-mediated cleavage at two positions that are a preselected distance from each other. In certain embodiments, the two points of cleavage are on opposite strands of the target nucleic acid. In certain embodiments, the two cleavage points form a blunt ended break, and in other embodiments, they are offset so that the DNA ends comprise one or two overhangs (e.g., one or more 5′ overhangs and/or one or more 3′ overhangs). In certain embodiments, each cleavage event is a nick. In certain embodiments, the nicks are close enough together that they form a break that is recognized by the double stranded break machinery (as opposed to being recognized by, e.g., the SSBr machinery). In certain embodiments, the nicks are far enough apart that they create an overhang that is a substrate for HDR, i.e., the placement of the breaks mimics a DNA substrate that has experienced some resection. For instance, in certain embodiments the nicks are spaced to create an overhang that is a substrate for processive resection. In certain embodiments, the two breaks are spaced within 25-65 nucleotides of each other. The two breaks may be, e.g., about 25, 30, 35, 40, 45, 50, 55, 60, or 65 nucleotides of each other. The two breaks may be, e.g., at least about 25, 30, 35, 40, 45, 50, 55, 60, or 65 nucleotides of each other. The two breaks may be, e.g., at most about 30, 35, 40, 45, 50, 55, 60, or 65 nucleotides of each other. In certain embodiments, the two breaks are about 25-30, 30-35, 35-40, 40-45, 45-50, 50-55, 55-60, or 60-65 nucleotides of each other.

In certain embodiments, the break that mimics a resected break comprises a 3′ overhang (e.g., generated by a DSB and a nick, where the nick leaves a 3′ overhang), a 5′ overhang (e.g., generated by a DSB and a nick, where the nick leaves a 5′ overhang), a 3′ and a 5′ overhang (e.g., generated by three cuts), two 3′ overhangs (e.g., generated by two nicks that are offset from each other), or two 5′ overhangs (e.g., generated by two nicks that are offset from each other).

In certain embodiments in which two gRNAs (independently, unimolecular (or chimeric) or modular gRNA) complexing with Cas9 nickases induce two single strand breaks for the purpose of inducing HDR-mediated alteration, the closer nick is between 0-200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, or 75 to 100 bp) away from the target position and the two nicks can ideally be within 25-65 by of each other (e.g., 25 to 50, 25 to 45, 25 to 40, 25 to 35, 25 to 30, 30 to 55, 30 to 50, 30 to 45, 30 to 40, 30 to 35, 35 to 55, 35 to 50, 35 to 45, 35 to 40, 40 to 55, 40 to 50, 40 to 45 bp, 45 to 50 bp, 50 to 55 bp, 55 to 60 bp, or 60 to 65 bp) and no more than 100 bp away from each other (e.g., no more than 90, 80, 70, 60, 50, 40, 30, 20, 10, or 5 bp away from each other). In certain embodiments, the cleavage site is between 0-100 bp (e.g., 0 to 75, 0 to 50, 0 to 25, 25 to 100, 25 to 75, 25 to 50, 50 to 100, 50 to 75, or 75 to 100 bp) away from the target position.

In certain embodiments, two gRNAs, e.g., independently, unimolecular (or chimeric) or modular gRNA, are configured to position a double-strand break on both sides of a target position. In certain embodiments, three gRNAs, e.g., independently, unimolecular (or chimeric) or modular gRNA, are configured to position a double strand break (i.e., one gRNA complexes with a cas9 nuclease) and two single strand breaks or paired single stranded breaks (i.e., two gRNAs complex with Cas9 nickases) on either side of the target position. In certain embodiments, four gRNAs, e.g., independently, unimolecular (or chimeric) or modular gRNA, are configured to generate two pairs of single stranded breaks (i.e., two pairs of two gRNAs complex with Cas9 nickases) on either side of the target position. The double strand break(s) or the closer of the two single strand nicks in a pair can ideally be within 0-500 bp of the target position (e.g., no more than 450, 400, 350, 300, 250, 200, 150, 100, 50 or 25 bp from the target position). When nickases are used, the two nicks in a pair are, in certain embodiments, within 25-65 bp of each other (e.g., between 25 to 55, 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, 40 to 45 bp, 45 to 50 bp, 50 to 55 bp, 55 to 60 bp, or 60 to 65 bp) and no more than 100 bp away from each other (e.g., no more than 90, 80, 70, 60, 50, 40, 30, or 20 or 10 bp).

When two gRNAs are used to target Cas9 molecules to breaks, different combinations of Cas9 molecules are envisioned. In certain embodiments, a first gRNA is used to target a first Cas9 molecule to a first target position, and a second gRNA is used to target a second Cas9 molecule to a second target position. In certain embodiments, the first Cas9 molecule creates a nick on the first strand of the target nucleic acid, and the second Cas9 molecule creates a nick on the opposite strand, resulting in a double stranded break (e.g., a blunt ended cut or a cut with overhangs).

Different combinations of nickases can be chosen to target one single stranded break to one strand and a second single stranded break to the opposite strand. When choosing a combination, one can take into account that there are nickases having one active RuvC-like domain, and nickases having one active HNH domain. In certain embodiments, a RuvC-like domain cleaves the non-complementary strand of the target nucleic acid molecule. In certain embodiments, an HNH-like domain cleaves a single stranded complementary domain, e.g., a complementary strand of a double stranded nucleic acid molecule. Generally, if both Cas9 molecules have the same active domain (e.g., both have an active RuvC domain or both have an active HNH domain), one can choose two gRNAs that bind to opposite strands of the target. In more detail, in certain embodiments a first gRNA is complementary with a first strand of the target nucleic acid and binds a nickase having an active RuvC-like domain and causes that nickase to cleave the strand that is non-complementary to that first gRNA, i.e., a second strand of the target nucleic acid; and a second gRNA is complementary with a second strand of the target nucleic acid and binds a nickase having an active RuvC-like domain and causes that nickase to cleave the strand that is non-complementary to that second gRNA, i.e., the first strand of the target nucleic acid. Conversely, in certain embodiments, a first gRNA is complementary with a first strand of the target nucleic acid and binds a nickase having an active HNH domain and causes that nickase to cleave the strand that is complementary to that first gRNA, i.e., a first strand of the target nucleic acid; and a second gRNA is complementary with a second strand of the target nucleic acid and binds a nickase having an active HNH domain and causes that nickase to cleave the strand that is complementary to that second gRNA, i.e., the second strand of the target nucleic acid. In another arrangement, if one Cas9 molecule has an active RuvC-like domain and the other Cas9 molecule has an active HNH domain, the gRNAs for both Cas9 molecules can be complementary to the same strand of the target nucleic acid, so that the Cas9 molecule with the active RuvC-like domain can cleave the non-complementary strand and the Cas9 molecule with the HNH domain can cleave the complementary strand, resulting in a double stranded break.

11.6 Homology Arms of the Donor Template

A homology arm should extend at least as far as the region in which end resection may occur, e.g., in order to allow the resected single stranded overhang to find a complementary region within the donor template. The overall length could be limited by parameters such as plasmid size or viral packaging limits. In certain embodiments, a homology arm does not extend into repeated elements, e.g., Alu repeats or LINE repeats.

Exemplary homology arm lengths include at least 50, 100, 250, 500, 750, 1000, 2000, 3000, 4000, or 5000 nucleotides. In certain embodiments, the homology arm length is 50-100, 100-250, 250-500, 500-750, 750-1000, 1000-2000, 2000-3000, 3000-4000, or 4000-5000 nucleotides.

A template nucleic acid, as that term is used herein, refers to a nucleic acid sequence which can be used in conjunction with a Cas9 molecule and a gRNA molecule to alter the structure of a CCR5 or a CXCR4 target position. In certain embodiments, the CCR5 or CXCR4 target position can be a site between two nucleotides, e.g., adjacent nucleotides, on the target nucleic acid into which one or more nucleotides is added. Alternatively, the CCR5 or CXCR4 target position may comprise one or more nucleotides that are altered by a template nucleic acid.

In certain embodiments, the target nucleic acid is modified to have some or all of the sequence of the template nucleic acid, typically at or near cleavage site(s). In certain embodiments, the template nucleic acid is single stranded. In certain embodiments, the template nucleic acid is double stranded. In certain embodiments, the template nucleic acid is DNA, e.g., double stranded DNA. In certain embodiments, the template nucleic acid is single stranded DNA. In certain embodiments, the template nucleic acid is encoded on the same vector backbone, e.g. AAV genome, plasmid DNA, as the Cas9 and gRNA. In certain embodiments, the template nucleic acid is excised from a vector backbone in vivo, e.g., it is flanked by gRNA recognition sequences. In certain embodiments, the template nucleic acid comprises endogenous genomic sequence.

In certain embodiments, the template nucleic acid alters the structure of the target position by participating in an HDR event. In certain embodiments, the template nucleic acid alters the sequence of the target position. In certain embodiments, the template nucleic acid results in the incorporation of a modified, or non-naturally occurring base into the target nucleic acid.

Typically, the template sequence undergoes a breakage mediated or catalyzed recombination with the target sequence. In certain embodiments, the template nucleic acid includes sequence that corresponds to a site on the target sequence that is cleaved by an eaCas9 mediated cleavage event. In certain embodiments, the template nucleic acid includes sequence that corresponds to both a first site on the target sequence that is cleaved in a first Cas9 mediated event, and a second site on the target sequence that is cleaved in a second Cas9 mediated event.

A template nucleic acid typically comprises the following components:

[5′ homology arm]-[replacement sequence]-[3′ homology arm].

The homology arms provide for recombination into the chromosome, thus replacing the undesired element, e.g., a mutation or signature, with the replacement sequence. In certain embodiments, the homology arms flank the most distal cleavage sites.

In certain embodiments, the 3′ end of the 5′ homology arm is the position next to the 5′ end of the replacement sequence. In certain embodiments, the 5′ homology arm can extend at least 10, 20, 30, 40, 50, 100, 200, 300, 400, 500, 600, 700, 800, 900, 1000, 1500, 2000, 3000, 4000, or 5000 nucleotides 5′ from the 5′ end of the replacement sequence.

In certain embodiments, the 5′ end of the 3′ homology arm is the position next to the 3′ end of the replacement sequence. In certain embodiments, the 3′ homology arm can extend at least 10, 20, 30, 40, 50, 100, 200, 300, 400, 500, 600, 700, 800, 900, 1000, 1500, 2000, 3000, 4000, or 5000 nucleotides 3′ from the 3′ end of the replacement sequence.

In certain embodiments, to alter one or more nucleotides at a CCR5 or a CXCR4 target position, the homology arms, e.g., the 5′ and 3′ homology arms, may each comprise about 1000 bp of sequence flanking the most distal gRNAs (e.g., 1000 bp of sequence on either side of the CCR5 or CXCR4 target position).

In certain embodiments, one or both homology arms may be shortened to avoid including certain sequence repeat elements, e.g., Alu repeats or LINE elements. For example, a 5′ homology arm may be shortened to avoid a sequence repeat element. In certain embodiments, a 3′ homology arm may be shortened to avoid a sequence repeat element. In certain embodiments, both the 5′ and the 3′ homology arms may be shortened to avoid including certain sequence repeat elements.

In certain embodiments, template nucleic acids for altering the sequence of a CCR5 or a CXCR4 target position may be designed for use as a single-stranded oligonucleotide, e.g., a single-stranded oligodeoxynucleotide (ssODN). When using a ssODN, 5′ and 3′ homology arms may range up to about 200 bp in length, e.g., at least 25, 50, 75, 100, 125, 150, 175, or 200 bp in length. Longer homology arms are also contemplated for ssODNs as improvements in oligonucleotide synthesis continue to be made. In certain embodiments, a longer homology arm is made by a method other than chemical synthesis, e.g., by denaturing a long double stranded nucleic acid and purifying one of the strands, e.g., by affinity for a strand-specific sequence anchored to a solid substrate.

In certain embodiments, alt-HDR proceeds more efficiently when the template nucleic acid has extended homology 5′ to the nick (i.e., in the 5′ direction of the nicked strand). Accordingly, in certain embodiments, the template nucleic acid has a longer homology arm and a shorter homology arm, wherein the longer homology arm can anneal 5′ of the nick. In certain embodiments, the arm that can anneal 5′ to the nick is at least 25, 50, 75, 100, 125, 150, 175, or 200, 300, 400, 500, 600, 700, 800, 900, 1000, 1500, 2000, 3000, 4000, or 5000 nucleotides from the nick or the 5′ or 3′ end of the replacement sequence. In certain embodiments, the arm that can anneal 5′ to the nick is at least about 10%, about 20%, about 30%, about 40%, or about 50% longer than the arm that can anneal 3′ to the nick. In certain embodiments, the arm that can anneal 5′ to the nick is at least 2×, 3×, 4×, or 5× longer than the arm that can anneal 3′ to the nick. Depending on whether a ssDNA template can anneal to the intact strand or the nicked strand, the homology arm that anneals 5′ to the nick may be at the 5′ end of the ssDNA template or the 3′ end of the ssDNA template, respectively.

Similarly, in certain embodiments, the template nucleic acid has a 5′ homology arm, a replacement sequence, and a 3′ homology arm, such that the template nucleic acid has extended homology to the 5′ of the nick. For example, the 5′ homology arm and 3′ homology arm may be substantially the same length, but the replacement sequence may extend farther 5′ of the nick than 3′ of the nick. In certain embodiments, the replacement sequence extends at least about 10%, about 20%, about 30%, about 40%, about 50%, 2x, 3x, 4x, or 5x further to the 5′ end of the nick than the 3′ end of the nick.

In certain embodiments, alt-HDR proceeds more efficiently when the template nucleic acid is centered on the nick. Accordingly, in certain embodiments, the template nucleic acid has two homology arms that are essentially the same size. For instance, the first homology arm of a template nucleic acid may have a length that is within about 10%, about 9%, about 8%, about 7%, about 6%, about 5%, about 4%, about 3%, about 2%, or about 1% of the second homology arm of the template nucleic acid.

Similarly, in certain embodiments, the template nucleic acid has a 5′ homology arm, a replacement sequence, and a 3′ homology arm, such that the template nucleic acid extends substantially the same distance on either side of the nick. For example, the homology arms may have different lengths, but the replacement sequence may be selected to compensate for this. For example, the replacement sequence may extend further 5′ from the nick than it does 3′ of the nick, but the homology arm 5′ of the nick is shorter than the homology arm 3′ of the nick, to compensate. The converse is also possible, e.g., that the replacement sequence may extend further 3′ from the nick than it does 5′ of the nick, but the homology arm 3′ of the nick is shorter than the homology arm 5′ of the nick, to compensate.

11.7 Template Nucleic Acids

In certain embodiments, the template nucleic acid is double stranded. In certain embodiments, the template nucleic acid is single stranded. In certain embodiments, the template nucleic acid comprises a single stranded portion and a double stranded portion. In certain embodiments, the template nucleic acid comprises about 50 to 100 bp, e.g., 55 to 95, 60 to 90, 65 to 85, or 70 to 80 bp, homology on either side of the nick and/or replacement sequence. In certain embodiments, the template nucleic acid comprises about 50, 55, 60, 65, 70, 75, 80, 85, 90, 95, or 100 bp homology 5′ of the nick or replacement sequence, 3′ of the nick or replacement sequence, or both 5′ and 3′ of the nick or replacement sequences.

In certain embodiments, the template nucleic acid comprises about 150 to 200 bp, e.g., 155 to 195, 160 to 190, 165 to 185, or 170 to 180 bp, homology 3′ of the nick and/or replacement sequence. In certain embodiments, the template nucleic acid comprises about 150, 155, 160, 165, 170, 175, 180, 185, 190, 195, or 200 bp homology 3′ of the nick or replacement sequence. In certain embodiments, the template nucleic acid comprises less than about 100, 90, 80, 70, 60, 50, 40, 30, 20, 15, or 10 bp homology 5′ of the nick or replacement sequence.

In certain embodiment, the template nucleic acid comprises about 150 to 200 bp, e.g., 155 to 195, 160 to 190, 165 to 185, or 170 to 180 bp, homology 5′ of the nick and/or replacement sequence. In certain embodiment, the template nucleic acid comprises about 150, 155, 160, 165, 170, 175, 180, 185, 190, 195, or 200 bp homology 5′ of the nick or replacement sequence. In certain embodiments, the template nucleic acid comprises less than about 100, 90, 80, 70, 60, 50, 40, 30, 20, 15, or 10 bp homology 3′ of the nick or replacement sequence.

In certain embodiments, the template nucleic acid comprises a nucleotide sequence, e.g., of one or more nucleotides, that can be added to or can template a change in the target nucleic acid. In other embodiments, the template nucleic acid comprises a nucleotide sequence that may be used to modify the target position.

The template nucleic acid may comprise a replacement sequence. In certain embodiments, the template nucleic acid comprises a 5′ homology arm. In certain embodiments, the template nucleic acid comprises a 3′ homology arm.

In certain embodiments, the template nucleic acid is linear double stranded DNA. The length may be, e.g., about 150-200 bp, e.g., about 150, 160, 170, 180, 190, or 200 bp. The length may be, e.g., at least 150, 160, 170, 180, 190, or 200 bp. In certain embodiments, the length is no greater than 150, 160, 170, 180, 190, or 200 bp. In certain embodiments, a double stranded template nucleic acid has a length of about 160 bp, e.g., about 155-165, 150-170, 140-180, 130-190, 120-200, 110-210, 100-220, 90-230, or 80-240 bp.

The template nucleic acid can be linear single stranded DNA. In certain embodiments, the template nucleic acid is (i) linear single stranded DNA that can anneal to the nicked strand of the target nucleic acid, (ii) linear single stranded DNA that can anneal to the intact strand of the target nucleic acid, (iii) linear single stranded DNA that can anneal to the plus strand of the target nucleic acid, (iv) linear single stranded DNA that can anneal to the minus strand of the target nucleic acid, or more than one of the preceding. The length may be, e.g., about 150-200 nucleotides, e.g., about 150, 160, 170, 180, 190, or 200 nucleotides. The length may be, e.g., at least 150, 160, 170, 180, 190, or 200 nucleotides. In certain embodiments, the length is no greater than 150, 160, 170, 180, 190, or 200 nucleotides. In certain embodiments, a single stranded template nucleic acid has a length of about 160 nucleotides, e.g., about 155-165, 150-170, 140-180, 130-190, 120-200, 110-210, 100-220, 90-230, or 80-240 nucleotides.

In certain embodiments, the template nucleic acid is circular double stranded DNA, e.g., a plasmid. In certain embodiments, the template nucleic acid comprises about 500 to 1000 bp of homology on either side of the replacement sequence and/or the nick. In certain embodiments, the template nucleic acid comprises about 300, 400, 500, 600, 700, 800, 900, 1000, 1500, or 2000 bp of homology 5′ of the nick or replacement sequence, 3′ of the nick or replacement sequence, or both 5′ and 3′ of the nick or replacement sequence. In certain embodiments, the template nucleic acid comprises at least 300, 400, 500, 600, 700, 800, 900, 1000, 1500, or 2000 bp of homology 5′ of the nick or replacement sequence, 3′ of the nick or replacement sequence, or both 5′ and 3′ of the nick or replacement sequence. In certain embodiments, the template nucleic acid comprises no more than 300, 400, 500, 600, 700, 800, 900, 1000, 1500, or 2000 bp of homology 5′ of the nick or replacement sequence, 3′ of the nick or replacement sequence, or both 5′ and 3′ of the nick or replacement sequence.

In certain embodiments, one or both homology arms may be shortened to avoid including certain sequence repeat elements, e.g., Alu repeats, LINE elements. For example, a 5′ homology arm may be shortened to avoid a sequence repeat element, while a 3′ homology arm may be shortened to avoid a sequence repeat element. In certain embodiments, both the 5′ and the 3′ homology arms may be shortened to avoid including certain sequence repeat elements.

In certain embodiments, the template nucleic acid is an adenovirus vector, e.g., an AAV vector, e.g., a ssDNA molecule of a length and sequence that allows it to be packaged in an AAV capsid. The vector may be, e.g., less than 5 kb and may contain an ITR sequence that promotes packaging into the capsid. The vector may be integration-deficient. In certain embodiments, the template nucleic acid comprises about 150 to 1000 nucleotides of homology on either side of the replacement sequence and/or the nick. In certain embodiments, the template nucleic acid comprises about 100, 150, 200, 300, 400, 500, 600, 700, 800, 900, 1000, 1500, or 2000 nucleotides 5′ of the nick or replacement sequence, 3′ of the nick or replacement sequence, or both 5′ and 3′ of the nick or replacement sequence. In certain embodiments, the template nucleic acid comprises at least 100, 150, 200, 300, 400, 500, 600, 700, 800, 900, 1000, 1500, or 2000 nucleotides 5′ of the nick or replacement sequence, 3′ of the nick or replacement sequence, or both 5′ and 3′ of the nick or replacement sequence. In certain embodiments, the template nucleic acid comprises at most 100, 150, 200, 300, 400, 500, 600, 700, 800, 900, 1000, 1500, or 2000 nucleotides 5′ of the nick or replacement sequence, 3′ of the nick or replacement sequence, or both 5′ and 3′ of the nick or replacement sequence.

In certain embodiments, the template nucleic acid is a lentiviral vector, e.g., an IDLV (integration deficiency lentivirus). In certain embodiments, the template nucleic acid comprises about 500 to 1000 bp of homology on either side of the replacement sequence and/or the nick. In certain embodiments, the template nucleic acid comprises about 300, 400, 500, 600, 700, 800, 900, 1000, 1500, or 2000 bp of homology 5′ of the nick or replacement sequence, 3′ of the nick or replacement sequence, or both 5′ and 3′ of the nick or replacement sequence. In certain embodiments, the template nucleic acid comprises at least 300, 400, 500, 600, 700, 800, 900, 1000, 1500, or 2000 bp of homology 5′ of the nick or replacement sequence, 3′ of the nick or replacement sequence, or both 5′ and 3′ of the nick or replacement sequence. In certain embodiments, the template nucleic acid comprises no more than 300, 400, 500, 600, 700, 800, 900, 1000, 1500, or 2000 bp of homology 5′ of the nick or replacement sequence, 3′ of the nick or replacement sequence, or both 5′ and 3′ of the nick or replacement sequence.

In certain embodiments, the template nucleic acid comprises one or more mutations, e.g., silent mutations, that prevent Cas9 from recognizing and cleaving the template nucleic acid. The template nucleic acid may comprise, e.g., at least 1, 2, 3, 4, 5, 10, 20, or 30 silent mutations relative to the corresponding sequence in the genome of the cell to be altered. In certain embodiments, the template nucleic acid comprises at most 2, 3, 4, 5, 10, 20, 30, or 50 silent mutations relative to the corresponding sequence in the genome of the cell to be altered. In certain embodiments, the cDNA comprises one or more mutations, e.g., silent mutations that prevent Cas9 from recognizing and cleaving the template nucleic acid. The template nucleic acid may comprise, e.g., at least 1, 2, 3, 4, 5, 10, 20, or 30 silent mutations relative to the corresponding sequence in the genome of the cell to be altered. In certain embodiments, the template nucleic acid comprises at most 2, 3, 4, 5, 10, 20, 30, or 50 silent mutations relative to the corresponding sequence in the genome of the cell to be altered.

In certain embodiments, the 5′ and 3′ homology arms each comprise a length of sequence flanking the nucleotides corresponding to the replacement sequence. In certain embodiments, a template nucleic acid comprises a replacement sequence flanked by a 5′ homology arm and a 3′ homology arm each independently comprising 10 or more, 20 or more, 50 or more, 100 or more, 150 or more, 200 or more, 250 or more, 300 or more, 350 or more, 400 or more, 450 or more, 500 or more, 550 or more, 600 or more, 650 or more, 700 or more, 750 or more, 800 or more, 850 or more, 900 or more, 1000 or more, 1100 or more, 1200 or more, 1300 or more, 1400 or more, 1500 or more, 1600 or more, 1700 or more, 1800 or more, 1900 or more, or 2000 or more nucleotides. In certain embodiments, a template nucleic acid comprises a replacement sequence flanked by a 5′ homology arm and a 3′ homology arm each independently comprising at least 50, 100, or 150 nucleotides, but not long enough to include a repeated element. In certain embodiments, a template nucleic acid comprises a replacement sequence flanked by a 5′ homology arm and a 3′ homology arm each independently comprising 5 to 100, 10 to 150, or 20 to 150 nucleotides. In certain embodiments, the replacement sequence optionally comprises a promoter and/or polyA signal.

11.8 Single-Strand Annealing

Single strand annealing (SSA) is another DNA repair process that repairs a double-strand break between two repeat sequences present in a target nucleic acid. Repeat sequences utilized by the SSA pathway are generally greater than 30 nucleotides in length. Resection at the break ends occurs to reveal repeat sequences on both strands of the target nucleic acid. After resection, single strand overhangs containing the repeat sequences are coated with RPA protein to prevent the repeats sequences from inappropriate annealing, e.g., to themselves. RAD52 binds to and each of the repeat sequences on the overhangs and aligns the sequences to enable the annealing of the complementary repeat sequences. After annealing, the single-strand flaps of the overhangs are cleaved. New DNA synthesis fills in any gaps, and ligation restores the DNA duplex. As a result of the processing, the DNA sequence between the two repeats is deleted. The length of the deletion can depend on many factors including the location of the two repeats utilized, and the pathway or processivity of the resection.

In contrast to HDR pathways, SSA does not require a template nucleic acid to alter a target nucleic acid sequence. Instead, the complementary repeat sequence is utilized.

11.9 Other DNA Repair Pathways

11.9.1 SSBR (Single Strand Break Repair)

Single-stranded breaks (SSB) in the genome are repaired by the SSBR pathway, which is a distinct mechanism from the DSB repair mechanisms discussed above. The SSBR pathway has four major stages: SSB detection, DNA end processing, DNA gap filling, and DNA ligation. A more detailed explanation is given in Caldecott 2008, and a summary is given here.

In the first stage, when a SSB forms, PARP1 and/or PARP2 recognize the break and recruit repair machinery. The binding and activity of PARP1 at DNA breaks is transient and it seems to accelerate SSBr by promoting the focal accumulation or stability of SSBr protein complexes at the lesion. Arguably the most important of these SSBr proteins is XRCC1, which functions as a molecular scaffold that interacts with, stabilizes, and stimulates multiple enzymatic components of the SSBr process including the protein responsible for cleaning the DNA 3′ and 5′ ends. For instance, XRCC1 interacts with several proteins (DNA polymerase beta, PNK, and three nucleases, APE1, APTX, and APLF) that promote end processing. APE1 has endonuclease activity. APLF exhibits endonuclease and 3′ to 5′ exonuclease activities. APTX has endonuclease and 3′ to 5′ exonuclease activity.

This end processing is an important stage of SSBR since the 3′- and/or 5′-termini of most, if not all, SSBs are ‘damaged.’ End processing generally involves restoring a damaged 3′-end to a hydroxylated state and and/or a damaged 5′ end to a phosphate moiety, so that the ends become ligation-competent. Enzymes that can process damaged 3′ termini include PNKP, APE1, and TDP1. Enzymes that can process damaged 5′ termini include PNKP, DNA polymerase beta, and APTX. LIG3 (DNA ligase III) can also participate in end processing. Once the ends are cleaned, gap filling can occur.

At the DNA gap filling stage, the proteins typically present are PARP1, DNA polymerase beta, XRCC1, FEN1 (flap endonuclease 1), DNA polymerase delta/epsilon, PCNA, and LIG1. There are two ways of gap filling, the short patch repair and the long patch repair. Short patch repair involves the insertion of a single nucleotide that is missing. At some SSBs, “gap filling” might continue displacing two or more nucleotides (displacement of up to 12 bases have been reported). FEN1 is an endonuclease that removes the displaced 5′-residues. Multiple DNA polymerases, including Polβ, are involved in the repair of SSBs, with the choice of DNA polymerase influenced by the source and type of SSB.

In the fourth stage, a DNA ligase such as LIG1 (Ligase I) or LIG3 (Ligase III) catalyzes joining of the ends. Short patch repair uses Ligase III and long patch repair uses Ligase I.

Sometimes, SSBR is replication-coupled. This pathway can involve one or more of CtIP, MRN, ERCC1, and FEN1. Additional factors that may promote SSBR include: aPARP, PARP1, PARP2, PARG, XRCC1, DNA polymerase b, DNA polymerase d, DNA polymerase e, PCNA, LIG1, PNK, PNKP, APE1, APTX, APLF, TDP1, LIG3, FEN1, CtIP, MRN, and ERCC1.

9.9.2 MMR (Mismatch Repair)

Cells contain three excision repair pathways: MMR, BER, and NER. The excision repair pathways have a common feature in that they typically recognize a lesion on one strand of the DNA, then exo/endonucleases remove the lesion and leave a 1-30 nucleotide gap that is sub-sequentially filled in by DNA polymerase and finally sealed with ligase. A more complete picture is given in Li, Cell Research (2008) 18:85-98, and a summary is provided here.

Mismatch repair (MMR) operates on mispaired DNA bases.

The MSH2/6 or MSH2/3 complexes both have ATPases activity that plays an important role in mismatch recognition and the initiation of repair. MSH2/6 preferentially recognizes base-base mismatches and identifies mispairs of 1 or 2 nucleotides, while MSH2/3 preferentially recognizes larger ID mispairs.

hMLH1 heterodimerizes with hPMS2 to form hMutLa which possesses an ATPase activity and is important for multiple steps of MMR. It possesses a PCNA/replication factor C (RFC)-dependent endonuclease activity which plays an important role in 3′ nick-directed MMR involving EXO1. (EXO1 is a participant in both HR and MMR.) It regulates termination of mismatch-provoked excision. Ligase I is the relevant ligase for this pathway. Additional factors that may promote MMR include: EXO1, MSH2, MSH3, MSH6, MLH1, PMS2, MLH3, DNA Pol d, RPA, HMGB1, RFC, and DNA ligase I.

11.9.3 Base Excision Repair (BER)

The base excision repair (BER) pathway is active throughout the cell cycle; it is responsible primarily for removing small, non-helix-distorting base lesions from the genome. In contrast, the related Nucleotide Excision Repair pathway (discussed in the next section) repairs bulky helix-distorting lesions. A more detailed explanation is given in Caldecott, Nature Reviews Genetics 9, 619-631 (August 2008), and a summary is given here.

Upon DNA base damage, base excision repair (BER) is initiated and the process can be simplified into five major steps: (a) removal of the damaged DNA base; (b) incision of the subsequent a basic site; (c) clean-up of the DNA ends; (d) insertion of the desired nucleotide into the repair gap; and (e) ligation of the remaining nick in the DNA backbone. These last steps are similar to the SSBR.

In the first step, a damage-specific DNA glycosylase excises the damaged base through cleavage of the N-glycosidic bond linking the base to the sugar phosphate backbone. Then AP endonuclease-1 (APE1) or bifunctional DNA glycosylases with an associated lyase activity incised the phosphodiester backbone to create a DNA single strand break (SSB). The third step of BER involves cleaning-up of the DNA ends. The fourth step in BER is conducted by Polβ that adds a new complementary nucleotide into the repair gap and in the final step XRCC1/Ligase III seals the remaining nick in the DNA backbone. This completes the short-patch BER pathway in which the majority (˜80%) of damaged DNA bases are repaired. However, if the 5′ ends in step 3 are resistant to end processing activity, following one nucleotide insertion by Pol β there is then a polymerase switch to the replicative DNA polymerases, Pol δ/ε, which then add ˜2-8 more nucleotides into the DNA repair gap. This creates a 5′ flap structure, which is recognized and excised by flap endonuclease-1 (FEN-1) in association with the processivity factor proliferating cell nuclear antigen (PCNA). DNA ligase I then seals the remaining nick in the DNA backbone and completes long-patch BER. Additional factors that may promote the BER pathway include: DNA glycosylase, APE1, Polb, Pold, Pole, XRCC1, Ligase III, FEN-1, PCNA, RECQL4, WRN, MYH, PNKP, and APTX.

11.9.4 Nucleotide Excision Repair (NER)

Nucleotide excision repair (NER) is an important excision mechanism that removes bulky helix-distorting lesions from DNA. Additional details about NER are given in Marteijn et al., Nature Reviews Molecular Cell Biology 15, 465-481 (2014), and a summary is given here. NER a broad pathway encompassing two smaller pathways: global genomic NER (GG-NER) and transcription coupled repair NER (TC-NER). GG-NER and TC-NER use different factors for recognizing DNA damage. However, they utilize the same machinery for lesion incision, repair, and ligation.

Once damage is recognized, the cell removes a short single-stranded DNA segment that contains the lesion. Endonucleases XPF/ERCC1 and XPG (encoded by ERCC5) remove the lesion by cutting the damaged strand on either side of the lesion, resulting in a single-strand gap of 22-30 nucleotides. Next, the cell performs DNA gap filling synthesis and ligation. Involved in this process are: PCNA, RFC, DNA Pol δ, DNA Pol ε or DNA Pol κ, and DNA ligase I or XRCC1/Ligase III. Replicating cells tend to use DNA pol ε and DNA ligase I, while non-replicating cells tend to use DNA Pol δ, DNA Pol κ, and the XRCC1/Ligase III complex to perform the ligation step.

NER can involve the following factors: XPA-G, POLH, XPF, ERCC1, XPA-G, and LIG1. Transcription-coupled NER (TC-NER) can involve the following factors: CSA, CSB, XPB, XPD, XPG, ERCC1, and TTDA. Additional factors that may promote the NER repair pathway include XPA-G, POLH, XPF, ERCC1, XPA-G, LIG1, CSA, CSB, XPA, XPB, XPC, XPD, XPF, XPG, TTDA, UVSSA, USP7, CETN2, RAD23B, UV-DDB, CAK subcomplex, RPA, and PCNA.

11.9.5 Interstrand Crosslink (ICL)

A dedicated pathway called the ICL repair pathway repairs interstrand crosslinks. Interstrand crosslinks, or covalent crosslinks between bases in different DNA strand, can occur during replication or transcription. ICL repair involves the coordination of multiple repair processes, in particular, nucleolytic activity, translesion synthesis (TLS), and HDR. Nucleases are recruited to excise the ICL on either side of the crosslinked bases, while TLS and HDR are coordinated to repair the cut strands. ICL repair can involve the following factors: endonucleases, e.g., XPF and RAD51C, endonucleases such as RAD51, translesion polymerases, e.g., DNA polymerase zeta and Rev1), and the Fanconi anemia (FA) proteins, e.g., FancJ.

11.9.6 Other Pathways

Several other DNA repair pathways exist in mammals.

Translesion synthesis (TLS) is a pathway for repairing a single stranded break left after a defective replication event and involves translesion polymerases, e.g., DNA polβ and Rev1.

Error-free postreplication repair (PRR) is another pathway for repairing a single stranded break left after a defective replication event.

11.10 Targeted Knockdown

Unlike CRISPR/Cas-mediated gene knockout, which permanently eliminates expression by mutating the gene (e.g., a CCR5 or CXCR4 gene) at the DNA level, CRISPR/Cas knockdown allows for temporary reduction of gene expression through the use of artificial transcription factors. Mutating key residues in both DNA cleavage domains of the Cas9 protein (e.g. the D10A and H840A mutations) results in the generation of a catalytically inactive Cas9 (eiCas9 which is also known as dead Cas9 or dCas9) molecule. A catalytically inactive Cas9 complexes with a gRNA and localizes to the DNA sequence specified by that gRNA's targeting domain, however, it does not cleave the target DNA. Fusion of the dCas9 to an effector domain, e.g., a transcription repression domain, enables recruitment of the effector to any DNA site specified by the gRNA. Although an enzymatically inactive (eiCas9) Cas9 molecule itself can block transcription when recruited to early regions in the coding sequence, more robust repression can be achieved by fusing a transcriptional repression domain (for example KRAB, SID or ERD) to the Cas9 and recruiting it to the target knockdown position, e.g., within 1000 bp of sequence 3′ of the start codon or within 500 bp of a promoter region 5′ of the start codon of a gene (e.g., a CCR5 or CXCR4 gene). It is likely that targeting DNAseI hypersensitive sites (DHSs) of the promoter may yield more efficient gene repression or activation because these regions are more likely to be accessible to the Cas9 protein and are also more likely to harbor sites for endogenous transcription factors. Especially for gene repression, it is contemplated herein that blocking the binding site of an endogenous transcription factor would aid in downregulating gene expression. In certain embodiments, one or more eiCas9 molecules may be used to block binding of one or more endogenous transcription factors. In certain embodiments, an eiCas9 molecule can be fused to a chromatin modifying protein. Altering chromatin status can result in decreased expression of the target gene. One or more eiCas9 molecules fused to one or more chromatin modifying proteins may be used to alter chromatin status.

In certain embodiments, a gRNA molecule can be targeted to a known transcription response elements (e.g., promoters, enhancers, etc.), a known upstream activating sequences (UAS), and/or sequences of unknown or known function that are suspected of being able to control expression of the target DNA.

CRISPR/Cas-mediated gene knockdown can be used to reduce expression of an unwanted allele or transcript. In certain embodiments, permanent destruction of the gene is not ideal. In these embodiments, site-specific repression may be used to temporarily reduce or eliminate expression. In certain embodiments, the off-target effects of a Cas-repressor may be less severe than those of a Cas-nuclease as a nuclease can cleave any DNA sequence and cause mutations whereas a Cas-repressor may only have an effect if it targets the promoter region of an actively transcribed gene. However, while nuclease-mediated knockout is permanent, repression may only persist as long as the Cas-repressor is present in the cells. Once the repressor is no longer present, it is likely that endogenous transcription factors and gene regulatory elements would restore expression to its natural state.

11.11 Examples of gRNAs in Genome Editing Methods

gRNA molecules as described herein can be used with Cas9 molecules that generate a double strand break or a single strand break to alter the sequence of a target nucleic acid, e.g., a target position or target genetic signature. gRNA molecules useful in these methods are described below.

In certain embodiments, the gRNA, e.g., a chimeric gRNA, is configured such that it comprises one or more of the following properties;

(a) it can position, e.g., when targeting a Cas9 molecule that makes double strand breaks, a double strand break (i) within 50, 100, 150, 200, 250, 300, 350, 400, 450, or 500 nucleotides of a target position, or (ii) sufficiently close that the target position is within the region of end resection;

(b) it has a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and

(c)(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. aureus, or N. meningitidis tail and proximal domain, or a sequence that differs by no more than 1, 2, 3, 4, 5; 6, 7, 8, 9 or 10 nucleotides therefrom;

(c)(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. aureus, or N meningitidis gRNA, or a sequence that differs by no more than 1, 2, 3, 4, 5; 6, 7, 8, 9 or 10 nucleotides therefrom;

(c)(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. aureus, or N meningitidis gRNA, or a sequence that differs by no more than 1, 2, 3, 4, 5; 6, 7, 8, 9 or 10 nucleotides therefrom;

(c)(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. aureus, or N meningitidis tail domain, or a sequence that differs by no more than 1, 2, 3, 4, 5; 6, 7, 8, 9 or 10 nucleotides therefrom; or

(c)(v) the tail domain comprises 15, 20, 25, 30, 35, 40 nucleotides or all of the corresponding portions of a naturally occurring tail domain, e.g., a naturally occurring S. pyogenes, S. aureus, or N. meningitidis tail domain.

In certain embodiments, the gRNA is configured such that it comprises properties: a and b(i); a and b(ii); a and b(iii); a and b(iv); a and b(v); a and b(vi); a and b(vii); a and b(viii); a and b(ix); a and b(x); a and b(xi); a and c; a, b, and c; a(i), b(i), and c(i); a(i), b(i), and c(ii); a(i), b(ii), and c(i); a(i), b(ii), and c(ii); a(i), b(iii), and c(i); a(i), b(iii), and c(ii); a(i), b(iv), and c(i); a(i), b(iv), and c(ii); a(i), b(v), and c(i); a(i), b(v), and c(ii); a(i), b(vi), and c(i); a(i), b(vi), and c(ii); a(i), b(vii), and c(i); a(i), b(vii), and c(ii); a(i), b(viii), and c(i); a(i), b(viii), and c(ii); a(i), b(ix), and c(i); a(i), b(ix), and c(ii); a(i), b(x), and c(i); a(i), b(x), and c(ii); a(i), b(xi), or c(i); a(i), b(xi), and c(ii).

In certain embodiments, the gRNA, e.g., a chimeric gRNA, is configured such that it comprises one or more of the following properties;

(a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;

(b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides; and

(c)(i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. aureus, or N. meningitidis tail and proximal domain, or a sequence that differs by no more than 1, 2, 3, 4, 5, 6, 7, 8, 9 or 10 nucleotides therefrom;

(c)(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. aureus, or N. meningitidis gRNA, or a sequence that differs by no more than 1, 2, 3, 4, 5; 6, 7, 8, 9 or 10 nucleotides therefrom;

(c)(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. aureus, or N meningitidis gRNA, or a sequence that differs by no more than 1, 2, 3, 4, 5; 6, 7, 8, 9 or 10 nucleotides therefrom;

(c)(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. aureus, or N meningitidis tail domain, or a sequence that differs by no more than 1, 2, 3, 4, 5; 6, 7, 8, 9 or 10 nucleotides therefrom; or

(c)(v) the tail domain comprises 15, 20, 25, 30, 35, 40 nucleotides or all of the corresponding portions of a naturally occurring tail domain, e.g., a naturally occurring S. pyogenes, S. aureus, or N. meningitidis tail domain.

In certain embodiments, the gRNA is configured such that it comprises properties: a and b(i); a and b(ii); a and b(iii); a and b(iv); a and b(v); a and b(vi); a and b(vii); a and b(viii); a and b(ix); a and b(x); a and b(xi); a and c; a, b, and c; a(i), b(i), and c(i); a(i), b(i), and c(ii); a(i), b(ii), and c(i); a(i), b(ii), and c(ii); a(i), b(iii), and c(i); a(i), b(iii), and c(ii); a(i), b(iv), and c(i); a(i), b(iv), and c(ii); a(i), b(v), and c(i); a(i), b(v), and c(ii); a(i), b(vi), and c(i); a(i), b(vi), and c(ii); a(i), b(vii), and c(i); a(i), b(vii), and c(ii); a(i), b(viii), and c(i); a(i), b(viii), and c(ii); a(i), b(ix), and c(i); a(i), b(ix), and c(ii); a(i), b(x), and c(i); a(i), b(x), and c(ii); a(i), b(xi), and c(i); a(i), b(xi), and c(ii).

In certain embodiments, the gRNA is used with a Cas9 nickase molecule having HNH activity, e.g., a Cas9 molecule having the RuvC activity inactivated, e.g., a Cas9 molecule having a mutation at D10, e.g., the D10A mutation. In certain embodiments, the gRNA is used with a Cas9 nickase molecule having RuvC activity, e.g., a Cas9 molecule having the HNH activity inactivated, e.g., a Cas9 molecule having a mutation at 840, e.g., the H840A. In certain embodiments, the gRNAs are used with a Cas9 nickase molecule having RuvC activity, e.g., a Cas9 molecule having the HNH activity inactivated, e.g., a Cas9 molecule having a mutation at N863, e.g., the N863A mutation. In certain embodiments, the gRNAs are used with a Cas9 nickase molecule having RuvC activity, e.g., a Cas9 molecule having the HNH activity inactivated, e.g., a Cas9 molecule having a mutation at N580, e.g., the N580A mutation.

In certain embodiments, a pair of gRNAs, e.g., a pair of chimeric gRNAs, comprising a first and a second gRNA, is configured such that they comprises one or more of the following properties;

(a) one or both of the gRNAs can position, e.g., when targeting a Cas9 molecule that makes single strand breaks, a single strand break within (i) 50, 100, 150, 200, 250, 300, 350, 400, 450, or 500 nucleotides of a target position, or (ii) sufficiently close that the target position is within the region of end resection;

(b) one or both have a targeting domain of at least 16 nucleotides, e.g., a targeting domain of (i) 16, (ii), 17, (iii) 18, (iv) 19, (v) 20, (vi) 21, (vii) 22, (viii) 23, (ix) 24, (x) 25, or (xi) 26 nucleotides;

(c) (i) the proximal and tail domain, when taken together, comprise at least 15, 18, 20, 25, 30, 31, 35, 40, 45, 49, 50, or 53 nucleotides, e.g., at least 15, 18, 20, 25, 30, 31, 35, 40, 45, 49, 50, or 53 nucleotides from a naturally occurring S. pyogenes, S. aureus, or N. meningitidis tail and proximal domain, or a sequence that differs by no more than 1, 2, 3, 4, 5; 6, 7, 8, 9 or 10 nucleotides therefrom;

(c)(ii) there are at least 15, 18, 20, 25, 30, 31, 35, 40, 45, 49, 50, or 53 nucleotides 3′ to the last nucleotide of the second complementarity domain, e.g., at least 15, 18, 20, 25, 30, 31, 35, 40, 45, 49, 50, or 53 nucleotides from the corresponding sequence of a naturally occurring S. pyogenes, S. aureus, or N meningitidis gRNA, or a sequence that differs by no more than 1, 2, 3, 4, 5; 6, 7, 8, 9 or 10 nucleotides therefrom;

(c)(iii) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain, e.g., at least 16, 19, 21, 26, 31, 32, 36, 41, 46, 50, 51, or 54 nucleotides from the corresponding sequence of a naturally occurring S. pyogenes, S. aureus, or N meningitidis gRNA, or a sequence that differs by no more than 1, 2, 3, 4, 5; 6, 7, 8, 9 or 10 nucleotides therefrom;

(c)(iv) the tail domain is at least 10, 15, 20, 25, 30, 35 or 40 nucleotides in length, e.g., it comprises at least 10, 15, 20, 25, 30, 35 or 40 nucleotides from a naturally occurring S. pyogenes, S. aureus, or N meningitidis tail domain; or, or a sequence that differs by no more than 1, 2, 3, 4, 5; 6, 7, 8, 9 or 10 nucleotides therefrom; or

(c)(v) the tail domain comprises 15, 20, 25, 30, 35, 40 nucleotides or all of the corresponding portions of a naturally occurring tail domain, e.g., a naturally occurring S. pyogenes, S. aureus, or N. meningitidis tail domain;

(d) the gRNAs are configured such that, when hybridized to target nucleic acid, they are separated by 0-50, 0-100, 0-200, at least 10, at least 20, at least 30 or at least 50 nucleotides;

(e) the breaks made by the first gRNA and second gRNA are on different strands; and

(f) the PAMs are facing outwards.

In certain embodiments, one or both of the gRNAs is configured such that it comprises properties: a and b(i); a and b(ii); a and b(iii); a and b(iv); a and b(v); a and b(vi); a and b(vii); a and b(viii); a and b(ix); a and b(x); a and b(xi); a and c; a, b, and c; a(i), b(i), and c(i); a(i), b(i), and c(ii); a(i), b(i), c, and d; a(i), b(i), c, and e; a(i), b(i), c, d, and e; a(i), b(ii), and c(i); a(i), b(ii), and c(ii); a(i), b(ii), c, and d; a(i), b(ii), c, and e; a(i), b(ii), c, d, and e; a(i), b(iii), and c(i); a(i), b(iii), and c(ii); a(i), b(iii), c, and d; a(i), b(iii), c, and e; a(i), b(iii), c, d, and e; a(i), b(iv), and c(i); a(i), b(iv), and c(ii); a(i), b(iv), c, and d; a(i), b(iv), c, and e; a(i), b(iv), c, d, and e; a(i), b(v), and c(i); a(i), b(v), and c(ii); a(i), b(v), c, and d; a(i), b(v), c, and e; a(i), b(v), c, d, and e; a(i), b(vi), and c(i); a(i), b(vi), and c(ii); a(i), b(vi), c, and d; a(i), b(vi), c, and e; a(i), b(vi), c, d, and e; a(i), b(vii), and c(i); a(i), b(vii), and c(ii); a(i), b(vii), c, and d; a(i), b(vii), c, and e; a(i), b(vii), c, d, and e; a(i), b(viii), and c(i); a(i), b(viii), and c(ii); a(i), b(viii), c, and d; a(i), b(viii), c, and e; a(i), b(viii), c, d, and e; a(i), b(ix), and c(i); a(i), b(ix), and c(ii); a(i), b(ix), c, and d; a(i), b(ix), c, and e; a(i), b(ix), c, d, and e; a(i), b(x), and c(i); a(i), b(x), and c(ii); a(i), b(x), c, and d; a(i), b(x), c, and e; a(i), b(x), c, d, and e; a(i), b(xi), and c(i); a(i), b(xi), and c(ii); a(i), b(xi), c, and d; a(i), b(xi), c, and e; a(i), b(xi), c, d, and e.

In certain embodiments, the gRNAs are used with a Cas9 nickase molecule having HNH activity, e.g., a Cas9 molecule having the RuvC activity inactivated, e.g., a Cas9 molecule having a mutation at D10, e.g., the D10A mutation. In certain embodiments, the gRNAs are used with a Cas9 nickase molecule having RuvC activity, e.g., a Cas9 molecule having the HNH activity inactivated, e.g., a Cas9 molecule having a mutation at H840, e.g., the H840A mutation. In certain embodiments, the gRNAs are used with a Cas9 nickase molecule having RuvC activity, e.g., a Cas9 molecule having the HNH activity inactivated, e.g., a Cas9 molecule having a mutation at N863, e.g., the N863A mutation. In certain embodiments, the gRNAs are used with a Cas9 nickase molecule having RuvC activity, e.g., a Cas9 molecule having the HNH activity inactivated, e.g., a Cas9 molecule having a mutation at N580, e.g., the N580A mutation.

### 12. Target Cells

Cas9 molecules and gRNA molecules, e.g., a Cas9 molecule/gRNA molecule complex, can be used to manipulate a cell, e.g., to edit a target nucleic acid, in a wide variety of cells.

In certain embodiments, a cell is manipulated by altering or editing (e.g., introducing a mutation in) the CCR5 gene, e.g., as described herein. In certain embodiments, the expression of the CCR5 gene is altered or modulated, e.g., in vivo. In certain embodiments, the expression of the CCR5 gene is altered or modulated, e.g., ex vivo.

In certain embodiments, a cell is manipulated by altering or editing (e.g., introducing a mutation in) the CXCR4 gene, e.g., as described herein. In certain embodiments, the expression of the CXCR4 gene is altered or modulated, e.g., in vivo. In certain embodiments, the expression of the CXCR4 gene is altered or modulated, e.g., ex vivo.

In certain embodiments, a cell is manipulated by altering or editing (e.g., introducing a mutation in) both the CCR5 and the CXCR4 genes, e.g., as described herein. In certain embodiments, the expression of both the CCR5 and the CXCR4 genes is altered or modulated, e.g., in vivo. In certain embodiments, the expression of both the CCR5 and the CXCR4 genes is altered or modulated, e.g., ex vivo.

The Cas9 and gRNA molecules described herein can be delivered to a target cell. In certain embodiments, the target cell is a circulating blood cell, e.g., a T cell (e.g., a CD4⁺ T cell, a CD8⁺ T cell, a helper T cell, a regulatory T cell, a cytotoxic T cell, a memory T cell, a T cell precursor or a natural killer T cell), a B cell (e.g., a progenitor B cell, a Pre B cell, a Pro B cell, a memory B cell, a plasma B cell), a monocyte, a megakaryocyte, a neutrophil, an eosinophil, a basophil, a mast cell, a reticulocyte, a lymphoid progenitor cell, a myeloid progenitor cell, a gut-associated lymphoid tissue (GALT) cell, a dendritic cell, a macrophage, a microglial cell,or a hematopoietic stem cell. In certain embodiments, the target cell is a bone marrow cell, (e.g., a lymphoid progenitor cell, a myeloid progenitor cell, an erythroid progenitor cell, a hematopoietic stem cell, or a mesenchymal stem cell). In certain embodiments, the target cell is a CD4⁺ T cell. In certain embodiments, the target cell is a lymphoid progenitor cell (e.g. a common lymphoid progenitor (CLP) cell). In certain embodiments, the target cell is a myeloid progenitor cell (e.g. a common myeloid progenitor (CMP) cell). In certain embodiments, the target cell is a hematopoietic stem cell (e.g. a long term hematopoietic stem cell (LT-HSC), a short term hematopoietic stem cell (ST-HSC), a multipotent progenitor (MPP) cell, a lineage restricted progenitor (LRP) cell).

In certain embodiments, the target cell is manipulated ex vivo by editing (e.g., introducing a mutation in) the CCR5 gene and/or modulating the expression of the CCR5 gene, and administered to the subject. In certain embodiments, the target cell is manipulated ex vivo by editing (e.g., introducing a mutation in) the CXCR4 gene and/or modulating the expression of the CXCR4 gene, and administered to the subject. In certain embodiments, the target cell is manipulated ex vivo by editing (e.g., introducing a mutation in) both the CCR5 and the CXCR4 gene and/or modulating the expression of the both the CCR5 and the CXCR4 gene, and administered to the subject. Sources of target cells for ex vivo manipulation may include, by way of example, the subject's blood, the subject's cord blood, or the subject's bone marrow. Sources of target cells for ex vivo manipulation may also include, by way of example, heterologous donor blood, cord blood, or bone marrow.

In certain embodiments, a CD4⁺T cell is removed from the subject, manipulated ex vivo as described above, and the CD4⁺T cell is returned to the subject. In certain embodiments, a lymphoid progenitor cell is removed from the subject, manipulated ex vivo as described above, and the lymphoid progenitor cell is returned to the subject. In certain embodiments, a myeloid progenitor cell is removed from the subject, manipulated ex vivo as described above, and the myeloid progenitor cell is returned to the subject. In certain embodiments, a hematopoietic stem cell is removed from the subject, manipulated ex vivo as described above, and the hematopoietic stem cell is returned to the subject.

A suitable cell can also include a stem cell such as, by way of example, an embryonic stem cell, an induced pluripotent stem cell, a neuronal stem cell and a mesenchymal stem cell. In certain embodiments, the cell is an induced pluripotent stem cells (iPS) cell or a cell derived from an iPS cell, e.g., an iPS cell generated from the subject, modified as described above and differentiated into a clinically relevant cell such as e.g, a CD4⁺ T cell, a lymphoid progenitor cell, myeloid progenitor cell, a macrophage, dendritic cell, gut associated lymphoid tissue or a hematopoietic stem cell. In certain embodiments, AAV is used to transduce the target cells, e.g., the target cells described herein.

### 13. Delivery, Formulations and Routes of Administration

The components, e.g., a Cas9 molecule, one or more gRNA molecules (e.g., a Cas9 molecule/gRNA molecule complex), and a donor template nucleic acid, or all three, can be delivered, formulated, or administered in a variety of forms, see, e.g., Tables 6 and 7. In certain embodiments, the Cas9 molecule, one or more gRNA molecules (e.g., two gRNA molecules) are present together in a genome editing system. In certain embodiments, the sequence encoding the Cas9 molecule and the sequence(s) encoding the two or more (e.g., 2, 3, 4, or more) different gRNA molecules are present on the same nucleic acid molecule, e.g., an AAV vector. In certain embodiments, two sequences encoding the Cas9 molecules and the sequences encoding the two or more (e.g., 2, 3, 4, or more) different gRNA molecules are present on the same nucleic acid molecule, e.g., an AAV vector. When a Cas9 or gRNA component is encoded as DNA for delivery, the DNA can typically include a control region, e.g., comprising a promoter, to effect expression. Useful promoters for Cas9 molecule sequences include CMV, EFS, EF-1a, MSCV, PGK, and CAG, the Skeletal Alpha Actin promoter, the Muscle Creatine Kinase promoter, the Dystrophin promoter, the Alpha Myosin Heavy Chain promoter, and the Smooth Muscle Actin promoter. In certain embodiments, the promoter is a constitutive promoter. In certain embodiments, the promoter is a tissue specific promoter. Useful promoters for gRNAs include T7.H1, EF-la, 7SK, U6, U1 and tRNA promoters. Promoters with similar or dissimilar strengths can be selected to tune the expression of components. Sequences encoding a Cas9 molecule can comprise a nuclear localization signal (NLS), e.g., an SV40 NLS. In certain embodiments, the sequence encoding a Cas9 molecule comprise at least two nuclear localization signals. In certain embodiments a promoter for a Cas9 molecule or a gRNA molecule can be, independently, inducible, tissue specific, or cell specific. Table 6 provides examples of how the components can be formulated, delivered, or administered.

TABLE 6
Elements

| | | Donor | |
|---|---|---|---|
| | | Template | |
| Cas9 | gRNA | Nucleic | |
| Molecule(s) | Molecule(s) | Acid | Comments |
| DNA | DNA | DNA | In certain embodiments, a Cas9 |
| | | | molecule (e.g., an eaCas9 or eiCas9 |
| | | | molecule) and a gRNA are transcribed |
| | | | from DNA. In certain embodiments, |
| | | | they are encoded on separate |
| | | | molecules. In certain embodiments, the |
| | | | donor template is provided as a separate |
| | | | DNA molecule. |
| DNA | DNA | | In certain embodiments, a Cas9 |
| | | | molecule (e.g., an eaCas9 or eiCas9 |
| | | | molecule) and a gRNA are transcribed |
| | | | from DNA. In certain embodiments, |
| | | | they are encoded on separate |
| | | | molecules. In certain embodiments, the |
| | | | donor template is provided on the same |
| | | | DNA molecule that encodes the gRNA. |
| DNA | | DNA | In certain embodiments, a Cas9 |
| | | | molecule (e.g., an eaCas9 or eiCas9 |
| | | | molecule) and a gRNA are transcribed |
| | | | from DNA, here from a single |
| | | | molecule. In certain embodiments, the |
| | | | donor template is provided as a separate |
| | | | DNA molecule. |
| DNA | DNA | | In certain embodiments, a Cas9 |
| | | | molecule (e.g., an eaCas9 or eiCas9 |
| | | | molecule), and a gRNA are transcribed |
| | | | from DNA. In certain embodiments, |
| | | | they are encoded on separate |
| | | | molecules. In certain embodiments, the |
| | | | donor template is provided on the same |
| | | | DNA molecule that encodes the Cas9. |
| DNA | RNA | DNA | In certain embodiments, a Cas9 |
| | | | molecule (e.g., an eaCas9 or eiCas9 |
| | | | molecule) is transcribed from DNA, and |
| | | | a gRNA is provided as in vitro |
| | | | transcribed or synthesized RNA. In |
| | | | certain embodiments, the donor |
| | | | template is provided as a separate DNA |
| | | | molecule. |
| DNA | RNA | | In certain embodiments, a Cas9 |
| | | | molecule (e.g., an eaCas9 or eiCas9 |
| | | | molecule) is transcribed from DNA, and |
| | | | a gRNA is provided as in vitro |
| | | | transcribed or synthesized RNA. In |
| | | | certain embodiments t, the donor |
| | | | template is provided on the same DNA |
| | | | molecule that encodes the Cas9. |
| mRNA | RNA | DNA | In certain embodiments, a Cas9 |
| | | | molecule (e.g., an eaCas9 or eiCas9 |
| | | | molecule) is translated from in vitro |
| | | | transcribed mRNA, and a gRNA is |
| | | | provided as in vitro transcribed or |
| | | | synthesized RNA. In certain |
| | | | embodiments, the donor template is |
| | | | provided as a DNA molecule. |
| mRNA | DNA | DNA | In certain embodiments, a Cas9 |
| | | | molecule (e.g., an eaCas9 or eiCas9 |
| | | | molecule) is translated from in vitro |
| | | | transcribed mRNA, and a gRNA is |
| | | | transcribed from DNA. In certain |
| | | | embodiments, the donor template is |
| | | | provided as a separate DNA molecule. |
| mRNA | DNA | | In certain embodiments, a Cas9 |
| | | | molecule (e.g., an eaCas9 or eiCas9 |
| | | | molecule) is translated from in vitro |
| | | | transcribed mRNA, and a gRNA is |
| | | | transcribed from DNA. In certain |
| | | | embodiments, the donor template is |
| | | | provided on the same DNA molecule |
| | | | that encodes the gRNA. |
| Protein | DNA | DNA | In certain embodiments, a Cas9 |
| | | | molecule (e.g., an eaCas9 or eiCas9 |
| | | | molecule) is provided as a protein, and a |
| | | | gRNA is transcribed from DNA. In |
| | | | certain embodiments, the donor |
| | | | template is provided as a separate DNA |
| | | | molecule. |
| Protein | DNA | | In certain embodiments, a Cas9 |
| | | | molecule (e.g., an eaCas9 or eiCas9 |
| | | | molecule) is provided as a protein, and a |
| | | | gRNA is transcribed from DNA. In |
| | | | certain embodiments, the donor |
| | | | template is provided on the same DNA |
| | | | molecule that encodes the gRNA. |
| Protein | RNA | DNA | In certain embodiments (e.g., an eaCas9 |
| | | | or eiCas9 molecule) is provided as a |
| | | | protein, and a gRNA is provided as |
| | | | transcribed or synthesized RNA. This |
| | | | delivery method is referred to as “RNP |
| | | | delivery”. In certain embodiments, the |
| | | | donor template is provided as a DNA |
| | | | molecule. |

Table 7 summarizes various delivery methods for the components of a Cas system, e.g., the Cas9 molecule component and the gRNA molecule component, as described herein.

TABLE 7

| | | Delivery | | | |
|---|---|---|---|---|---|
| | | into Non- | Duration | | Type of |
| | | Dividing | of | Genome | Molecule |
| Delivery Vector/Mode | | Cells | Expression | Integration | Delivered |
| Physical (e.g., electroporation, | | YES | Transient | NO | Nucleic Acids |
| particle gun, Calcium | | | | | and Proteins |
| Phosphate transfection, cell | | | | | |
| compression or squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

13.1 DNA-Based Delivery of a Cas9 Molecule and or One or More gRNA Molecule

Nucleic acid compositions encoding Cas9 molecules (e.g., eaCas9 molecules or eiCas9 molecules), gRNA molecules, a donor template nucleic acid, or any combination (e.g., two or all) thereof can be administered to subjects or delivered into cells by art-known methods or as described herein. For example, Cas9-encoding and/or gRNA-encoding DNA, as well as donor template nucleic acids can be delivered, e.g., by vectors (e.g., viral or non-viral vectors), non-vector based methods (e.g., using naked DNA or DNA complexes), or a combination thereof.

Nucleic acid compositions encoding Cas9 molecules (e.g., eaCas9 molecules or eiCas9 molecules) and/or gRNA molecules can be conjugated to molecules (e.g., N-acetylgalactosamine) promoting uptake by the target cells (e.g., the target cells described herein). Donor template molecules can likewise be conjugated to molecules (e.g., N-acetylgalactosamine) promoting uptake by the target cells (e.g., the target cells described herein).

In certain embodiments, the Cas9- and/or gRNA-encoding DNA is delivered by a vector (e.g., viral vector/virus or plasmid).

Vectors can comprise a sequence that encodes a Cas9 molecule and/or a gRNA molecule, and/or a donor template with high homology to the region (e.g., target sequence) being targeted. In certain embodiments, the donor template comprises all or part of a target sequence. Exemplary donor templates are a repair template, e.g., a gene correction template, or a gene mutation template, e.g., point mutation (e.g., single nucleotide (nt) substitution) template). A vector can also comprise a sequence encoding a signal peptide (e.g., for nuclear localization, nucleolar localization, or mitochondrial localization), fused, e.g., to a Cas9 molecule sequence. For example, the vectors can comprise a nuclear localization sequence (e.g., from SV40) fused to the sequence encoding the Cas9 molecule.

One or more regulatory/control elements, e.g., promoters, enhancers, introns, polyadenylation signals, a Kozak consensus sequences, internal ribosome entry sites (IRES), a 2A sequence, and splice acceptor or donor can be included in the vectors. In certain embodiments, the promoter is recognized by RNA polymerase II (e.g., a CMV promoter). In other embodiments, the promoter is recognized by RNA polymerase III (e.g., a U6 promoter). In certain embodiments, the promoter is a regulated promoter (e.g., inducible promoter). In certain embodiments, the promoter is a constitutive promoter. In certain embodiments, the promoter is a tissue specific promoter. In certain embodiments, the promoter is a viral promoter. In certain embodiments, the promoter is a non-viral promoter.

In certain embodiments, the vector or delivery vehicle is a viral vector (e.g., for generation of recombinant viruses). In certain embodiments, the virus is a DNA virus (e.g., dsDNA or ssDNA virus). In certain embodiments, the virus is an RNA virus (e.g., an ssRNA virus). In certain embodiments, the virus infects dividing cells. In other embodiments, the virus infects non-dividing cells. Exemplary viral vectors/viruses include, e.g., retroviruses, lentiviruses, adenovirus, adeno-associated virus (AAV), vaccinia viruses, poxviruses, and herpes simplex viruses.

In certain embodiments, the virus infects dividing cells. In other embodiments, the virus infects non-dividing cells. In certain embodiments, the virus infects both dividing and non-dividing cells. In certain embodiments, the virus can integrate into the host genome. In certain embodiments, the virus is engineered to have reduced immunity, e.g., in human. In certain embodiments, the virus is replication-competent. In other embodiments, the virus is replication-defective, e.g., having one or more coding regions for the genes necessary for additional rounds of virion replication and/or packaging replaced with other genes or deleted. In certain embodiments, the virus causes transient expression of the Cas9 molecule or molecules and/or the gRNA molecule or molecules. In other embodiments, the virus causes long-lasting, e.g., at least 1 week, 2 weeks, 1 month, 2 months, 3 months, 6 months, 9 months, 1 year, 2 years, or permanent expression, of the Cas9 molecule or molecules and/or the gRNA molecule or molecules. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In certain embodiments, the viral vector recognizes a specific cell type or tissue. For example, the viral vector can be pseudotyped with a different/alternative viral envelope glycoprotein; engineered with a cell type-specific receptor (e.g., genetic modification(s) of one or more viral envelope glycoproteins to incorporate a targeting ligand such as a peptide ligand, a single chain antibody, or a growth factor); and/or engineered to have a molecular bridge with dual specificities with one end recognizing a viral glycoprotein and the other end recognizing a moiety of the target cell surface (e.g., a ligand-receptor, monoclonal antibody, avidin-biotin and chemical conjugation).

Exemplary viral vectors/viruses include, e.g., retroviruses, lentiviruses, adenovirus, adeno-associated virus (AAV), vaccinia viruses, poxviruses, and herpes simplex viruses.

In certain embodiments, the Cas9- and/or gRNA-encoding sequence is delivered by a recombinant retrovirus. In certain embodiments, the retrovirus (e.g., Moloney murine leukemia virus) comprises a reverse transcriptase, e.g., that allows integration into the host genome. In certain embodiments, the retrovirus is replication-competent. In other embodiments, the retrovirus is replication-defective, e.g., having one of more coding regions for the genes necessary for additional rounds of virion replication and packaging replaced with other genes, or deleted.

In certain embodiments, the Cas9- and/or gRNA-encoding nucleic acid sequence is delivered by a recombinant lentivirus. In certain embodiments, the donor template nucleic acid is delivered by a recombinant retrovirus. For example, the lentivirus is replication-defective, e.g., does not comprise one or more genes required for viral replication.

In certain embodiments, the Cas9- and/or gRNA-encoding nucleic acid sequence is delivered by a recombinant adenovirus. In certain embodiments, the donor template nucleic acid is delivered by a recombinant adenovirus. In certain embodiments, the adenovirus is engineered to have reduced immunity in human.

In certain embodiments, the Cas9- and/or gRNA-encoding nucleic acid sequence is delivered by a recombinant AAV. In certain embodiments, the donor template nucleic acid is delivered by a recombinant AAV. In certain embodiments, the AAV does not incorporate its genome into that of a host cell, e.g., a target cell as describe herein. In certain embodiments, the AAV can incorporate at least part of its genome into that of a host cell, e.g., a target cell as described herein. In certain embodiments, the AAV is a self-complementary adeno-associated virus (scAAV), e.g., a scAAV that packages both strands which anneal together to form double stranded DNA. AAV serotypes that may be used in the disclosed methods, include AAV1, AAV2, modified AAV2 (e.g., modifications at Y444F, Y500F, Y730F and/or S662V), AAV3, modified AAV3 (e.g., modifications at Y705F, Y731F and/or T492V), AAV4, AAV5, AAV6, modified AAV6 (e.g., modifications at S663V and/or T492V), AAV8, AAV 8.2, AAV9, AAV rh10, and pseudotyped AAV, such as AAV2/8, AAV2/5 and AAV2/6 can also be used in the disclosed methods. In certain embodiments, an AAV capsid that can be used in the methods described herein is a capsid sequence from serotype AAV1, AAV2, AAV3, AAV4, AAV5, AAV6, AAV7, AAV8, AAV9, AAV.rh8, AAV.rh10, AAV.rh32/33, AAV.rh43, AAV.rh64R1, or AAV7m8.

In certain embodiments, the Cas9- and/or gRNA-encoding nucleic acid sequence is delivered in a re-engineered AAV capsid, e.g., with about 50% or greater, e.g., about 60% or greater, about 70% or greater, about 80% or greater, about 90% or greater, or about 95% or greater, sequence homology with a capsid sequence from serotypes AAV1, AAV2, AAV3, AAV4, AAV5, AAV6, AAV7, AAV8, AAV9, AAV.rh8, AAV.rh10, AAV.rh32/33, AAV.rh43, or AAV.rh64R1.

In certain embodiments, the Cas9- and/or gRNA-encoding nucleic acid sequence is delivered by a chimeric AAV capsid. In certain embodiments, the donor template nucleic acid is delivered by a chimeric AAV capsid. Exemplary chimeric AAV capsids include, but are not limited to, AAV9i1, AAV2i8, AAV-DJ, AAV2G9, AAV2i8G9, or AAV8G9.

In certain embodiments, the AAV is a self-complementary adeno-associated virus (scAAV), e.g., a scAAV that packages both strands which anneal together to form double stranded DNA.

In certain embodiments, the Cas9- and/or gRNA-encoding DNA is delivered by a hybrid virus, e.g., a hybrid of one or more of the viruses described herein. In certain embodiments, the hybrid virus is hybrid of an AAV (e.g., of any AAV serotype), with a Bocavirus, B19 virus, porcine AAV, goose AAV, feline AAV, canine AAV, or MVM.

A packaging cell is used to form a virus particle that is capable of infecting a target cell. Exemplary packaging cells include 293 cells, which can package adenovirus, and ψ2 or PA317 cells, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed, e.g., components for a Cas9 molecule, e.g., two Cas9 components. For example, an AAV vector used in gene therapy typically only possesses inverted terminal repeat (ITR) sequences from the AAV genome which are required for packaging and gene expression in the host or target cell. The missing viral functions can be supplied in trans by the packaging cell line and/or plasmid containing E2A, E4, and VA genes from adenovirus, and plasmid encoding Rep and Cap genes from AAV, as described in “Triple Transfection Protocol.” Henceforth, the viral DNA is packaged in a cell line, which contains a helper plasmid encoding the other AAV genes, namely rep and cap, but lacking ITR sequences. In certain embodiments, the viral DNA is packaged in a producer cell line, which contains E1A and/or E1B genes from adenovirus. The cell line is also infected with adenovirus as a helper. The helper virus (e.g., adenovirus or HSV) or helper plasmid promotes replication of the AAV vector and expression of AAV genes from the helper plasmid with ITRs. The helper plasmid is not packaged in significant amounts due to a lack of ITR sequences. Contamination with adenovirus can be reduced by, e.g., heat treatment to which adenovirus is more sensitive than AAV.

In certain embodiments, the viral vector is capable of cell type and/or tissue type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as peptide ligands, single chain antibodies, growth factors); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In certain embodiments, the viral vector achieves cell type specific expression. For example, a tissue-specific promoter can be constructed to restrict expression of the transgene (Cas9 and gRNA) to only the target cell. The specificity of the vector can also be mediated by microRNA-dependent control of transgene expression. In certain embodiments, the viral vector has increased efficiency of fusion of the viral vector and a target cell membrane. For example, a fusion protein such as fusion-competent hemagglutin (HA) can be incorporated to increase viral uptake into cells. In certain embodiments, the viral vector has the ability of nuclear localization. For example, a virus that requires the breakdown of the nuclear envelope (during cell division) and therefore can not infect a non-diving cell can be altered to incorporate a nuclear localization peptide in the matrix protein of the virus thereby enabling the transduction of non-proliferating cells.

In certain embodiments, the Cas9- and/or gRNA-encoding nucleic acid sequence is delivered by a non-vector based method (e.g., using naked DNA or DNA complexes). For example, the DNA can be delivered, e.g., by organically modified silica or silicate (Ormosil), electroporation, transient cell compression or squeezing (e.g., as described in Lee, et al, 2012, Nano Lett 12: 6322-27), gene gun, sonoporation, magnetofection, lipid-mediated transfection, dendrimers, inorganic nanoparticles, calcium phosphates, or a combination thereof.

In certain embodiments, delivery via electroporation comprises mixing the cells with the Cas9-and/or gRNA-encoding DNA in a cartridge, chamber or cuvette and applying one or more electrical impulses of defined duration and amplitude. In certain embodiments, delivery via electroporation is performed using a system in which cells are mixed with the Cas9- and/or gRNA-encoding DNA in a vessel connected to a device (e.g, a pump) which feeds the mixture into a cartridge, chamber or cuvette wherein one or more electrical impulses of defined duration and amplitude are applied, after which the cells are delivered to a second vessel.

In certain embodiments, the Cas9- and/or gRNA-encoding nucleic acid sequence is delivered by a combination of a vector and a non-vector based method. In certain embodiments, the donor template nucleic acid is delivered by a combination of a vector and a non-vector based method. For example, virosomes combine liposomes combined with an inactivated virus (e.g., HIV or influenza virus), which can result in more efficient gene transfer, e.g., in respiratory epithelial cells than either viral or liposomal methods alone.

In certain embodiments, the delivery vehicle is a non-viral vector. In certain embodiments, the non-viral vector is an inorganic nanoparticle. Exemplary inorganic nanoparticles include, e.g., magnetic nanoparticles (e.g., Fe₃MnO₂) and silica. The outer surface of the nanoparticle can be conjugated with a positively charged polymer (e.g., polyethylenimine, polylysine, polyserine) which allows for attachment (e.g., conjugation or entrapment) of payload. In certain embodiments, the non-viral vector is an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating.

Exemplary lipids for gene transfer are shown below in Table 8.

TABLE 8
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)propyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)- | GAP-DLRIE | Cationic |
| 1-propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3- | MDRIE | Cationic |
| bis(tetradecyloxy)-1-propanaminium bromide | | |
| Dimyristooxypropyl dimethyl hydroxyethyl ammonium | DMRI | Cationic |
| bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3- | diC14-amidine | Cationic |
| tetradecylaminopropionamidine | | |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9- | CDAN | Cationic |
| diamine | | |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Exemplary polymers for gene transfer are shown below in Table 9.

TABLE 9
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

In certain embodiments, the vehicle has targeting modifications to increase target cell update of nanoparticles and liposomes, e.g., cell specific antigens, monoclonal antibodies, single chain antibodies, aptamers, polymers, sugars (e.g., N-acetylgalactosamine (GalNAc)), and cell penetrating peptides. In certain embodiments, the vehicle uses fusogenic and endosome-destabilizing peptides/polymers. In certain embodiments, the vehicle undergoes acid-triggered conformational changes (e.g., to accelerate endosomal escape of the cargo). In certain embodiments, a stimuli-cleavable polymer is used, e.g., for release in a cellular compartment. For example, disulfide-based cationic polymers that are cleaved in the reducing cellular environment can be used.

In certain embodiments, the delivery vehicle is a biological non-viral delivery vehicle. In certain embodiments, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific tissues, bacteria having modified surface proteins to alter target tissue specificity). In certain embodiments, the vehicle is a genetically modified bacteriophage (e.g., engineered phages having large packaging capacity, less immunogenic, containing mammalian plasmid maintenance sequences and having incorporated targeting ligands). In certain embodiments, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In certain embodiments, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes—subject (i.e., patient) derived membrane-bound nanovesicle (30 -100 nm) of endocytic origin (e.g., can be produced from various cell types and can therefore be taken up by cells without the need of for targeting ligands).

In certain embodiments, one or more nucleic acid molecules (e.g., DNA molecules) other than the components of a Cas system, e.g., the Cas9 molecule component or components and/or the gRNA molecule component or components described herein, are delivered. In certain embodiments, the nucleic acid molecule is delivered at the same time as one or more of the components of the Cas system are delivered. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In certain embodiments, the nucleic acid molecule is delivered by a different means than one or more of the components of the Cas system, e.g., the Cas9 molecule component and/or the gRNA molecule component, are delivered. The nucleic acid molecule can be delivered by any of the delivery methods described herein. For example, the nucleic acid molecule can be delivered by a viral vector, e.g., an integration-deficient lentivirus, and the Cas9 molecule component or components and/or the gRNA molecule component or components can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

13.2 Delivery of a RNA Encoding a Cas9 Molecule

RNA encoding Cas9 molecules (e.g., eaCas9 molecules or eiCas9 molecules) and/or gRNA molecules, can be delivered into cells, e.g., target cells described herein, by art-known methods or as described herein. For example, Cas9-encoding and/or gRNA-encoding RNA can be delivered, e.g., by microinjection, electroporation, transient cell compression or squeezing (e.g., as described in Lee, et al., 2012, Nano Lett 12: 6322-27), lipid-mediated transfection, peptide-mediated delivery, or a combination thereof. Cas9-encoding and/or gRNA-encoding RNA can be conjugated to molecules to promote uptake by the target cells (e.g., target cells described herein).

In certain embodiments, delivery via electroporation comprises mixing the cells with the RNA encoding Cas9 molecules (e.g., eaCas9 molecules, eiCas9 molecules or eiCas9 fusion proteins) and/or gRNA molecules with or without donor template nucleic acid molecules, in a cartridge, chamber or cuvette and applying one or more electrical impulses of defined duration and amplitude. In certain embodiments, delivery via electroporation is performed using a system in which cells are mixed with the RNA encoding Cas9 molecules (e.g., eaCas9 molecules, eiCas9 molecules or eiCas9 fusion protiens) and/or gRNA molecules with or without donor template nucleic acid molecules, in a vessel connected to a device (e.g., a pump) which feeds the mixture into a cartridge, chamber or cuvette wherein one or more electrical impulses of defined duration and amplitude are applied, after which the cells are delivered to a second vessel. Cas9-encoding and/or gRNA-encoding RNA can be conjugated to molecules to promote uptake by the target cells (e.g., target cells described herein).

13.3 Delivery of a Cas9 Molecule Protein

Cas9 molecules (e.g., eaCas9 molecules or eiCas9 molecules) can be delivered into cells by art-known methods or as described herein. For example, Cas9 protein molecules can be delivered, e.g., by microinjection, electroporation, transient cell compression or squeezing (e.g., as described in Lee, et al, 2012, Nano Lett 12: 6322-27), lipid-mediated transfection, peptide-mediated delivery, or a combination thereof. Delivery can be accompanied by DNA encoding a gRNA or by a gRNA. Cas9 protein can be conjugated to molecules promoting uptake by the target cells (e.g., target cells described herein).

In certain embodiments, delivery via electroporation comprises mixing the cells with the Cas9 molecules (e.g., eaCas9 molecules, eiCas9 molecules or eiCas9 fusion protiens) and/or gRNA molecules with or without donor nucleic acid, in a cartridge, chamber or cuvette and applying one or more electrical impulses of defined duration and amplitude. In certain embodiments, delivery via electroporation is performed using a system in which cells are mixed with the Cas9 molecules (e.g., eaCas9 molecules, eiCas9 molecules or eiCas9 fusion protiens) and/or gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel. Cas9-encoding and/or gRNA-encoding RNA can be conjugated to molecules to promote uptake by the target cells (e.g., target cells described herein).

13. 4 RNP Delivery of Cas9 Molecule Protein and gRNA

In certain embodiments, the Cas9 molecule and gRNA molecule are delivered to target cells via Ribonucleoprotein (RNP) delivery. In certain embodiments, the Cas9 molecule is provided as a protein, and the gRNA molecule is provided as transcribed or synthesized RNA. The gRNA molecule can be generated by chemical synthesis. In certain embodiments, the gRNA molecule forms a RNP complex with the Cas9 molecule protein under suitable condition prior to delivery to the target cells. The RNP complex can be delivered to the target cells by any suitable methods known in the art, e.g., by electroporation, lipid-mediated transfection, protein or DNA-based shuttle, mechanical force, or hydraulic force. In certain embodiments, the RNP complex is delivered to the target cells by electroporation.

13.5 Route of Administration

Systemic modes of administration include oral and parenteral routes. Parenteral routes include, by way of example, intravenous, intrarterial, intraosseous, intramuscular, intradermal, subcutaneous, intranasal and intraperitoneal routes. Components administered systemically may be modified or formulated to target the components to cells of the blood and bone marrow.

Local modes of administration include, by way of example, intra-bone marrow, intrathecal, and intra-cerebroventricular routes. In certain embodiments, significantly smaller amounts of the components (compared with systemic approaches) may exert an effect when administered locally (for example, intra-bone marrow) compared to when administered systemically (for example, intravenously). Local modes of administration can reduce or eliminate the incidence of potentially toxic side effects that may occur when therapeutically effective amounts of a component are administered systemically.

In certain embodiments, components described herein are delivered by intra-bone marrow injection. Injections may be made directly into the bone marrow compartment of one or more than one bone. In certain embodiments, nanoparticle or viral, e.g., AAV vector, delivery is via intra-bone marrow injection.

Administration may be provided as a periodic bolus or as continuous infusion from an internal reservoir or from an external reservoir (for example, from an intravenous bag). Components may be administered locally, for example, by continuous release from a sustained release drug delivery device.

In addition, components may be formulated to permit release over a prolonged period of time. A release system can include a matrix of a biodegradable material or a material which releases the incorporated components by diffusion. The components can be homogeneously or heterogeneously distributed within the release system. A variety of release systems may be useful, however, the choice of the appropriate system can depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used for intraocular injection. Typically the microspheres are composed of a polymer of lactic acid and glycolic acid, which are structured to form hollow spheres. The spheres can be approximately 15-30 microns in diameter and can be loaded with components described herein.

13.6 Bi-Modal or Differential Delivery of Components

Separate delivery of the components of a Cas system, e.g., the Cas9 molecule component or components and the gRNA molecule component or components, and more particularly, delivery of the components by differing modes, can enhance performance, e.g., by improving tissue specificity and safety.

In certain embodiments, the Cas9 molecule or molecules and the gRNA molecule or molecules are delivered by different modes, or as sometimes referred to herein as differential modes. Different or differential modes, as used herein, refer modes of delivery that confer different pharmacodynamic or pharmacokinetic properties on the subject component molecule, e.g., a Cas9 molecule or molecules or gRNA molecule or molecules, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components, e.g., a Cas9 molecule and a gRNA molecule, can be delivered by modes that differ in terms of resulting half-life or persistence of the delivered component within the body, or in a particular compartment, tissue or organ. In certain embodiments, a gRNA molecule can be delivered by such modes. The Cas9 molecule component can be delivered by a mode which results in less persistence or less exposure to the body or a particular compartment or tissue or organ. In certain embodiments, two Cas9 molecules can by delivered by modes that differ in terms of resulting half-life or persistence of the delivered component within the body, or in a particular compartment, tissue or organ. In certain embodiments, two or more gRNA molecules can by delivered by modes that differ in terms of resulting half-life or persistence of the delivered component within the body, or in a particular compartment, tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a Cas9 molecule, is delivered in a transient manner, for example as mRNA or as protein, ensuring that the full Cas9 molecule/gRNA molecule complex is only present and active for a short period of time.

In certain embodiments, the second component, two Cas9 molecules, is delivered in a transient manner, for example as mRNA or as protein, ensuring that the full Cas9/gRNA complex is only present and active for a short period of time. In certain embodiments, the second components, two Cas9 molecules, are delivered at two separate time points, e.g. a first Cas9 molecule delivered at one time point and a second Cas9 molecule delivered at a second time point, for example as mRNA or as protein, ensuring that the full Cas9/gRNA complexes are only present and active for a short period of time.

Furthermore, the components can be delivered in different molecular form or with different delivery vectors that complement one another to enhance safety and tissue specificity.

Use of differential delivery modes can enhance performance, safety and efficacy. E.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MEW molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA molecule is delivered by a first delivery mode that results in a first spatial, e.g., tissue, distribution. A second component, e.g., a Cas9 molecule is delivered by a second delivery mode that results in a second spatial, e.g., tissue, distribution. Two distinct second components, e.g., two distinct Cas9 molecules, are delivered by two distinct delivery modes that result in a second and third spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. The third mode comprises a second element selected from the group. In certain embodiments, the first mode of delivery comprises a first targeting element, e.g., a cell specific receptor or an antibody, and the second mode of delivery does not include that element. In embodiment, the second mode of delivery comprises a second targeting element, e.g., a second cell specific receptor or second antibody. In embodiment, the third mode of delivery comprises a second targeting element, e.g., a second cell specific receptor or second antibody.

When the Cas9 molecule or molecules are delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA molecule or molecules and the Cas9 molecule or molecules are packaged in separated delivery vehicles with distinct but overlapping tissue tropism, the fully functional complex is only be formed in the tissue that is targeted by both vectors.

In certain embodiments, components designed to alter (e.g., introduce a mutation into CCR5 or CXCR4) in one target position are delivered prior to, concurrent with, or subsequent to components designed to alter (e.g., introduce a mutation into CCR5 or CXCR4) a second target position.

13.7 Ex Vivo Delivery

In certain embodiments, each component of the genome editing system described in Table 6 are introduced into a cell which is then introduced into the subject, e.g., cells are removed from a subject, manipulated ex vivo and then introduced into the subject. Methods of introducing the components can include, e.g., any of the delivery methods described in Table 7.

### 14. Modified Nucleosides, Nucleotides, and Nucleic Acids

Modified nucleosides and modified nucleotides can be present in nucleic acids, e.g., particularly gRNA, but also other forms of RNA, e.g., mRNA, RNAi, or siRNA. As described herein, “nucleoside” is defined as a compound containing a five-carbon sugar molecule (a pentose or ribose) or derivative thereof, and an organic base, purine or pyrimidine, or a derivative thereof. As described herein, “nucleotide” is defined as a nucleoside further comprising a phosphate group.

Modified nucleosides and nucleotides can include one or more of:

(i) alteration, e.g., replacement, of one or both of the non-linking phosphate oxygens and/or of one or more of the linking phosphate oxygens in the phosphodiester backbone linkage;

(ii) alteration, e.g., replacement, of a constituent of the ribose sugar, e.g., of the 2′ hydroxyl on the ribose sugar;

(iii) wholesale replacement of the phosphate moiety with “dephospho” linkers;

(iv) modification or replacement of a naturally occurring nucleobase;

(v) replacement or modification of the ribose-phosphate backbone;

(vi) modification of the 3′ end or 5′ end of the oligonucleotide, e.g., removal, modification or replacement of a terminal phosphate group or conjugation of a moiety; and

(vii) modification of the sugar.

The modifications listed above can be combined to provide modified nucleosides and nucleotides that can have two, three, four, or more modifications. For example, a modified nucleoside or nucleotide can have a modified sugar and a modified nucleobase. In certain embodiments, every base of a gRNA is modified, e.g., all bases have a modified phosphate group, e.g., all are phosphorothioate groups. In certain embodiments, all, or substantially all, of the phosphate groups of a unimolecular or modular gRNA molecule are replaced with phosphorothioate groups.

In certain embodiments, modified nucleotides, e.g., nucleotides having modifications as described herein, can be incorporated into a nucleic acid, e.g., a “modified nucleic acid.” In certain embodiments, the modified nucleic acids comprise one, two, three or more modified nucleotides. In certain embodiments, at least 5% (e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%) of the positions in a modified nucleic acid are a modified nucleotides.

Unmodified nucleic acids can be prone to degradation by, e.g., cellular nucleases. For example, nucleases can hydrolyze nucleic acid phosphodiester bonds. Accordingly, in certain embodiments, the modified nucleic acids described herein can contain one or more modified nucleosides or nucleotides, e.g., to introduce stability toward nucleases.

In certain embodiments, the modified nucleosides, modified nucleotides, and modified nucleic acids described herein can exhibit a reduced innate immune response when introduced into a population of cells, both in vivo and ex vivo. The term “innate immune response” includes a cellular response to exogenous nucleic acids, including single stranded nucleic acids, generally of viral or bacterial origin, which involves the induction of cytokine expression and release, particularly the interferons, and cell death. In certain embodiments, the modified nucleosides, modified nucleotides, and modified nucleic acids described herein can disrupt binding of a major groove interacting partner with the nucleic acid. In certain embodiments, the modified nucleosides, modified nucleotides, and modified nucleic acids described herein can exhibit a reduced innate immune response when introduced into a population of cells, both in vivo and ex vivo, and also disrupt binding of a major groove interacting partner with the nucleic acid.

14.1 Definitions of Chemical Groups

As used herein, “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 12, from 1 to about 8, from 1 to about 6, from 1 to about 4, or from 1 to about 3 carbon atoms.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In certain embodiments, aryl groups have from 6 to about 20 carbon atoms.

As used herein, “alkenyl” refers to an aliphatic group containing at least one double bond.

As used herein, “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl.

As used herein, “arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

As used herein, “cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl.

As used herein, “heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl.

As used herein, “heteroaryl” refers to a monovalent radical of a heteroaromatic ring system. Examples of heteroaryl moieties include, but are not limited to, imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

14.2 Phosphate Backbone Modifications

14.2.1 The Phosphate Group

In certain embodiments, the phosphate group of a modified nucleotide can be modified by replacing one or more of the oxygens with a different substituent. Further, the modified nucleotide, e.g., modified nucleotide present in a modified nucleic acid, can include the wholesale replacement of an unmodified phosphate moiety with a modified phosphate as described herein. In certain embodiments, the modification of the phosphate backbone can include alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. In certain embodiments, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or OR (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligoribonucleotide diastereomers. In certain embodiments, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

14.2.2 Replacement of the Phosphate Group

The phosphate group can be replaced by non-phosphorus containing connectors. In certain embodiments, the charge phosphate group can be replaced by a neutral moiety.

Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

14.2.3 Replacement of the Ribophosphate Backbone

Scaffolds that can mimic nucleic acids can also be constructed wherein the phosphate linker and ribose sugar are replaced by nuclease resistant nucleoside or nucleotide surrogates. In certain embodiments, the nucleobases can be tethered by a surrogate backbone. Examples can include, without limitation, the morpholino, cyclobutyl, pyrrolidine and peptide nucleic acid (PNA) nucleoside surrogates.

14.3 Sugar Modifications

The modified nucleosides and modified nucleotides can include one or more modifications to the sugar group. For example, the 2′ hydroxyl group (OH) can be modified or replaced with a number of different “oxy” or “deoxy” substituents. In certain embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom.

Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In certain embodiments, the “oxy”-2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In certain embodiments, the “oxy”-2′ hydroxyl group modification can include the methoxyethyl group (MOE), (OCH₂CH₂OCH₃, e.g., a PEG derivative).

“Deoxy” modifications can include hydrogen (i.e. deoxyribose sugars, e.g., at the overhang portions of partially ds RNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the 1′ position on the sugar, e.g., alpha-nucleosides. The modified nucleic acids can also include “abasic” sugars, which lack a nucleobase at C-1′. These abasic sugars can also be further modified at one or more of the constituent sugar atoms. The modified nucleic acids can also include one or more sugars that are in the L form, e.g. L-nucleosides.

Generally, RNA includes the sugar group ribose, which is a 5-membered ring having an oxygen. Exemplary modified nucleosides and modified nucleotides can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In certain embodiments, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replaced with α-L-threofuranosyl-(3′→2′)).

14.4 Modifications on the Nucleobase

The modified nucleosides and modified nucleotides described herein, which can be incorporated into a modified nucleic acid, can include a modified nucleobase. Examples of nucleobases include, but are not limited to, adenine (A), guanine (G), cytosine (C), and uracil (U). These nucleobases can be modified or wholly replaced to provide modified nucleosides and modified nucleotides that can be incorporated into modified nucleic acids. The nucleobase of the nucleotide can be independently selected from a purine, a pyrimidine, a purine or pyrimidine analog. In certain embodiments, the nucleobase can include, for example, naturally-occurring and synthetic derivatives of a base.

14.4.1 Uracil

In certain embodiments, the modified nucleobase is a modified uracil. Exemplary nucleobases and nucleosides having a modified uracil include without limitation pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s2U), 5-aminomethyl-2-thio-uridine (nm⁵s2U), 5-methylaminomethyl-uridine (mnm⁵U), 5-methylaminomethyl-2-thio-uridine (mnm⁵s2U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm ⁵s2U), 5-propynyl-uridine, 1-propynyl-pseudouridine, 5-taurinomethyl-uridine (τcm⁵U), 1-taurinomethyl-pseudouridine, 5-taurinomethyl-2-thio-uridine(τm⁵s2U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s2U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(i sopentenylaminomethyl)-2-thio-uridine (inm⁵s2U), a-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s2Um), 5-methoxycarbonylmethyl-2′-O-methyl-uridine (mcm ⁵Um), 5-carbamoylmethyl-2′-O-methyl-uridine (ncm ⁵Um), 5-carboxymethylaminomethyl-2′-O-methyl-uridine (cmnm ⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

4.4.2 Cytosine

In certain embodiments, the modified nucleobase is a modified cytosine. Exemplary nucleobases and nucleosides having a modified cytosine include without limitation 5-aza-cytidine, 6-aza-cytidine, pseudoisocytidine, 3-methyl-cytidine (m³C), N4-acetyl-cytidine (act), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoi socytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k²C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f ⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

14.4.3 Adenine

In certain embodiments, the modified nucleobase is a modified adenine. Exemplary nucleobases and nucleosides having a modified adenine include without limitation 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenosine, 7-deaza-8-aza-adenosine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenosine (m²A), N6-methyl-adenosine (m⁶A), 2-methylthio-N6-methyl-adenosine (ms2m⁶A), N6-isopentenyl-adenosine (i⁶A), 2-methylthio-N6-isopentenyl-adenosine (ms²⁻⁶₁A), N6-(cis-hydroxyisopentenyl)adenosine (io⁶A), 2-methylthio-N6-(cis-hydroxyisopentenyl)adenosine (ms2io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms2hn⁶A), N6-acetyl-adenosine (ac⁶A), 7-methyl-adenosine, 2-methylthio-adenosine, 2-methoxy-adenosine, a-thio-adenosine, 2′-O-methyl-adenosine (Am), N⁶,2′-O-dimethyl-adenosine (m⁶Am), N⁶-Methyl-2′-deoxyadenosine, N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

14.4.4 Guanine

In certain embodiments, the modified nucleobase is a modified guanine. Exemplary nucleobases and nucleosides having a modified guanine include without limitation inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OHyW), undermodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m′G), N2-methyl-guanosine (m²G), N2,N2-dimethyl-guanosine (m² ₂G), N2,7-dimethyl-guanosine (m²,7G), N2, N2,7-dimethyl-guanosine (m²,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m′Gm), N2,7-dimethyl-2′-O-methyl-guanosine (m²,7Gm), 2′-O-methyl-inosine (Im), 1,2′-O-dimethyl-inosine (m′Im), O⁶-phenyl-2′-deoxyinosine, 2′-O-ribosylguanosine (phosphate) (Gr(p)), 1-thio-guanosine, O⁶-methyl-guanosine, O⁶-Methyl-2′-deoxyguanosine, 2′-F-ara-guanosine, and 2′-F-guanosine.

14.5 Exemplary Modified gRNAs

In certain embodiments, the modified nucleic acids can be modified gRNAs. It is to be understood that any of the gRNAs described herein can be modified in accordance with this section, including any gRNA that comprises a targeting domain comprising a nucleotide sequence selected from SEQ ID NOS: 208 to 8407.

As discussed above, it was found that the gRNA component of the CRISPR/Cas system (e.g., a CRISPR/Cas9 system) is more efficient at editing genes in certain circulatory cell types (e.g., T cells) ex vivo when it has been modified at or near its 5′ end (e.g., when the 5′ end of a gRNA is modified by the inclusion of a eukaryotic mRNA cap structure or cap analog). In certain embodiments, these and other modified gRNAs described herein exhibit enhanced stability with certain cell types (e.g., circulatory cells, such as T cells) and that this might be responsible for the observed improvements.

The presently disclosed subject matter encompasses the realization that the improvements observed with a 5′ capped gRNA can be extended to gRNAs that have been modified in other ways to achieve the same type of structural or functional result (e.g., by the inclusion of modified nucleosides or nucleotides, or when an in vitro transcribed gRNA is modified by treatment with a phosphatase such as calf intestinal alkaline phosphatase to remove the 5′ triphosphate group). In certain embodiments, the modified gRNAs described herein may contain one or more modifications (e.g., modified nucleosides or nucleotides) which introduce stability toward nucleases (e.g., by the inclusion of modified nucleosides or nucleotides and/or a 3′ polyA tract).

Thus, in one aspect, methods, genome editing system and compositions discussed herein provide methods, genome editing system and compositions for gene editing of certain cells (e.g., ex vivo gene editing) by using gRNAs which have been modified at or near their 5′ end (e.g., within 1-10, 1-5, or 1-2 nucleotides of their 5′ end).

In certain embodiments, the 5′ end of the gRNA molecule lacks a 5′ triphosphate group. In certain embodiments, the 5′ end of the targeting domain lacks a 5′ triphosphate group. In certain embodiments, the 5′ end of the gRNA molecule includes a 5′ cap. In certain embodiments, the 5′ end of the targeting domain includes a 5′ cap. In certain embodiments, the gRNA molecule lacks a 5′ triphosphate group. In certain embodiments, the gRNA molecule comprises a targeting domain and the 5′ end of the targeting domain lacks a 5′ triphosphate group. In certain embodiments, gRNA molecule includes a 5′ cap. In certain embodiments, the gRNA molecule comprises a targeting domain and the 5′ end of the targeting domain includes a 5′ cap.

In certain embodiments, the 5′ end of a gRNA is modified by the inclusion of a eukaryotic mRNA cap structure or cap analog (e.g., without limitation, a G(5′)ppp(5′)G cap analog, a m7G(5′)ppp(5′)G cap analog, or a 3′-O-Me-m7G(5′)ppp(5′)G anti reverse cap analog (ARCA)). In certain embodiments, the 5′ cap comprises a modified guanine nucleotide that is linked to the remainder of the gRNA molecule via a 5′-5′ triphosphate linkage. In certain embodiments, the 5′ cap analogcomprises two optionally modified guanine nucleotides that are linked via a 5′-5′ triphosphate linkage. In certain embodiments, the 5′ end of the gRNA molecule has the chemical formula:

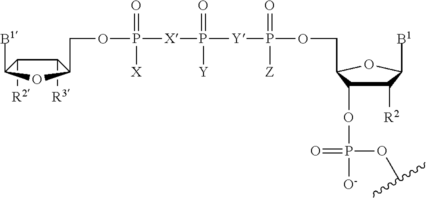

wherein:
each of B¹ and B¹′ is independently

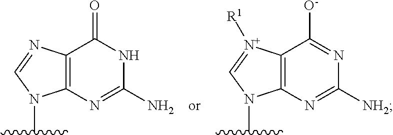

each R¹ is independently C₁₋₄ alkyl, optionally substituted by a phenyl or a 6-membered heteroaryl;
each of R², R²′, and R³′ is independently H, F, OH, or O—C₁₋₄ alkyl;
each of X, Y, and Z is independently O or S; and
each of X′ and Y′ is independently O or CH₂.

In certain embodiments, each R¹ is independently —CH₃, —CH₂CH₃, or —CH₂C₆H₅.

In certain embodiments, R¹ is —CH₃.

In certain embodiments, B¹′ is

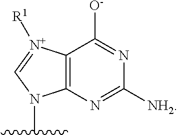

In certain embodiments, each of R², R²′, and R³′ is independently H, OH, or O—CH₃.

In certain embodiments, each of X, Y, and Z is O.

In certain embodiments, X′ and Y′ are O.

In certain embodiments, the 5′ end of the gRNA molecule has the chemical formula:

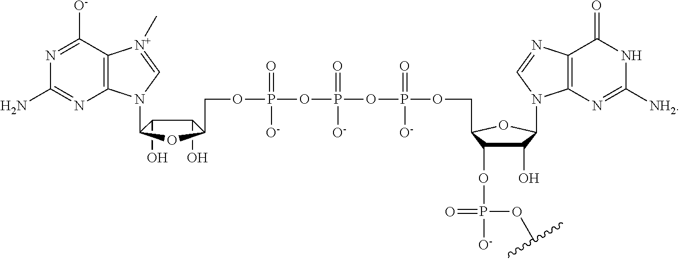

In certain embodiments, the 5′ end of the gRNA molecule has the chemical formula:

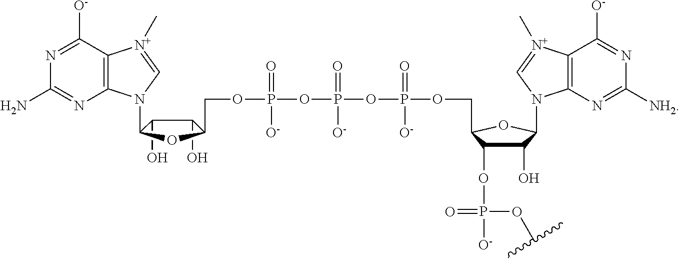

In certain embodiments, the 5′ end of the gRNA molecule has the chemical formula:

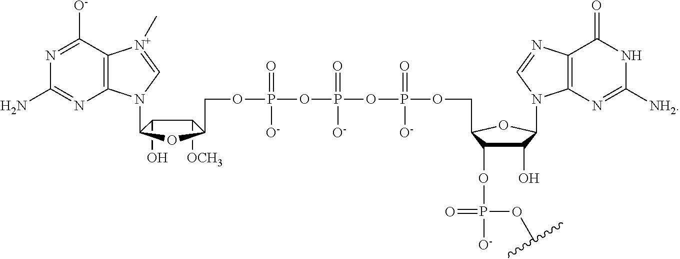

In certain embodiments, the 5′ end of the gRNA molecule has the chemical formula:

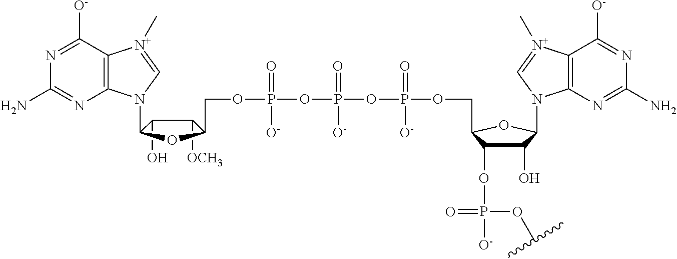

In certain embodiments, X is S, and Y and Z are O.

In certain embodiments, Y is S, and X and Z are O.

In certain embodiments, Z is S, and X and Y are O.

In certain embodiments, the phosphorothioate is the Sp diastereomer.

In certain embodiments, X′ is CH₂, and Y′ is O.

In certain embodiments, X′ is O, and Y′ is CH₂.

In certain embodiments, the 5′ cap comprises two optionally modified guanine nucleotides that are linked via an optionally modified 5′-5′ tetraphosphate linkage.

In certain embodiments, the 5′ end of the gRNA molecule has the chemical formula:

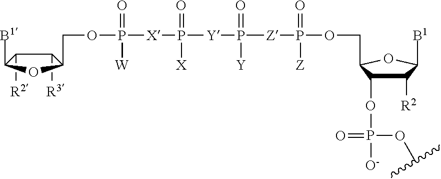

wherein:
each of B¹ and B¹′ is independently

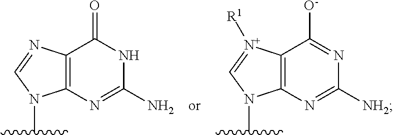

each R¹ is independently C₁₋₄ alkyl, optionally substituted by a phenyl or a 6-membered heteroaryl;
each of R², R²′, and R³′ is independently H, F, OH, or O—C₁₋₄ alkyl;
each of W, X, Y, and Z is independently O or S; and
each of X′, Y′, and Z′ is independently O or CH₂.

In certain embodiments, each R¹ is independently —CH₃, —CH₂CH₃, or —CH₂C₆H₅.

In certain embodiments, R¹ is —CH₃.

In certain embodiments, B¹′ is

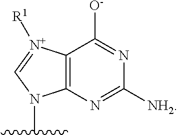

In certain embodiments, each of R², R²′, and R³′ is independently H, OH, or O—CH₃.

In certain embodiments, each of W, X, Y, and Z is O.

In certain embodiments, each of X′, Y′, and Z′ are O.

In certain embodiments, X′ is CH₂, and Y′ and Z′ are O.

In certain embodiments, Y′ is CH₂, and X′ and Z′ are O.

In certain embodiments, Z′ is CH₂, and X′ and Y′ are O.

In certain embodiments, the 5′ cap comprises two optionally modified guanine nucleotides that are linked via an optionally modified 5′-5′ pentaphosphate linkage.

In certain embodiments, the 5′ end of the gRNA molecule has the chemical formula:

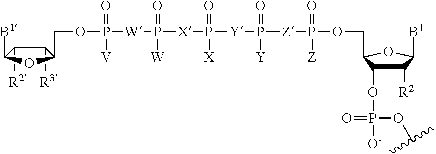

wherein:
each of B¹ and B¹′ is independently

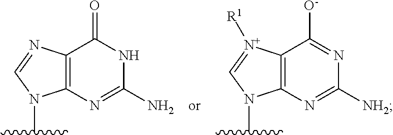

each R¹ is independently C₁₋₄ alkyl, optionally substituted by a phenyl or a 6-membered heteroaryl;
each of R², R²′, and R³′ is independently H, F, OH, or O—C₁₋₄ alkyl;
each of V, W, X, Y, and Z is independently O or S; and
each of W′, X′, Y′, and Z′ is independently O or CH₂.

In certain embodiments, each R¹ is independently —CH₃, —CH₂CH₃, or —CH₂C₆H₅.

In certain embodiments, R¹ is —CH₃.

In certain embodiments, B¹′ is

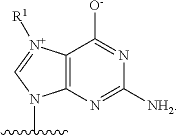

In certain embodiments, each of R², R²′, and R³′ is independently H, OH, or O—CH₃.

In certain embodiments, each of V, W, X, Y, and Z is O.

In certain embodiments, each of W′, X′, Y′, and Z′ is O.

As used herein, the term “5′ cap” encompasses traditional mRNA 5′ cap structures but also analogs of these. For example, in addition to the 5′ cap structures that are encompassed by the chemical structures shown above, one may use, e.g., tetraphosphate analogs having a methylene-bis(phosphonate) moiety (e.g., see Rydzik, A M et al., (2009) Org Biomol Chem 7(22):4763-76), analogs having a sulfur substitution for a non-bridging oxygen (e.g., see Grudzien-Nogalska, E. et al, (2007) RNA 13(10): 1745-1755), N7-benzylated dinucleoside tetraphosphate analogs (e.g., see Grudzien, E. et al., (2004) RNA 10(9): 1479-1487), or anti-reverse cap analogs (e.g., see U.S. Pat. No. 7,074,596 and Jemielity, J. et al., (2003) RNA 9(9): 1 108-1 122 and Stepinski, J. et al., (2001) RNA 7(10):1486-1495). The present application also encompasses the use of cap analogs with halogen groups instead of OH or OMe (e.g., see U.S. Pat. No. 8,304,529); cap analogs with at least one phosphorothioate (PS) linkage (e.g., see U.S. Pat. No. 8,153,773 and Kowalska, J. et al., (2008) RNA 14(6): 1 1 19-1131); and cap analogs with at least one boranophosphate or phosphoroselenoate linkage (e.g., see U.S. Pat. No. 8,519,110); and alkynyl-derivatized 5′ cap analogs (e.g., see U.S. Pat. No. 8,969,545).

In general, the 5′ cap can be included during either chemical synthesis or in vitro transcription of the gRNA. In certain embodiments, a 5′ cap is not used and the gRNA (e.g., an in vitro transcribed gRNA) is instead modified by treatment with a phosphatase (e.g., calf intestinal alkaline phosphatase) to remove the 5′ triphosphate group.

The presently disclosed subject matter also provides for methods, genome editing system and compositions for gene editing by using gRNAs which comprise a 3′ polyA tail (also called a polyA tract herein). Such gRNAs may, for example, be prepared by adding a polyA tail to a gRNA molecule precursor using a polyadenosine polymerase following in vitro transcription of the gRNA molecule precursor. For example, in certain embodiments, a polyA tail may be added enzymatically using a polymerase such as E. coli polyA polymerase (E-PAP). gRNAs including a polyA tail may also be prepared by in vitro transcription from a DNA template. In certain embodiments, a polyA tail of defined length is encoded on a DNA template and transcribed with the gRNA via an RNA polymerase (such as T7 RNA polymerase). gRNAs with a polyA tail may also be prepared by ligating a polyA oligonucleotide to a gRNA molecule precursor following in vitro transcription using an RNA ligase or a DNA ligase with or without a splinted DNA oligonucleotide complementary to the gRNA molecule precursor and the polyA oligonucleotide. For example, in certain embodiments, a polyA tail of defined length is synthesized as a synthetic oligonucleotide and ligated on the 3′ end of the gRNA with either an RNA ligase or a DNA ligase with or without a splinted DNA oligonucleotide complementary to the guide RNA and the polyA oligonucleotide. gRNAs including the polyA tail may also be prepared synthetically, in one or several pieces that are ligated together by either an RNA ligase or a DNA ligase with or without one or more splinted DNA oligonucleotides.

In certain embodiments, the polyA tail is comprised of fewer than 50 adenine nucleotides, for example, fewer than 45 adenine nucleotides, fewer than 40 adenine nucleotides, fewer than 35 adenine nucleotides, fewer than 30 adenine nucleotides, fewer than 25 adenine nucleotides or fewer than 20 adenine nucleotides. In certain embodiments the polyA tail is comprised of between 5 and 50 adenine nucleotides, for example between 5 and 40 adenine nucleotides, between 5 and 30 adenine nucleotides, between 10 and 50 adenine nucleotides, or between 15 and 25 adenine nucleotides. In certain embodiments, the polyA tail is comprised of about 20 adenine nucleotides.

The presently disclosed subject matter also provides for methods, genome editing system and compositions for gene editing (e.g., ex vivo gene editing) by using gRNAs which include one or more modified nucleosides or nucleotides that are described herein.

While some of the exemplary modifications discussed in this section may be included at any position within the gRNA sequence, in certain embodiments, a gRNA comprises a modification at or near its 5′ end (e.g., within 1-10, 1-5, or 1-2 nucleotides of its 5′ end). In certain embodiments, a gRNA comprises a modification at or near its 3′ end (e.g., within 1-10, 1-5, or 1-2 nucleotides of its 3′ end). In certain embodiments, a gRNA comprises both a modification at or near its 5′ end and a modification at or near its 3′ end.

In certain embodiments, a gRNA molecule (e.g., an in vitro transcribed gRNA) comprises a targeting domain which is complementary with a target domain from a gene expressed in a eukaryotic cell, wherein the gRNA molecule is modified at its 5′ end and comprises a 3′ polyA tail. The gRNA molecule may, for example, lack a 5′ triphosphate group (e.g., the 5′ end of the targeting domain lacks a 5′ triphosphate group). In certain embodiments, a gRNA (e.g., an in vitro transcribed gRNA) is modified by treatment with a phosphatase (e.g., calf intestinal alkaline phosphatase) to remove the 5′ triphosphate group and comprises a 3′ polyA tail as described herein. The gRNA molecule may alternatively include a 5′ cap (e.g., the 5′ end of the targeting domain includes a 5′ cap). In certain embodiments, a gRNA (e.g., an in vitro transcribed gRNA) contains both a 5′ cap structure or cap analog and a 3′ polyA tail as described herein. In certain embodiments, the 5′ cap comprises a modified guanine nucleotide that is linked to the remainder of the gRNA molecule via a 5′-5′ triphosphate linkage. In certain embodiments, the 5′ cap comprises two optionally modified guanine nucleotides that are linked via an optionally modified 5′-5′ triphosphate linkage (e.g., as described above). In certain embodiments, the polyA tail is comprised of between 5 and 50 adenine nucleotides, for example between 5 and 40 adenine nucleotides, between 5 and 30 adenine nucleotides, between 10 and 50 adenine nucleotides, between 15 and 25 adenine nucleotides, fewer than 30 adenine nucleotides, fewer than 25 adenine nucleotides or about 20 adenine nucleotides.

In certain embodiments, the presently disclosed subject matter provides for a gRNA molecule comprising a targeting domain which is complementary with a target domain from a gene expressed in a eukaryotic cell, wherein the gRNA molecule comprises a 3′ polyA tail which is comprised of fewer than 30 adenine nucleotides (e.g., fewer than 25 adenine nucleotides, between 15 and 25 adenine nucleotides, or about 20 adenine nucleotides). In certain embodiments, these gRNA molecules are further modified at their 5′ end (e.g., the gRNA molecule is modified by treatment with a phosphatase to remove the 5′ triphosphate group or modified to include a 5′ cap as described herein).

In certain embodiments, gRNAs can be modified at a 3′ terminal U ribose. In certain embodiments, the 5′ end and a 3′ terminal U ribose of the gRNA are modified (e.g., the gRNA is modified by treatment with a phosphatase to remove the 5′ triphosphate group or modified to include a 5′ cap as described herein).

For example, the two terminal hydroxyl groups of the U ribose can be oxidized to aldehyde groups and a concomitant opening of the ribose ring to afford a modified nucleoside as shown below:

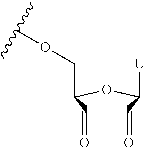

wherein “U” can be an unmodified or modified uridine.

In certain embodiments, the 3′ terminal U can be modified with a 2′3′ cyclic phosphate as shown below:

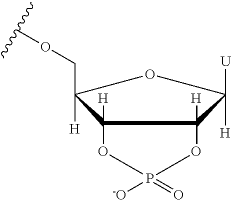

wherein “U” can be an unmodified or modified uridine.

In certain embodiments, the gRNA molecules may contain 3′ nucleotides which can be stabilized against degradation, e.g., by incorporating one or more of the modified nucleotides described herein. In this embodiment, e.g., uridines can be replaced with modified uridines, e.g., 5-(2-amino)propyl uridine, and 5-bromo uridine, or with any of the modified uridines described herein; adenosines, cytidines and guanosines can be replaced with modified adenosines, cytidines and guanosines, e.g., with modifications at the 8-position, e.g., 8-bromo guanosine, or with any of the modified adenosines, cytidines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclylamino, arylamino, diarylamino, heteroarylamino, diheteroarylamino, or amino acid); or cyano (—CN). In certain embodiments, the phosphate backbone can be modified as described herein, e.g., with a phosphothioate group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

In certain embodiments, a gRNA can include “locked” nucleic acids (LNA) in which the 2′ OH-group can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclylamino, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclylamino, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with a-L-threofuranosyl-(3′→2′)).

Generally, gRNA molecules include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA molecule are deoxynucleotides.

14.6 miRNA Binding Sites

microRNAs (or miRNAs) are naturally occurring cellular 19-25 nucleotide long noncoding RNAs. They bind to nucleic acid molecules having an appropriate miRNA binding site, e.g., in the 3′ UTR of an mRNA, and down-regulate gene expression. In certain embodiments, this down regulation occurs by either reducing nucleic acid molecule stability or inhibiting translation. An RNA species disclosed herein, e.g., an mRNA encoding Cas9 can comprise an miRNA binding site, e.g., in its 3′UTR. The miRNA binding site can be selected to promote down regulation of expression is a selected cell type. By way of example, the incorporation of a binding site for miR-122, a microRNA abundant in liver, can inhibit the expression of the gene of interest in the liver.

### EXAMPLES

The following Examples are merely illustrative and are not intended to limit the scope or content of the invention in any way.

### Example 1

### Evaluation of Candidate Guide RNAs (gRNAs)

The suitability of candidate gRNAs can be evaluated as described in this example. Although described for a chimeric gRNA, the approach can also be used to evaluate modular gRNAs.

Cloning gRNAs into Vectors

For each gRNA, a pair of overlapping oligonucleotides is designed and obtained. Oligonucleotides are annealed and ligated into a digested vector backbone containing an upstream U6 promoter and the remaining sequence of a long chimeric gRNA. Plasmid is sequence-verified and prepped to generate sufficient amounts of transfection-quality DNA. Alternate promoters maybe used to drive in vivo transcription (e.g. H1 promoter) or for in vitro transcription (e.g., a T7 promoter).

Cloning gRNAs in Linear dsDNA Molecule (STITCHR)

For each gRNA, a single oligonucleotide is designed and obtained. The U6 promoter and the gRNA scaffold (e.g. including everything except the targeting domain, e.g., including sequences derived from the crRNA and tracrRNA, e.g., including a first complementarity domain; a linking domain; a second complementarity domain; a proximal domain; and a tail domain) are separately PCR amplified and purified as dsDNA molecules. The gRNA-specific oligonucleotide is used in a PCR reaction to stitch together the U6 and the gRNA scaffold, linked by the targeting domain specified in the oligonucleotide. Resulting dsDNA molecule (STITCHR product) is purified for transfection. Alternate promoters may be used to drive in vivo transcription (e.g., H1 promoter) or for in vitro transcription (e.g., T7 promoter). Any gRNA scaffold may be used to create gRNAs compatible with Cas9s from any bacterial species.

Initial gRNA Screen

Each gRNA to be tested is transfected, along with a plasmid expressing Cas9 and a small amount of a GFP-expressing plasmid into human cells. In preliminary experiments, these cells can be immortalized human cell lines such as 293T, K562 or U205. Alternatively, primary human cells may be used. In this case, cells may be relevant to the eventual therapeutic cell target (e.g., a circulating blood cell, e.g., a T cell (e.g., a CD4+ T cell, a CD8+ T cell, a helper T cell, a regulatory T cell, a cytotoxic T cell, a memory T cell, a T cell precursor or a natural killer T cell)). The use of primary cells similar to the potential therapeutic target cell population may provide important information on gene targeting rates in the context of endogenous chromatin and gene expression.

Transfection may be performed using lipid transfection (such as Lipofectamine or Fugene) or by electroporation (such as Lonza Nucleofection). Following transfection, GFP expression can be determined either by fluorescence microscopy or by flow cytometry to confirm consistent and high levels of transfection. These preliminary transfections can comprise different gRNAs and different targeting approaches (17-mers, 20-mers, nuclease, dual-nickase, etc.) to determine which gRNAs/combinations of gRNAs give the greatest activity.

Efficiency of cleavage with each gRNA may be assessed by measuring NHEJ-induced indel formation at the target locus by a T7E1-type assay or by sequencing. Alternatively, other mismatch-sensitive enzymes, such as Cell/Surveyor nuclease, may also be used.

For the T7E1 assay, PCR amplicons are approximately 500-700 bp with the intended cut site placed asymmetrically in the amplicon. Following amplification, purification and size-verification of PCR products, DNA is denatured and re-hybridized by heating to 95° C. and then slowly cooling. Hybridized PCR products are then digested with T7 Endonuclease I (or other mismatch-sensitive enzyme) which recognizes and cleaves non-perfectly matched DNA. If indels are present in the original template DNA, when the amplicons are denatured and re-annealed, this results in the hybridization of DNA strands harboring different indels and therefore lead to double-stranded DNA that is not perfectly matched. Digestion products may be visualized by gel electrophoresis or by capillary electrophoresis. The fraction of DNA that is cleaved (density of cleavage products divided by the density of cleaved and uncleaved) may be used to estimate a percent NHEJ using the following equation: % NHEJ=(1-(1-fraction cleaved)^{1/2}). The T7E1 assay is sensitive down to about 2-5% NHEJ.

Sequencing may be used instead of, or in addition to, the T7E1 assay. For Sanger sequencing, purified PCR amplicons are cloned into a plasmid backbone, transformed, miniprepped and sequenced with a single primer. Sanger sequencing may be used for determining the exact nature of indels after determining the NHEJ rate by T7E1.

Sequencing may also be performed using next generation sequencing techniques. When using next generation sequencing, amplicons may be 300-500 bp with the intended cut site placed asymmetrically. Following PCR, next generation sequencing adapters and barcodes (for example Illumina multiplex adapters and indexes) may be added to the ends of the amplicon, e.g., for use in high throughput sequencing (for example on an Illumina MiSeq). This method allows for detection of very low NHEJ rates.

### Example 2

### Assessment of Gene Targeting by NHEJ

The gRNAs that induce the greatest levels of NHEJ in initial tests can be selected for further evaluation of gene targeting efficiency. In this case, cells are derived from disease subjects and, therefore, harbor the relevant mutation.

Following transfection (usually 2-3 days post-transfection,) genomic DNA may be isolated from a bulk population of transfected cells and PCR may be used to amplify the target region. Following PCR, gene targeting efficiency to generate the desired mutations (either knockout of a target gene or removal of a target sequence motif) may be determined by sequencing. For Sanger sequencing, PCR amplicons may be 500-700 bp long. For next generation sequencing, PCR amplicons may be 300-500 bp long. If the goal is to knockout gene function, sequencing may be used to assess what percent of alleles have undergone NHEJ-induced indels that result in a frameshift or large deletion or insertion that would be expected to destroy gene function. If the goal is to remove a specific sequence motif, sequencing may be used to assess what percent of alleles have undergone NHEJ-induced deletions that span this sequence.

### Example 3

### Screening of gRNAs for CCR5

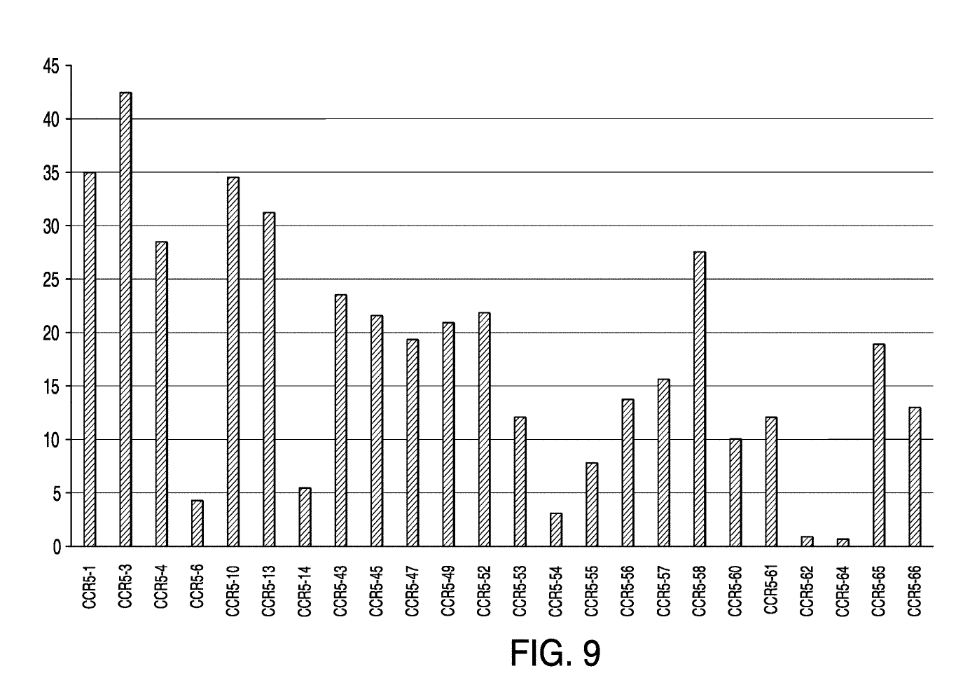

In order to identify gRNAs with the highest on target NHEJ efficiency, 24 S. pyogenes gRNAs were selected for testing (Table 10). A DNA plasmid comprised of an exemplary gRNA (including the target region and appropriate TRACR sequence) under the control of a U6 promoter was generated by restriction enzyme cloning. This DNA template was subsequently transfected into 293 cells using Lipofectamine 3000 along with a DNA plasmid encoding the appropriate Cas9 downstream of a CMV promoter. Genomic DNA was isolated from the cells 48-72 hours post transfection. To determine the rate of modification at the CCR5 gene, the target region was amplified using a locus PCR with the following primers (CCR5 exon 3 5′ primer: TATCAAGTGTCAAGTCCAATCTATGACATC (SEQ ID NO: 8410); CCR5 exon 3 3′ primer: GGAAATTCTTCCAGAATTGATACTGACTG (SEQ ID NO: 8411). After PCR amplification, a T7E1 assay was performed on the PCR product. Briefly, this assay involves melting the PCR product followed by a re-annealing step. If gene modification has occurred, there can exist double stranded products that are not perfect matches due to some frequency of insertions or deletions. These double stranded products are sensitive to cleavage by a T7 endonuclease 1 enzyme at the site of mismatch. Therefore, the efficiency of cutting by the Cas9/gRNA complex can be determined by analyzing the amount of T7E1 cleavage. The formula that is used to provide a measure of % NHEJ from the T7E1 cutting is the following: 100*(1-((1-(fraction cleaved))\hat{0}0.5)). The results of this analysis are shown in FIG. 9.

TABLE 10

| gRNA | Targeting Domain Sequence |
|---|---|
| CCR5-U1 | GCCUCCGCUCUACUCAC (SEQ ID NO: 230) |
| CCR5-U3 | GCCGCCCAGUGGGACUU (SEQ ID NO: 211) |
| CCR5-U4 | GCAUAGUGAGCCCAGAA (SEQ ID NO: 216) |
| CCR5-U6 | GCCUUUUGCAGUUUAUC (SEQ ID NO: 246) |
| CCR5-U10 | GACAAUCGAUAGGUACC (SEQ ID NO: 233) |
| CCR5-U13 | GACAAGUGUGAUCACUU (SEQ ID NO: 272) |
| CCR5-U14 | GGUACCUAUCGAUUGUC (SEQ ID NO: 248) |
| CCR5-U43 | GCUGCCGCCCAGUGGGACUU (SEQ ID NO: 335) |
| CCR5-U45 | GGUACCUAUCGAUUGUCAGG (SEQ ID NO: 315) |
| CCR5-U47 | GCAGCAUAGUGAGCCCAGAA (SEQ ID NO: 279) |
| CCR5-U49 | GUGAGUAGAGCGGAGGCAGG (SEQ ID NO: 314) |
| CCR5-U52 | AUGUGUCAACUCUUGAC (SEQ ID NO: 231) |
| CCR5-U53 | UUGACAGGGCUCUAUUUUAU (SEQ ID NO: 212) |
| CCR5-U54 | ACAGGGCUCUAUUUUAU (SEQ ID NO: 266) |
| CCR5-U55 | UCAUCCUCCUGACAAUCGAU (SEQ ID NO: 328) |
| CCR5-U56 | UCCUCCUGACAAUCGAU (SEQ ID NO: 209) |
| CCR5-U57 | CCUGACAAUCGAUAGGUACC (SEQ ID NO: 294) |
| CCR5-U58 | GGUGACAAGUGUGAUCACUU (SEQ ID NO: 334) |
| CCR5-U60 | CCAGGUACCUAUCGAUUGUC (SEQ ID NO: 309) |
| CCR5-U61 | ACCUAUCGAUUGUCAGG (SEQ ID NO: 253) |
| CCR5-U62 | UCAGCCUUUUGCAGUUUAUC (SEQ ID NO: 307) |
| CCR5-U64 | CACAUUGAUUUUUUGGC (SEQ ID NO: 243) |
| CCR5-U65 | AGUAGAGCGGAGGCAGG (SEQ ID NO: 252) |
| CCR5-U66 | CCUGCCUCCGCUCUACUCAC (SEQ ID NO: 291) |

### Example 4

### Assessment of Gene Targeting in Hematopoietic Stem Cells

Transplantation of autologous CD34⁺hematopoietic stem cells (HSCs) that have been genetically modified to prevent expression of the wild-type CCR5 gene product prevents entry of the HIV virus HSC progeny that are normally susceptible to HIV infection (e.g., macrophages and CD4 T-lymphocytes). Clinically, transplantation of HSCs that contain a genetic mutation in the coding sequence for the CCR5 chemokine receptor has been shown to control HIV infection long-term (Hütter et. al, New England Journal of Medicine, 2009; 360(7):692-698). Genome editing with the CRISPR/Cas9 platform precisely alters endogenous gene targets by creating an indel at the targeted cut site that can lead to knock down of gene expression at the edited locus. In this Example, genome editing in human mobilized peripheral blood CD34⁺ HSCs after co-delivery of Cas9 with gRNA targeting the CCR5 locus was evaluated to induce gene editing in CD34⁺ cells.

Human CD34⁺ HSCs cells from mobilized peripheral blood (AllCells) were thawed into StemSpan Serum-Free Expansion Medium (SFEM™, StemCell Technologies) containing 100 ng/mL each of the following cytokines: human stem cell factor (SCF), thrombopoietin (TPO), and flt-3 ligand (FL) (all from Peprotech). Cells were grown for 3 days in a humidified incubator and 5% CO₂ 20% O₂. On day 3, media was replaced with fresh Stemspan-SFEM™ supplemented with human SCF, TPO, FL and 40 nM of the small molecule UM171(Xcess Bio), a human HSC self-renewal agonist which has been shown to support robust expansion of human HSCs (Fares et. al, Science, 2014; 345(6203):1509-1512). The published use of UM171 involved prolonged exposure of HSCs to the small molecule for ex vivo expansion of HSCs. In the current experiment, HSCs were exposed to UM171 for 2 hours before and 24 hours after delivery of Cas9 and gRNA plasmid DNA. This UM171 treatment protocol was based on pilot studies performed by the inventors that indicated acute pre-treatment with UM171 before lentivirus vector mediated gene delivery improved HSC viability compared to HSCs treated with vehicle (dimethylsulfoxide, DMSO, Sigma) alone. After the 2-hour pretreatment with UM171, 1 million CD34⁻ HSCs were Nucleofected™ with the Amaxa™ 4D Nucleofector™ device (Lonza), Program EO100 using components of the P3 Primary Cell 4D-Nucleofector Kit™ (Lonza) according to the manufacturer's instructions. Briefly, one million cells were suspended in Nucleofector™ solution and the following amounts of plasmid DNA were added to the cell suspension: 1250 ng plasmid expressing CCR5 gRNA (CCR5-U43) from the human U6 promoter and 3750 ng plasmid expressing wild-type S. pyogenes Cas 9 transcriptionally regulated by the CMV promoter. After Nucleofection™, cells were plated into Stemspan-SFEM™ supplemented with SCF, TPO, FL and 40 nM UM171. After overnight incubation, HSCs were plated in Stemspan-SFEM™ plus cytokines without UM171. At 96 hours after Nucleofection™, CD34⁺ cells were counted for by trypan blue exclusion and divided into 3 portions for the following analyses: a) flow cytometry analysis for assessment of viability by co-staining with 7-Aminoactinomycin-D (7-AAD) and allophycocyanin (APC)-conjugated Annexin-V antibody (ebioscience); b) flow cytometry analysis for maintenance of HSC phenotype (after co-staining with phycoerythrin (PE)-conjugated anti-human CD34 antibody and fluorescein isothicyanate (FITC)-conjugated anti-human CD90, both from BD Bioscience; c) hematopoietic colony forming cell (CFC) analysis by plating 1500 cells in semi-solid methylcellulose based Methocult medium (StemCell Technologies) that supports differentiation of erythroid and myeloid blood cell colonies from HSCs and serves as a surrogate assay to evaluate HSC multipotency and differentiation potential ex vivo; d) genomic DNA analysis for detection of editing at the CCR5 locus. Genomic DNA was extracted from HSCs 96 hours after Nucleofection™, and CCR5 locus-specific PCR reactions were performed.

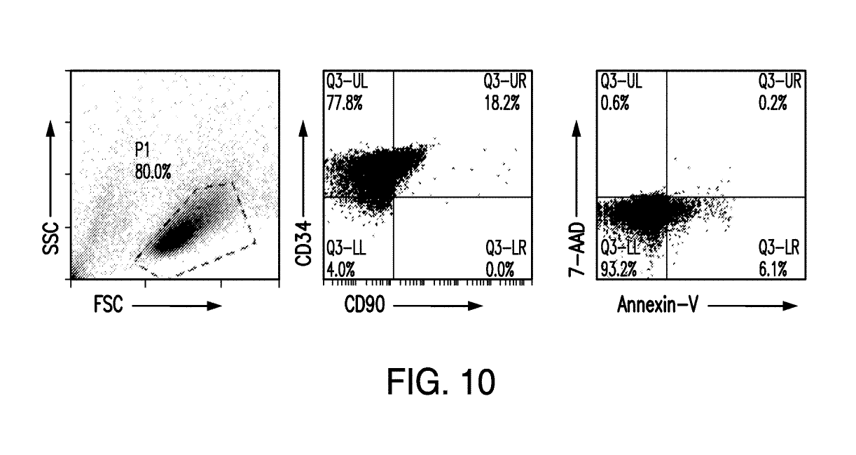

HSCs that were Nucleofected™ with Cas9 and CCR5 gRNA plasmids after pre-treatment with UM171 exhibited >93% viability (7-AAD⁻ AnnexinV⁻) and maintained co-expression of CD34 and CD90, as determined by flow cytometry analysis (FIG. 10). In addition, the UM171-treated Nucleofected™ cells were able to divide, as there was no difference in the total cell number between nucleofected UM171 treated cells and unelectroporated HSCs (Table 11). In contrast, HSCs Nucleofected™ without UM171 pre-treatment had decreased viability and cell did not expand in culture.

Table 11 shows that UM171 preserved CD34⁺ HSC viability after Nucleofection™ with wild type Cas9 and CCR5-U43 gRNA plasmid DNA (96 hours)

TABLE 11

| | Fold change in cell |
|---|---|
| | number of CD34⁺ cells |
| Condition | (96 hours vs. time 0 cell number) |
| No Nucleofection ™ | 1.6 |
| Nucleofection ™ + UM171 treatment | 1.5 |
| Nucleofection ™ + vehicle treatment | 0.6 |

In order to detect indels at the CCR5 locus, T7E1 assays were performed on CCR5 locus-specific PCR products that were amplified from genomic DNA samples from Nucleofected™ CD34⁺ HSCs and then percentage of indels detected at the CCR5 locus was calculated. Twenty percent indels was detected in the genomic DNA from CD34⁺ HSCs Nucleofected™ with Cas9 and CCR5 gRNA plasmids after pre-treatment with UM171.

To evaluate maintenance of HSC potency and differentiation potential, two weeks after plating CD34⁺ HSCs in CFC assays, hematopoietic activity was quantified based on scoring the HSC progeny by enumerating the total number of hematopoietic colony forming units (CFU) and the frequencies of specific blood cell phenotypes, including: mixed myeloid/erythroid (Granulocyte-erythroid-monocyte macrophage, CFU-GEMM), myeloid (CFU-macrophage (M), granulocyte-macrophage (CFU-GM)) and erythroid (CFU-E) colonies. CD34⁺ HSCs that were Nucleofected™ after UM171 pre-treatment maintained CFC potential compared to un-Nucleofected™ HSCs (Table 12). In contrast, CD34⁺ HSCs that were Nucleofected™ without UM171 pre-treatment had reduced CFC potential (lower total CFC counts and reduced numbers of mixed-phenotype colonies (CFU-GEMM) and erythroid colonies (CFU-E)) in comparison to un-Nucleofected™ CD34⁺ HSCs.

Table 12 shows that UM171 preserved CD34⁺ HSC viability after Nucleofection™ with wild-type Cas9 and CCR5 -U43 gRNA plasmid DNA (two weeks).

TABLE 12

| | Number of colony forming units per | | | | | |
|---|---|---|---|---|---|---|
| | 1500 CD34⁺ HSCs plated | | | | | |
| Condition | E | G | M | GM | GEMM | Total |
| No Nucleofaction ™ | 64 | 3 | 88 | 5 | 11 | 171 |
| Nucleofection ™ + UM171 | 92 | 40 | 64 | 32 | 20 | 228 |
| Nucleofaction ™ + vehicle | 18 | 22 | 6 | 1 | 1 | 28 |

Delivery of co-delivery wild-type S. pyogenes Cas9 and a single CCR5 gRNA plasmid DNA supported 20% genome editing of CD34⁺ HSCs, without loss of cell viability, multipotency, self-renewal and differentiation potential. Pre-treatment and short-term (24-hour) co-culture with the HSC self-renewal agonist UM171 was critical for maintenance of HSC survival and proliferation after Nucleofection™ with Cas9/gRNA DNA. Clinically, transplantation of HSCs that contain a genetic mutation in the CCR5 gene generated by CRISPR/Cas9 related methods can be used to achieve long term control of HIV infection.

### Example 5

### Assessment of Genome Editing at the CXCR4 Genetic Locus in Hematopoietic Stem Cells

Transplantation of autologous CD34⁺ hematopoietic stem cells (HSCs, also known as hematopoietic stem/progenitor cells or HSPCs) that have been genetically modified to prevent expression of the wild-type CXCR4 gene product prevents entry of the HIV virus HSC progeny that are normally susceptible to HIV infection (e.g., macrophages and CD4 T-lymphocytes). Genome editing with the CRISPR/Cas9 platform precisely alters endogenous gene targets by creating an indel at the targeted cut site that can lead to knock down of gene expression at the edited locus. In this Example, genome editing in human mobilized peripheral blood CD34⁺ HSCs after co-delivery of Cas9 with gRNA targeting the CXCR4 locus was evaluated to induce gene editing in CD34⁺ cells. Streptococcus pyogenes (S. pyogenes) and Staphylococcus aureus (S. aureus) Cas9 variants paired with CXCR4 gRNAs were used in this example.

Human CD34⁺ HSCs cells from mobilized peripheral blood (AllCells) were thawed into StemSpan Serum-Free Expansion Medium (SFEM, StemCell Technologies) containing 100 ng/mL each of the following cytokines: human stem cell factor (SCF), thrombopoietin (TPO), and flt-3 ligand (FL) (all from Peprotech). Cells were grown for 3 days in a humidified incubator and 5% CO₂ 20% O₂. On day 3, media was replaced with fresh Stemspan-SFEM supplemented with human SCF, TPO, FL±40 nM of the small molecule UM171 (Xcess Bio), a human HSC self-renewal agonist which has been shown to support robust expansion of human HSCs (Fares et. al, SCIENCE, 2014; 345(6203):1509-1512). The published use of UM171 involved prolonged exposure of HSCs to the small molecule for ex vivo expansion of HSCs. In the current experiment, HSCs were exposed to UM171 for 2 hours before and 24 hours after delivery of Cas9 and gRNA plasmid DNA. This UM171 treatment protocol was based on the pilot studies that indicated acute pre-treatment with UM171 before lentivirus vector mediated gene delivery improved HSC viability compared to HSCs treated with vehicle (dimethysulfoxide, DMSO, Sigma) alone. After the 2-hour pretreatment with UM171, 200,000 CD34⁺ HSCs were Nucleofected™ with the Amaxa™ 4D Nucleofector™ device (Lonza), using components of the P3 Primary Cell 4D-Nucleofector Kit™ (Lonza) according to the manufacturer's instructions. Briefly, 200,000 CD34⁺ cells were suspended in Nucleofector™ solution and the following amounts of plasmid DNA were added to the cell suspension: 250 ng plasmid expressing S. pyogenes CXCR4 gRNA (CXCR4-231; targeting domain sequence: GCGCUUCUGGUGGCCCU) or S. aureus CXCR4 gRNA (CXCR4-836; targeting domain sequence: GCUCCAAGGAAAGCAUAGAGGA) from the human U6 promoter each paired with 750 ng plasmid expressing either wild-type S. pyogenes Cas9 or S. aureus Cas9, each regulated by the CMV promoter. After Nucleofection™, cells were plated into Stemspan-SFEM™ supplemented with SCF, TPO, FL with or without 40 nM UM171. After overnight incubation, HSCs were plated in Stemspan-SFEM™ plus cytokines without UM171. At 96 hours after Nucleofection™, CD34⁺ cells were counted for by trypan blue exclusion and divided into 3 portions for the following analyses: a) flow cytometry analysis for assessment of viability by co-staining with 7-Aminoactinomycin-D (7-AAD) and allophycocyanin (APC)-conjugated Annexin-V antibody (ebioscience); b) flow cytometry analysis for maintenance of HSC phenotype (after co-staining with phycoerythrin (PE)-conjugated anti-human CD34 antibody and fluorescein isothicyanate (FITC)-conjugated anti-human CD90, both from BD Bioscience; c) hematopoietic colony forming cell (CFC) analysis by plating 1500 cells in semi-solid methylcellulose based Methocult medium (StemCell Technologies) that supports differentiation of erythroid and myeloid blood cell colonies from HSCs and serves as a surrogate assay to evaluate HSC multipotency and differentiation potential ex vivo; d) genomic DNA analysis for detection of editing at the CXCR4 locus. Genomic DNA was extracted from HSCs 96 hours after Nucleofection™, and CXCR4 locus-specific PCR reactions were performed.

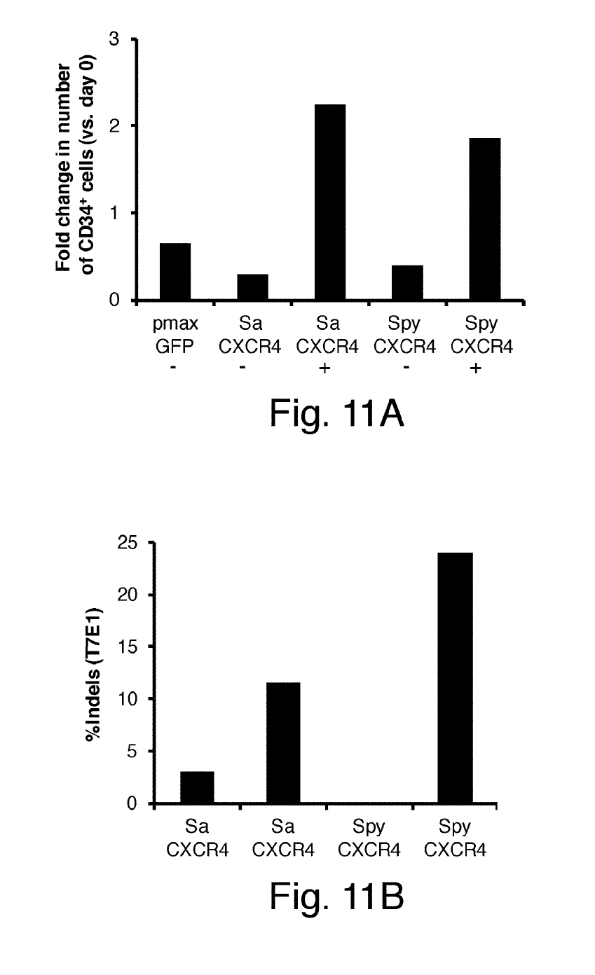

HSCs that were Nucleofected™ with Cas9 and CXCR4 gRNA (CXCR4-231) plasmids after pre-treatment with UM171 exhibited >95% viability (7-AAD⁻ AnnexinV⁻) and maintained co-expression of CD34 and CD90, as determined by flow cytometry analysis. In addition, the UM171-treated Nucleofected™ cells proliferated, as there was an increase in cell number similar to the level achieved with unelectroporated HSCs (FIG. 11A). In contrast, HSCs Nucleofected™ without UM171 pre-treatment had decreased viability and the cell number decreased in culture relative to untreated control cells.

In order to detect indels at the CXCR4 locus, T7E1 assays were performed on CXCR4 locus-specific PCR products that were amplified from genomic DNA samples from Nucleofected™ CD34⁺ HSCs and then calculated the percentage of NHEJ detected at the CXCR4 locus (FIG. 11B). HSCs pre-treated with UM171 exhibited a higher fold-expansion and higher percentage of genome editing at the CXCR4 locus after delivery of S. aureus or S. pyogenes Cas9 and CXCR4 gRNAs compared to HSCs that were not pre-treated with UM171.

To evaluate maintenance of HSC potency and differentiation potential, two weeks after plating CD34⁺ HSCs in CFC assays, hematopoietic activity was quantified based on scoring the HSC progeny by enumerating the total number of hematopoietic colony forming units (CFU) and the frequencies of specific blood cell phenotypes, including: mixed myeloid/erythroid (Granulocyte-erythroid-monocyte macrophage, CFU-GEMM), myeloid (CFU-macrophage (M), granulocyte-macrophage (CFU-GM)) and erythroid (CFU-E) colonies. CD34⁺ HSCs that were pre-treated with UM171 and Nucleofected™ with either S. aureus Cas9 and CXCR4-836 gRNA or S. pyogenes Cas9 and CXCR4-231 gRNA maintained CFC potential compared to un-Nucleofected™ HSCs (Table 13). In contrast, CD34⁺ HSCs that were Nucleofected™ with either Cas9 variant paired with CXCR4 gRNA without UM171 pre-treatment had reduced CFC potential (lower total CFC counts and reduced numbers of mixed-phenotype colonies (CFU-GEMM) and erythroid colonies (CFU-E) in comparison to un-Nucleofected™ CD34⁺ HSCs.

TABLE 13
UM171 preserves CD34⁺ HSC viability after Nucleofection ™
S. aureus (Sa) Cas9 and S. pyogenes (Spy) Cas9
paired with CXCR4 gRNA plasmid DNA (two weeks).

| | Number of colony forming units per | | | | | |
|---|---|---|---|---|---|---|
| | 1500 CD34⁺ HSCs plated | | | | | |
| Condition | E | G | M | GM | GEMM | Total |
| No Nucleofection ™ | 64 | 3 | 88 | 5 | 11 | 171 |
| Sa Cas9 + CXCR4-836 gRNA | 67 | 45 | 29 | 19 | 19 | 212 |
| Nucleofection ™ + UM171 | | | | | | |
| Spy Cas9 + CXCR4-231 gRNA | 60 | 29 | 61 | 27 | 13 | 173 |
| Nucleofection ™ + UM171 | | | | | | |
| Sa Cas9 + CXCR4-836 gRNA | 13 | 1 | 6 | 1 | 0 | 2 |
| Nucleofection ™ + vehicle | | | | | | |
| Spy Cas9 + CXCR4-231 gRNA | 12 | 2 | 4 | 2 | 2 | 1 |
| Nucleofection ™ + vehicle | | | | | | |

Co-delivery wild-type S. pyogenes Cas9 and CXCR4-231 gRNA plasmid DNA or S. aureus Cas9 and CXCR4-836 gRNA supported up to 25% genome editing of CD34⁺ HSCs, without loss of cell viability, multipotency, self-renewal and differentiation potential. Pre-treatment and short-term (24-hour) co-culture with the HSC self-renewal agonist UM171 was critical for maintenance of HSC survival and proliferation after Nucleofection™ with Cas9/gRNA DNA. Clinically, transplantation of HSCs that contain a genetic mutation in the CXCR4 gene generated by CRISPR/Cas9 related methods could be used to achieve long-term control of HIV infection.

### Example 6

### Assessment of Multiplex Gene Targeting at the Ccr5 and Cxcr4 Genetic Loci in Hematopoietic Stem Cells

Transplantation of autologous CD34⁺ hematopoietic stem cells (HSCs, also known as hematopoietic stem/progenitor cells or HSPCs) that have been genetically modified to prevent expression of the wild-type CXCR4 or the CCR5 gene product prevents entry of the HIV virus HSC progeny that are normally susceptible to HIV infection (e.g., macrophages and CD4 T-lymphocytes). Multiplex genome editing with the CRISPR/Cas9 platform precisely alters more than one endogenous gene targets by creating indels at two different cut sites can lead to knock down of gene expression at multiple edited loci. In this Example, multiplex genome editing in human mobilized peripheral blood CD34⁺HSCs after co-delivery of wild-type S. pyogenes Cas9 with one gRNA targeting the CXCR4 locus and one gRNA targeting the CCR5 locus was evaluated to induce multiplex gene editing in CD34⁺ cells.

Human CD34⁺ HSCs cells from mobilized peripheral blood (AllCells) were thawed into StemSpan Serum-Free Expansion Medium (SFEM™, StemCell Technologies) containing 100 ng/mL each of the following cytokines: human stem cell factor (SCF), thrombopoietin (TPO), and flt-3 ligand (FL) (all from Peprotech). Cells were grown for 3 days in a humidified incubator and 5% CO₂ 20% O₂. On day 3, media was replaced with fresh Stemspan-SFEM™ supplemented with human SCF, TPO, FL and 40 nM of the small molecule UM171(Xcess Bio), a human HSC self-renewal agonist that has been shown to support robust expansion of human HSCs (Fares et. al, Science, 2014; 345(6203):1509-1512). The published use of UM171 involved prolonged exposure of HSCs to the small molecule for ex vivo expansion of HSCs. In the current experiment, HSCs were exposed to UM171 for 2 hours before and 24 hours after delivery of Cas9 and gRNA plasmid DNA. This UM171 treatment protocol was based on the pilot studies that indicated acute pre-treatment with UM171 before lentivirus vector mediated gene delivery improved HSC viability compared to HSCs treated with vehicle (dimethysulfoxide, DMSO, Sigma) alone. After the 2-hour pretreatment with UM171, 200,000 CD34⁺ HSCs were Nucleofected™ with the Amaxa™ 4D Nucleofector™ device (Lonza), using components of the P3 Primary Cell 4D-Nucleofector Kit™ (Lonza) according to the manufacturer's instructions. Briefly, 200,000 CD34⁺ cells were respended in Nucleofector™ solution and the following amounts of plasmid DNA were added to the cell suspension: 250 ng plasmid expressing S. pyogenes CXCR4 gRNA (CXCR4-231) from the human U6 promoter, 250 ng plasmid expressing S. pyogenes CCR5 gRNA (CCR5-43) from the human U6 promoter and 750 ng plasmid expressing wild-type S. pyogenes Cas9 regulated by the CMV promoter. After Nucleofection™, cells were replated into Stemspan-SFEM supplemented with SCF, TPO, FL and UM171. After overnight incubation, HSCs were replated in Stemspan-SFEM™ plus cytokines alone without UM171. At 96 hours after Nucleofection™, CD34⁺ cells were counted by trypan blue exclusion and divided into 3 portions for the following analyses: a) flow cytometry analysis for assessment of viability by co-staining with 7-Aminoactinomycin-D (7-AAD) and allophycocyanin (APC)-conjugated Annexin-V antibody (ebioscience); b) flow cytometry analysis for maintenance of HSC phenotype (after co-staining with phycoerythrin (PE)-conjugated anti-human CD34 antibody and fluorescein isothicyanate (FITC)-conjugated anti-human CD90, both from BD Bioscience; c) hematopoietic colony forming cell (CFC) analysis by plating 1500 cells in semi-solid methylcellulose based Methocult™ medium (StemCell Technologies) that supports differentiation of erythroid and myeloid blood cell colonies from HSCs and serves as a surrogate assay to evaluate HSC multipotency and differentiation potential ex vivo; d) genomic DNA analysis for detection of editing at the CXCR4 and CCR5 loci. Genomic DNA was extracted from HSCs 96 hours after Nucleofection™, and CXCR4 and CCR5 locus-specific PCR reactions were performed.

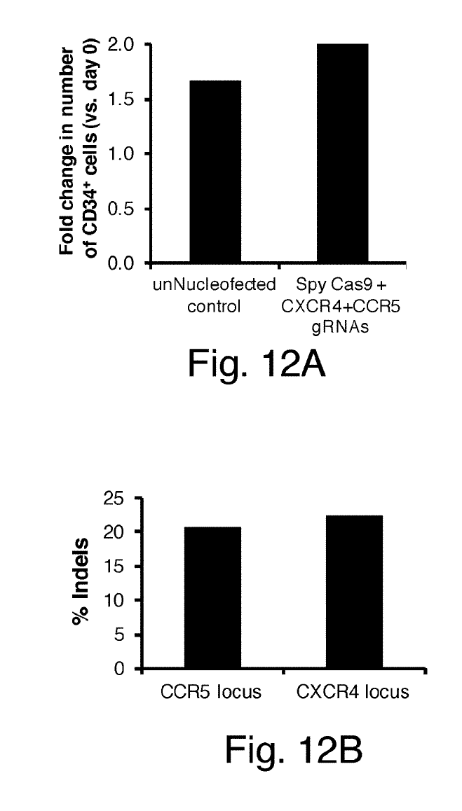

HSCs that were Nucleofected™ with Cas9 and CXCR4 (CXCR4-231) and CCR5 (CCR5-43) gRNA plasmids exhibited >90% viability (7-AAD⁻ AnnexinV⁻) and maintained co-expression of CD34 and CD90, as determined by flow cytometry analysis. In addition, Nucleofected™ cells were able to proliferate, as there was an increase in cell number with a fold-expansion similar to the level achieved in unelectroporated HSCs (FIG. 12A).

In order to detect indels at the CXCR4 and CCR5 loci, T7E1 assays were performed on CXCR4 andCCR5 locus-specific PCR products that were amplified from genomic DNA samples from Nucleofected™ CD34⁺ HSCs and the percentages of indels detected at the CXCR4 and CCR5 genomic loci were calculated. Up to 22% genome editing was detected at the two targeted loci in genomic DNA from CD34⁺ HSCs (FIG. 12B).

To evaluate maintenance of HSC potency and differentiation potential, two weeks after plating CD34⁺ HSCs in CFC assays, hematopoietic activity was quantified based on scoring the HSC progeny by enumerating the total number of hematopoietic colony forming units (CFU) and the frequencies of specific blood cell phenotypes, including: mixed myeloid/erythroid (Granulocyte-erythroid-monocyte macrophage, CFU-GEMM), myeloid (CFU-macrophage (M), granulocyte-macrophage (CFU-GM)) and erythroid (CFU-E) colonies. CD34⁺ HSCs that were Nucleofected™ CD34⁺ HSCs maintained CFC potential compared to un-Nucleofected™ HSCs (Table 14).

TABLE 14
Hematopoietic colony forming potential of un-Nucleofected ™
and Nucleofected ™ CD34⁺ HSCs (2 weeks).

| | Number of colony forming units per | | | | | |
|---|---|---|---|---|---|---|
| | 1500 CD34⁺ HSCs plated | | | | | |
| Condition | E | G | M | GM | GEMM | Total |
| No Nucleofaction ™ | 64 | 3 | 88 | 5 | 11 | 171 |
| Nucleofaction ™ with | 76 | 41 | 73 | 19 | 8 | 217 |
| S. pyogenes Cas9 + CXCR4 | | | | | | |
| gRNA and CCR5 gRNA | | | | | | |

Co-delivery wild-type S. pyogenes Cas9, CXCR4 gRNA, and CCR5 gRNA expressing DNA plasmids supported up efficient genome editing at the two targeted loci, without loss of cell viability, multioptency, self-renewal and differentiation potential. Clinically, transplantation of HSCs that contain genetic mutations in both the CCR5 and CXCR4 genes generated by CRISPR/Cas9 related multiplexing methods could be used to achieve long-term control of HIV infection.

### Example 7

### Modification of gRNA by Addition of 5′ Cap and 3′ Poly-A Tail Improves Increases Genome Editing at Target Genetic Loci and Improves CD34+ Cell Viability and Survival

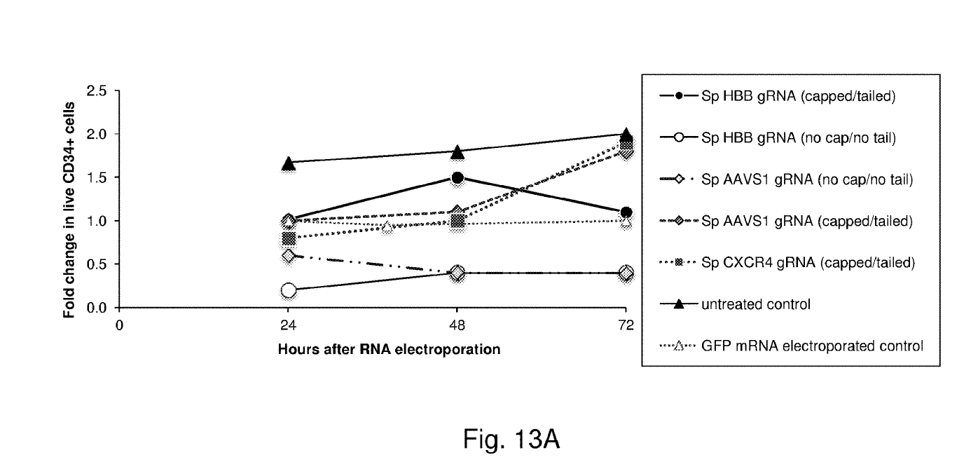

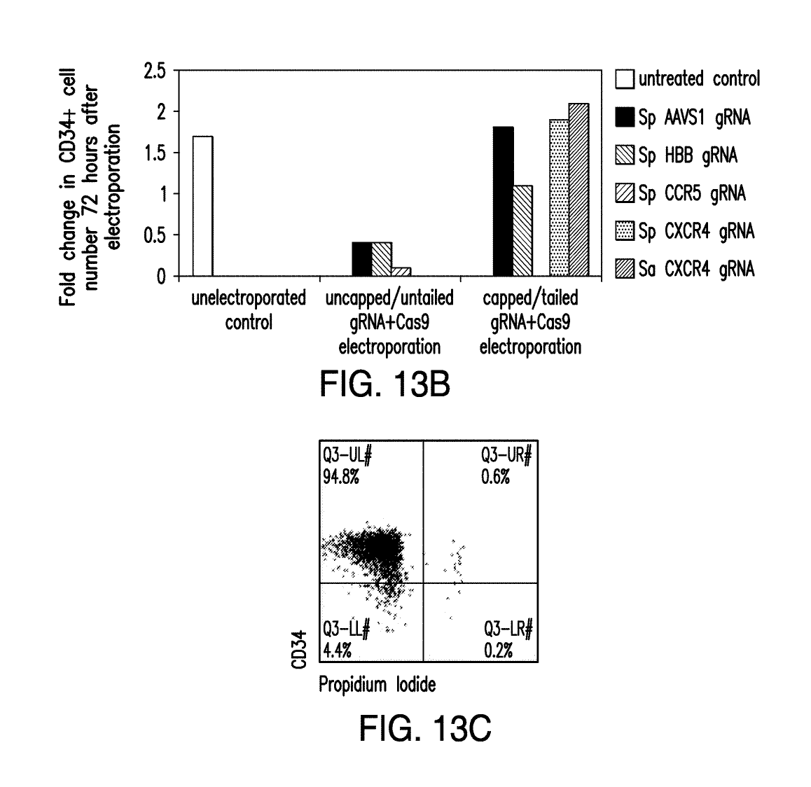

During virus-host co-evolution, viral RNA capping that mimics capping of mRNA evolved to allow viral RNA to escape detection from the cell's innate immune system (Delcroy et al., 2012, NATURE REVIEWS MICROBIOLOGY, 10:51-65). Toll-like receptors in hematopoietic stem/progenitor cells sense the presence of foreign single and double stranded RNA that can lead to innate immune response, cell senescence, and programmed cell death (Kaj aste-Rudnitski and Naldini, 2015, HUMAN GENE THERAPY, 26:201-209). Results from initial experiments showed that human hematopoietic stem/progenitor cells electroporated with unmodified target specific gRNA and Cas9 mRNA led to reduced cell survival, proliferation potential, multipotency (e.g., loss of erythroid differentiation potential and skewed myeloid differentiation potential) compared to cells electroporated with GFP mRNA alone. In order to address this issue, it was hypothesized that cell senescence and apoptosis was due to the target cell sensing of foreign nucleic acid and induction of an innate immune response and subsequent induction of programmed cell death and loss of proliferative and differentiation potential. Toward optimization of genome editing in hematopoietic/stem progenitor cells and to test this hypothesis, human CD34⁺ cells from mobilized peripheral blood and bone marrow were electroporated (using the Maxcyte device) with S. pyogenes Cas9 mRNA co-delivered with HBB or AAVS1 targeted gRNA in vitro transcribed with or without the addition of a 5′ cap and 3′ poly-A tail. Human CD34⁺ cells that were electroporated with Cas9 paired with a single uncapped and untailed HBB or AAVS1 gRNA exhibited decreased proliferation potential over 3 days in culture compared to cells that were electroporated with the same gRNA sequence that was in vitro transcribed to have a 5′ cap and a 3′ polyA tail (FIG. 13A). Other capped and tailed gRNAs (targeted to HBB, AAVS1, CXCR4, and CCR5 loci) delivered with Cas9 mRNA did not negatively impact HSPC viability, proliferation, or multipotency, as determined by comparison of the fold expansion of total live CD34⁺ cells over three days after delivery. Importantly, there was no difference in the proliferative potential of CD34⁺ cells contacted with capped and tailed gRNA and Cas9 mRNA compared to cells contacted with GFP mRNA or cells that were untreated. Analysis of cell viability (by co-staining with either 7-aminoactinomycin D or propidium iodide with AnnexinV antibody followed by flow cytometry analysis) at seventy-two hours after contacting Cas9 mRNA and gRNAs indicated that cells that contacted capped and tailed gRNAs expanded in culture and maintained viability HSPCs that contacted uncapped and tailed gRNAs exhibited a decrease in viable cell number (FIG. 13B). Viable cells (propidium iodide negative) that contacted capped and tailed gRNAs also maintained expression of the CD34 cell surface marker (FIG. 13C).

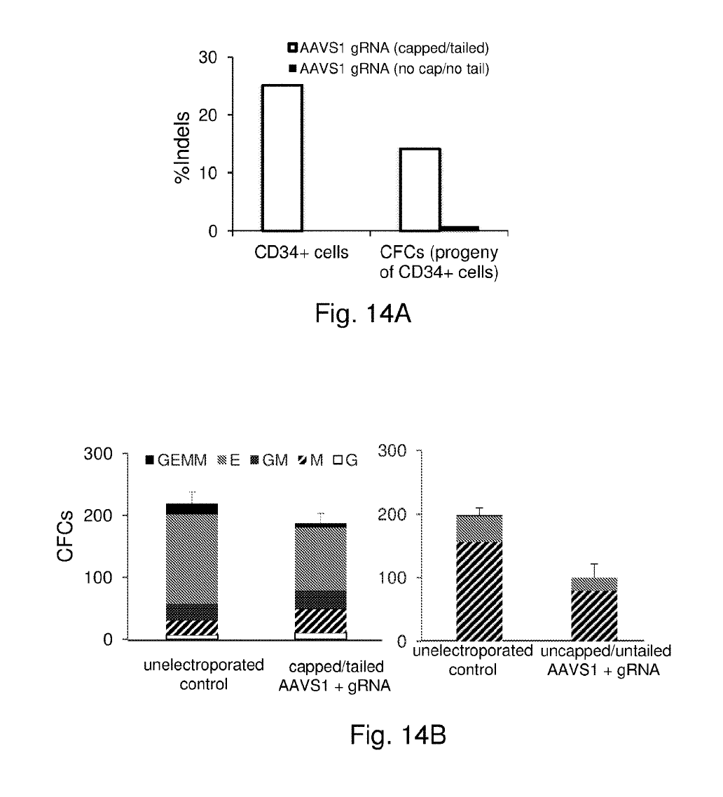

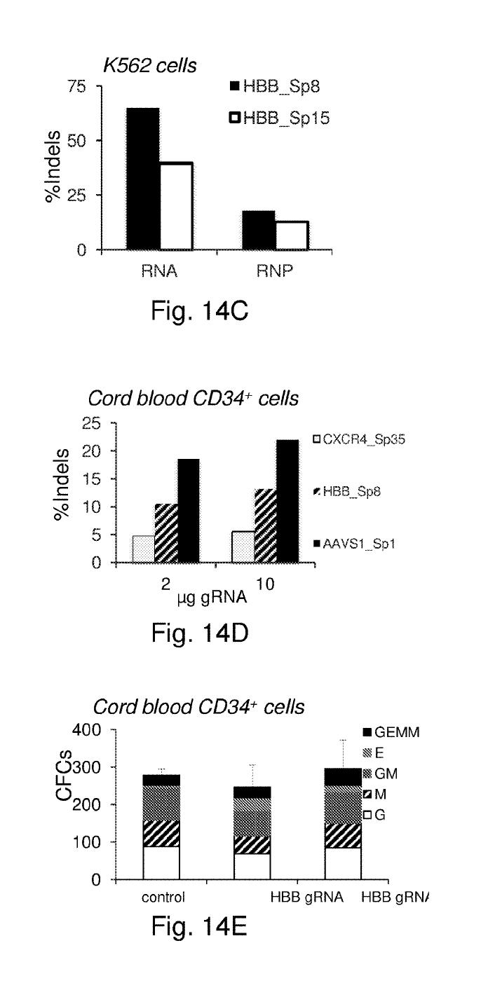

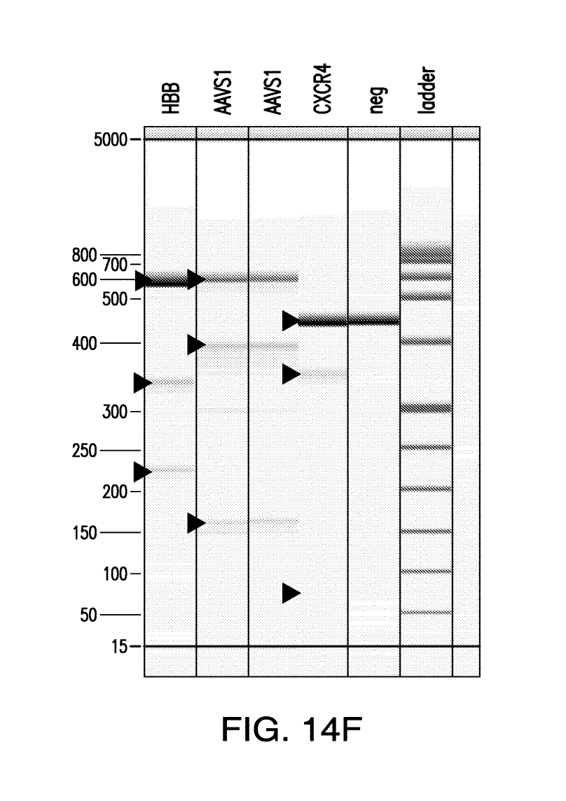

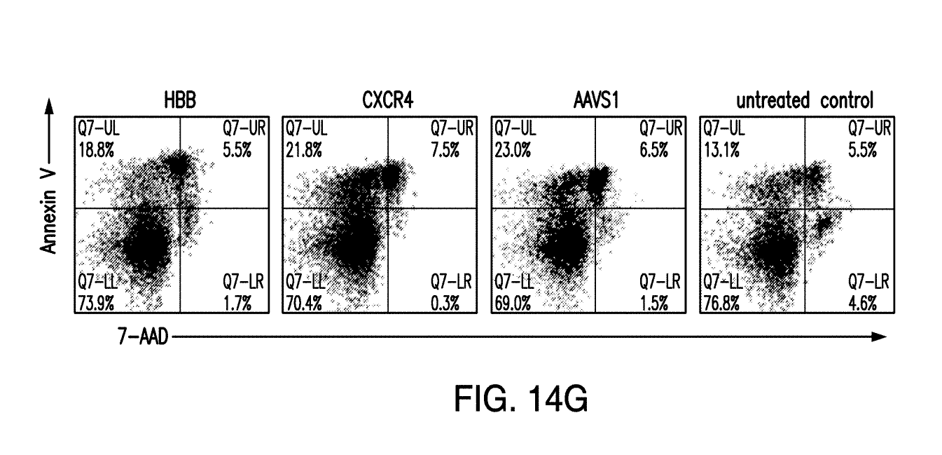

In addition to the improved survival, target cells that contacted capped and tailed AAVS1 specific gRNA also exhibited a higher percentage of on-target genome editing (% indels) compared to cells that contacted Cas9 mRNA and uncapped/untailed gRNAs (FIG. 14A). In addition, a higher level of targeted editing was detected in the progeny of CD34⁺ cells that contacted Cas9 mRNA with capped/tailed gRNA compared to the progeny of CD34⁺ cells that contacted Cas9 mRNA with uncapped/untailed gRNA (FIG. 14A, CFCs). Delivery of uncapped/untailed gRNA also reduced the ex vivo hematopoietic potential of CD34⁺ cells, as determined in colony forming cell (CFC) assays. Cells that contacted uncapped an untailed gRNAs with Cas9 mRNA exhibited a loss in total colony forming potential (e.g., potency) and a reduction in the diversity of colony subtype (e.g. loss of erythroid and progenitor potential and skewing toward myeloid macrophage phenotype in progeny)(FIG. 14B). In contrast, cells that contacted capped and tailed gRNAs maintained CFC potential both with respect to the total number of colonies differentiated from the CD34+ cells and with respect to colony diversity (detected of mixed hematopoietic colonies [GEMMs] and erythroid colonies [E]).

Next capped and tailed HBB specific gRNAs were co-delivered with either Cas9 mRNA or complexed with Cas9 ribonucleoprotein (RNP) and then electroporated into K562 cells, a erythroleukemia cell line that been shown to mimic certain characteristics of HSPCs. Co-delivery of capped and tailed gRNA with Cas9 mRNA or RNP led to high level of genome editing at the HBB locus, as determined by T7E1 assay analysis of HBB locus PCR products (FIG. 14C). Next, 3 different capped and tailed gRNAs (targeting the HBB, AAVS1, and CXCR4 loci) were co-delivered with S. pyogenes Cas9 mRNA into CD34⁺ cells isolated from umbilical cord blood (CB). Here, different amounts of gRNA (2 or 10 μg gRNA plus 10 μg of S. pyogenes Cas9 mRNA) were electroporated into the cells and the percentages of genome editing evaluated at target loci by T7E1 assay analysis of locus PCR products. In contrast, no cleavage was detected at the HBB locus in the genomic DNA from CB CD34⁺ cells that were electroporated with uncapped and untailed HBB gRNA with Cas9 mRNA. The results indicated that CB CD34⁺ cells electroporated with Cas9 mRNA and capped and tailed gRNAs maintained proliferative potential and colony forming potential. Five to 20% indels were detected at target loci and the amount of capped and tailed gRNA co-delivered with the Cas9 mRNA did not impact the percentage of targeted editing (FIG. 14D).

A representative gel image of the indicated locus specific PCR products after T7E1 assay was performed shows cleavage at the targeted loci in CB CD34⁺ cells 72 hours after delivery of capped and tailed locus-specific gRNAs (AAVS1, HBB, and CXCR4 gRNAs) co-delivered with S. pyogense Cas9 mRNA by electroporation (Maxcyte device)(FIG. 14F). Importantly, there was no difference in the viability of the cells electroporated with capped and tailed AAVS1-specific gRNA, HBB-specific gRNA, or CXCR4-specific gRNA co-delivered with S. pyogenes Cas9 mRNA compared to cells that did not contact Cas9 mRNA or gRNA (i.e., untreated control). Live cells are indicated by negative staining for 7-AAD and AnnexinV as determined by flow cytometry analysis (bottom left quadrants of flow cytometry plots, FIG. 14G). CB CD34⁺ cells electroporated with capped and tailed AAVS1 specific gRNA, HBB-specific gRNA, or CXCR4-specific gRNA co-delivered with S. pyogenes Cas9 mRNA maintained ex vivo hematopoietic colony forming potential as determined by CFC assays. The representation ex vivo hematopoietic potential in CFC assays for cells that contacted HBB-specific gRNA and Cas9 is shown in FIG. 14E.

### Example 8

### Assessment of Gene Editing by S. aureus Cas9/gRNAs Targeting the Human CCR5 Locus in Human K562 Cells

To identify gRNAs that efficiently target disruption of the human CCR5 gene, eleven gRNAs were selected from a larger list of gRNAs obtained from in silico prediction of gRNAs with S. aureus specific PAM sequences. In silico predicted gRNAs were tiered according to the strategy described in Section 8. An abbreviated list of eleven gRNAs with the lowest predictive off-target scores were selected for subsequent screening experiments, based on proximity to the naturally occurring de1ta32 mutation in CCR5 that has been associated with resistance to HIV. The target-specific complementary region of the selected list of eleven gRNAs are depicted in Table 15. Table 15 depicts the gRNA target-specific complementary sequences evaluated in Example 8.

TABLE 15

| | | Target-specific complementary |
|---|---|---|
| gRNA ID | Size | sequence |
| CCR5_Sa1 | 22 | GCCUAUAAAAUAGAGCCCUGUC (SEQ ID |
| | | NO: 480) |
| CCR5_Sa2 | 22 | AUACAGUCAGUAUCAAUUCUGG (SEQ ID |
| | | NO: 1000) |
| CCR5_Sa3 | 20 | GUGGUGACAAGUGUGAUCAC (SEQ ID |
| | | NO: 488) |
| CCR5_Sa4 | 24 | CCAUACAGUCAGUAUCAAUUCUGG (SEQ ID |
| | | NO: 1002) |
| CCR5_Sa5 | 24 | AAGCCUAUAA AAUAGAGCCCUGUC (SEQ ID |
| | | NO: 482) |
| CCR5_Sa6 | 24 | UGGGGUGGUG ACAAGUGUGAUCAC (SEQ ID |
| | | NO: 492) |
| CCR5_Sa7 | 22 | GGGUGGUGACAAGUGUGAUCAC (SEQ ID |
| | | NO: 490) |
| CCR5_Sa8 | 18 | GGUGACAAGUGUGAUCAC (SEQ ID |
| | | NO: 486) |
| CCR5_Sa9 | 22 | GCCUUUUGCAGUUUAUCAGGAU (SEQ ID |
| | | NO: 512) |
| CCR5_Sa10 | 24 | GCUCUAUUUUAUAGGCUUCUUCUC (SEQ ID |
| | | NO: 535) |
| CCR5_Sa11 | 24 | GCUCUUCAGCCUUUUGCAGUUUAU (SEQ ID |
| | | NO: 521) |

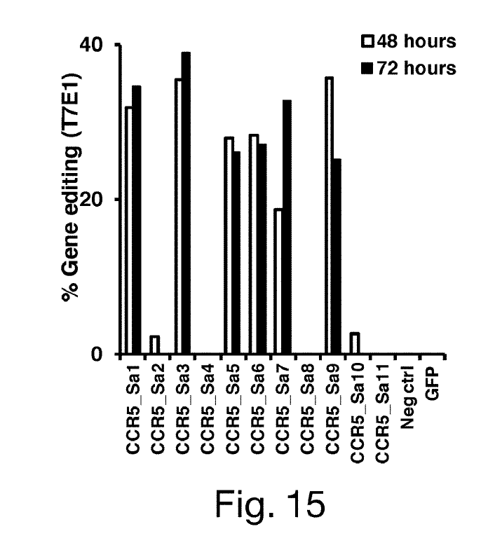

A DNA plasmid encoding an S. aureus expression cassette (AF002) and a gRNA-specific STITCHR product, which is a DNA molecule consisting of a U6 promoter driving expression of the chimeric gRNA (i.e., a target-specific complementary sequence and the S. aureus gRNA scaffold), were electroporated into K562 cells using the Amaxa Nucleofector system and the program and protocol for K562 cells per the manufacturer's instructions. In brief, 750 ng S. aureus Cas9 plasmid DNA and 250 ng of STITCHR product were used for each gRNA. Forty-eight and 72 hours after electroporation, gDNA was isolated from nucleofected K562 cells and CCR5 specific PCRs were performed followed by T7E1 endonuclease assay on the CCR5 PCR product to evaluate NHEJ at the target site. Six out of the 11 screened gRNAs led to >20% indels in CCR5 (FIG. 15). From this data set, two gRNAs with the highest activity, CCR5_Sa1 and CCR5_Sa3, which supported about 35% and about 39% indels in K562 cells, respectively, were selected for use in subsequent testing of Cas9 RNP experiments in primary human T lymphocytes and CD34⁺HSCs.

### Example 9

### Assessment of Multiplex Gene Targeting at the Ccr5 and Cxcr4 Genetic Loci in Human T Lymphocytes with S. pyogenes and S. aureus Wild-Type Cas9 and DJOA Nickase Ribonucleoprotein Complexes Delivered by Electroporation

While transplantation of autologous CD34⁺ HSCs genetically modified to prevent expression of the wild-type CXCR4 or the CCR5 would provide a long-term cure to HIV infection, the myeloablative conditioning associated with HSC transplantation destroys host adaptive immunity until long-term engraftment is achieved until the T cell pool is reconstituted after HSC engraftment is acheived, which can take several months. This delay in adaptive immune reconstitution puts patients at risk for the development of opportunistic infections during the acute phase of early engraftment following HSC transplantation. One strategy to prevent this gap in adaptive immunity and to restore T cell function in HIV infected patients before HSC engraftment is stabilized, is to disrupt expression of HIV co-receptors CCR5 and CXCR4 in uninfected T lymphocytes collected from the patient (when patient is on HAART therapy and viral load is low) during or before collection of HSCs for transplantation. In this clinical scenario, HIV-resistant autologous T cells and HIV-resistant autologous HSCs would be co-infused into the original patient to support both short-term and long-term hematopoietic reconstitution. Alternatively, if a suitable HLA-matched or HLA-identical allogeneic HSC and T cell donor is identified for the patient, then the allogeneic donor T lymphocytes and HSCs could be modified with Cas9 RNP targeting disruption of CXCR4 and/or CCR5 HIV co-receptors to support immune and hematopoietic reconstitution. Electroporation of Cas9 RNPs, in which Cas9 protein is complexed with gRNAs targeting CCR5 and/or CXCR4 into HIV-negative patient T lymphocytes (including long-lived T memory stem cells) would support disruption of the HIV co-receptors leading to HIV resistance. In this Example, single and multiplex genome editing in human T lymphocytes after electroporation of Cas9 RNP targeting the CXCR4 locus, the CCR5 locus, or both simultaneously (multiplexing) was evaluated after electroporation of CXCR4 and CCR5 gRNAs that were in vitro transcribed and modified to have an ARCA cap at the 5′ end and a polyA (20A) tail at the 3′ end. Modified gRNAs compatible with S. pyogenes or S. aureus Cas9 were complexed with wild-type Cas9 protein or D10A nickase (for dual nickase strategy of gene disruption). The sgRNAs, gRNA pairs targeting the same locus, or two sgRNAs targeting different loci (i.e., both CXCR4 and CCR5 multiplexing) tested in this experiment are depicted in Table 16. Table 16 depicts experimental design associated with Example 9 to evaluate gene editing as determined by T7E1 endonuclease assay analysis of the CXCR4 and CCR5 loci after electroporation of primary human T lymphocytes with S. aureus and/or S. pyogenes RNPs.

TABLE 16

| | SEQ ID NO. | | SEQ ID NO. | | | |
|---|---|---|---|---|---|---|
| | for the | | for the | | | |
| | Targeting | | Targeting | | | |
| 1^{st} gRNA | domain of | 2^{nd} gRNA | domain of | Cas9 | % editing | % editing |
| (Species) | 1^{st} gRNA | (Species) | 2^{nd} gRNA | variant | at CXCR4 | at CCR5 |
| CXCR4-371 | 4118 | — | | WT | 3.63 | |
| (S. aureus) | | | | | | |
| CXCR4-836 | 4604 | — | | WT | 38.58 | |
| (S. aureus) | | | | | | |
| CXCR4-231 | 3973 | — | | WT | 14.16 | |
| (S. pyogenes) | | | | | | |
| CCR5_Sa1 | 480 | — | | WT | | 5.61 |
| (S. aureus) | | | | | | |
| CCR5_Sa3 | 488 | — | | WT | | 1.98 |
| (S. aureus) | | | | | | |
| CCR5_U43 | 335 | — | | WT | | 4.02 |
| (S. pyogenes) | | | | | | |
| CCR5_Sa1 | 480 | CCR5_Sa3 | 488 | D10A | | 1.73 |
| (S. aureus) | | (S. aureus) | | | | |
| CCR5_Sa1 | 480 | CCR5_U43 | 335 | WT | 5.33 | 1.56 |
| (S. aureus) | | (S. pyogenes) | | | | |
| CCR5_U43 | 335 | CXCR4-231 | 3973 | WT | 4.54 | 4.17 |
| (S. pyogenes) | | (S. pyogenes) | | | | |
| CCR5_U43 | 335 | CXCR4-836 | 4604 | WT | 18.93 | 2.80 |
| (S. pyogenes) | | (S. aureus) | | | | |
| CCR5_Sa3 | 488 | CXCR4-836 | 4604 | WT | 9.70 | 1.29 |
| (S. aureus) | | (S. aureus) | | | | |
| CCR5_Sa1 | 480 | CXCR4-371 | 4118 | WT | 4.47 | 1.04 |
| (S. aureus) | | (S. aureus) | | | | |

Human CD4⁺ T lymphocytes were sorted and expanded from umbilical cord blood MNCs and then culture in T cell media (Ex Vivo 15 with L-glutamine and recombinant transferrin w/o phenol red and gentamicin supplemented with 5% human AB serum, 1.6 mg/mL N-acetylcysteine, 2 mM L-alanyl-L-glutamine, human IL7 and IL15). Cells were activated with anti-human CD3 and CD28 immunomagnetic beads and then cultured without beads to expand the activated T cells. For RNP electroporation, 5 μg RNP (for sgRNA experiments) and 10 μg RNPs (5 μg of each RNP×2 for multiplex experiments) were added to 200,000 T lymphocytes. RNP was electroporated into T lymphocytes using the Amaxa Nucleofector system per the manufacturer's instructions. Seventy-two hours after electroporation, cells were collected and analyzed for gene disruption as determined by T7E1 analysis (see Table 16)

Of the three CXCR4 targets evaluated, at the CXCR4 locus, the S. aureus RNP complexed to CXCR4_836 gRNA was the most effective gRNA in T lymphocytes, with ˜40% gene disruption detected. Of the three CCR5 targeting gRNAs evaluated, the S. aureus RNP complexed to CCR5_Sa1 led to the highest level of gene disruption in this experiment (˜5.5%). A dual D10A nickase approach targeting CCR5, in which CCR5_Sa1and CCR5_Sa3 complexed RNPs were simultaneously electroporated into T lymphocytes led to ˜2% gene disruption of this locus. In addition, CXCR4 andCCR5 targeting RNPs were multiplexed (i.e., co-delivered to human T lymphocytes simultaneously). In four samples in which different target combinations of CXCR4 and CCR5 RNPs were multiplexed, gene disruption was detected at both targeted loci.

In summary, these data show that S. aureus and S. pyogenes Cas9 RNP complexed to modified gRNAs and electroporated into T lymphocytes supported targeted gene disruption of HIV co-receptors, including multiplex and simultaneous gene editing of CXCR4 and CCR5 within the same cell.

### Example 10

### Assessment of Multiplex Gene Targeting at the CCR5 and CXCR4 Genetic Loci in Human Cord Blood CD34⁺ HSCs with S. pyogenes and S. aureus Wild-Type Cas9 and D10A Nickase Ribonucleoprotein Complexes Delivered by Electroporation.

Transplantation of autologous CD34⁺ hematopoietic stem cells (HSCs, also known as hematopoietic stem/progenitor cells or HSPCs) that have been edited to disrupt expression of CXCR4 or CCR5 gene products would prevent entry of the HIV virus HSC progeny that are normally susceptible to HIV infection (e.g., macrophages and CD4 T lymphocytes). Multiplex genome editing with the Cas9 RNP complexed to modified gRNAs precisely alters more than one endogenous gene targets by creating indels at two different cut sites can lead to knock down of gene expression at multiple edited loci. In this Example, single target and multiplex genome editing was evaluated in human umbilical cord blood (CB) CD34⁺ cells after electroporation of wild-type S. pyogenes Cas9, wild-type S. aureus Cas9 or D10A nickase. Briefly, Cas9 protein was complexed with modified sgRNAs targeting CXCR4 or CCR5. Single RNPs targeting one gene (5 μg each per 200,000 cells either CXCR4 or CCR5) or 2 RNPs (5 μg each both targeting CCR5 or multiplex editing of CXCR4 and CCR5 in the same cells) were electroporated into CD34+ HSCs. Seventy-two hours after electroporation of RNP with the Amaxa nucleofector system, CD34⁺ HSCs were collected, gDNA isolated and CXCR4 and CCR5 PCR products analyzed by T7E1 endonuclease assay to evaluate targeted disruption of these HIV co-receptors (Table 19). Table 19 depicts experimental design associated with Example 10 to evaluate gene editing determined by T7E1 endonuclease assay analysis of the at CXCR4 and CCR5 loci after electroporation of primary human CD34⁺ HSCs with S. aureus and/or S. pyogenes RNPs.

TABLE 19
Cas9 RNP mediated gene editing of CXCR4 and
CCR5 loci human CD34⁺ HSCs

| | | | | % |
|---|---|---|---|---|
| gRNA 1 | gRNA 2 | Cas9 | % editing | editing at |
| (Species) | (Species) | variant | at CXCR4 | CCR5 |
| CXCR4_371 | — | WT | — | — |
| (S. aureus) | | | | |
| CXCR4_836 | — | WT | 57.1 | — |
| (S. aureus) | | | | |
| CXCR4_231 | — | WT | 20.54 | — |
| (S. pyogenes) | | | | |
| CCR5_Sa1 | — | WT | — | 8.75 |
| (S. aureus) | | | | |
| CCR5_Sa3 | — | WT | — | 0.44 |
| (S. aureus) | | | | |
| CCR5_U43 | — | WT | — | 3.68 |
| (S. pyogenes) | | | | |
| CCR5_Sa1 | CCR5_Sa3 | D10A | — | 0.30 |
| (S. aureus) | (S. aureus) | | | |
| CCR5_U43 | CXCR4_231 | WT | 9.84 | 4.36 |
| (S. pyogenes) | (S. pyogenes) | | | |
| CCR5_Sa1 | CXCR4_836 | WT | 39.04 | 5.24 |
| (S. aureus) | (S. aureus) | | | |
| CCR5_Sa3 | CXCR4_836 | WT | 34.82 | 0.35 |
| (S. aureus) | (S. aureus) | | | |
| CCR5_Sa1 | CXCR4_371 | WT | 0.00 | 5.48 |
| (S. aureus) | (S. aureus) | | | |

Of the three CXCR4 targets evaluated, at the CXCR4 locus, the S. aureus RNP complexed to CXCR4_836 gRNA was the most effective gRNA in HSCs, with 57% gene disruption detected by T7E1 endonuclease assay analysis. Indels were also detected after electroporation of S. pyogenes RNP complexed to CXCR4-231 (20% indels). Of the three CCR5 targeting gRNAs evaluated, the S. aureus RNP complexed to CCR5_Sa1 led to the highest level of indels in this experiment in human HSCs (˜9%). A dual D10A nickase approach targeting CCR5, in which CCR5_Sa1 and CCR5_Sa3 complexed RNPs were simultaneously electroporated into HSCs led to <1% indels at this locus. In addition, CXCR4 and CCR5 targeting RNPs were multiplexed (i.e., co-delivered to human HSCs simultaneously). In four samples in which different target combinations of CXCR4 and CCR5 RNPs were multiplexed, gene disruption was detected at both targeted loci in HSCs. For the multiplex experiment, co-electroporation the combination of CCR5_Sa1 complexed to S. aureus RNP and CXCR4_836 gRNA complexed to S. aureus RNP led to 5% indels at CCR5 and 30% indels at CXCR4.

In summary, these data show that S. aureus and S. pyogenes Cas9 RNP complexed to modified gRNAs and electroporated into human HSCs supported targeted gene disruption of HIV co-receptors, including multiplex and simultaneous gene editing of CXCR4 and CCR5 within the same cell.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A genome editing system comprising a first gRNA molecule comprising a first targeting domain that is complementary with a target sequence of a CCR5 gene and a second gRNA molecule comprising a second targeting domain that is complementary with a target sequence of a CXCR4 gene.

2. The genome editing system of claim 1, wherein the first targeting domain and the second targeting domain are selected from the group consisting of:
(a) a first targeting domain comprising a nucleotide sequence selected from SEQ ID NOS: 476 to 1569 and 1947 to 3663, and a second targeting domain comprising a nucleotide sequence selected from SEQ ID NOS: 4064 to 5208, and 5921 to 8355;
(b) a first targeting domain comprising a nucleotide sequence selected from SEQ ID NOS: 208 to 475, and 1614 to 1946, and a second targeting domain comprising a nucleotide sequence selected from SEQ ID NOS: 3740 to 4063, and 5241 to 5920;
(c) a first targeting domain comprising a nucleotide sequence selected from SEQ ID NOS: 476 to 1569 and 1947 to 3663, and a second targeting domain comprising a nucleotide sequence selected from SEQ ID NOS: 3740 to 4063, and 5241 to 5920;
(d) a first targeting domain comprising a nucleotide sequence selected from SEQ ID NOS: 208 to 475, and 1614 to 1946, and a second targeting domain comprising a nucleotide sequence selected from SEQ ID NOS: 4064 to 5208, and 5921 to 8355; and
(e) a first targeting domain comprising a nucleotide sequence selected from SEQ ID NOS: 335, 480, 482, 486, 488, 490, 492, 512, 521, 535, 1000, and 1002, and a second targeting domain comprising a nucleotide sequence selected from SEQ ID NO: 3973, 4118, and 4604.

3. The genome editing system of claim 1, wherein the first targeting domain and the second targeting domain are selected from the group consisting of:
(a) a first targeting domain comprising the nucleotide sequence set forth in SEQ ID NO: 335, and a second targeting domain comprising the nucleotide sequence set forth in SEQ ID NO: 3973;
(b) a first targeting domain comprising the nucleotide sequence set forth in SEQ ID NO: 335, and a second targeting domain comprising the nucleotide sequence set forth in SEQ ID NO: 4604;
(c) a first targeting domain comprising the nucleotide sequence set forth in SEQ ID NO: 488, and a second targeting domain comprising the nucleotide sequence set forth in SEQ ID NO: 4604; and
(d) a first targeting domain comprising the nucleotide sequence set forth in SEQ ID NO: 480, and a second targeting domain comprising the nucleotide sequence set forth in SEQ ID NO: 4118.

4. The genome editing system of claim 1, wherein one or both of the first and second gRNA molecules are modified at its 5′ end.

5. The genome editing system of claim 4, wherein the modification comprises an inclusion of a 5′ cap.

6. The genome editing system of claim 5, wherein the 5′ cap comprises a 3 ‘-O-Me-m7G(5′)ppp(5′)G anti reverse cap analog (ARCA).

7. The genome editing system of claim 1, wherein one or both of the first and second gRNA molecules comprise a 3’ polyA tail that is comprised of about 10 to about 30 adenine nucleotides.

8. The genome editing system of claim 7, wherein the 3′ polyA tail is comprised of 20 adenine nucleotides.

9. The genome editing system of claim 1, further comprising a first Cas9 molecule and a second Cas9 molecule that are configured to form complexes with the first and second gRNAs.

10. The genome editing system of claim 9, wherein at least one of the first and second Cas9 molecules comprises an S. pyogenes Cas9 molecule or an S. aureus Cas9 molecule.

11. The genome editing system of claim 9, wherein at least one of the first and second Cas9 molecules comprises a wild-type Cas9 molecule, a mutant Cas9 molecule, or a combination thereof.

12. The genome editing system of claim 11, wherein the mutant Cas9 molecule comprises a D10A mutation.

13. The genome editing system of claim 1, further comprising an oligonucleotide donor encoding a de132 mutation in the CCR5 gene.

14. A genome editing system comprising a gRNA molecule comprising a targeting domain that is complementary with a target sequence of a CCR5 gene.

15. The genome editing system of claim 14, wherein the targeting domain comprises a nucleotide sequence selected from SEQ ID NOS: 476 to 1569 and 1947 to 3663.

16. The genome editing system of claim 14, wherein the targeting domain comprises a nucleotide sequence selected from SEQ ID NOS: 208 to 475, and 1614 to 1946.

17. The genome editing system of claim 14, wherein the targeting domain comprises a nucleotide sequence selected from SEQ ID NOS: 335, 480, 482, 486, 488, 490, 492, 512, 521,535, 1000, and 1002.

18. A genome editing system comprising a gRNA molecule comprising a targeting domain that is complementary with a target sequence of a CXCR4 gene.

19. The genome editing system of claim 18, wherein the targeting domain comprises a nucleotide sequence selected from SEQ ID NOS: 4064 to 5208, and 5921 to 8355.

20. The genome editing system of claim 18, wherein the targeting domain comprises a nucleotide sequence selected from SEQ ID NOS: 3740 to 4063, and 5241 to 5920.

21. The genome editing system of claim 18, wherein the targeting domain comprises a nucleotide sequence selected from SEQ ID NOS: 3973, 4118, and 4604.

22. A composition comprising a first gRNA molecule comprising a first targeting domain that is complementary with a target sequence of a CCR5 gene, and a second gRNA molecule comprising a second targeting domain that is complementary with a target sequence of a CXCR4 gene.

23. A composition comprising a gRNA molecule comprising a targeting domain that is complementary with a target sequence of a CCR5 gene.

24. A composition comprising a gRNA molecule comprising a targeting domain that is complementary with a target sequence of a CXCR4 gene.

25. A vector comprising a polynucleotide encoding one gRNA molecule comprising a targeting domain that is complementary with a target sequence of a CCR5 gene.

26. A vector comprising a gRNA molecule comprising a targeting domain that is complementary with a target sequence of a CXCR4 gene.

27. A vector comprising a polynucleotide encoding at least one of a first gRNA molecule comprising a first targeting domain that is complementary with a target sequence of a CCR5 gene, and a second gRNA molecule comprising a second targeting domain that is complementary with a target sequence of a CXCR4 gene.

28. A method of altering a CCR5 gene in a cell, comprising administering to the cell one of:
(i) a genome editing system comprising a gRNA molecule comprising a targeting domain that is complementary with a target sequence of a CCR5 gene, and at least a Cas9 molecule;
(ii) a genome editing system comprising a polynucleotide encoding one gRNA molecule comprising a targeting domain that is complementary with a target sequence of a CCR5 gene, and a polynucleotide encoding a Cas9 molecule; and
(iii) a composition comprising one gRNA molecule comprising a targeting domain that that is complementary with a target sequence of a CCR5 gene, and at least a Cas9 molecule.

29. A method of altering a CXCR4 gene in a cell, comprising administering to the cell one of:
(i) a genome editing system comprising one gRNA molecule comprising a targeting domain that is complementary with a target sequence of a CXCR4 gene, and at least a Cas9 molecule;
(ii) a genome editing system comprising a polynucleotide encoding one gRNA molecule comprising a targeting domain that is complementary with a target sequence of a CXCR4 gene, and a polynucleotide encoding a Cas9 molecule; and
(iii) a composition comprising one gRNA molecule comprising a targeting domain that is complementary with a target sequence of a CXCR4 gene, and at least a Cas9 molecule.

30. A method of altering a CCR5 gene and a CXCR4 gene in a cell, comprising administering to the cell one of:
(i) a genome editing system comprising a first gRNA molecule comprising a first targeting domain that is complementary with a target sequence of a CCR5 gene, a second gRNA molecule comprising a second targeting domain that is complementary with a target sequence of a CXCR4 gene, and at least a Cas9 molecule;
(ii) a genome editing system comprising a polynucleotide encoding a first gRNA molecule comprising a first targeting domain that is complementary with a target sequence of a CCR5 gene, a polynucleotide encoding a second gRNA molecule comprising a second targeting domain that is complementary with a target sequence of a CXCR4 gene, and a polynucleotide encoding a Cas9 molecule; and
(iii) a composition comprising a first gRNA molecule comprising a first targeting domain that is complementary with a target sequence of a CCR5 gene, a second gRNA molecule comprising a second targeting domain that is complementary with a target sequence of a CXCR4 gene, and at least a Cas9 molecule.

31. A method of treating or preventing HIV infection or AIDS in a subject, comprising administering to the subject one of:
(i) a genome editing system comprising one gRNA molecule comprising a targeting domain that is complementary with a target sequence of a CCR5 gene, and at least a Cas9 molecule;
(ii) a genome editing system comprising a polynucleotide encoding one gRNA molecule comprising a targeting domain that is complementary with a target sequence of a CCR5 gene, and a polynucleotide encoding a Cas9 molecule;
(iii) a composition comprising one gRNA molecule comprising a targeting that is complementary with a target sequence of a CCR5 gene, and at least a Cas9 molecule;
(iv) a genome editing system comprising one gRNA molecule comprising a targeting domain that is complementary with a target sequence of a CXCR4 gene, and at least a Cas9 molecule;
(v) a genome editing system comprising a polynucleotide encoding one gRNA molecule comprising a targeting domain that is complementary with a target sequence of a CXCR4 gene, and a polynucleotide encoding a Cas9 molecule;
(vi) a composition comprising one gRNA molecule comprising a targeting domain that is complementary with a target sequence of a CXCR4 gene, and at least a Cas9 molecule;
(vii) a genome editing system comprising a first gRNA molecule comprising a first targeting domain that is complementary with a target sequence of a CCR5 gene, a second gRNA molecule comprising a second targeting domain that is complementary with a target sequence of a CXCR4 gene, and at least a Cas9 molecule;
(viii) a genome editing system comprising a polynucleotide encoding a first gRNA molecule comprising a first targeting domain that is complementary with a target sequence of a CCR5 gene, a polynucleotide encoding a second gRNA molecule comprising a second targeting domain that is complementary with a target sequence of a CXCR4 gene, and a polynucleotide encoding a Cas9 molecule; and
(ix) a composition comprising a first gRNA molecule comprising a first targeting domain that is complementary with a target sequence of a CCR5 gene, a second gRNA molecule comprising a second targeting domain that is complementary with a target sequence of a CXCR4 gene, and at least a Cas9 molecule.

32. A method of preparing a cell for transplantation, comprising contacting the cell with one of:
(i) a genome editing system comprising one gRNA molecule comprising a targeting domain that is complementary with a target sequence of a CCR5 gene, and at least a Cas9 molecule;
(ii) a genome editing system comprising a polynucleotide encoding one gRNA molecule comprising a targeting domain that is complementary with a target sequence of a CCR5 gene, and a polynucleotide encoding a Cas9 molecule;
(iii) a composition comprising one gRNA molecule comprising a targeting that is complementary with a target sequence of a CCR5 gene, and at least a Cas9 molecule;
(iv) a genome editing system comprising one gRNA molecule comprising a targeting domain that is complementary with a target sequence of a CXCR4 gene, and at least a Cas9 molecule;
(v) a genome editing system comprising a polynucleotide encoding one gRNA molecule comprising a targeting domain that is complementary with a target sequence of a CXCR4 gene, and a polynucleotide encoding a Cas9 molecule;
(vi) a composition comprising one gRNA molecule comprising a targeting domain that is complementary with a target sequence of a CXCR4 gene, and at least a Cas9 molecule;
(vii) a genome editing system comprising a first gRNA molecule comprising a first targeting domain that is complementary with a target sequence of a CCR5 gene, a second gRNA molecule comprising a second targeting domain that is complementary with a target sequence of a CXCR4 gene, and at least a Cas9 molecule;
(viii) a genome editing system comprising a polynucleotide encoding a first gRNA molecule comprising a first targeting domain that is complementary with a target sequence of a CCR5 gene, a polynucleotide encoding a second gRNA molecule comprising a second targeting domain that is complementary with a target sequence of a CXCR4 gene, and a polynucleotide encoding a Cas9 molecule; and
(ix) a composition comprising a first gRNA molecule comprising a first targeting domain that is complementary with a target sequence of a CCR5 gene, a second gRNA molecule comprising a second targeting domain that is complementary with a target sequence of a CXCR4 gene, and at least a Cas9 molecule.

33. A cell comprising at least one edited allele of a CCR5 gene and at least one edited allele of a CXCR4 gene.

34. A composition, comprising a plurality of cells characterized by at least 4% editing of a CCR5 gene and 4% editing of a CXCR4 gene.
